(19) United States
(12) Patent Application Publication
(10) Pub. No.: US 2025/0136593 A1
(43) Pub. Date: May 1, 2025

(54) DEGRADATION AGENT AND USE THEREOF

(71) Applicants: HITGEN INC., Chengdu (CN); SICHUAN KELUN-BIOTECH BIOPHARMACEUTICAL CO., LTD., Chengdu (CN)

(72) Inventors: Chuan LIU, Chengdu (CN); Jinming LIU, Chengdu (CN); Dengfeng DOU, Chengdu (CN); Zhengfu TAI, Chengdu (CN); Jin LI, Chengdu (CN); Junyou GE, Chengdu (CN); Wei ZHANG, Chengdu (CN); Shuai XIA, Chengdu (CN); Sichuan XIANG, Chengdu (CN); Linfu LUO, Chengdu (CN); Jianbo SHEN, Chengdu (CN); Longying CAI, Chengdu (CN); Qiuxia CHEN, Chengdu (CN); Qian LIU, Chengdu (CN); Hui HUANG, Chengdu (CN); Xiaojiao YANG, Chengdu (CN); Qiang TIAN, Chengdu (CN); Hongmei SONG, Chengdu (CN)

(21) Appl. No.: 18/706,076

(22) PCT Filed: Nov. 15, 2022
(86) PCT No.: PCT/CN2022/131968

§ 371 Date: Apr. 30, 2024

(30) Foreign Application Priority Data
Nov. 17, 2021 (CN) 202111375053.8
Jun. 10, 2022 (CN) 202210653756.0

(51) Int. Cl. C07D 471/04; A61K 31/519; A61P 35/02

(52) U.S. Cl. CPC C07D 471/04; A61K 31/519; A61P 35/02

(57) ABSTRACT

A compound having a BCL-XL protein degradation function as shown in Formula I and a use thereof in preparation of a drug for diseases associated with BCL-XL activity.

X-Y-Z Formula I

### FIELD OF THE INVENTION

The present invention relates to a compound with BCL-XL protein degradation effect and use thereof in preparation of a medicament for a disease related to BCL-XL activity.

### BACKGROUND OF THE INVENTION

BCL-XL is an anti-apoptotic protein belonging to the BCL-2 family. This protein family comprises anti-apoptotic proteins (e.g., Bcl-2, Bcl-xL and Mel-1) and pro-apoptotic molecules (e.g., Bid, Bim, Bad, Bak and Bax). Although the anti-apoptotic BCL-XL protein has a low expression level in normal cells, it is found to be highly overexpressed in many different types of human tumors and is considered to be potentially related to the occurrence, development and drug resistance of tumors. Targeting BCL-XL has been pursued as a cancer treatment strategy.

Ubiquitin is a small molecule protein consisting of 76 amino acids with a highly conserved sequence that exists in eukaryotic cells. The main function of ubiquitin is to label target proteins. The labeled target proteins can be recognized and degraded by proteasome. This process is called the ubiquitin/proteasome system. Among them, E3 ubiquitin ligase (E3 ubiquitin-protein ligase) directly binds to proteins, determining the specificity of degradation. The degradation process of the ubiquitin/proteasome system mainly comprises the following four steps: 1) Ubiquitin activation: the carboxyl residue of ubiquitin combines with the sulfhydryl group of ubiquitin activating enzyme E1; 2) Ubiquitin cross-linking: E1 hands over the activated ubiquitin to a ubiquitin-conjugating enzyme E2 through a transesterification process; 3) Binding ubiquitin complex to target protein by E3: ubiquitin-protein ligase E3 links the ubiquitin bound to E2 to the target protein; if ubiquitin already exists on the protein, the ubiquitin bound to E2 can be directly linked to the target protein; 4) Proteasome degradation: proteasome recognizes the labeled target protein and hydrolyzes the target protein into peptide chains with a length of 7 to 8 amino acids, thereby completing degradation of the target protein.

Proteolysis targeting chimeric molecule is a bifunctional molecule that can bind to E3 ubiquitin ligase and target protein at the same time, ubiquitinating the target protein that cannot originally bind to E3, and performing selective degradation through ubiquitin/proteasome system, thereby controlling intracellular target protein level. Currently, E3 ubiquitin ligases mainly include CRBN, VHL, MDM2 and cIAP1.

### SUMMARY OF THE INVENTION

The present invention provides a compound with BCL-XL protein degradation effect and use thereof in preparation of a drug for a disease related to BCL-XL activity.

The present invention provides a compound represented by Formula I, or a deuterated compound thereof, or a stereoisomer thereof, or a tautomer thereof, or a polymorph thereof, or a solvate thereof, or a N-oxide thereof, or an isotopically labeled compound thereof, or a metabolite thereof, or a prodrug thereof, or a pharmaceutically acceptable salt thereof:

X-Y-Z Formula I
wherein,
X represents a group binding to a BCL-XL protein;
Y represents a connecting group;
Z represents a group binding to an E3 ubiquitin ligase.

According to some embodiments of the present invention, X is selected from

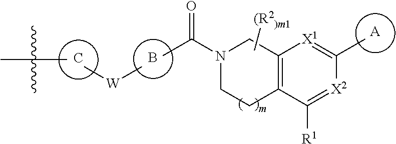

wherein,
Ring A is selected from C_{6˜10} aromatic ring or 6- to 10-membered aromatic heterocycle; in which the aromatic ring and aromatic heterocycle can be further substituted by one, two or three R^{A1};
each R^{A1} is independently selected from hydrogen, halogen, cyano, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{A2} or —C_{0˜4} alkylene-NR^{A2}R^{A3};
R^{A2} and R^{A3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
X¹ and X² are each independently selected from N or CR^{X1};
each R^{X1} is independently selected from hydrogen, halogen, cyano, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{X2} or —C_{0˜4} alkylene-NR^{X2}R^{X3};
R^{X2} and R^{X3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
R¹ is selected from hydrogen, halogen, cyano, —C_{1˜6} alkyl, —C_{2˜6} alkenyl, —C_{2˜6} alkynyl, -halogen-substituted C_{1˜6} alkyl, halogen-substituted —C_{2˜6} alkenyl, halogen-substituted —C_{2˜6} alkynyl, —C_{0˜4} alkylene-OR¹¹ or —C_{0˜4} alkylene-NR¹¹R¹²;
R¹¹ and R¹² are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
each R² is independently selected from hydrogen, halogen, cyano, ═O, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR²¹ or —C_{0˜4} alkylene-NR²¹R²²;
R²¹ and R²² are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
m1 is selected from 0, 1, 2 or 3;
m is selected from 0, 1 or 2;
Ring B is selected from C_{3˜10} cycloalkane, 3- to 10-membered heterocycloalkane, benzene ring, 5- to 6-membered aromatic heterocycle or 5- to 12-membered bridged ring; wherein, cycloalkane, heterocycloalkane, benzene ring, aromatic heterocycle and bridged ring can be further substituted by one, two or three R^{B1}; alternatively, Ring B is selected from C₃˜10 cycloalkane, 3- to 10-membered heterocycloalkane, benzene ring or 5- to 6-membered aromatic heterocycle; wherein, the cycloalkane, heterocycloalkane, benzene ring, and aromatic heterocycle can be further substituted by one, two, or three R^{B}1;
each R^{B1} is independently selected from hydrogen, halogen, cyano, ═O, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{B2} or —C_{0˜4} alkylene-NR^{B2}R^{B3};
R^{B2} and R^{B3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
W is selected from —C_{1˜6} alkylene-, —C_{1˜6} alkylene-O—, —O—C_{1˜6} alkylene-, —C_{2˜6} alkenylene-, —C_{2˜6} alkenylene-O—, —O—C_{2˜6} alkenylene-, —C_{2˜6} alkynylene-, —C_{2˜6} alkynylene-O— or —O—C_{2˜6} alkynylene-;
Ring C is selected from benzene ring or 5- to 6-membered aromatic heterocycle; wherein, the benzene ring and aromatic heterocycle can be further substituted by one, two or three R^{C1};
each R^{C1} is independently selected from hydrogen, halogen, cyano, —C_{1˜6} alkyl, —C_{2˜6} alkenyl, —C_{2˜6} alkynyl, halogen-substituted C_{1˜6} alkyl, halogen-substituted —C_{2˜6} alkenyl, halogen-substituted —C_{2˜6} alkynyl, —C_{0˜4} alkylene-OR^{C2} or —C_{0˜4} alkylene-NR^{C2}R^{C3};
R^{C2} and R^{C3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl.

According to some embodiments of the present invention, Ring A is selected from naphthalene ring or 8- to 10-membered aromatic heterocycle; wherein, the naphthalene ring and aromatic heterocycle can be further substituted by one, two or three R^{A1}, and each R^{A1} is independently selected from hydrogen, halogen, cyano or —C_{1˜6} alkyl.

In some embodiments, Ring A is selected from naphthalene ring, benzopyrimidine ring, or quinoline ring.

In some embodiments, Ring A is selected from naphthalene ring or quinoline ring.

In some embodiments, Ring A is selected from quinoline ring.

According to some embodiments of the present invention, X¹ and X² are each independently selected from N or CH.

In some embodiments, X¹ and X² are each independently selected from N.

According to some embodiments of the present invention, R¹ is selected from hydrogen, halogen, cyano, —C_{1˜6} alkyl or —NR¹¹R¹², and R¹¹ and R¹² are each independently selected from hydrogen or —C_{1˜6} alkyl.

In some embodiments, R¹ is selected from hydrogen or —NR¹¹R¹², and R¹¹ and R¹² are each independently selected from hydrogen or —C_{1˜6} alkyl.

In some embodiments, R¹ is selected from hydrogen,

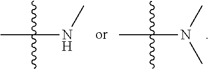

According to some embodiments of the present invention, m is selected from 1.

According to some embodiments of the present invention, Ring B is selected from 6-membered nitrogen-containing heterocycloalkane, benzene ring or 6-membered nitrogen-containing aromatic heterocycle, 5- to 6-membered cycloalkane or 5- to 8-membered bridged ring; wherein, the benzene ring, heterocycloalkane, aromatic heterocycle, cycloalkane and bridged ring can be further substituted by one, two or three R^{B1}; each R^{B1} is independently selected from hydrogen, halogen, cyano, ═O or —C_{1˜6} alkyl.

According to some embodiments of the present invention, Ring B is selected from 6-membered nitrogen-containing heterocycloalkane, benzene ring or 6-membered nitrogen-containing aromatic heterocycle, or 5- to 6-membered cycloalkane; wherein, the benzene ring, heterocycloalkane, aromatic heterocycle, and cycloalkane can be further substituted by one, two or three R^{B1}; each R^{B1} is independently selected from hydrogen, halogen, cyano, ═O or —C_{1˜6} alkyl.

In some embodiments, Ring B is selected from benzene ring, pyridine ring or

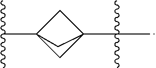 [SMILES: *C12CC(*)(C1)C2]

In some embodiments, Ring B is selected from benzene ring or pyridine ring.

In some embodiments, Ring B is selected from benzene ring.

According to some embodiments of the present invention, W is selected from —C_{1˜4} alkylene-, —C_{1˜4} alkylene-O—, —O—C_{1˜4} alkylene-, —C_{2˜4} alkenylene-, —C_{2˜4} alkenylene-O—, —O—C_{2˜4} alkenylene-, —C_{2˜4} alkynylene-, —C_{2˜4} alkynylene-O— or —O—C_{2˜4} alkynylene.

In some embodiments, W is selected from —C_{1˜4} alkylene-, —C_{1˜4} alkylene-O—, —O—C_{1˜4} alkylene-, —C_{2˜4} alkenylene-, —C_{2˜4} alkenylene-O— or —O—C_{2˜4} alkenylene-.

In some embodiments, W is selected from —C_{1˜3} alkylene, —C_{1˜3} alkylene-O—, —O—C_{1˜3} alkylene or —C_{2˜3} alkenylene.

In some embodiments, W is selected from ethylene,

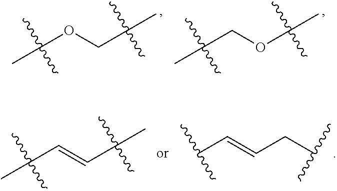

In some embodiments, W is selected from ethylene,

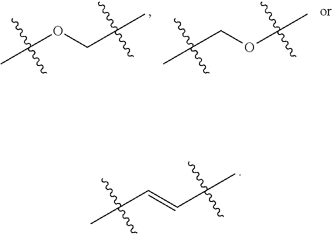

According to some embodiments of the present invention, Ring C is selected from benzene ring or 6-membered nitrogen-containing aromatic heterocycle; wherein, the benzene ring and aromatic heterocycle can be further substituted by one, two or three R^{C1}, each R^{C1} is independently selected from hydrogen, halogen, cyano or —C_{1˜6} alkyl. In some embodiments, Ring C is selected from benzene ring or pyridine ring.

According to some embodiments of the present invention, each R² is independently selected from halogen, cyano, ═O, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR²¹ or —C_{0˜4} alkylene-NR²¹R²²; preferably, each R² is independently selected from halogen, cyano, —C_{1˜6} alkyl, halogen-substituted —C_{1˜6} alkyl, halogen-substituted —C_{2˜6} alkenyl, —C_{0˜4} alkylene-OR²¹ or —C_{0˜4} alkylene-NR²¹R²²; further preferably, each R² is independently selected from —C_{0˜4} alkylene-NR²¹R²²;
preferably, m1 is selected from 0 or 1; further preferably, m1 is selected from 0. When m1 is 0, it means that R² does not exist, that is, the carbon vacancies on 6-membered nitrogen-containing heterocycle of

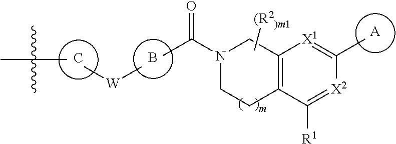

are all filled with H according to the principle of chemical bonding;
preferably, R²¹ and R²² are each independently selected from hydrogen, —C_{1˜6} alkyl, and halogen-substituted —C_{1˜6} alkyl; further preferably, R²¹ and R²² are each independently selected from hydrogen.

According to some embodiments of the present invention, Ring A is selected from naphthalene ring or 8- to 10-membered aromatic heterocycle; wherein, the naphthalene ring and aromatic heterocycle can be further substituted by one, two or three R^{A1};
X¹ and X² are each independently selected from N or CH;
R¹ is selected from hydrogen or —NR¹¹R¹²;
m is selected from 0 or 1;
Ring B is selected from 6-membered nitrogen-containing heterocycloalkane, benzene ring, 6-membered nitrogen-containing aromatic heterocycle, 5-membered bridged cycloalkane, or 5-membered cycloalkane; wherein, the benzene ring, aromatic heterocycle, cycloalkane, and bridged cycloalkane can be further substituted by one, two or three R^{B1}; or Ring B is selected from 6-membered nitrogen-containing heterocycloalkane, benzene ring, 6-membered nitrogen-containing aromatic heterocycle or 5-membered cycloalkane; wherein, the benzene ring, aromatic heterocycle, cycloalkane can be further substituted by one, two or three R^{B1};
W is selected from —C_{1˜4} alkylene-, —C_{1˜4} alkylene-O—, —O—C_{1˜4} alkylene-, —C_{2˜4} alkenylene-, —C_{2˜4} alkenylene-O—, —O—C_{2˜4} alkenylene-, —C_{2˜4} alkynylene-, —C_{2˜4} alkynylene-O— or —O—C_{2˜4} alkynylene-;
Ring C is selected from benzene ring or 6-membered nitrogen-containing aromatic heterocycle; wherein, benzene ring and aromatic heterocycle can be further substituted by one, two or three R^{C1}.

According to some embodiments of the present invention,
Ring A is selected from

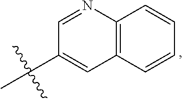 [SMILES: *c1cnc2ccccc2c1]

Ring A can also be selected from

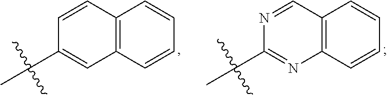 [SMILES: *c1ccc2ccccc2c1 ; *c1ncc2ccccc2n1]

X¹ and X² are selected from N or CH; or X¹ and X² are selected from N;
R¹ is selected from hydrogen,

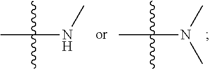

m is selected from 1;
m1 is selected from 0;
Ring B is selected from

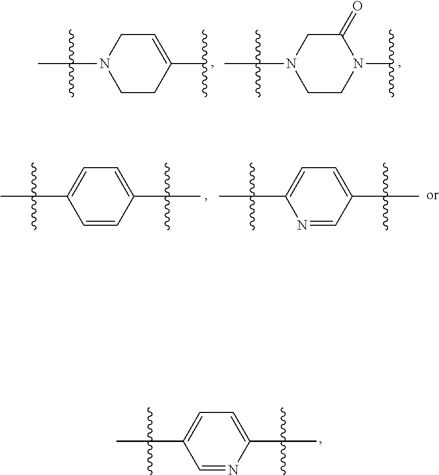 [SMILES: *C1=CCN(*)CC1 ; *N1CCN(*)C(=O)C1 ; *c1ccc(*)cc1 ; *c1ccc(*)nc1 ; *c1ccc(*)nc1]

Ring B can also be selected from

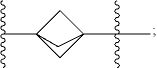 [SMILES: *C12CC(*)(C1)C2]

preferably, Ring B is selected from

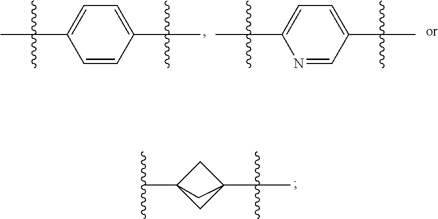 [SMILES: *c1ccc(*)cc1 ; *c1ccc(*)nc1 ; *C12CC(*)(C1)C2]

preferably, Ring B is selected from

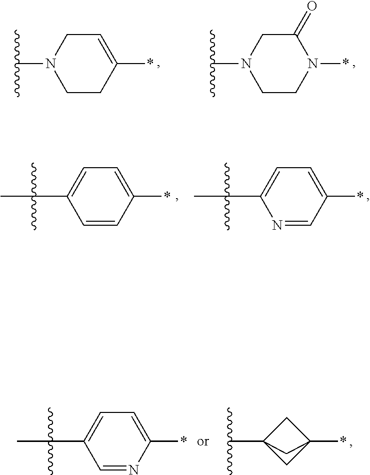

wherein * the end is linked to W, and the [symbol] end is linked to the carbonyl on the main chain X;
W is selected from ethylene,

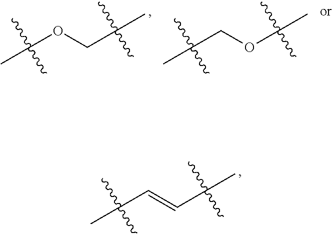

W can also be selected from

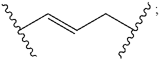 [SMILES: */C=C/C*]

preferably, W is selected from ethylene,

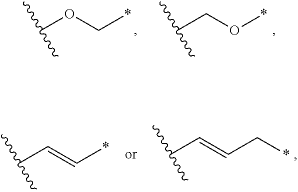

wherein, the * end is linked to the ring B, and the [symbol] end is linked to the ring C;
Ring C is selected from

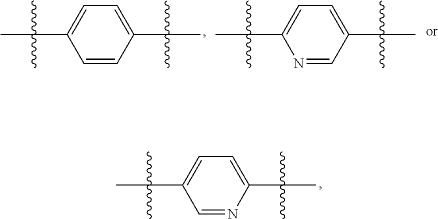 [SMILES: *c1ccc(*)cc1 ; *c1ccc(*)nc1 ; *c1ccc(*)nc1]

preferably, Ring C is selected from

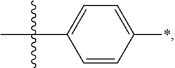

wherein the * end is linked to W and the [symbol] end is linked to Y.

According to some embodiments of the present invention, X is selected from

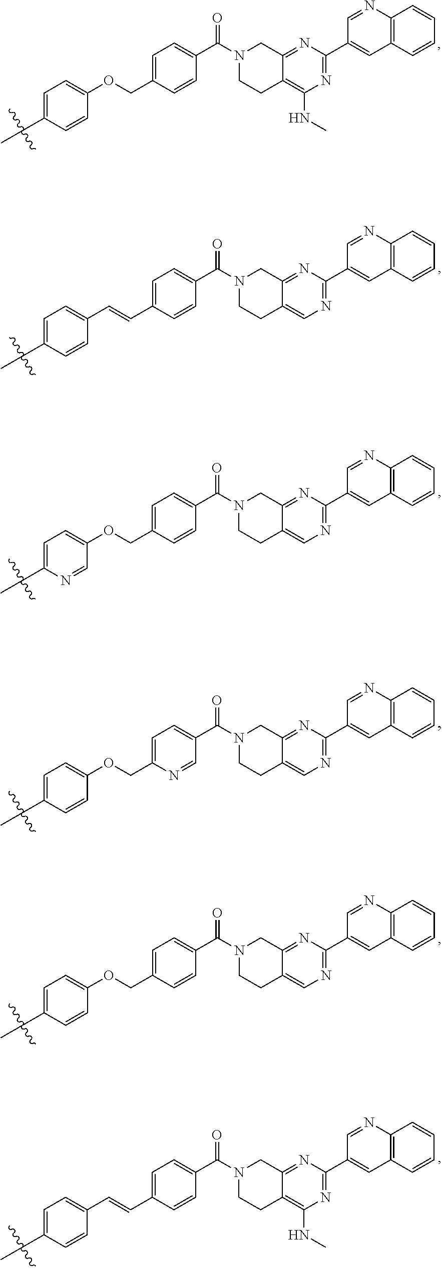 [SMILES: *c1ccc(OCc2ccc(C(=O)N3CCc4c(nc(-c5cnc6ccccc6c5)nc4NC)C3)cc2)cc1 ; *c1ccc(/C=C/c2ccc(C(=O)N3CCc4cnc(-c5cnc6ccccc6c5)nc4C3)cc2)cc1 ; *c1ccc(OCc2ccc(C(=O)N3CCc4cnc(-c5cnc6ccccc6c5)nc4C3)cc2)cn1 ; *c1ccc(OCc2ccc(C(=O)N3CCc4cnc(-c5cnc6ccccc6c5)nc4C3)cn2)cc1 ; *c1ccc(OCc2ccc(C(=O)N3CCc4cnc(-c5cnc6ccccc6c5)nc4C3)cc2)cc1 ; *c1ccc(/C=C/c2ccc(C(=O)N3CCc4c(nc(-c5cnc6ccccc6c5)nc4NC)C3)cc2)cc1]

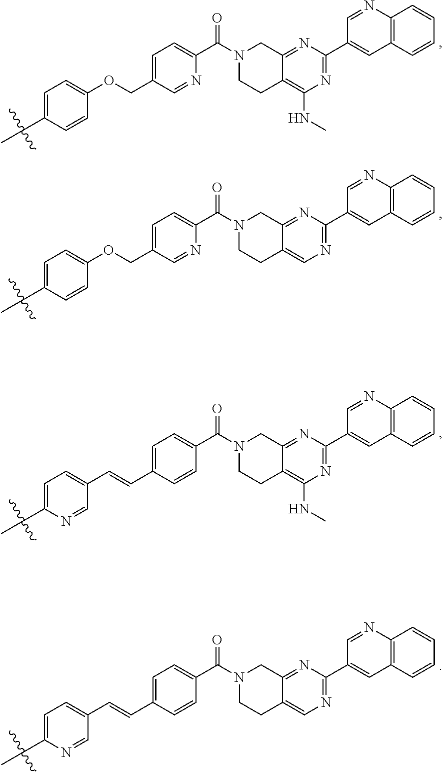 [SMILES: *c1ccc(OCc2ccc(C(=O)N3CCc4c(nc(-c5cnc6ccccc6c5)nc4NC)C3)nc2)cc1 ; *c1ccc(OCc2ccc(C(=O)N3CCc4cnc(-c5cnc6ccccc6c5)nc4C3)nc2)cc1 ; *c1ccc(/C=C/c2ccc(C(=O)N3CCc4c(nc(-c5cnc6ccccc6c5)nc4NC)C3)cc2)cn1 ; *c1ccc(/C=C/c2ccc(C(=O)N3CCc4cnc(-c5cnc6ccccc6c5)nc4C3)cc2)cn1]

X can also be selected from

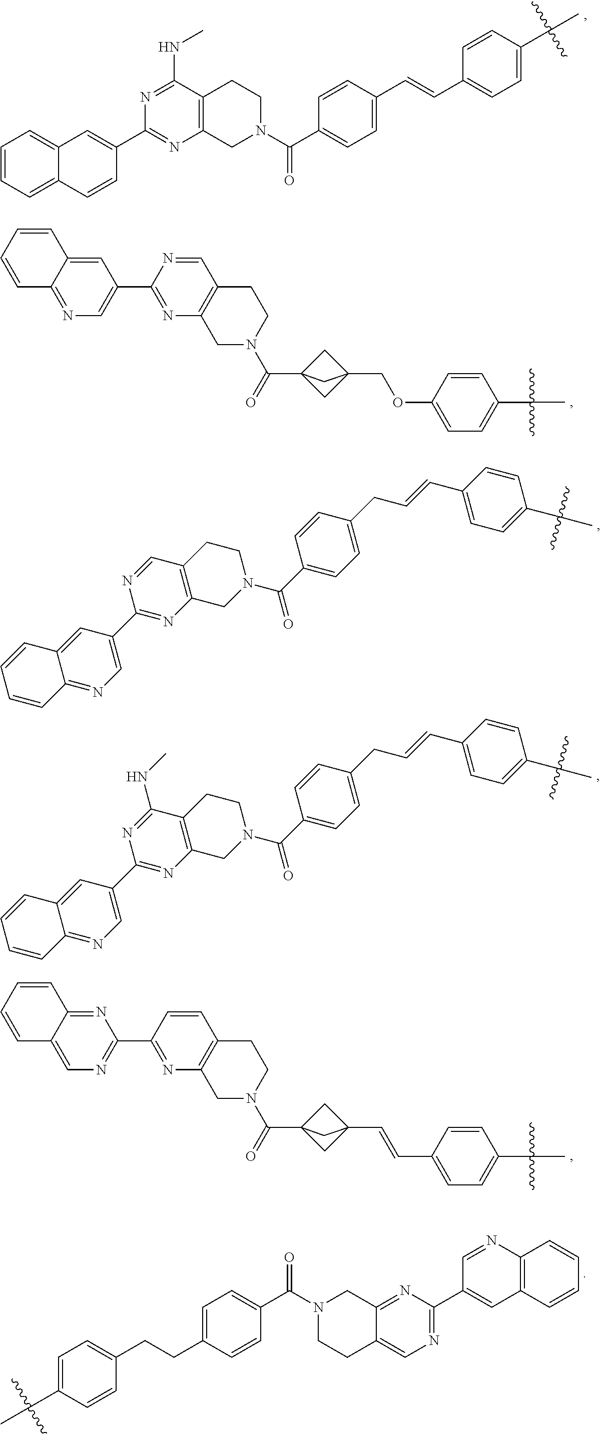

According to some embodiments of the present invention, X is selected from

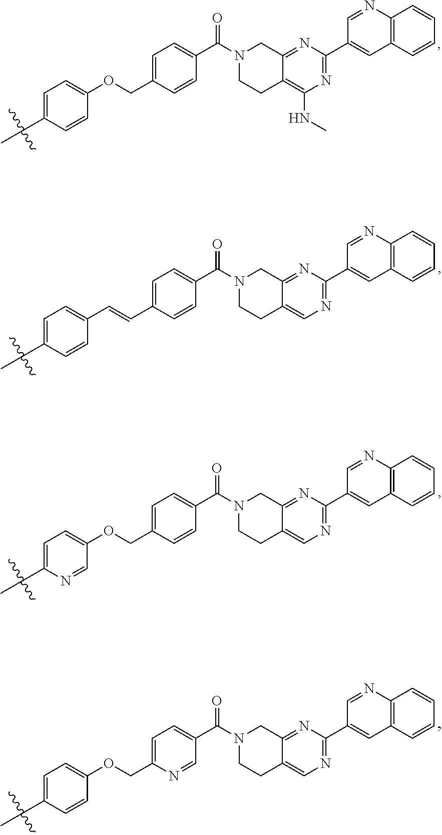 [SMILES: *c1ccc(OCc2ccc(C(=O)N3CCc4c(nc(-c5cnc6ccccc6c5)nc4NC)C3)cc2)cc1 ; *c1ccc(/C=C/c2ccc(C(=O)N3CCc4cnc(-c5cnc6ccccc6c5)nc4C3)cc2)cc1 ; *c1ccc(OCc2ccc(C(=O)N3CCc4cnc(-c5cnc6ccccc6c5)nc4C3)cc2)cn1 ; *c1ccc(OCc2ccc(C(=O)N3CCc4cnc(-c5cnc6ccccc6c5)nc4C3)cn2)cc1]

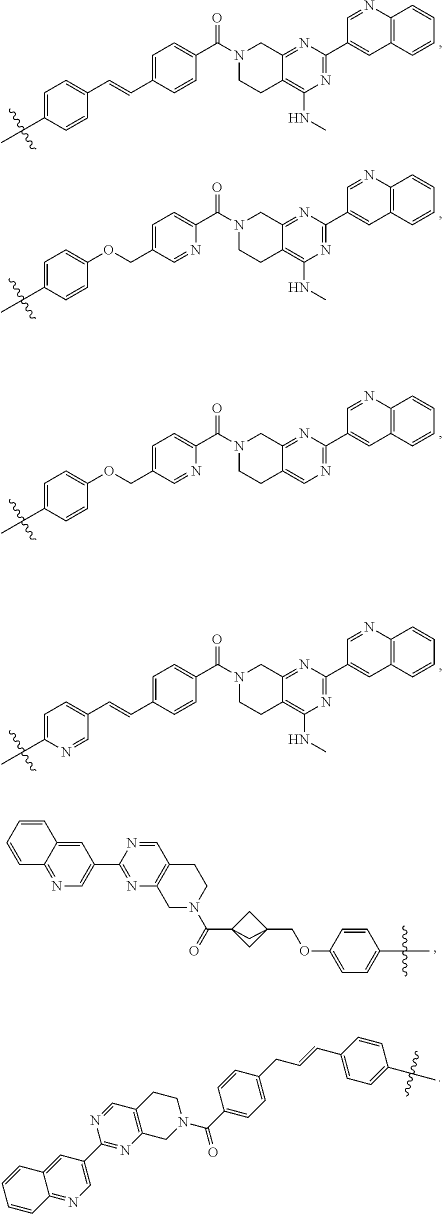 [SMILES: *c1ccc(/C=C/c2ccc(C(=O)N3CCc4c(nc(-c5cnc6ccccc6c5)nc4NC)C3)cc2)cc1 ; *c1ccc(OCc2ccc(C(=O)N3CCc4c(nc(-c5cnc6ccccc6c5)nc4NC)C3)nc2)cc1 ; *c1ccc(OCc2ccc(C(=O)N3CCc4cnc(-c5cnc6ccccc6c5)nc4C3)nc2)cc1 ; *c1ccc(/C=C/c2ccc(C(=O)N3CCc4c(nc(-c5cnc6ccccc6c5)nc4NC)C3)cc2)cn1 ; *c1ccc(OCC23CC(C(=O)N4CCc5cnc(-c6cnc7ccccc7c6)nc5C4)(C2)C3)cc1 ; *c1ccc(/C=C/Cc2ccc(C(=O)N3CCc4cnc(-c5cnc6ccccc6c5)nc4C3)cc2)cc1]

According to some embodiments of the present invention, X can also optionally be independently substituted by one or more (e.g., 2, 3, 4, 5, 6, 7, 8, 9 or 10) R^{A1′}.

According to some embodiments of the present invention, each R^{A1′} is independently selected from halogen, cyano, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{A2′} or —C_{0˜4} alkylene-NR^{A2′}R^{A3′};
R^{A2′} and R^{A3′} are each independently selected from hydrogen, —C_{1˜3} alkyl, —C_{2˜6} alkenyl, —C_{2˜6} alkynyl, halogen-substituted —C_{1˜3} alkyl, halogen-substituted —C_{2˜6} alkenyl, or halogen-substituted —C_{2˜6} alkynyl.

According to some embodiments of the present invention, each R^{A1′} is independently selected from halogen, cyano, —C_{1˜3} alkyl, —C_{2˜4} alkenyl, halogen-substituted —C_{1˜3} alkyl, halogen-substituted —C_{2˜4} alkenyl, —C_{0˜4} alkylene-OR^{A2′} or —C_{0˜4} alkylene-NR^{A2′}R^{A3′};
R^{A2′} and R^{A3′} are each independently selected from hydrogen, —C_{1˜3} alkyl, —C_{2˜4} alkenyl, halogen-substituted —C_{1˜3} alkyl, or halogen-substituted —C_{2˜4} alkenyl.

According to some embodiments of the present invention,
the Y is selected from -(L^{Y})_{q}-;
q is an integer from 1 to 30;
each L^{Y} is independently selected from structural fragments consisting of any one or more members selected from the group consisting of C(R)₂, C(O), O, S, S(O), S(O)₂, NR, —CR═CR—, —C≡C—, C_{3˜10} cycloalkane, 3- to 10-membered heterocycloalkane, C_{6˜10} aromatic ring, 5- to 10-membered aromatic heterocycle, 5- to 12-membered spiro ring, 5- to 12-membered spiro heterocycle, 5- to 12-membered bridged ring, and 5- to 12-membered bridged heterocycle; wherein the cycloalkane, heterocycloalkane, aromatic ring, aromatic heterocycle, spiro ring, spiro heterocycle, bridged ring, and bridged heterocycle can be further substituted by one, two, or three R^{YL};
each R^{YL} is independently selected from the group consisting of hydrogen, halogen, cyano, nitro, C_{1˜6} alkyl, halogen-substituted C_{1˜6} alkyl, —OR, —N(R)₂;
each R is independently selected from the group consisting of hydrogen, halogen, —C_{1˜6} alkyl, halogen-substituted —C_{1˜6} alkyl, —C_{0˜2} alkylene-(C_{3˜10} carbocyclic group), and —C_{0˜2} alkylene-(3- to 10-membered heterocycloalkyl).

According to some embodiments of the present invention, the q is 1 to 25; preferably, q is 1 to 20 (for example, 1 to 10); preferably, q is 2, 3, 4, 5, 6, 7, 8, 9, 10.

According to some embodiments of the present invention, Y is selected from the group consisting of:

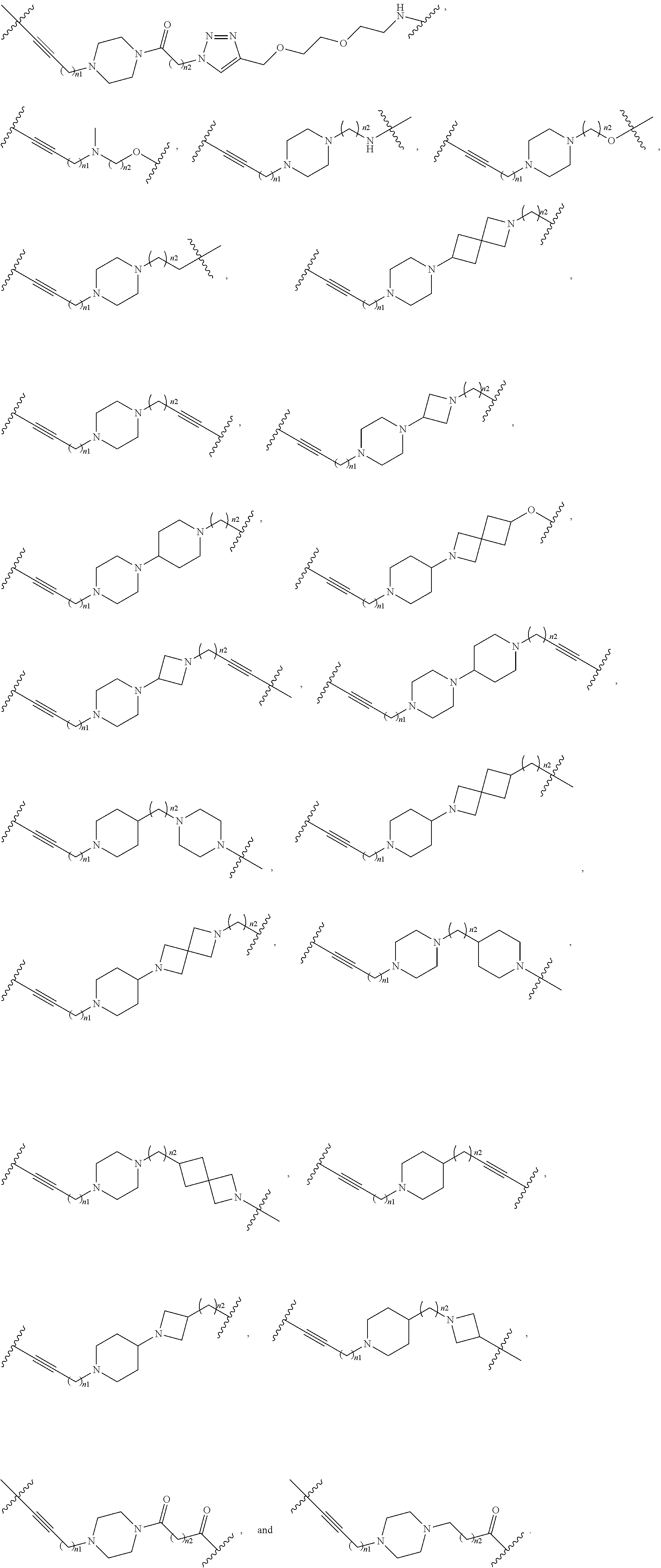

The Y can also be selected from

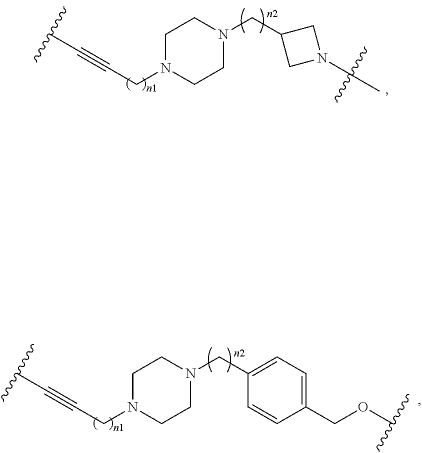

wherein n1 in each structural fragment is independently an integer selected from 0 to 10, and n2 in each structural fragment is independently an integer selected from 0 to 10.

According to some embodiments of the present invention, n1 in each structural fragment is independently selected from 0, 1, 2, 3, 4, 5, 6, 7, 8, and n2 in each structural fragment is independently selected from 0, 1, 2, 3, 4, 5, 6, 7, 8.

In some embodiments, Y is selected from

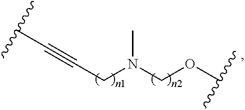

wherein n1 is selected from 1, and n2 is selected from 3, 4 or 5; or
Y is selected from

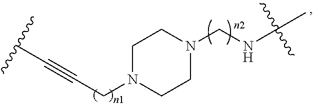

wherein n1 is selected from 1 and n2 is selected from 3, 4, 5, 6, 7 or 8; or
Y is selected from

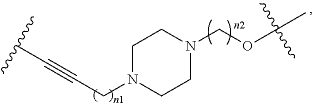

wherein n1 is selected from 1 and n2 is selected from 3, 4, 5, 6, 7 or 8; or
Y is selected from

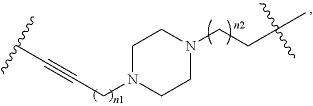

wherein n1 is selected from 1 and n2 is selected from 3, 4, 5 or 6; or
Y is selected from

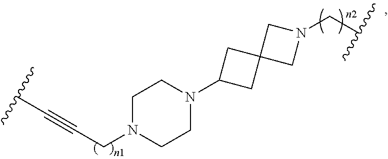

wherein n1 is selected from 1 and n2 is selected from 0 or 1; or
Y is selected from

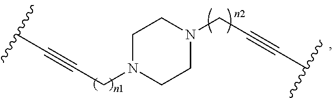

wherein n1 is selected from 1, 2, 3 or 4, and n2 is selected from 1; or
Y is selected from

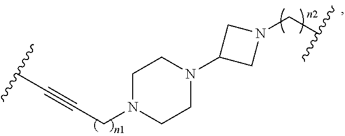

wherein n1 is selected from 1 and n2 is selected from 0, 1, 2 or 3; or
Y is selected from

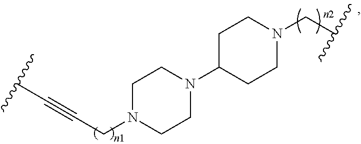

wherein n1 is selected from 1 and n2 is selected from 0, 1, 2 or 3; or
Y is selected from

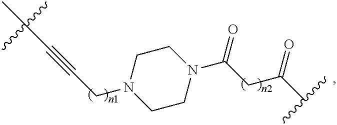

wherein n1 is selected from 1 and n2 is selected from 1, 2, 3, 4, 5, 6, 7 or 8; or
Y is selected from

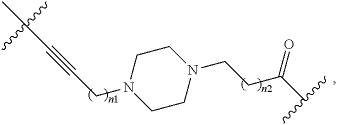

wherein n1 is selected from 1 and n2 is selected from 2, 3 or 4.

According to some embodiments of the present invention, Y is selected from

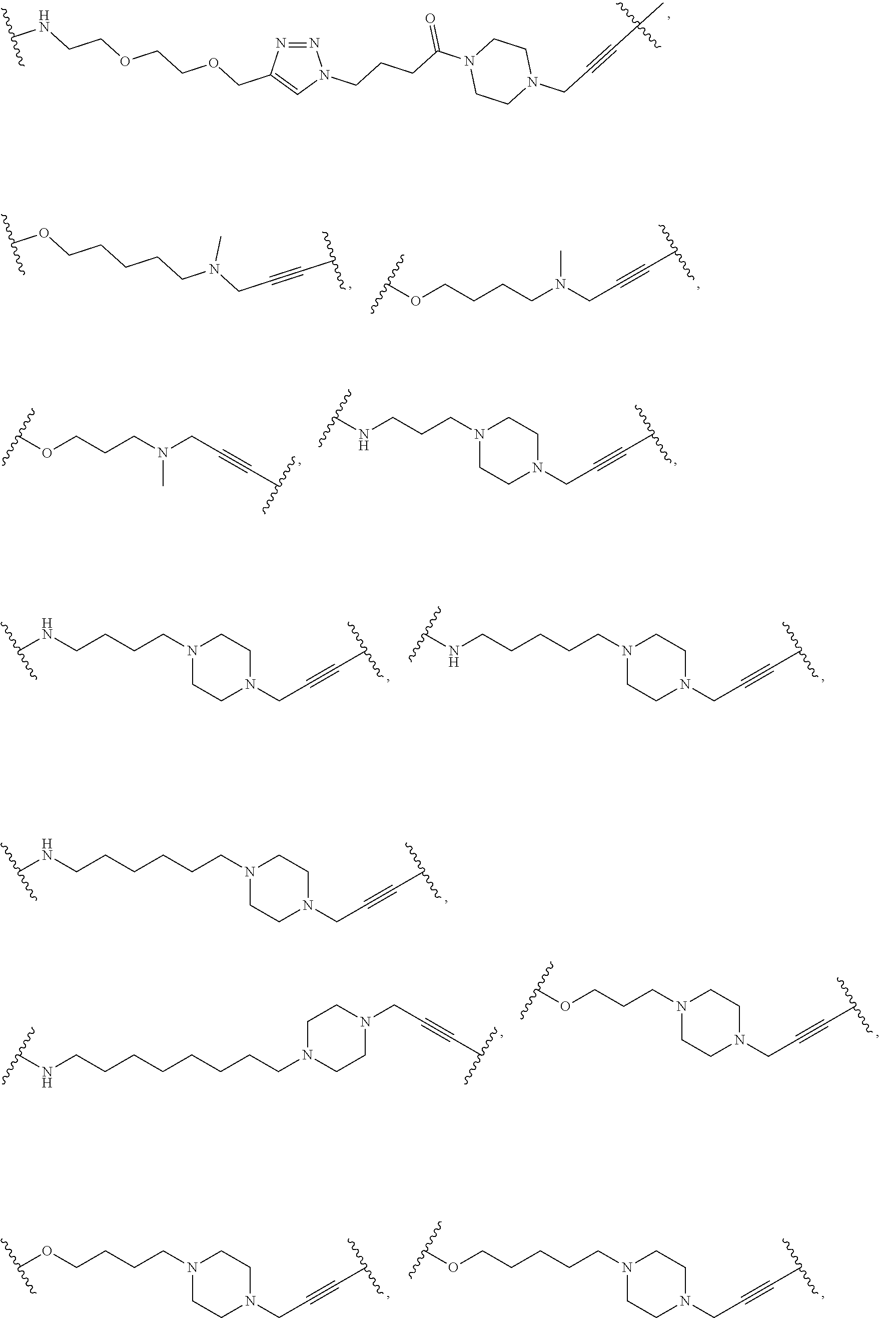

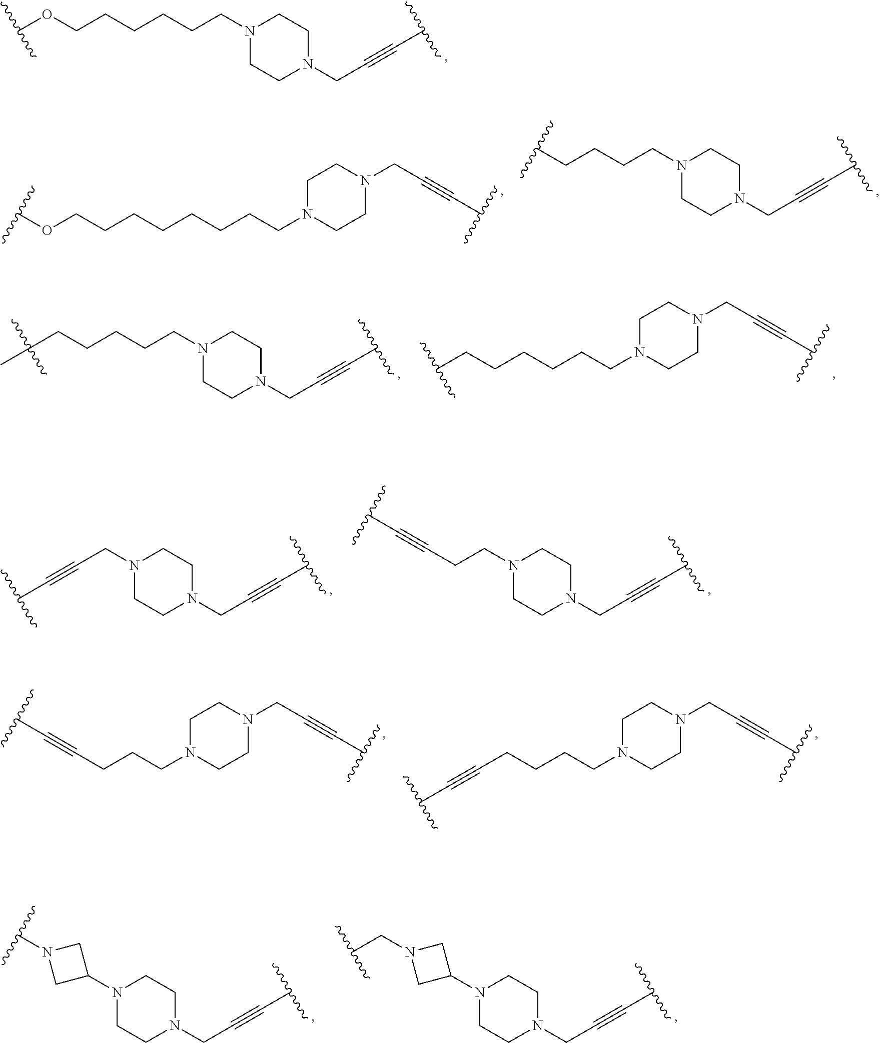

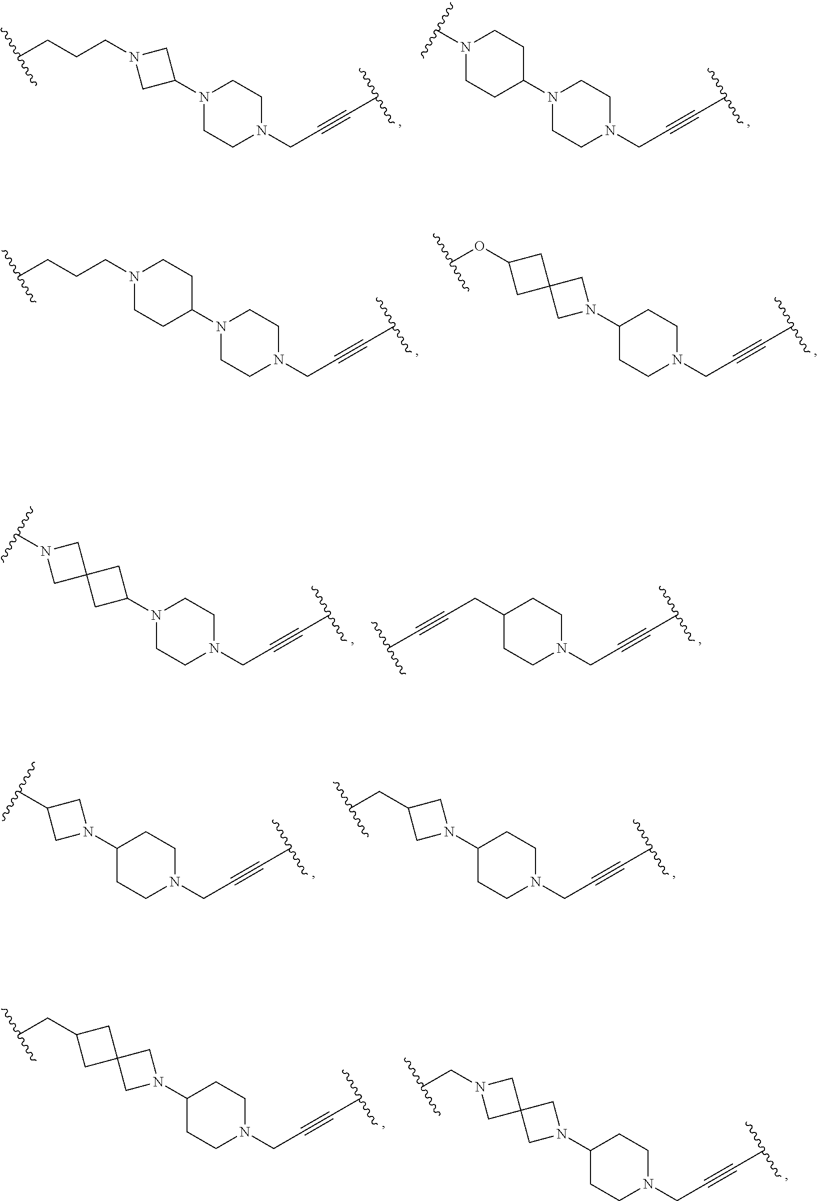

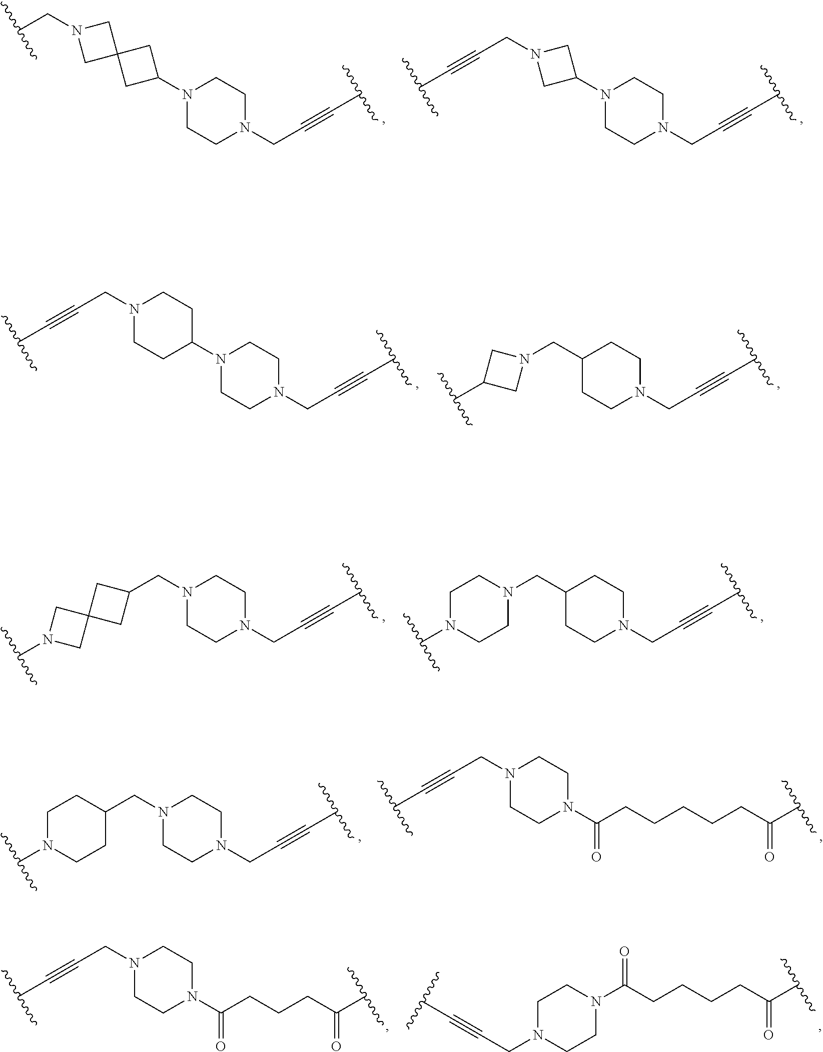

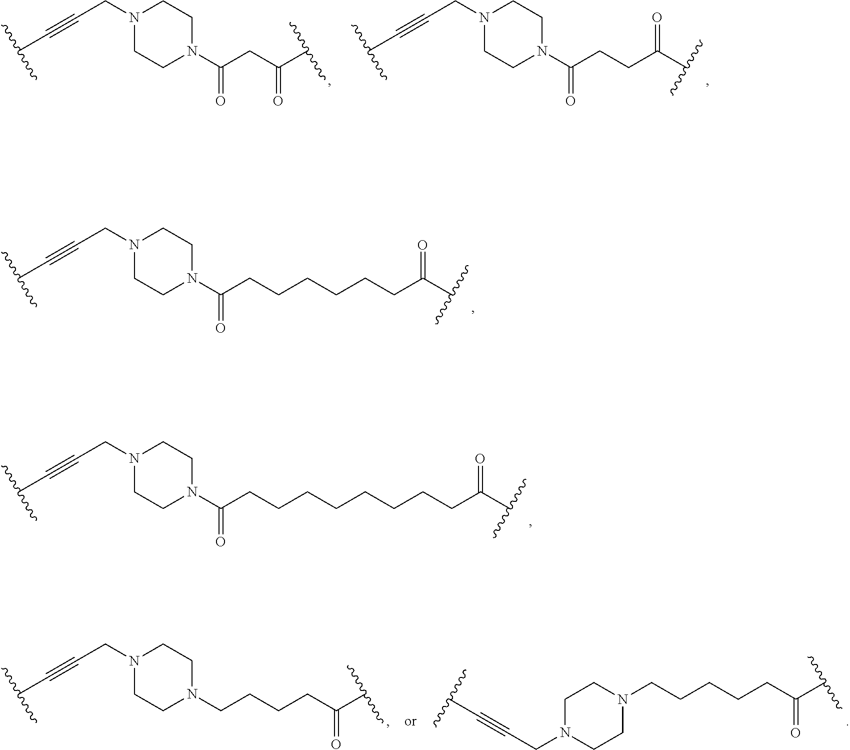 [SMILES: *C#CCN1CCN(C(=O)CC(*)=O)CC1 ; *C#CCN1CCN(C(=O)CCC(*)=O)CC1 ; *C#CCN1CCN(C(=O)CCCCCCC(*)=O)CC1 ; *C#CCN1CCN(C(=O)CCCCCCCCC(*)=O)CC1 ; *C#CCN1CCN(CCCCC(*)=O)CC1 ; *C#CCN1CCN(CCCCCC(*)=O)CC1]

The Y can also be selected from

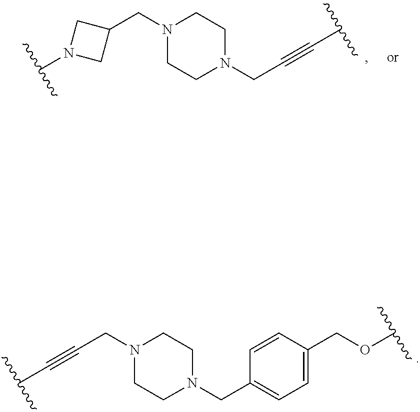 [SMILES: *C#CCN1CCN(CC2CN(*)C2)CC1 ; *C#CCN1CCN(Cc2ccc(CO*)cc2)CC1]

According to some embodiments of the present invention, the Y can also be optionally substituted independently by one or more (for example, 2, 3, 4, 5, 6, 7, 8, 9 or 10) R^{YL′}.

According to some embodiments of the present invention, R^{YL′} is independently selected from halogen, cyano, nitro, C_{1˜6} alkyl, halogen-substituted C_{1˜6} alkyl, —OR′, —N(R′)₂; each R′ is independently selected from hydrogen, halogen, —C_{1˜6} alkyl, halogen-substituted —C_{1˜6} alkyl, —C_{0˜2} alkylene-(C_{3˜10} carbocyclic group), —C_{0˜2} alkylene-(3- to 10-membered heterocycloalkyl).

According to some embodiments of the present invention, R_{YL′} is independently selected from halogen, cyano, nitro, C_{1˜6} alkyl, halogen-substituted C_{1˜6} alkyl, —OR′, —N(R′)₂; each R′ is independently selected from hydrogen, halogen, —C_{1˜6} alkyl, halogen-substituted —C_{1˜6} alkyl, —C_{0˜2} alkylene-(C_{3˜8} carbocyclic group), —C_{0˜2} alkylene-(3- to 8-membered heterocycloalkyl).

According to some embodiments of the present invention, R^{YL′} is independently selected from halogen, cyano, nitro, C_{1˜3} alkyl, halogen-substituted C_{1˜3} alkyl, —OR′ and —N(R′)₂; each R′ is independently selected from hydrogen, halogen, —C_{1˜3} alkyl, halogen-substituted —C_{1˜3} alkyl, —C_{0˜2} alkylene-(C_{3˜6} carbocyclic group) and —C_{0˜2} alkylene-(3- to 6-membered heterocycloalkyl).

In some embodiments of the present invention, the E3 ubiquitin ligase is selected from CRBN, von Hippel-Lindau (VHL), XIAP, MDM2, cIAP-1.

According to some embodiments of the present invention, Z is selected from

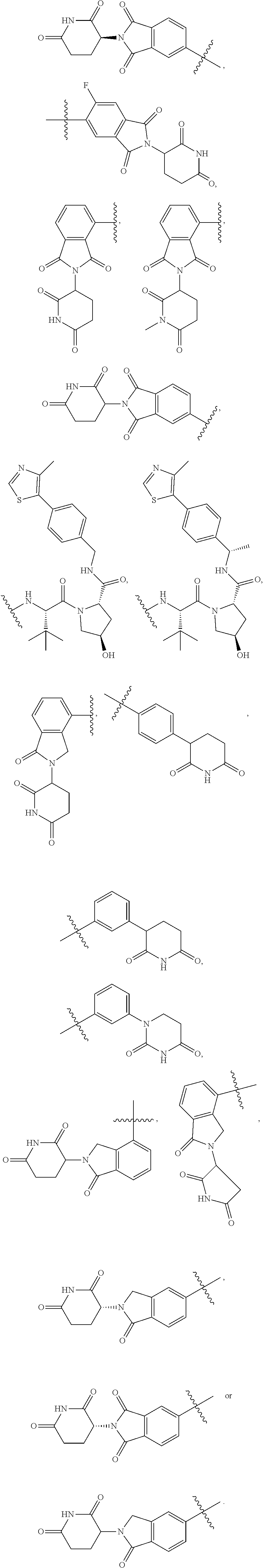

According to some embodiments of the present invention, Z is selected from

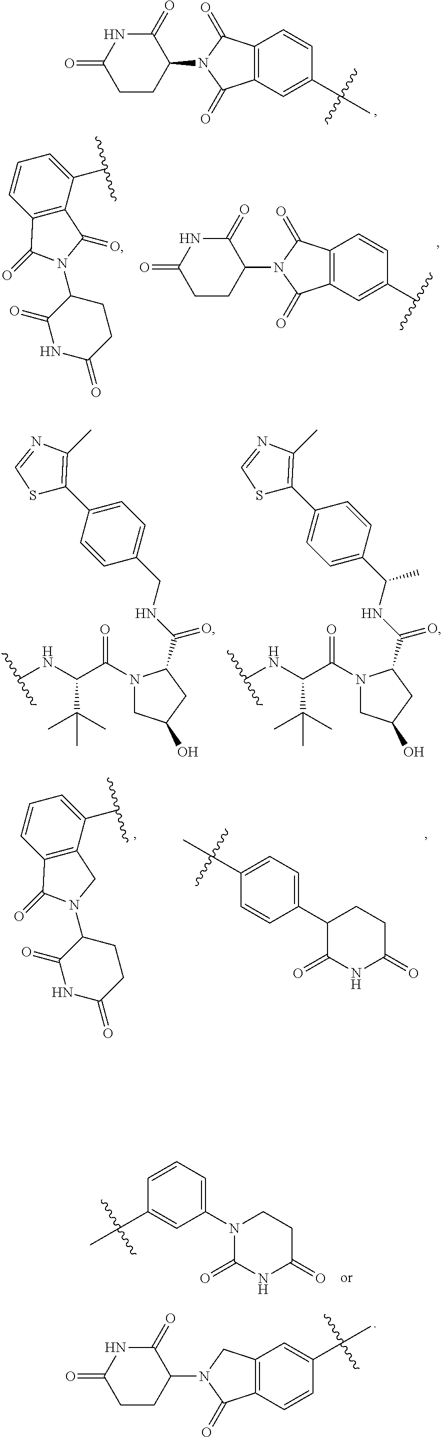

According to some embodiments of the present invention, Z is selected from

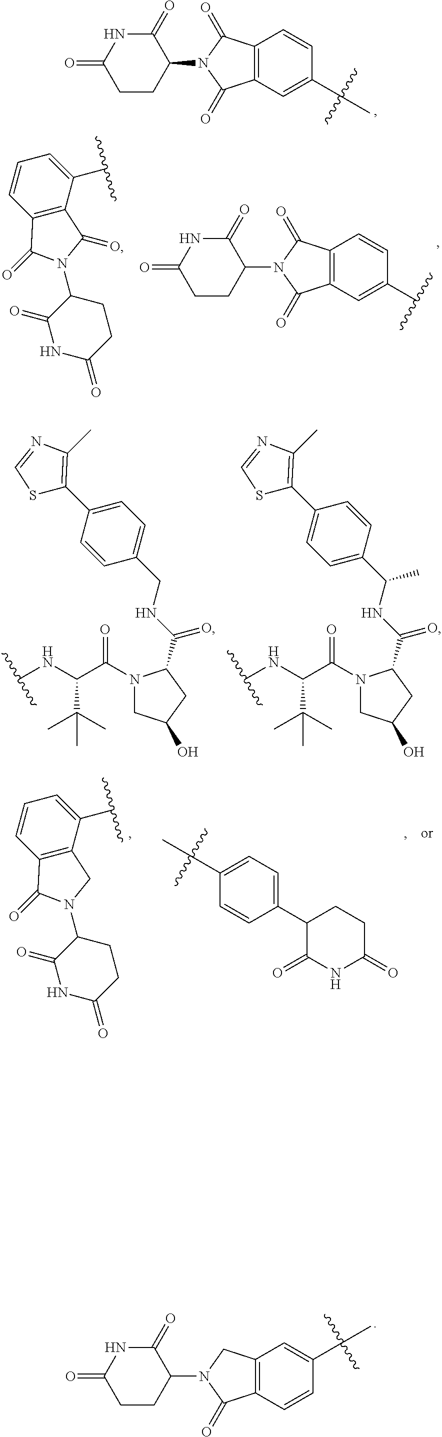

Z can also be selected from

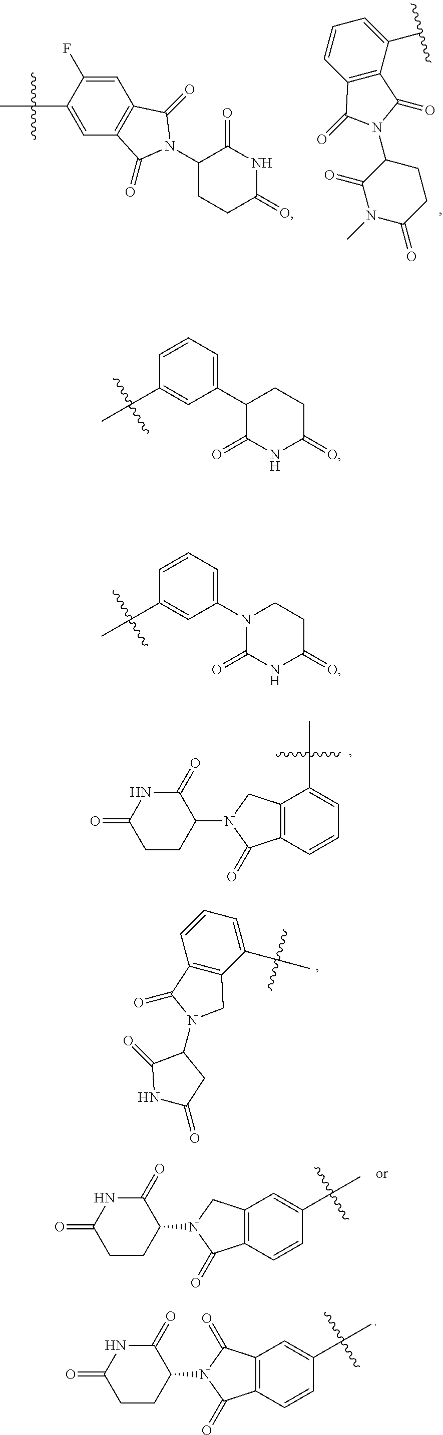

Z can also be selected from

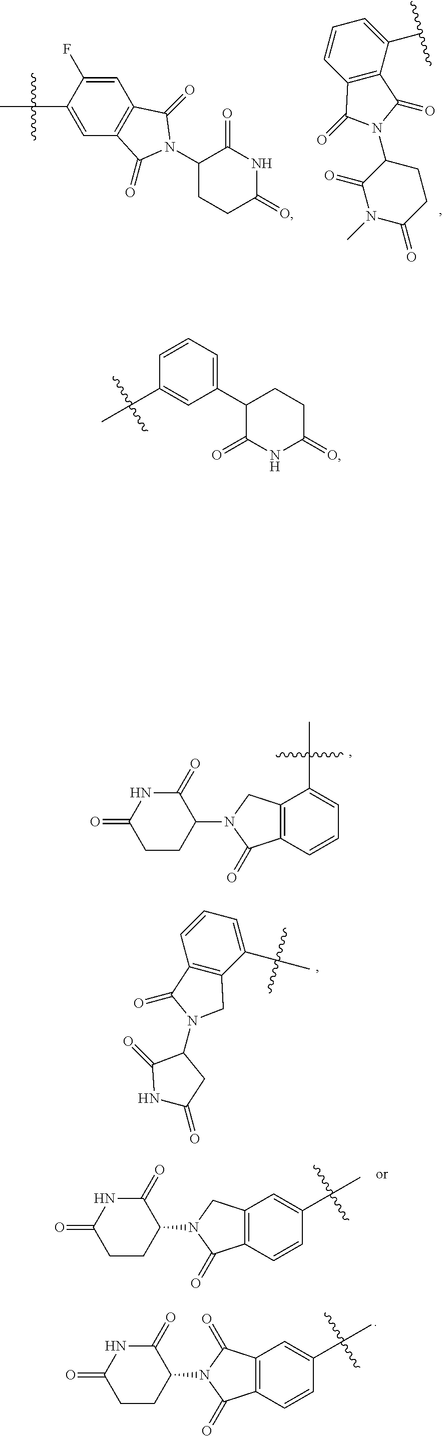

According to some embodiments of the present invention, Z is selected from

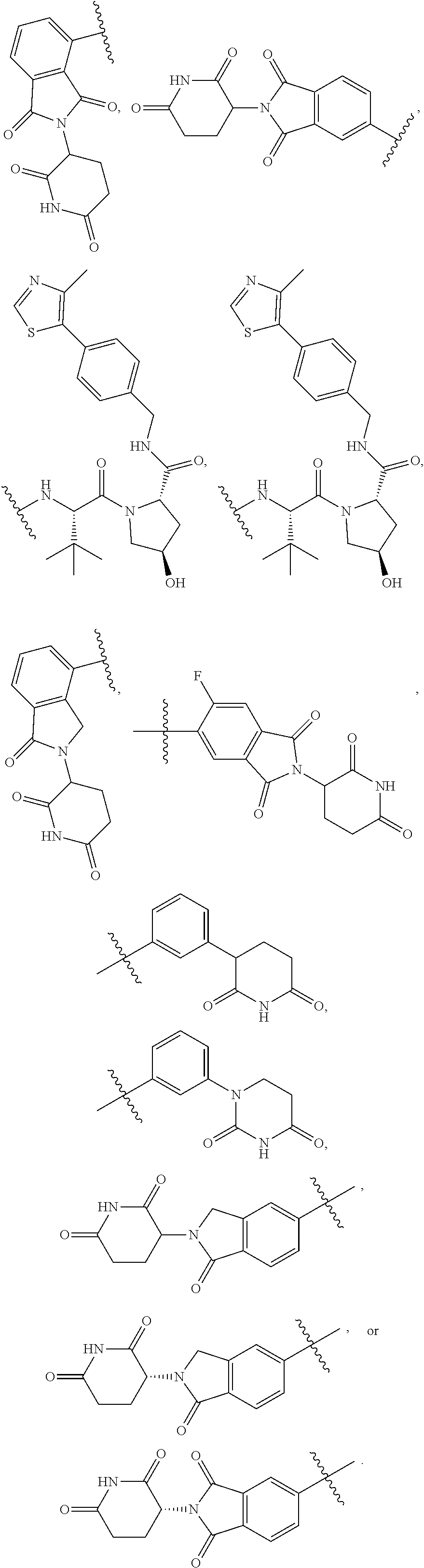

In some specific embodiments of the present invention, the compound of Formula I is specifically:

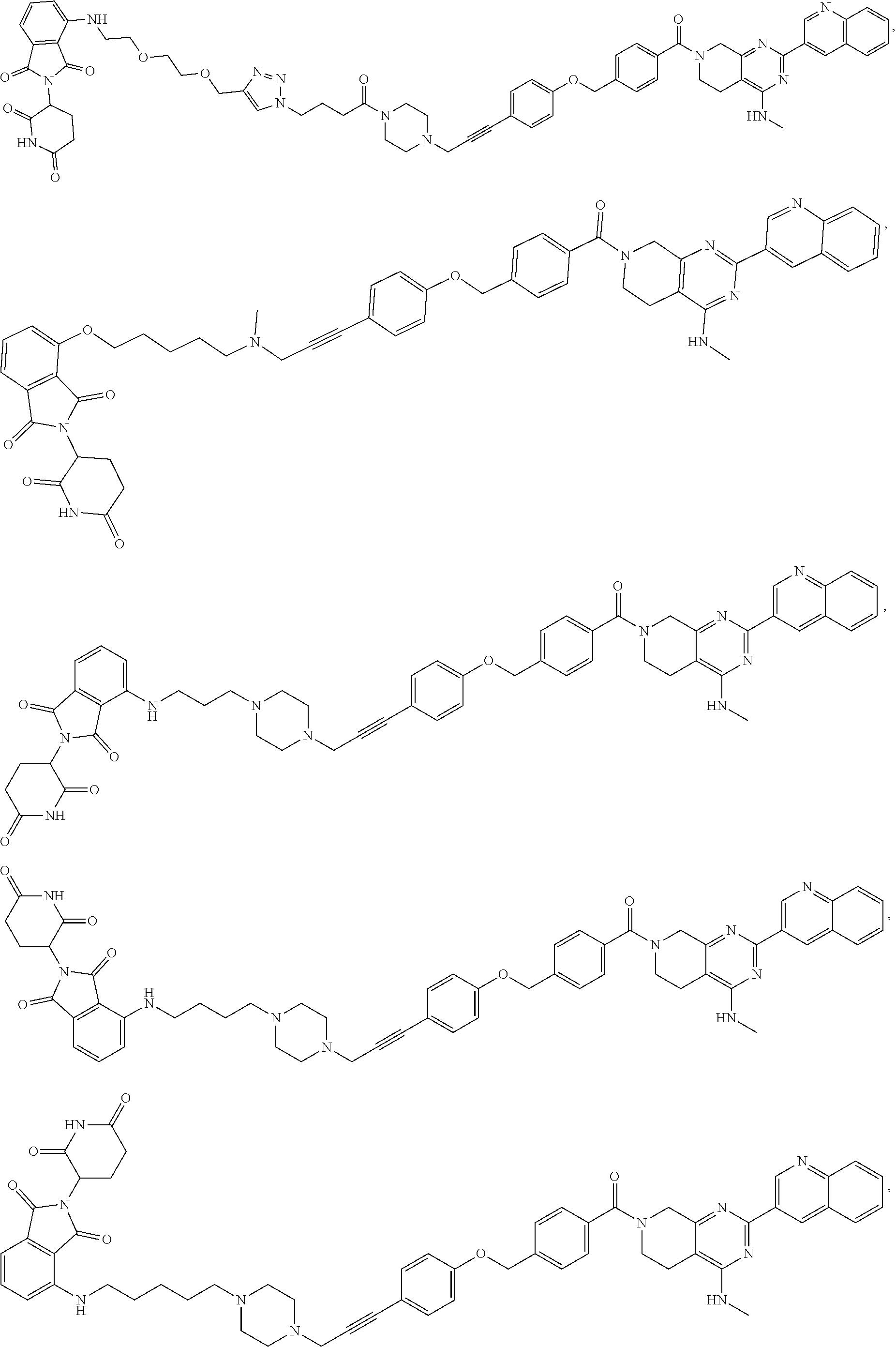

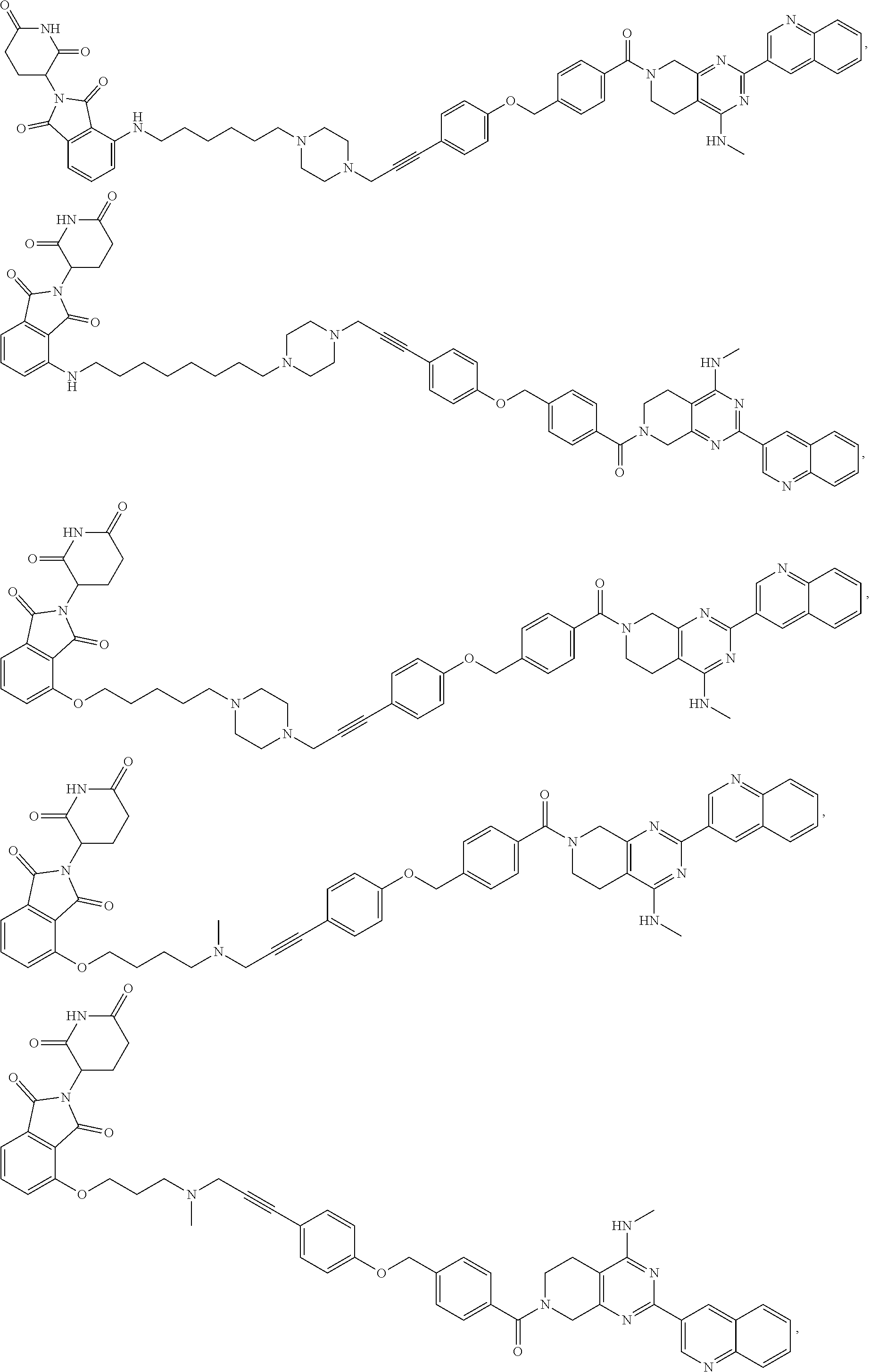

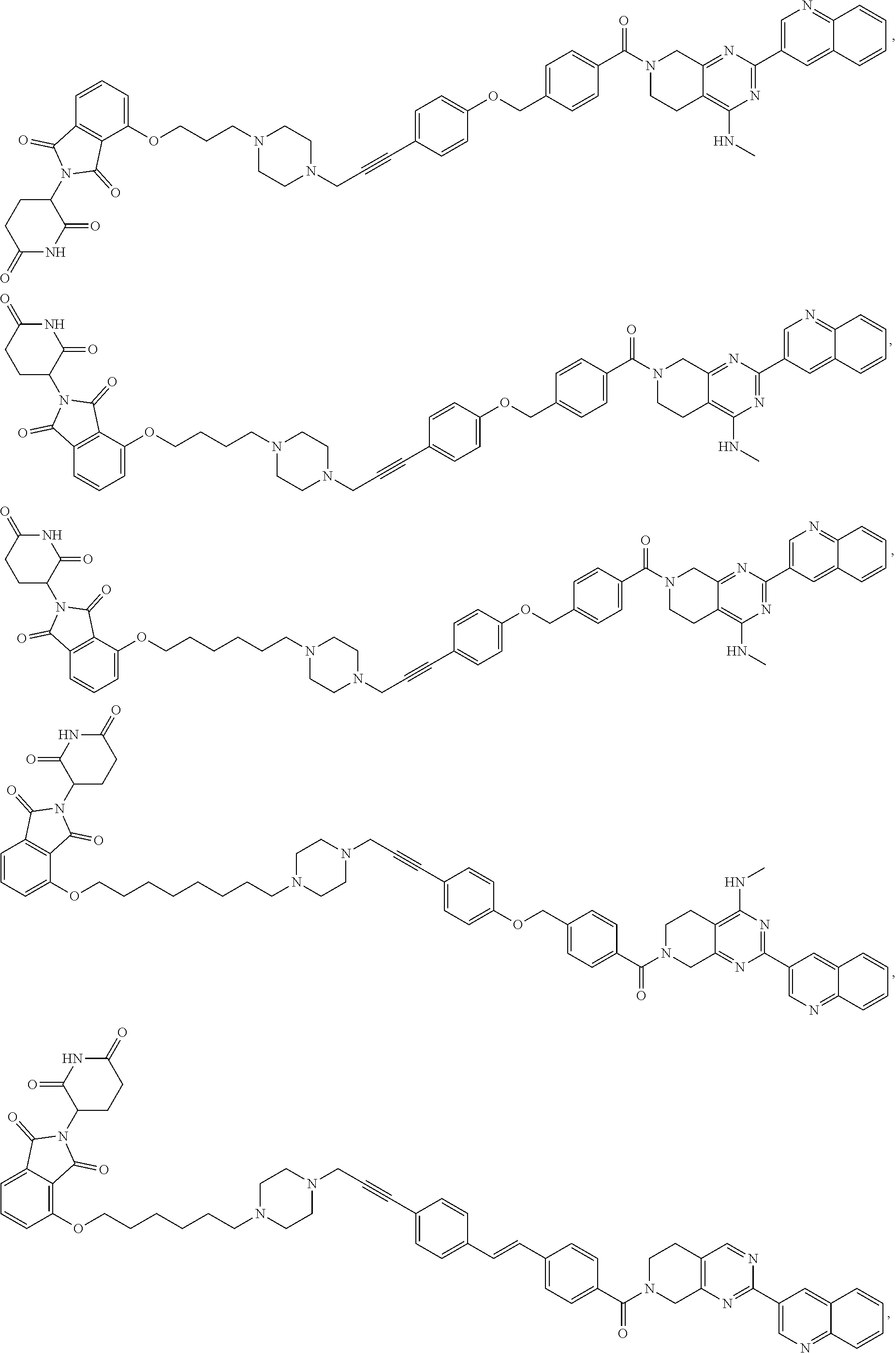

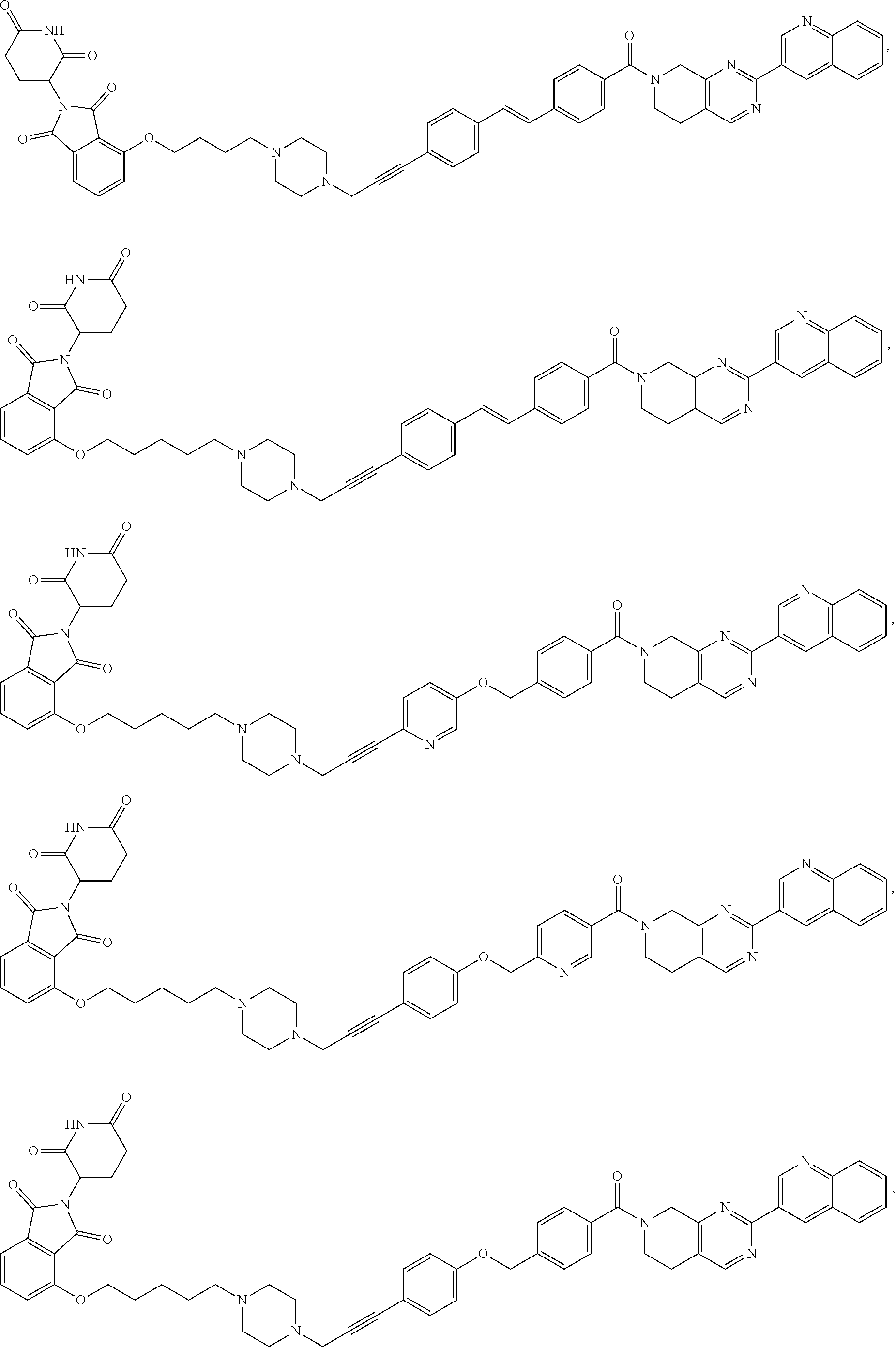

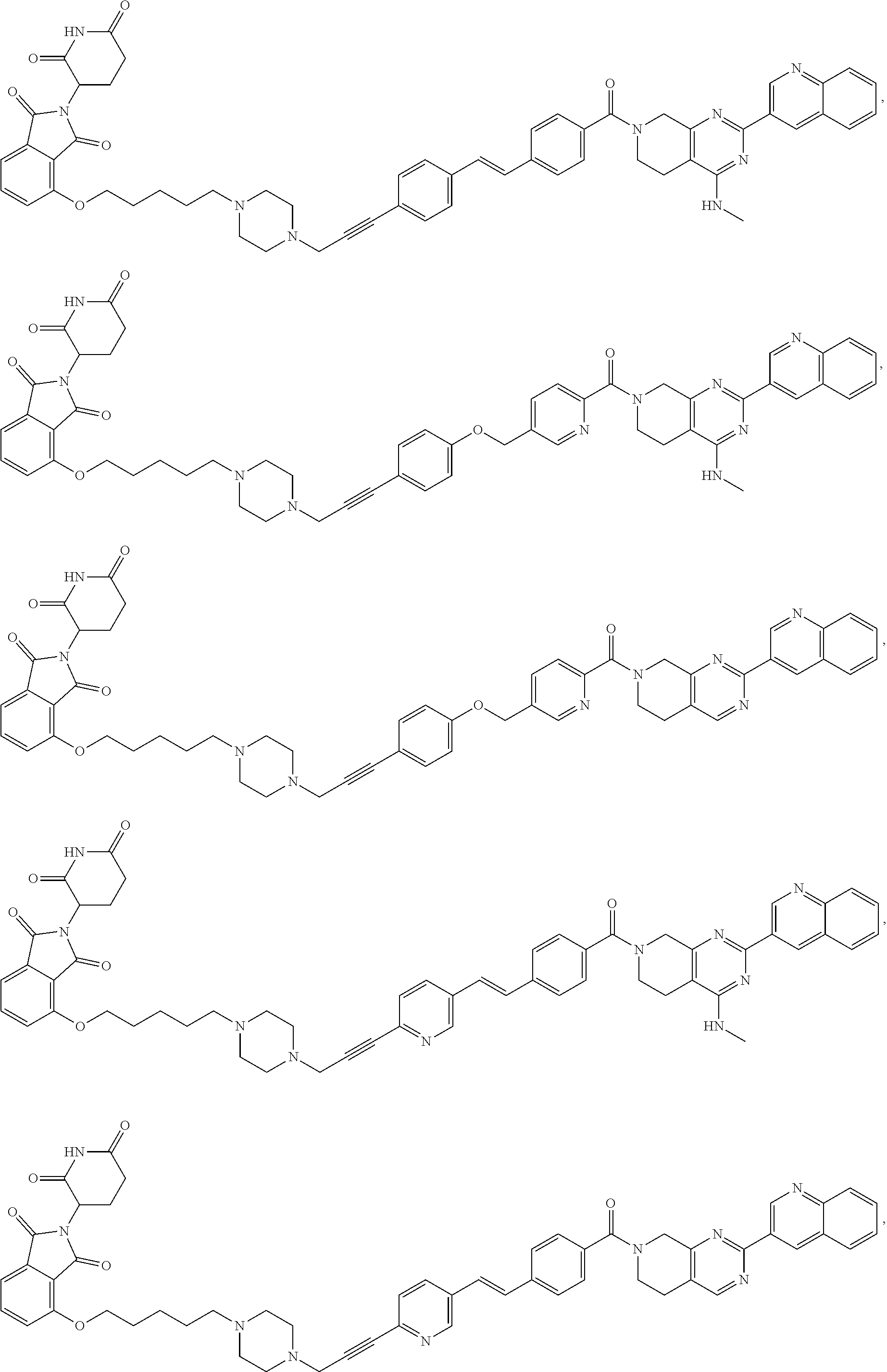

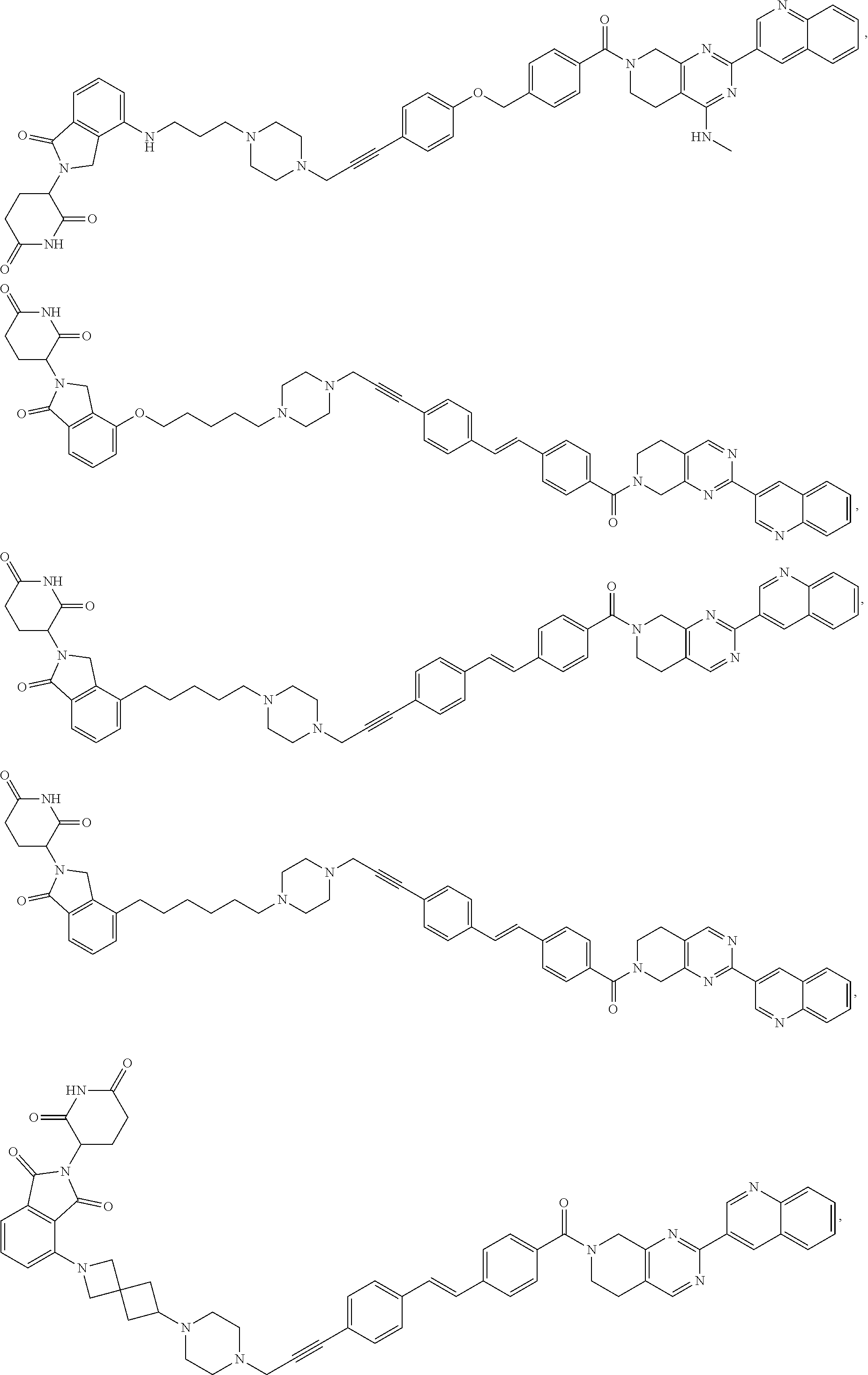

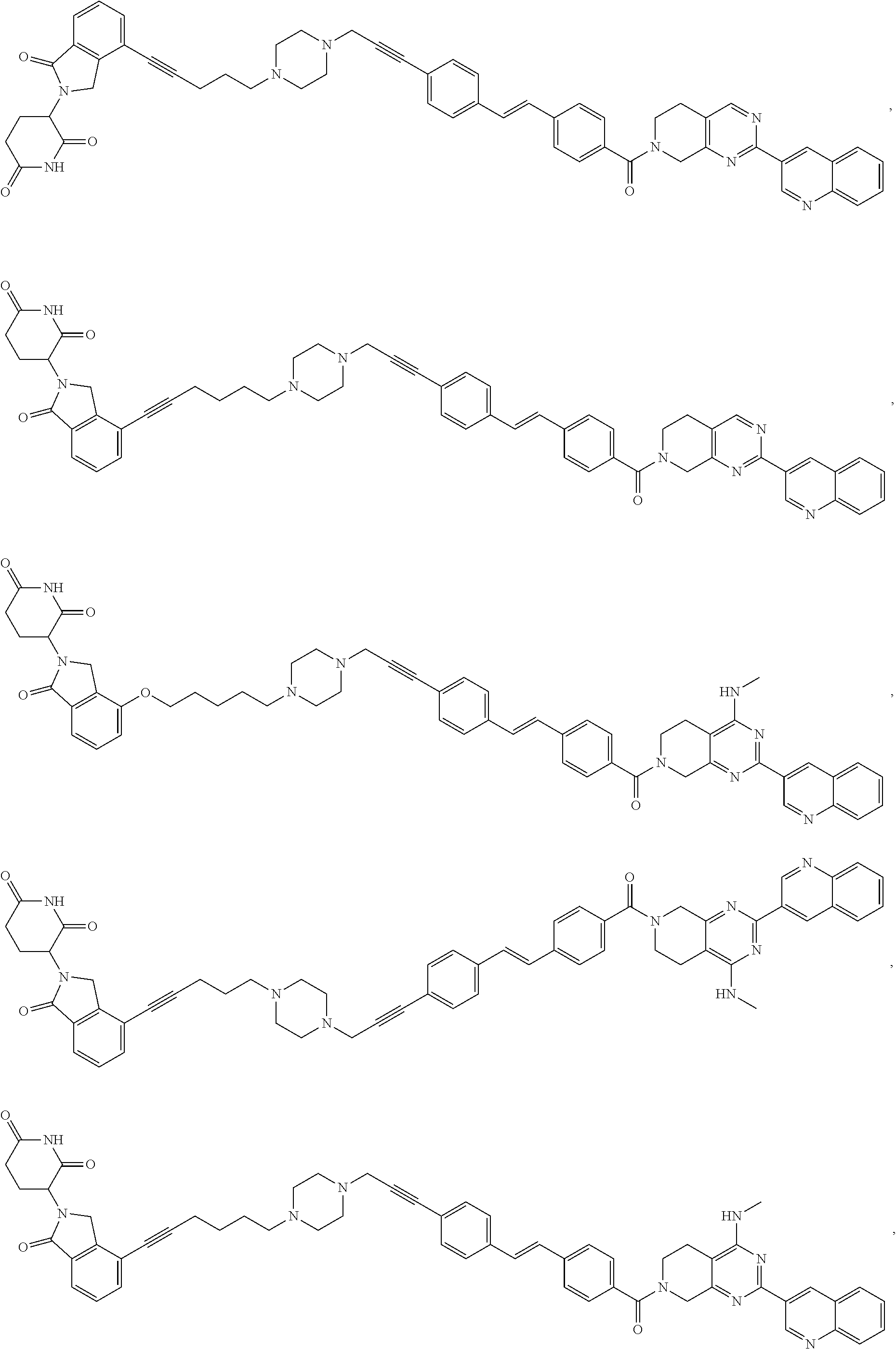

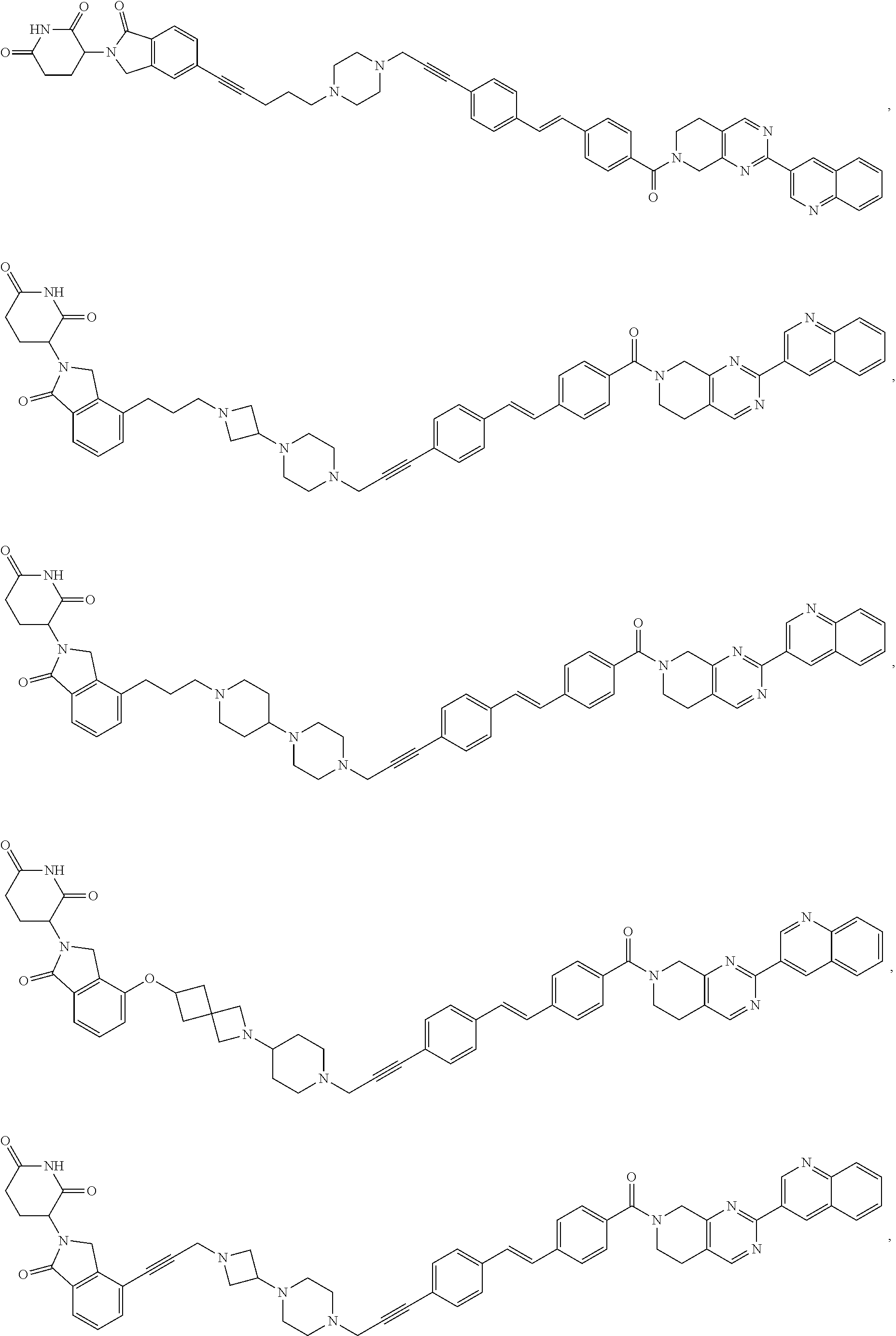

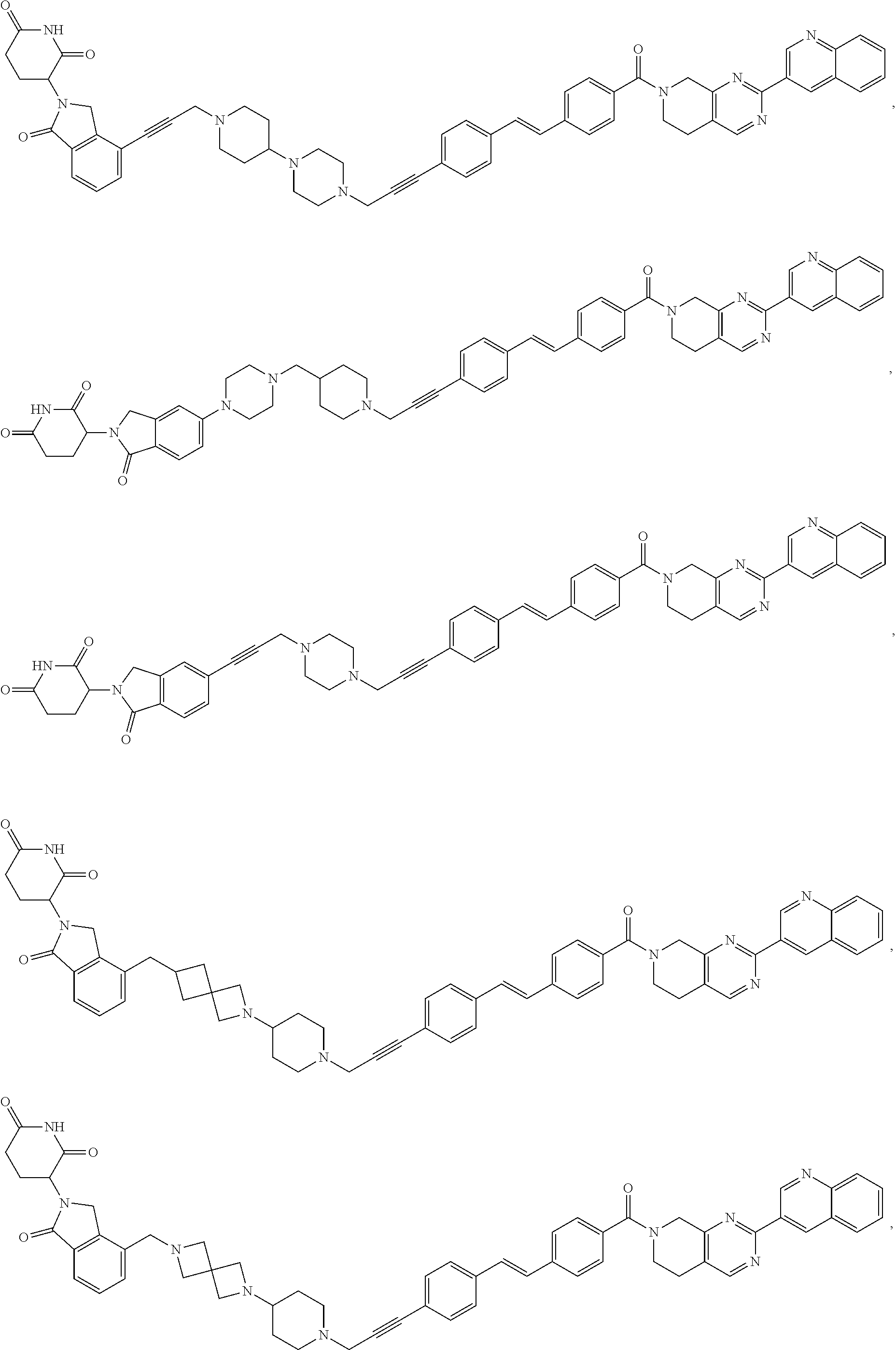

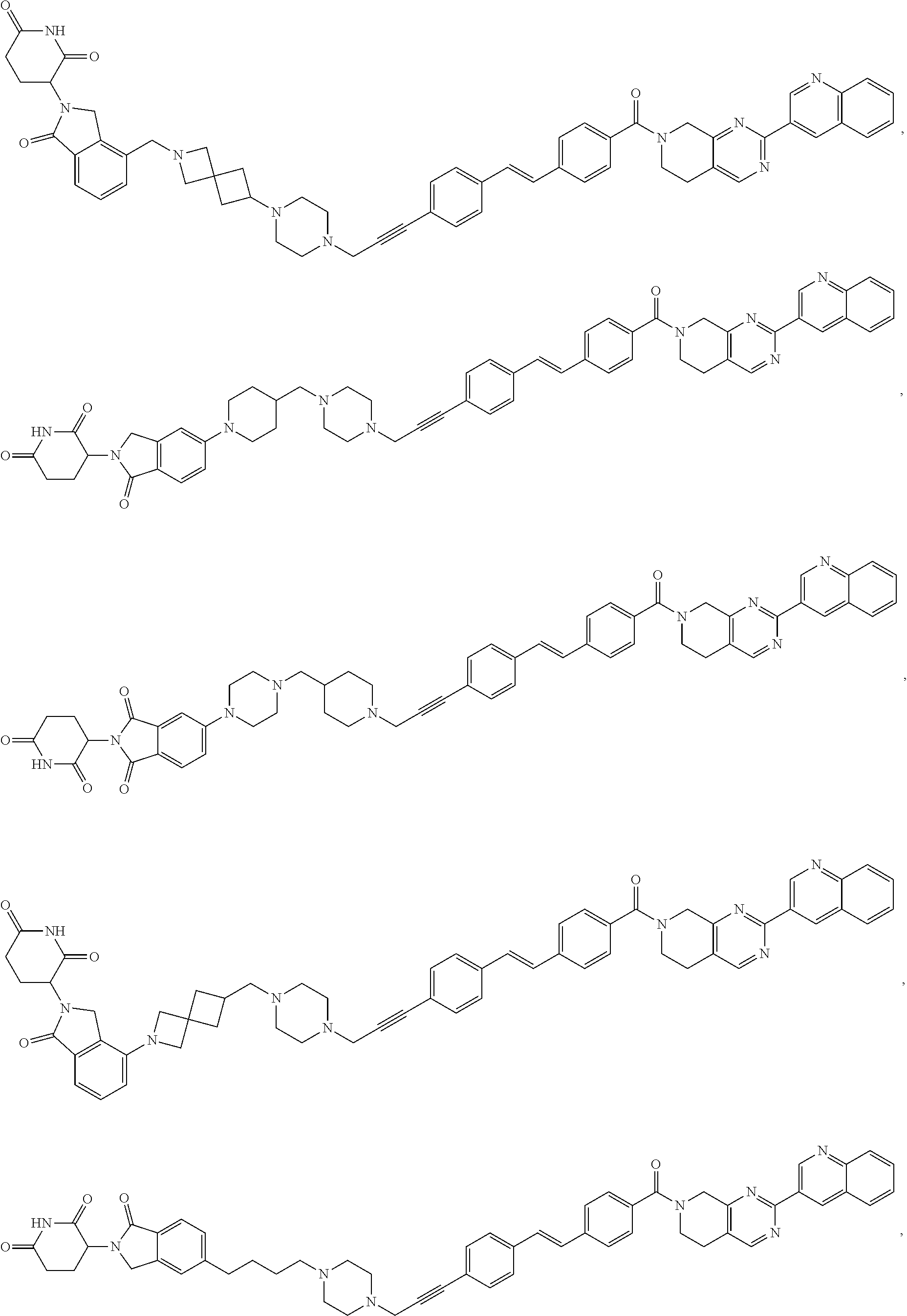

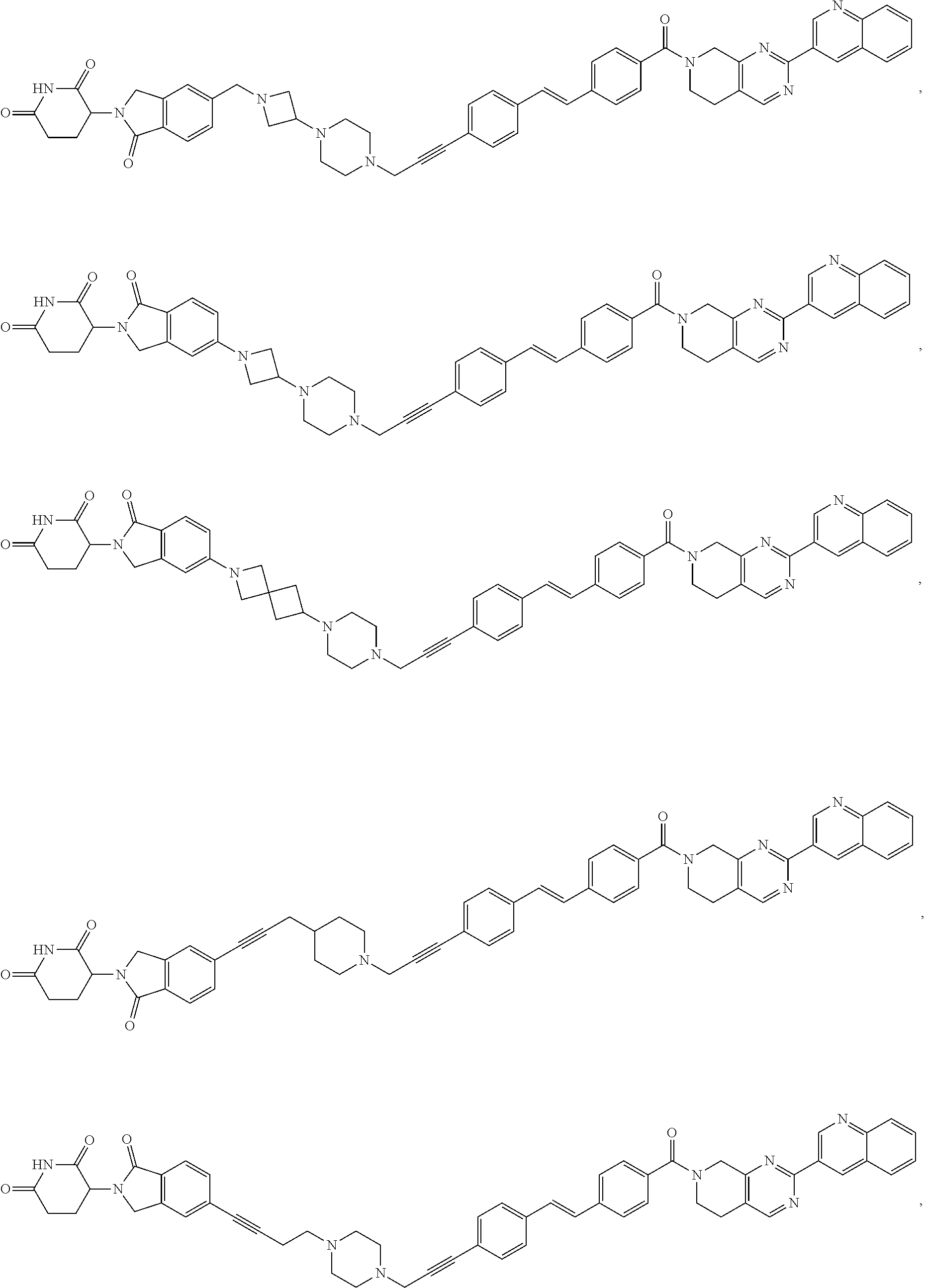

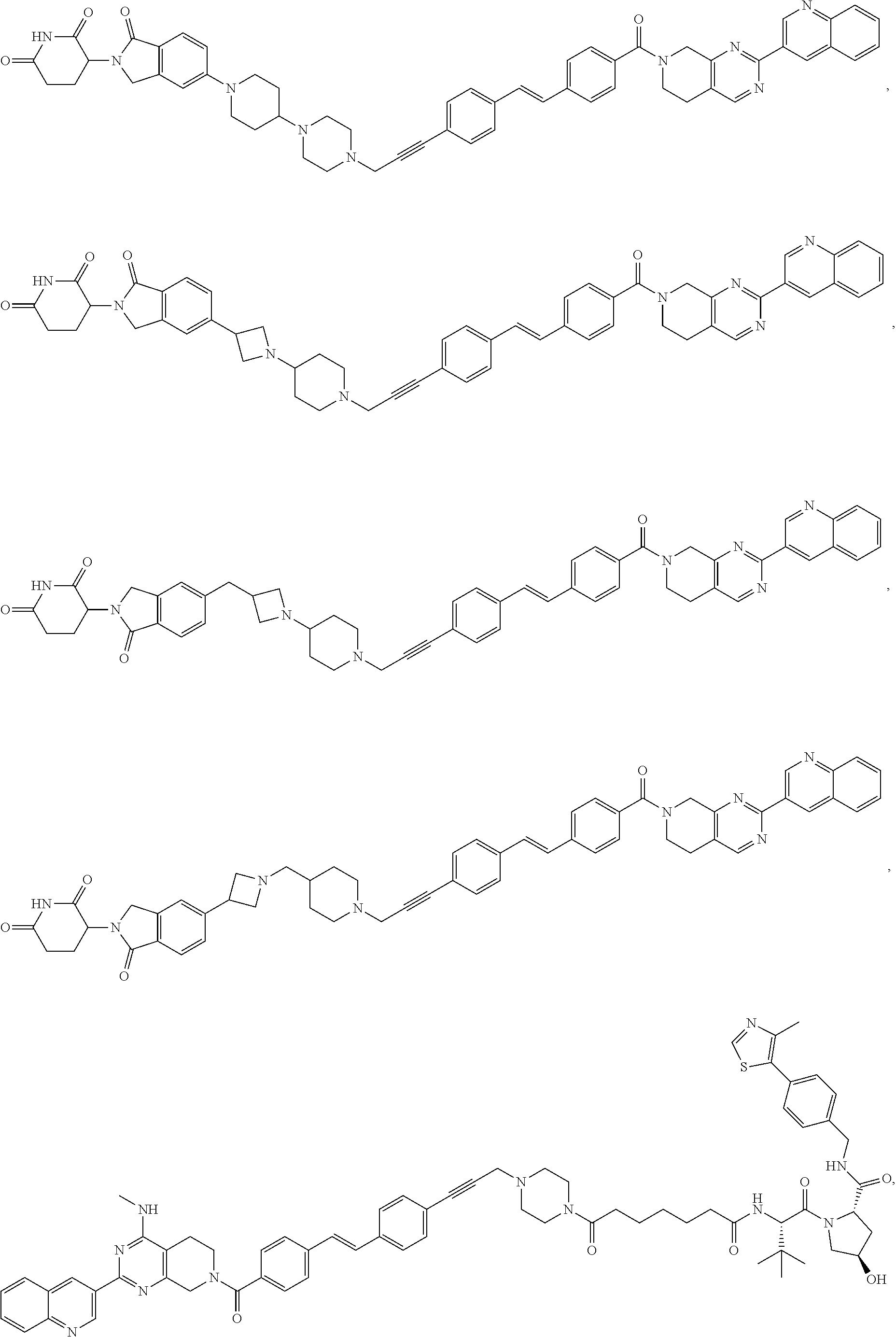

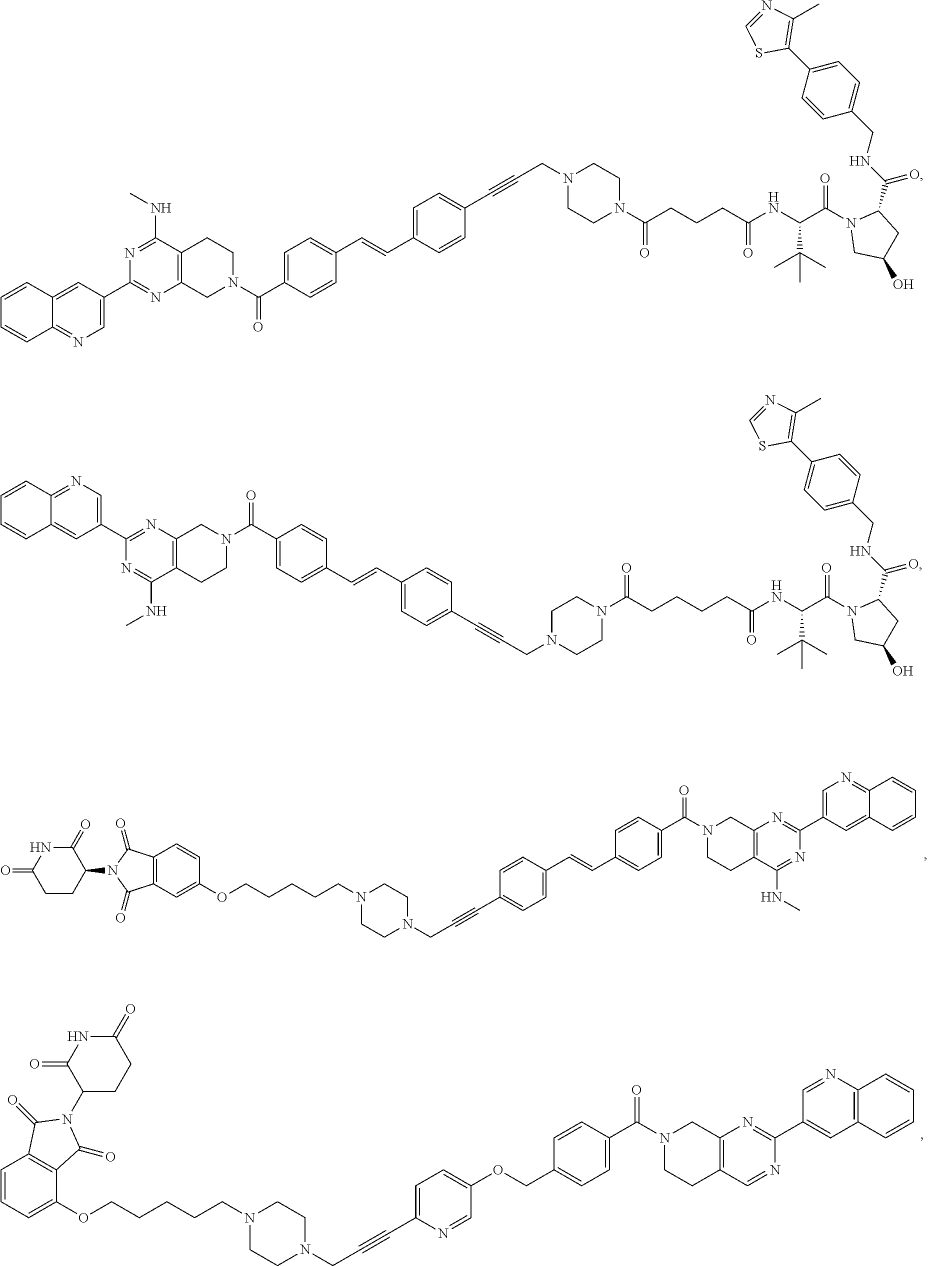

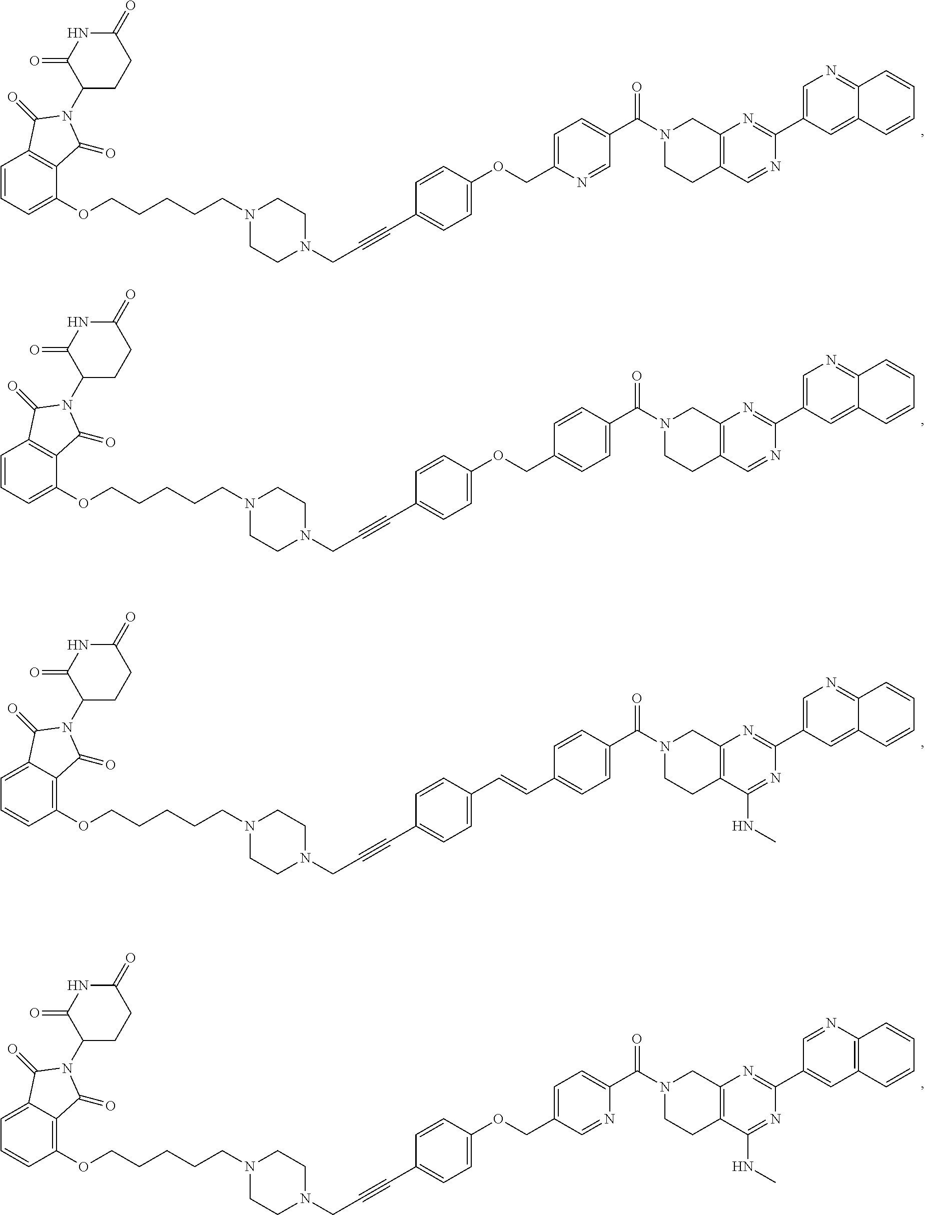 [SMILES: O=C1CCC(N2C(=O)c3cccc(OCCCCCN4CCN(CC#Cc5ccc(OCc6ccc(C(=O)N7CCc8cnc(-c9cnc%10ccccc%10c9)nc8C7)cn6)cc5)CC4)c3C2=O)C(=O)N1 ; O=C1CCC(N2C(=O)c3cccc(OCCCCCN4CCN(CC#Cc5ccc(OCc6ccc(C(=O)N7CCc8cnc(-c9cnc%10ccccc%10c9)nc8C7)cc6)cc5)CC4)c3C2=O)C(=O)N1 ; CNc1nc(-c2cnc3ccccc3c2)nc2c1CCN(C(=O)c1ccc(/C=C/c3ccc(C#CCN4CCN(CCCCCOc5cccc6c5C(=O)N(C5CCC(=O)NC5=O)C6=O)CC4)cc3)cc1)C2 ; CNc1nc(-c2cnc3ccccc3c2)nc2c1CCN(C(=O)c1ccc(COc3ccc(C#CCN4CCN(CCCCCOc5cccc6c5C(=O)N(C5CCC(=O)NC5=O)C6=O)CC4)cc3)cn1)C2]

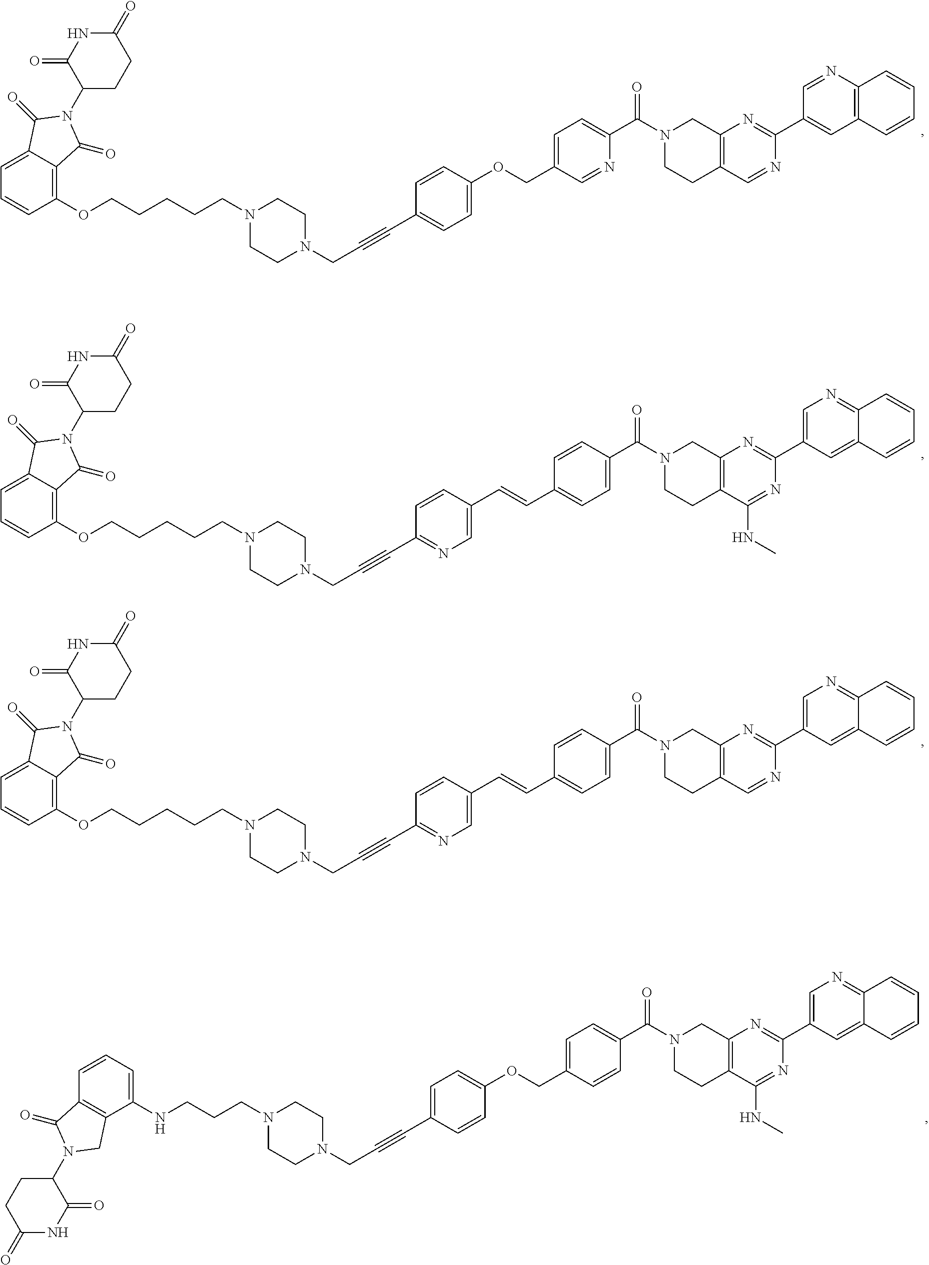 [SMILES: O=C1CCC(N2C(=O)c3cccc(OCCCCCN4CCN(CC#Cc5ccc(OCc6ccc(C(=O)N7CCc8cnc(-c9cnc%10ccccc%10c9)nc8C7)nc6)cc5)CC4)c3C2=O)C(=O)N1 ; CNc1nc(-c2cnc3ccccc3c2)nc2c1CCN(C(=O)c1ccc(/C=C/c3ccc(C#CCN4CCN(CCCCCOc5cccc6c5C(=O)N(C5CCC(=O)NC5=O)C6=O)CC4)nc3)cc1)C2 ; O=C1CCC(N2C(=O)c3cccc(OCCCCCN4CCN(CC#Cc5ccc(/C=C/c6ccc(C(=O)N7CCc8cnc(-c9cnc%10ccccc%10c9)nc8C7)cc6)cn5)CC4)c3C2=O)C(=O)N1 ; CNc1nc(-c2cnc3ccccc3c2)nc2c1CCN(C(=O)c1ccc(COc3ccc(C#CCN4CCN(CCCNc5cccc6c5CN(C5CCC(=O)NC5=O)C6=O)CC4)cc3)cc1)C2]

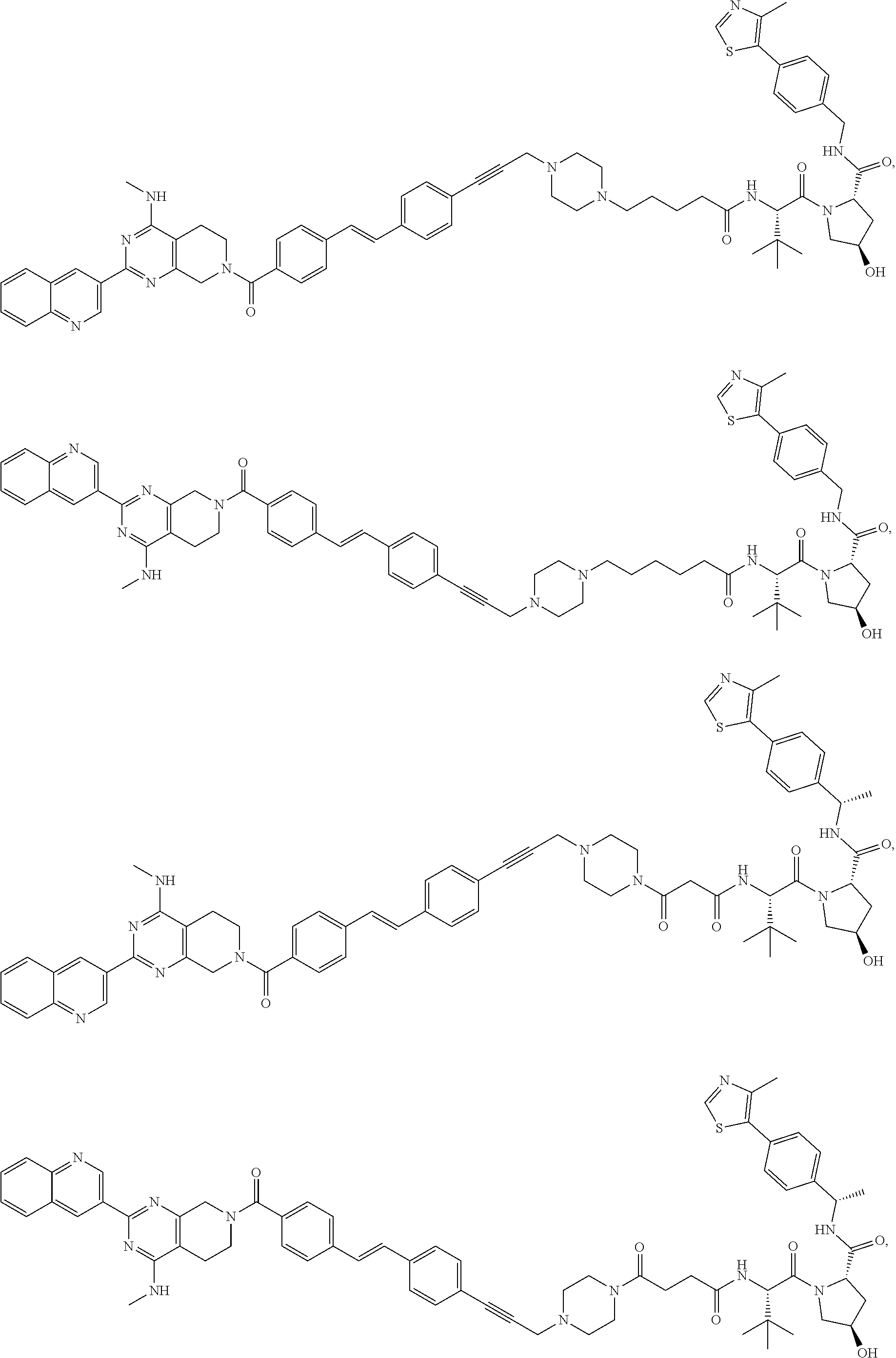

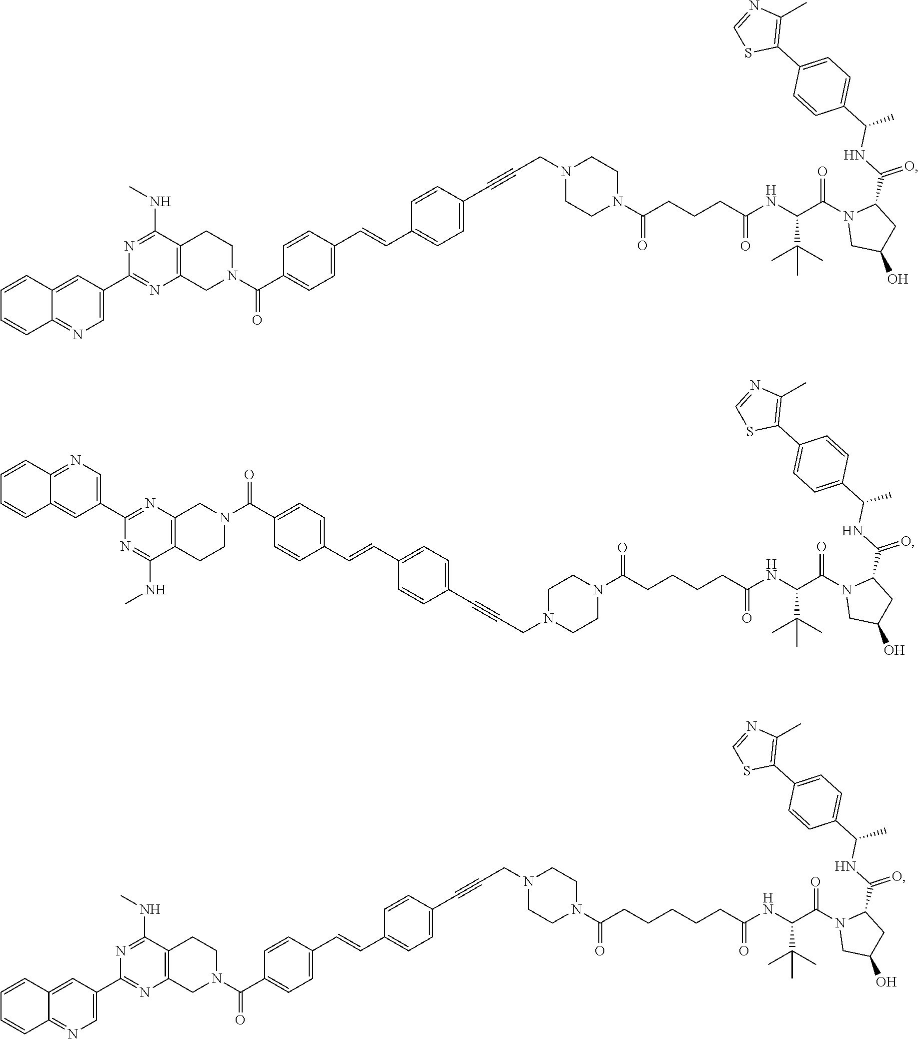

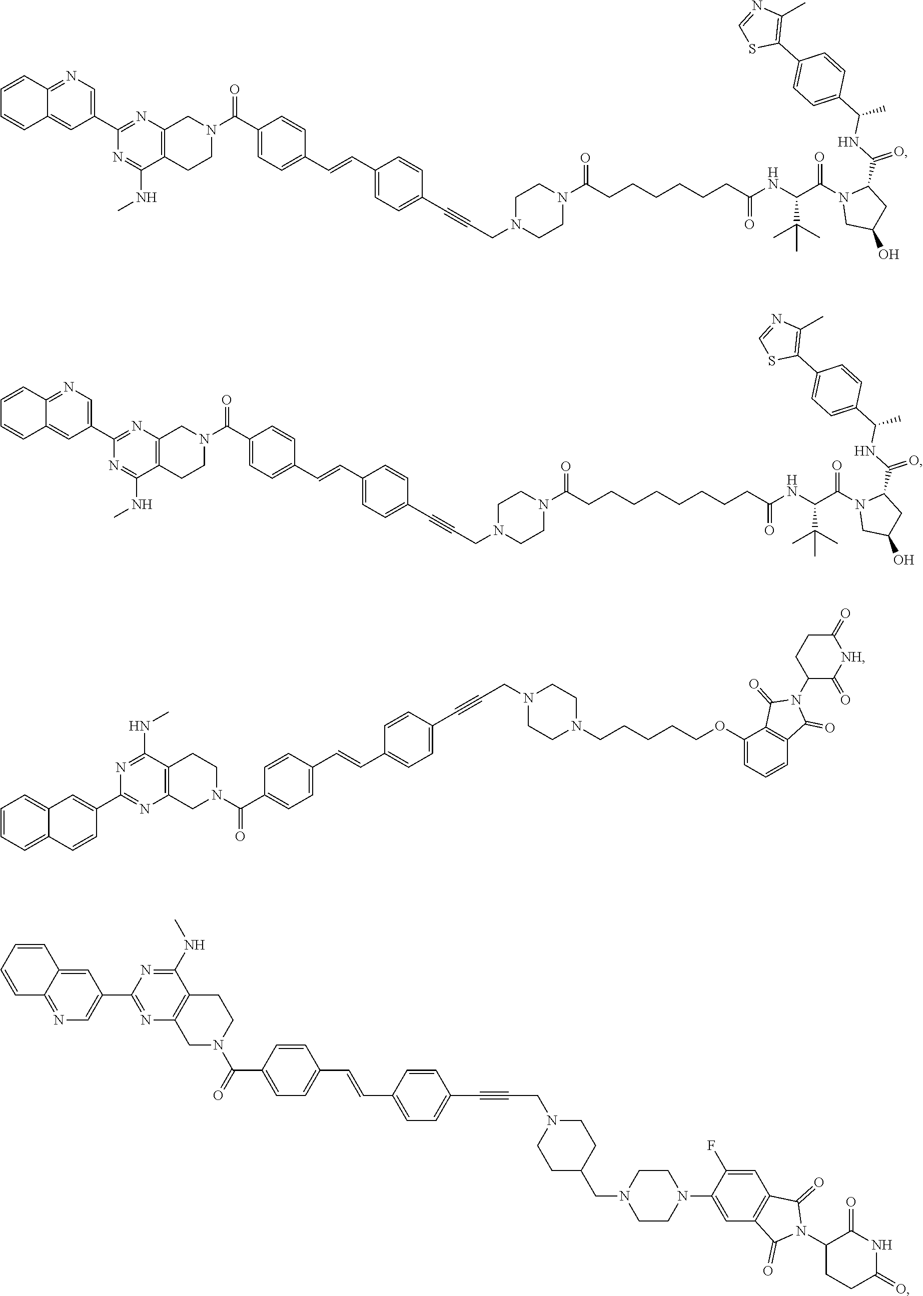

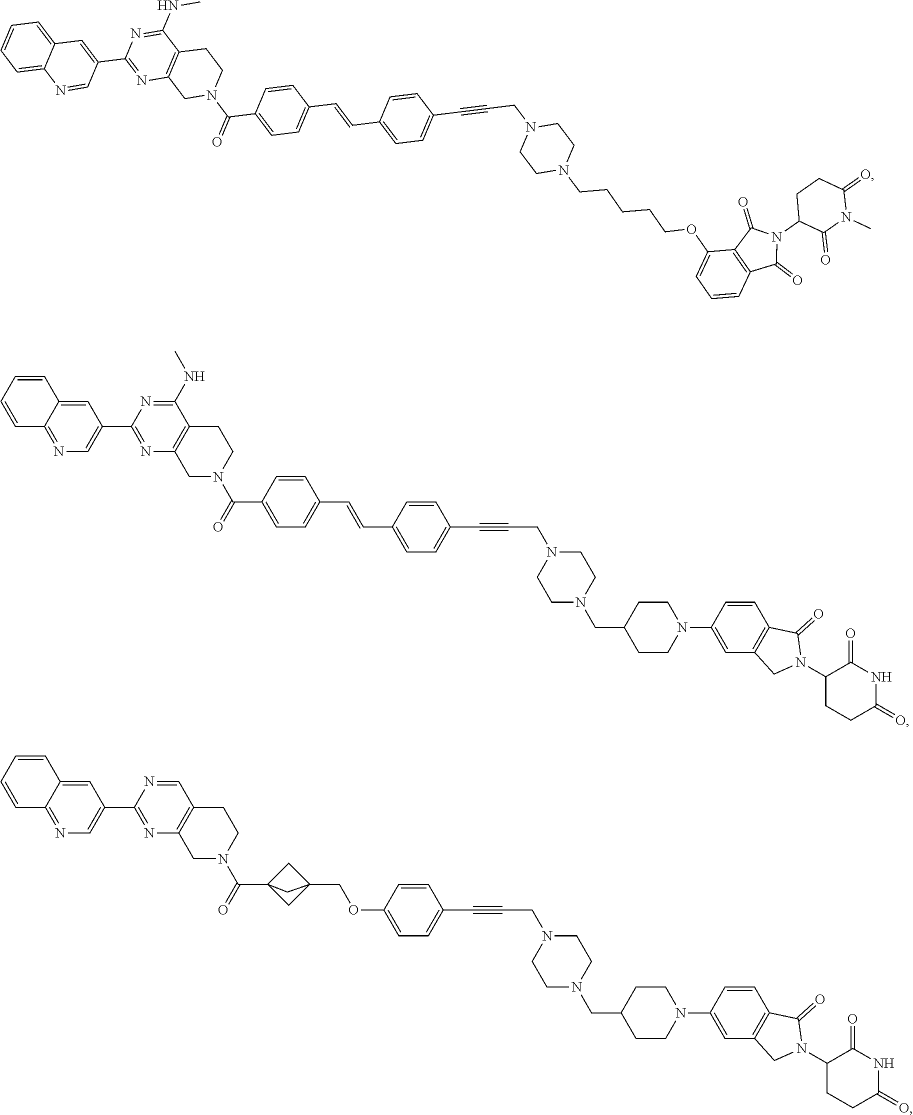 [SMILES: CNc1nc(-c2cnc3ccccc3c2)nc2c1CCN(C(=O)c1ccc(/C=C/c3ccc(C#CCN4CCN(CCCCCOc5cccc6c5C(=O)N(C5CCC(=O)N(C)C5=O)C6=O)CC4)cc3)cc1)C2 ; CNc1nc(-c2cnc3ccccc3c2)nc2c1CCN(C(=O)c1ccc(/C=C/c3ccc(C#CCN4CCN(CC5CCN(c6ccc7c(c6)CN(C6CCC(=O)NC6=O)C7=O)CC5)CC4)cc3)cc1)C2 ; O=C1CCC(N2Cc3cc(N4CCC(CN5CCN(CC#Cc6ccc(OCC78CC(C(=O)N9CCc%10cnc(-c%11cnc%12ccccc%12c%11)nc%10C9)(C7)C8)cc6)CC5)CC4)ccc3C2=O)C(=O)N1]

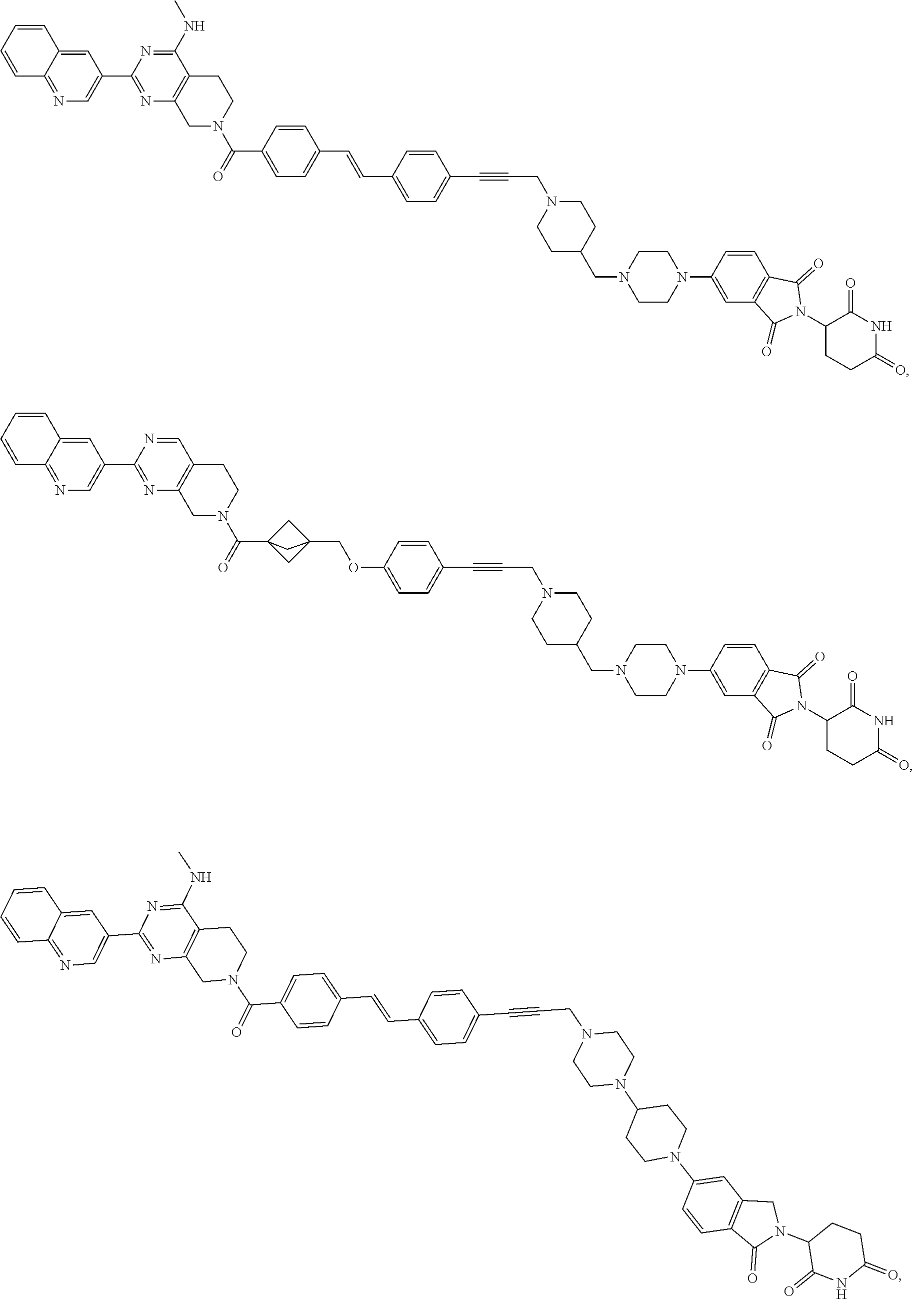 [SMILES: CNc1nc(-c2cnc3ccccc3c2)nc2c1CCN(C(=O)c1ccc(/C=C/c3ccc(C#CCN4CCC(CN5CCN(c6ccc7c(c6)C(=O)N(C6CCC(=O)NC6=O)C7=O)CC5)CC4)cc3)cc1)C2 ; O=C1CCC(N2C(=O)c3ccc(N4CCN(CC5CCN(CC#Cc6ccc(OCC78CC(C(=O)N9CCc%10cnc(-c%11cnc%12ccccc%12c%11)nc%10C9)(C7)C8)cc6)CC5)CC4)cc3C2=O)C(=O)N1 ; CNc1nc(-c2cnc3ccccc3c2)nc2c1CCN(C(=O)c1ccc(/C=C/c3ccc(C#CCN4CCN(C5CCN(c6ccc7c(c6)CN(C6CCC(=O)NC6=O)C7=O)CC5)CC4)cc3)cc1)C2]

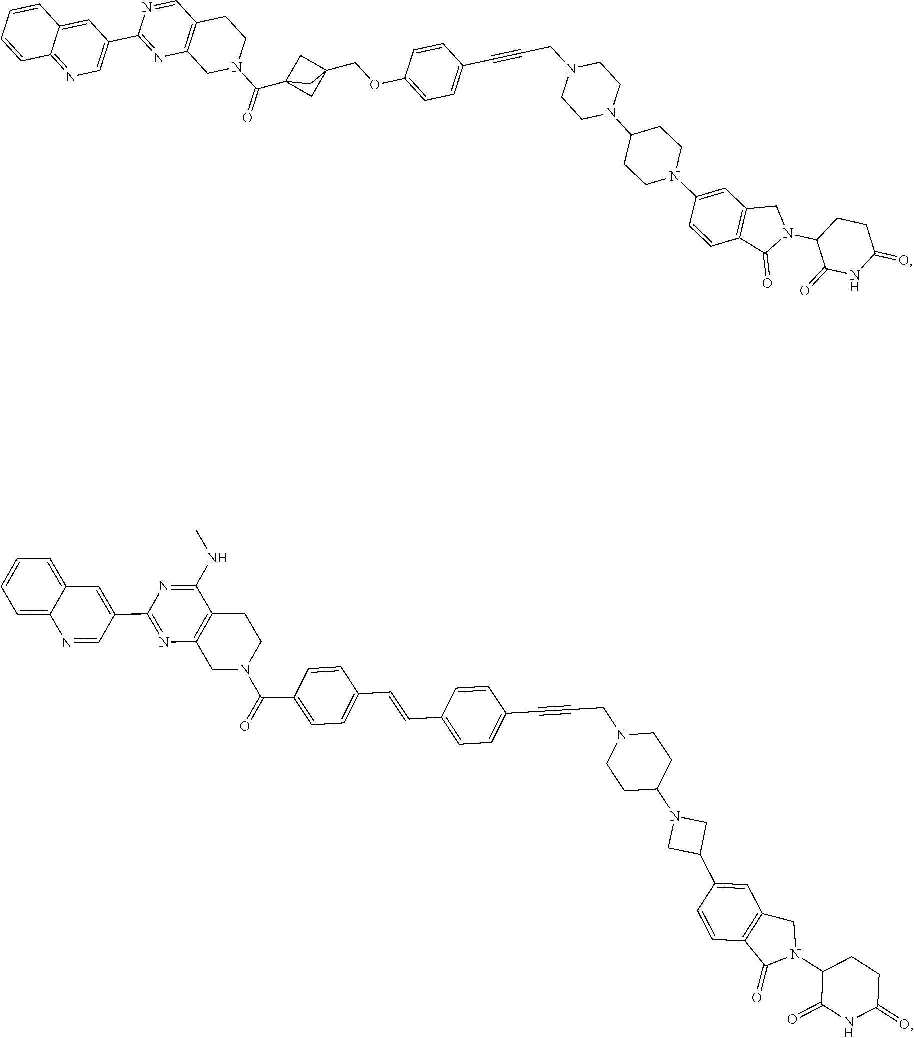 [SMILES: O=C1CCC(N2Cc3cc(N4CCC(N5CCN(CC#Cc6ccc(OCC78CC(C(=O)N9CCc%10cnc(-c%11cnc%12ccccc%12c%11)nc%10C9)(C7)C8)cc6)CC5)CC4)ccc3C2=O)C(=O)N1 ; CNc1nc(-c2cnc3ccccc3c2)nc2c1CCN(C(=O)c1ccc(/C=C/c3ccc(C#CCN4CCC(N5CC(c6ccc7c(c6)CN(C6CCC(=O)NC6=O)C7=O)C5)CC4)cc3)cc1)C2]

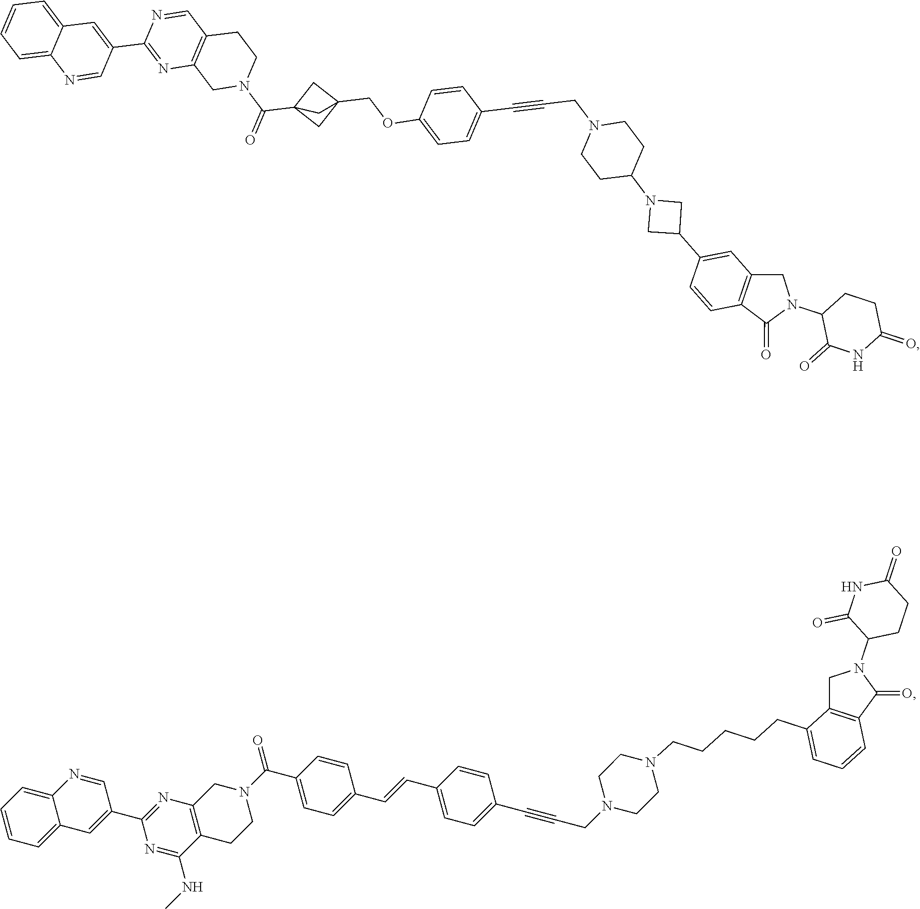 [SMILES: O=C1CCC(N2Cc3cc(C4CN(C5CCN(CC#Cc6ccc(OCC78CC(C(=O)N9CCc%10cnc(-c%11cnc%12ccccc%12c%11)nc%10C9)(C7)C8)cc6)CC5)C4)ccc3C2=O)C(=O)N1 ; CNc1nc(-c2cnc3ccccc3c2)nc2c1CCN(C(=O)c1ccc(/C=C/c3ccc(C#CCN4CCN(CCCCCc5cccc6c5CN(C5CCC(=O)NC5=O)C6=O)CC4)cc3)cc1)C2]

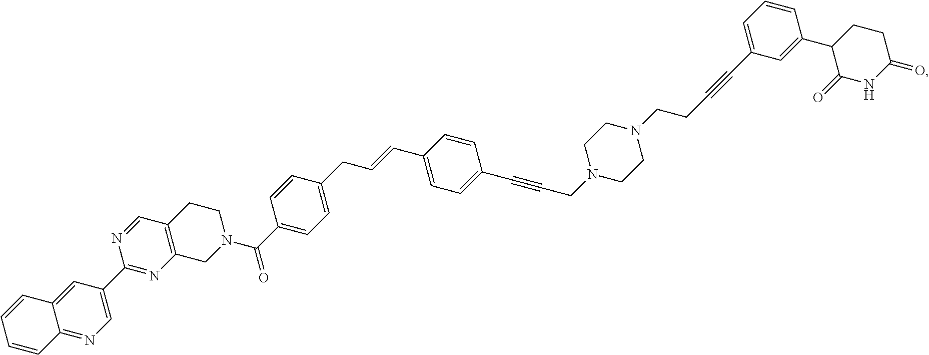 [SMILES: O=C1CCC(c2cccc(C#CCCN3CCN(CC#Cc4ccc(/C=C/Cc5ccc(C(=O)N6CCc7cnc(-c8cnc9ccccc9c8)nc7C6)cc5)cc4)CC3)c2)C(=O)N1]

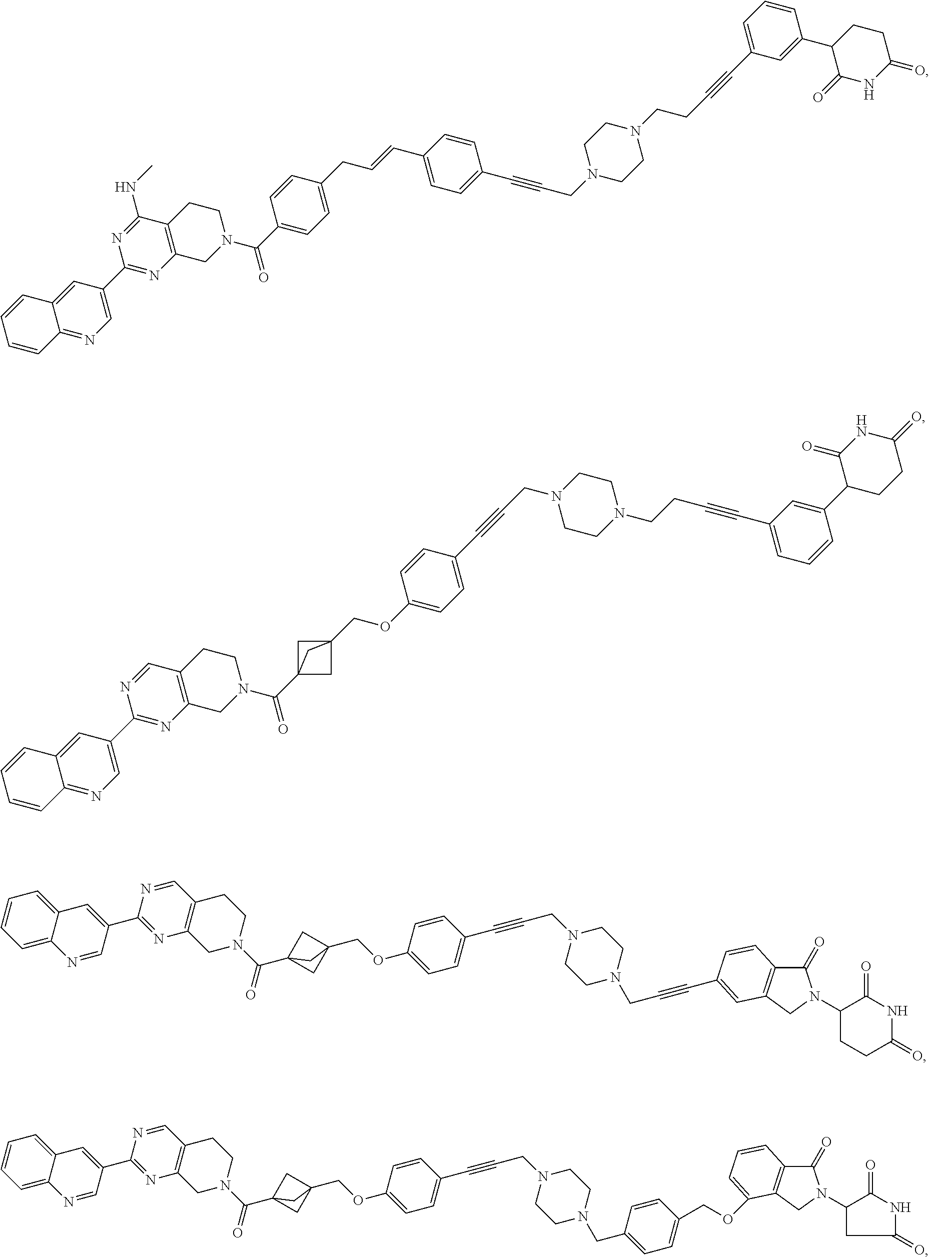 [SMILES: CNc1nc(-c2cnc3ccccc3c2)nc2c1CCN(C(=O)c1ccc(C/C=C/c3ccc(C#CCN4CCN(CCC#Cc5cccc(C6CCC(=O)NC6=O)c5)CC4)cc3)cc1)C2 ; O=C1CCC(c2cccc(C#CCCN3CCN(CC#Cc4ccc(OCC56CC(C(=O)N7CCc8cnc(-c9cnc%10ccccc%10c9)nc8C7)(C5)C6)cc4)CC3)c2)C(=O)N1 ; O=C1CCC(N2Cc3cc(C#CCN4CCN(CC#Cc5ccc(OCC67CC(C(=O)N8CCc9cnc(-c%10cnc%11ccccc%11c%10)nc9C8)(C6)C7)cc5)CC4)ccc3C2=O)C(=O)N1 ; O=C1CC(N2Cc3c(OCc4ccc(CN5CCN(CC#Cc6ccc(OCC78CC(C(=O)N9CCc%10cnc(-c%11cnc%12ccccc%12c%11)nc%10C9)(C7)C8)cc6)CC5)cc4)cccc3C2=O)C(=O)N1]

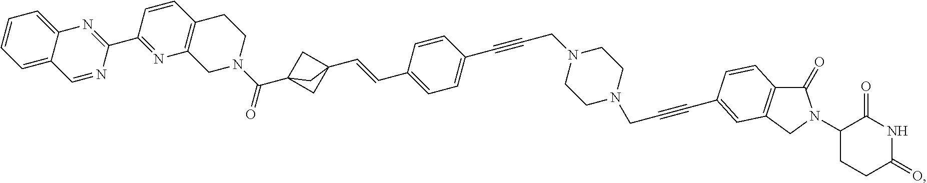 [SMILES: O=C1CCC(N2Cc3cc(C#CCN4CCN(CC#Cc5ccc(/C=C/C67CC(C(=O)N8CCc9ccc(-c%10ncc%11ccccc%11n%10)nc9C8)(C6)C7)cc5)CC4)ccc3C2=O)C(=O)N1]

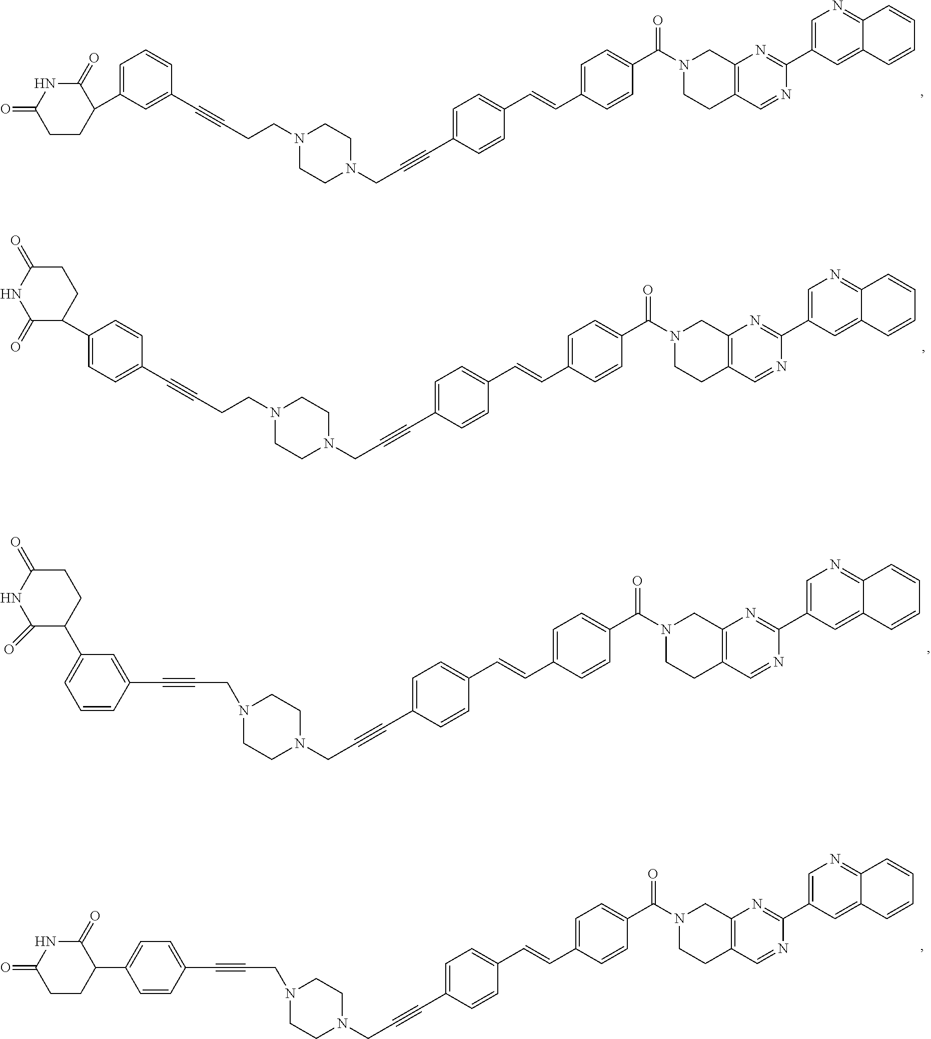 [SMILES: O=C1CCC(c2cccc(C#CCCN3CCN(CC#Cc4ccc(/C=C/c5ccc(C(=O)N6CCc7cnc(-c8cnc9ccccc9c8)nc7C6)cc5)cc4)CC3)c2)C(=O)N1 ; O=C1CCC(c2ccc(C#CCCN3CCN(CC#Cc4ccc(/C=C/c5ccc(C(=O)N6CCc7cnc(-c8cnc9ccccc9c8)nc7C6)cc5)cc4)CC3)cc2)C(=O)N1 ; O=C1CCC(c2cccc(C#CCN3CCN(CC#Cc4ccc(/C=C/c5ccc(C(=O)N6CCc7cnc(-c8cnc9ccccc9c8)nc7C6)cc5)cc4)CC3)c2)C(=O)N1 ; O=C1CCC(c2ccc(C#CCN3CCN(CC#Cc4ccc(/C=C/c5ccc(C(=O)N6CCc7cnc(-c8cnc9ccccc9c8)nc7C6)cc5)cc4)CC3)cc2)C(=O)N1]

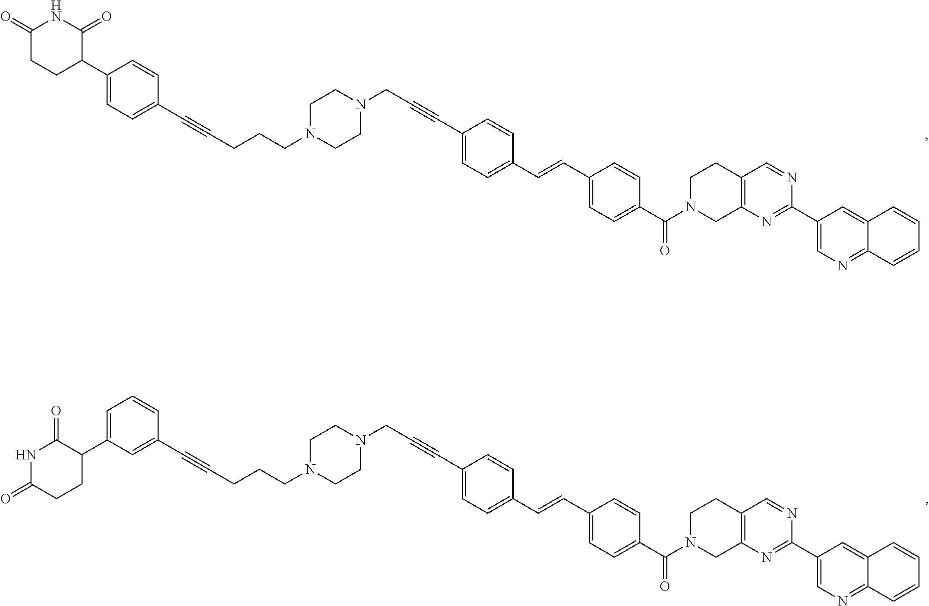 [SMILES: O=C1CCC(c2ccc(C#CCCCN3CCN(CC#Cc4ccc(/C=C/c5ccc(C(=O)N6CCc7cnc(-c8cnc9ccccc9c8)nc7C6)cc5)cc4)CC3)cc2)C(=O)N1 ; O=C1CCC(c2cccc(C#CCCCN3CCN(CC#Cc4ccc(/C=C/c5ccc(C(=O)N6CCc7cnc(-c8cnc9ccccc9c8)nc7C6)cc5)cc4)CC3)c2)C(=O)N1]

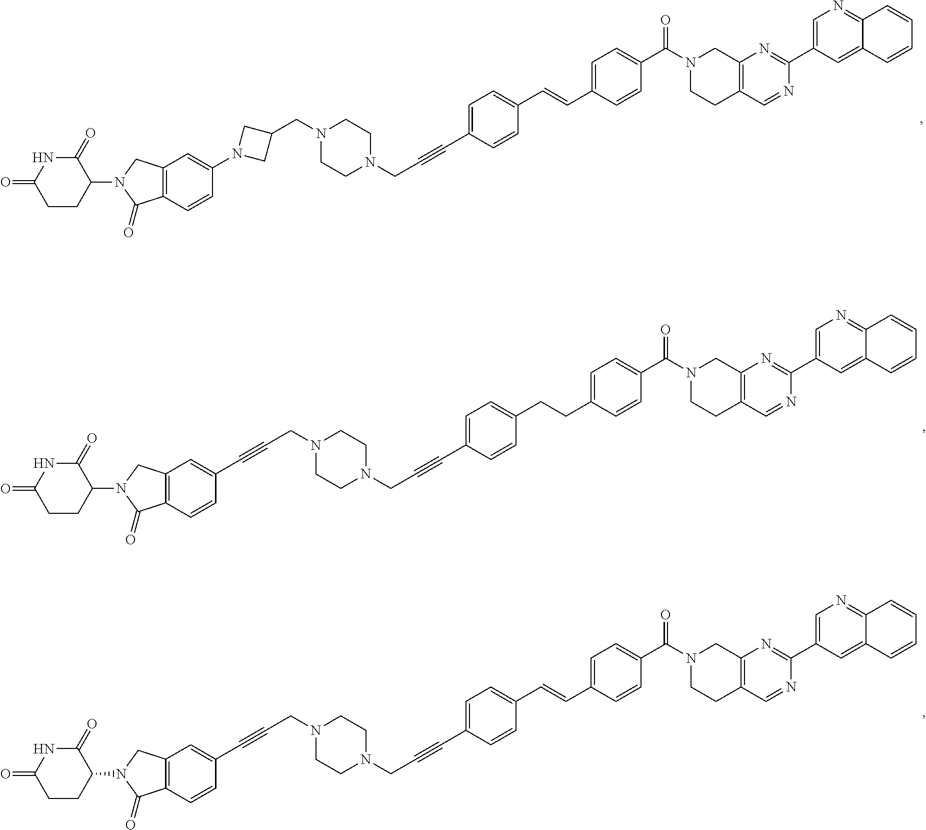 [SMILES: O=C1CCC(N2Cc3cc(N4CC(CN5CCN(CC#Cc6ccc(/C=C/c7ccc(C(=O)N8CCc9cnc(-c%10cnc%11ccccc%11c%10)nc9C8)cc7)cc6)CC5)C4)ccc3C2=O)C(=O)N1 ; O=C1CCC(N2Cc3cc(C#CCN4CCN(CC#Cc5ccc(CCc6ccc(C(=O)N7CCc8cnc(-c9cnc%10ccccc%10c9)nc8C7)cc6)cc5)CC4)ccc3C2=O)C(=O)N1 ; O=C1CC[C@@H](N2Cc3cc(C#CCN4CCN(CC#Cc5ccc(/C=C/c6ccc(C(=O)N7CCc8cnc(-c9cnc%10ccccc%10c9)nc8C7)cc6)cc5)CC4)ccc3C2=O)C(=O)N1]

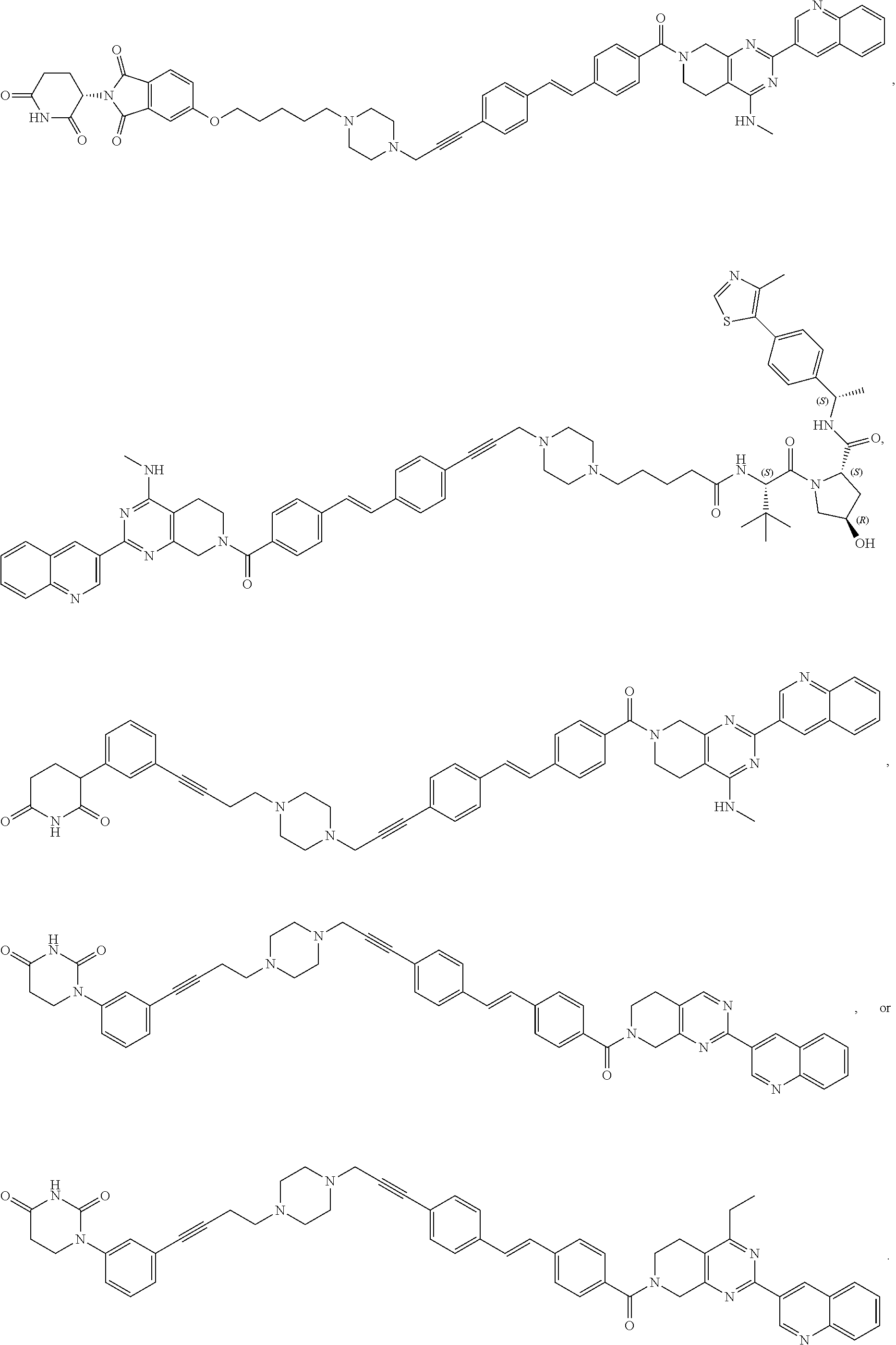

According to some embodiments of the present invention, the compound of Formula I may also be optionally and independently substituted by one or more (for example, 2, 3, 4, 5, 6, 7, 8, 9 or 10) R^{YL′}.

According to some embodiments of the present invention, each R^{YL′} is independently selected from the group consisting of halogen, cyano, nitro, C_{1˜6} alkyl, halogen-substituted C_{1˜6} alkyl, —OR′, and —N(R′)₂; each R′ is independently selected from the group consisting of hydrogen, halogen, —C_{1˜6} alkyl, halogen-substituted —C_{1˜6} alkyl, —C_{0˜2} alkylene-(C_{3˜10} carbocyclic group), and —C_{0˜2} alkylene-(3- to 10-membered heterocycloalkyl).

According to some embodiments of the present invention, each R^{YL′} is independently selected from the group consisting of halogen, cyano, nitro, C_{1˜6} alkyl, halogen-substituted C_{1˜6} alkyl, —OR′, and —N(R′)₂; each R′ is independently selected from the group consisting of hydrogen, halogen, —C_{1˜6} alkyl, halogen-substituted —C_{1˜6} alkyl, —C_{0˜2} alkylene-(C_{3˜8} carbocyclic group), and —C_{0˜2} alkylene-(3- to 8-membered heterocycloalkyl).

According to some embodiments of the present invention, each R^{YL′} is independently selected from the group consisting of halogen, cyano, nitro, C_{1˜3} alkyl, halogen-substituted C_{1˜3} alkyl, —OR′ and —N(R′)₂; each R′ is independently selected from the group consisting of hydrogen, halogen, —C_{1˜3} alkyl, halogen-substituted —C_{1˜3} alkyl, —C_{0˜2} alkylene-(C₃˜6 carbocyclic group) and —C_{0˜2} alkylene-(3- to 6-membered heterocycloalkyl).

The present invention also comprises technical solutions formed by any combination of various groups as described above in the present application, such as compounds of Formula I composed of X, Y and Z as described above, compounds of Formula I comprising X and Y as described above, compounds of Formula I comprising X and Z as described above, and compounds of Formula I comprising Y and Z as described above.

The present invention also provides a pharmaceutical composition, comprising a preparation made from any of the above-mentioned compounds, or deuterated compounds thereof, or stereoisomers thereof, or tautomers thereof, or polymorphs thereof, or solvates thereof, or N-oxides thereof, or isotopically labeled compounds thereof, or metabolites thereof, or prodrugs thereof, or pharmaceutically acceptable salts thereof.

The above pharmaceutical composition further comprises a pharmaceutically acceptable carrier, excipient, or vehicle.

The present invention also provides a use of any of the above-mentioned compounds, or deuterated compounds thereof, or stereoisomers thereof, or tautomers thereof, or polymorphs thereof, or solvates thereof, or N-oxides thereof, or isotopically labeled compounds thereof, or metabolites thereof, or prodrugs thereof, or pharmaceutically acceptable salts thereof, or the pharmaceutical composition in the preparation of a medicament for preventing and/or treating a disease related to BCL-XL activity.

The present invention also provides a use of any of the above-mentioned compounds, or deuterated compounds thereof, or stereoisomers thereof, or tautomers thereof, or polymorphs thereof, or solvates thereof, or N-oxides thereof, or isotopically labeled compounds thereof, or metabolites thereof, or prodrugs thereof, or pharmaceutically acceptable salts thereof, or the pharmaceutical composition in the preparation of a medicament for preventing and/or treating a cancer.

The present invention also provides any of the above-mentioned compounds, or deuterated compounds thereof, or stereoisomers thereof, or tautomers thereof, or polymorphs thereof, or solvates thereof, or N-oxides thereof, or isotopically labeled compounds thereof, or metabolites thereof, or prodrugs thereof, or pharmaceutically acceptable salts thereof, or the pharmaceutical composition for use in preventing and/or treating a disease associated with BCL-XL activity.

In some embodiments, the above-mentioned disease associated with BCL-XL activity is autoimmune disease, bladder cancer, brain cancer, breast cancer, bone marrow cancer, cervical cancer, colorectal cancer, esophageal cancer, hepatocellular carcinoma, follicular lymphoma, lymphatic malignancy of T cell or B cell origin, melanoma, myeloma, oral cancer, ovarian cancer, non-small cell lung cancer, prostate cancer, leukemia, small cell lung cancer or spleen cancer; the leukemia is preferably chronic lymphocytic leukemia, lymphoblastic leukemia, or granulocytic leukemia.

The present invention also provides any of the above-mentioned compounds, or deuterated compounds thereof, or stereoisomers thereof, or tautomers thereof, or polymorphs thereof, or solvates thereof, or N-oxides thereof, or isotopically labeled compounds thereof, or metabolites thereof, or prodrugs thereof, or pharmaceutically acceptable salts thereof, or the pharmaceutical composition for use in preventing and/or treating a cancer.

A method for preventing and/or treating a disease associated with BCL-XL activity, which comprises administering an effective amount of any of the above compounds, or deuterated compounds thereof, or stereoisomers thereof, or tautomers thereof, or polymorphs thereof, or solvates thereof, or N-oxides thereof, or isotopically labeled compounds thereof, or metabolites thereof, or prodrugs thereof, or pharmaceutically acceptable salts thereof, or the pharmaceutical composition to a subject in need thereof.

In some embodiments, the above-mentioned disease associated with BCL-XL activity is autoimmune disease, bladder cancer, brain cancer, breast cancer, bone marrow cancer, cervical cancer, colorectal cancer, esophageal cancer, hepatocellular carcinoma, follicular lymphoma, lymphatic malignancy of T cell or B cell origin, melanoma, myeloma, oral cancer, ovarian cancer, non-small cell lung cancer, prostate cancer, small cell lung cancer or spleen cancer; the leukemia is preferably chronic lymphocytic leukemia, lymphoblastic leukemia, or granulocytic leukemia.

A method for preventing and/or treating a cancer, which comprises administering an effective amount of any of the above compounds, or deuterated compounds thereof, or stereoisomers thereof, or tautomers thereof, or polymorphs thereof, or solvates thereof, or N-oxides thereof, or isotopically labeled compounds thereof, or metabolites thereof, or prodrugs thereof, or pharmaceutically acceptable salts thereof, or the pharmaceutical composition to a subject in need thereof.

The compounds and derivatives provided in the present invention may be named according to the nomenclature systems of IUPAC (International Union of Pure and Applied Chemistry) or CAS (Chemical Abstracts Service, Columbus, OH).

Definitions of terms used in the present invention: Unless otherwise stated, the initial definition provided for a group or term herein applies to the group or term in the entire description; for terms that are not specifically defined herein, their meanings should be that the skilled in the art can give them based on the invention content and context.

“Substitution” means that a hydrogen atom in a molecule is replaced by other different atom or group; or a lone pair of electrons of an atom in a molecule is replaced by other atom or group. For example, the lone pair of electrons on S atom can be replaced by O atom to form

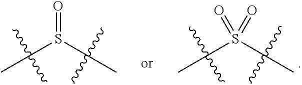

“Can be further substituted” means that “substitution” may but does not have to occur, and this description includes whether it occurs or not.

The minimum and maximum content of carbon atoms in a hydrocarbon group is indicated by a prefix, for example, the prefix C_{a˜b} alkyl refers to any alkyl containing “a” to “b” carbon atoms. Thus, for example, C_{1˜6} alkyl refers to alkyl containing 1 to 6 carbon atoms.

“Alkyl” refers to a saturated hydrocarbon chain having the specified number of member atoms. Alkyl groups can be straight or branched. Representative branched alkyl groups have one, two or three branches. Alkyl groups may be optionally substituted with one or more substituents as defined herein. Alkyl groups include methyl, ethyl, propyl (n-propyl and isopropyl), butyl (n-butyl, isobutyl and tert-butyl), pentyl (n-pentyl, isopentyl and neopentyl) and hexyl. Alkyl group can also be a part of other groups, such as —O(C_{1_6} alkyl).

“Alkylene” refers to a divalent saturated aliphatic hydrocarbon group having the specified number of member atoms. C_{a-b} alkylene refers to an alkylene group having a to b carbon atoms. Alkylene groups include branched and straight chain hydrocarbyl groups. For example, the term “propylene” can be exemplified by the following structure:

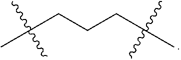 [SMILES: *CCC*]

Likewise, the term “dimethylbutylene” may be exemplified, for example, by any of the following structures:

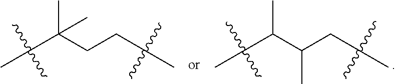

The C_{0˜4} alkylene group of the present invention can be C₀ alkylene group, C₁ alkylene group (e.g., —CH₂—), C₂ alkylene group (e.g., —CH₂CH₂—, etc.), C₃ alkylene group or C₄ alkylene group; C₀ alkylene means that the group here does not exist and is connected in the form of a chemical bond. For example, A-C₀ alkylene-B means A-B, that is, the group A and the group B are directly connected through a chemical bond.

The term “carbocyclic group” in the present invention refers to a saturated or non-aromatic partially saturated cyclic group having a single ring or multiple rings (fused, bridged, spiro) and multiple carbon atoms, but having no ring heteroatoms. The term “carbocyclic group” includes cycloalkenyl groups such as cyclohexenyl. Examples of monocarbocyclic groups include, for example, cyclopropyl, cyclobutyl, cyclohexyl, cyclopentyl, cyclooctyl, cyclopentenyl and cyclohexenyl. Examples of carbocyclic groups of the fused carbocyclic system include bicyclohexyl, bicyclopentyl, bicyclooctyl, etc. Two such bicycloalkyl polycyclic structures are exemplified and named below:

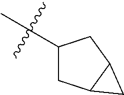 [SMILES: *C1CC2CC2C1]

bicyclohexyl and

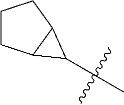 [SMILES: *C1C2CCCC12]

bicyclohexyl. Examples of carbocyclic groups of the bridged carbocyclic system include

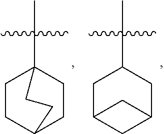

adamantyl, and the like. Examples of carbocyclic groups of the spirocarbocyclic system include

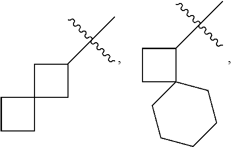 [SMILES: *C1CC2(CCC2)C1 ; *C1CCC12CCCCC2]

and the like. The term “carbocyclic group” also comprises the case of partially saturated cyclic group formed by the fusion of an aromatic ring and a non-aromatic ring, and the connection site can be located at a non-aromatic carbon atom or an aromatic carbon atom. Examples include 1,2,3,4-tetrahydronaphthalen-5-yl, and 5,6,7,8-tetrahydronaphthalen-5-yl.

“Cycloalkane” as used in the present invention refers to a saturated or non-aromatic partially saturated divalent cyclic alkane having a single ring or multiple rings (fused) and multiple carbon atoms, but having no ring heteroatoms. The term “cycloalkane” includes cyclic olefins such as cyclohexene. Examples of monocarbocyclic groups include, for example, cyclopropane, cyclobutane, cyclohexane, cyclopentane, cyclooctane, cyclopentene, cyclohexene, and the like. Examples of the fused cycloalkane system include bicyclohexane, bicyclopentane, bicyclooctane, and the like. The “cycloalkane” mentioned in the present invention includes, but is not limited to,

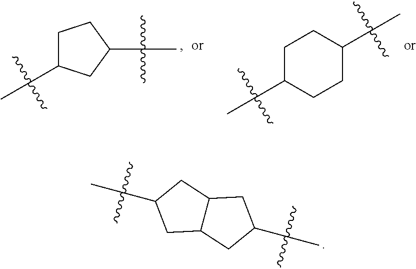 [SMILES: *C1CCC(*)C1 ; *C1CCC(*)CC1 ; *C1CC2CC(*)CC2C1]

The “heterocycloalkane” mentioned in the present invention refers to a saturated ring or a non-aromatic partially saturated divalent ring with a single ring or multiple rings (fused) containing at least one heteroatom; wherein the heteroatom refers to nitrogen atom, oxygen atom, sulfur atom, etc. Examples of heterocycloalkanes of monoheterocycloalkane systems are oxetane, azetidine, pyrrolidine, 2-oxo-pyrrolidine, tetrahydrofuran, tetrahydro-thiophene, pyrazolidine, imidazolidine, thiazolidine, piperidine, tetrahydropyran, tetrahydrothiopyran, piperazine, morpholine, thiomorpholine, 1,1-dioxo-thiomorpholine, azepane, diazepane, etc. Examples of fused heterocycloalkane systems include 8-aza-bicyclo[3.2.1]octane, quinuclidine, 8-oxa-3-aza-bicyclo[3.2.1]octane, 9-aza-bicyclo[3.3.1]nonane, etc. The term “heterocycloalkane” also includes the case where an aromatic ring is fused with a non-aromatic ring to form a partially saturated ring containing at least one heteroatom, in which the attachment point may be located at a non-aromatic carbon atom, an aromatic carbon atom or a heteroatom. The “heterocycloalkanes” mentioned in the present invention include, but are not limited to,

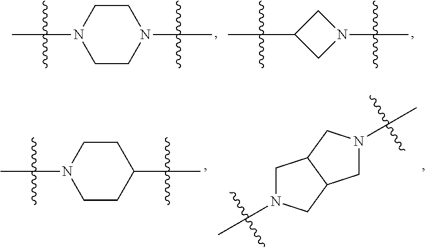 [SMILES: *N1CCN(*)CC1 ; *C1CN(*)C1 ; *C1CCN(*)CC1 ; *N1CC2CN(*)CC2C1]

etc.

The “spiro ring” mentioned in the present invention refers to a saturated or non-aromatic partially saturated bivalent ring formed by linking two or more rings having multiple carbon atoms and no ring heteroatom in the manner of spiro compound. Examples of spiro ring systems include

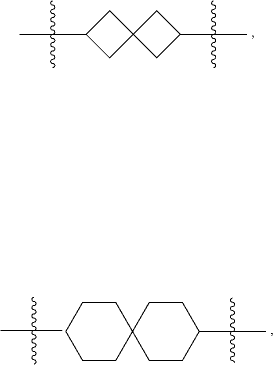

etc.

The “spiro heterocycle” mentioned in the present invention refers to a saturated ring or a non-aromatic partially saturated bivalent ring formed by linking two or more rings containing at least one heteroatom in the manner of spiro compound; and examples of spiro heterocyclic systems include

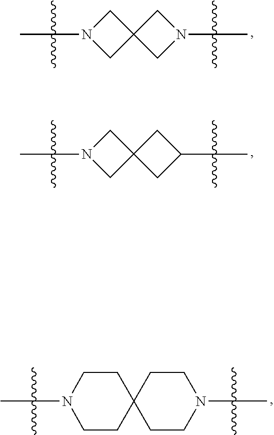 [SMILES: *N1CC2(C1)CN(*)C2 ; *C1CC2(C1)CN(*)C2 ; *N1CCC2(CC1)CCN(*)CC2]

etc.

The “bridged ring” mentioned in the present invention refers to a saturated or non-aromatic partially saturated bivalent ring formed by bridging multiple rings having multiple carbon atoms and no ring heteroatom. Examples of bridge-ring systems include

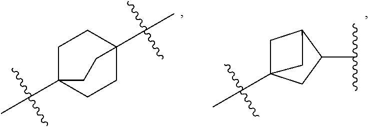 [SMILES: *C12CCC(*)(CC1)CC2 ; *C1CC2(*)CC1C2]

etc.

The “bridged heterocycle” mentioned in the present invention refers to a saturated ring or a non-aromatic partially saturated divalent ring formed by bridging multiple rings containing at least one heteroatom; examples of bridged heterocyclic systems include

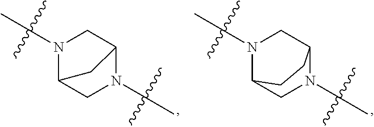 [SMILES: *N1CC2CC1CN2* ; *N1CC2CCC1CN2*]

etc.

The term “unsaturated” in the present invention refers to that a group or molecule contains a carbon-carbon double bond, carbon-carbon triple bond, carbon-oxygen double bond, carbon-sulfur double bond, carbon-nitrogen triple bond, etc.

The “alkenyl” mentioned in the present invention refers to a straight-chain or branched-chain hydrocarbon group with at least one vinyl unsaturated site (>C═C<). For example, C_{a-b} alkenyl refers to an alkenyl group having a to b carbon atoms and is intended to include, for example, vinyl, propenyl, isopropenyl, 1,3-butadienyl, and the like.

The “alkenylene” mentioned in the present invention refers to a straight-chain or branched divalent carbon chain having at least one carbon-carbon double bond. C_{a-b} alkenylene refers to an alkenylene group having a to b carbon atoms.

The “alkynyl” mentioned in the present invention refers to a straight-chain monovalent hydrocarbon group or a branched chain monovalent hydrocarbon group containing at least one triple bond. The term “alkynyl” is also intended to include those hydrocarbyl groups having one triple bond and one double bond. For example, C₂₋₆ alkynyl is intended to include ethynyl, propynyl, and the like.

The “alkynylene” mentioned in the present invention refers to a straight-chain divalent hydrocarbon group or a branched chain divalent hydrocarbon group containing at least one triple bond. C_{a-b} alkynylene refers to an alkynylene group having a to b carbon atoms.

The “heterocycloalkyl” mentioned in the present invention refers to a saturated ring or a non-aromatic partially saturated ring with a single ring or multiple rings (fused, bridged, spiro) containing at least one heteroatom; wherein heteroatom refers to nitrogen atom, oxygen atom, sulfur atom, etc. It usually represents a monovalent saturated or partially unsaturated monocyclic or polycyclic ring system with multiple ring atoms, which contains 1, 2 or 3 ring heteroatoms selected from N, O and S, and the remaining ring atoms are carbon atoms. Examples of heterocycloalkyl of monoheterocycloalkyl system are oxetanyl, azetidinyl, pyrrolidinyl, 2-oxo-pyrrolidin-3-yl, tetrahydrofuranyl, tetrahydrothienyl, pyrazolidinyl, imidazolidinyl, thiazolidinyl, piperidinyl, tetrahydropyranyl, tetrahydrothiopyranyl, piperazineheptyl, etc. Examples of heterocycloalkyl of fused heterocycloalkyl system include 8-aza-bicyclo[3.2.1]octyl, quinuclidinyl, 8-oxa-3-aza-bicyclo[3.2.1]octyl, 9-aza-bicyclo[3.3.1]nonyl, etc. Examples of heterocycloalkyl of bridged heterocycloalkyl system include

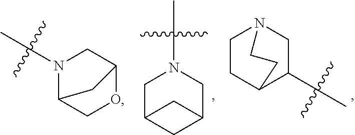

and the like. Examples of heterocycloalkyl of spiroheterocycloalkyl system include

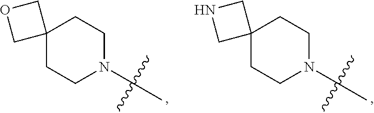 [SMILES: *N1CCC2(CC1)COC2 ; *N1CCC2(CC1)CNC2]

etc. Examples of partially saturated heterocycloalkyl are dihydrofuryl, imidazolinyl, tetrahydropyridyl or dihydropyranyl, and the like. The term “heterocycloalkyl” also includes the case where an aromatic ring is fused with a non-aromatic ring to form a partially saturated cyclic group containing at least one heteroatom, in which the attachment point can be located at a non-aromatic carbon atom, an aromatic carbon atom or a heteroatom; and examples include

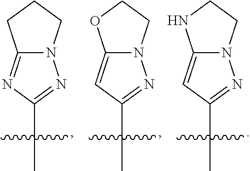

The “aromatic ring” mentioned in the present invention refers to an aromatic hydrocarbon group having multiple carbon atoms. Aryl usually refers to a monocyclic, bicyclic or tricyclic aryl group having multiple carbon atoms. Furthermore, the term “aryl” as used herein refers to an aromatic substituent that may be a single aromatic ring, or multiple aromatic rings fused together. Non-limiting examples include phenyl, naphthyl or tetrahydronaphthyl.

The “aromatic heterocycle” mentioned in the present invention refers to an aromatic unsaturated ring containing at least one heteroatom; wherein the heteroatom refers to nitrogen atom, oxygen atom, sulfur atom, etc. Aromatic heterocycles generally include aromatic monocyclic or bicyclic hydrocarbons that contain multiple ring atoms, one or more of which are heteroatoms selected from O, N, and S. Preferably, there are one to three heteroatoms. Heterocyclic aryl represents, for example: pyridyl, indolyl, quinoxalinyl, quinolyl, isoquinolinyl, benzothienyl, benzofuranyl, benzothienyl, benzothienyl, benzopyranyl, benzothiopyranyl, furyl, pyrrolyl, thiazolyl, oxazolyl, isoxazolyl, triazolyl, tetrazolyl, pyrazolyl, imidazolyl, thienyl, oxadiazolyl, benzimidazolyl, benzothiazolyl, benzoxazolyl.

The “halogen” mentioned in the present invention refers to fluorine, chlorine, bromine or iodine.

The “halogen-substituted alkyl” mentioned in the present invention means that one or more hydrogen atoms in alkyl are substituted by halogen; for example, “halogen-substituted C₁₋₄ alkyl”, which is an alkyl containing 1 to 4 carbon atoms in which the hydrogen atoms are substituted by one or more halogen atoms; for further example, monofluoromethyl, bisfluoromethyl, and trifluoromethyl.

The “halogen-substituted alkenyl” mentioned in the present invention means that one or more hydrogen atoms in alkenyl are substituted by halogen; for example, “halogen-substituted C₂₋₆ alkenyl”, which is an alkenyl containing 2 to 6 carbon atoms in which the hydrogen atoms are substituted by one or more halogen atoms; for further example, monofluorovinyl, bifluorovinyl, and trifluoropropenyl.

The “halogen-substituted alkynyl” mentioned in the present invention means that one or more hydrogen atoms in alkynyl are substituted by halogen; for example, “halogen-substituted C₂₋₆ alkynyl”, which is an alkynyl containing 2 to 6 carbon atoms in which the hydrogen atoms are substituted by one or more halogen atoms; for further example, monofluoroethynyl, bisfluoroethynyl, and trifluoropropynyl.

In the present invention, “—OR”, “—N(R)₂” and the like mean that the R group is connected to the oxygen atom or the nitrogen atom via a single bond.

In the present invention, “═O” means that the oxygen atom replaces two hydrogen atoms in a molecule through a double bond.

In the present invention, “—C(O)R”, “—S(O)₂R” and the like mean that the oxygen atom is connected with the carbon atom or the sulfur atom via a double bond, and the R group is connected with the oxygen atom or the sulfur atom via a single bond. For another example, “—S(O)(NH)R” means that the oxygen atom and the nitrogen atom are connected to the sulfur atom via double bonds, and the R group is connected to the sulfur atom via a single bond.

In the present invention, “ - - - ” and

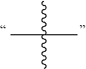

in a group are used to describe a position substituted by the group.

The “deuterated compound” of the present invention means that one or more hydrogen atoms in a molecule or group are replaced by deuterium atoms, in which the proportion of deuterium atoms is greater than the abundance of deuterium in nature.

The term “pharmaceutically acceptable” means that carrier, vehicle, diluent, excipient, and/or formed salt is generally chemically or physically compatible with the other ingredients constituting a pharmaceutical dosage form, and is physiologically compatible with a receptor.

The terms “salt” and “pharmaceutically acceptable salt” refer to an acidic and/or basic salt formed between the above-mentioned compound or stereoisomer thereof and an inorganic and/or organic acid and base, and also include zwitterionic salt (inner salt), also include quaternary ammonium salt, such as alkylammonium salt. These salts can be obtained directly from the final isolation and purification of the compound. They can also be obtained by appropriately mixing the above compound or stereoisomer thereof with a certain amount of acid or base (e.g., with equivalent amount). These salts may form a precipitate in a solution and be collected by filtration, or may be recovered after evaporation of solvent, or may be obtained by reacting in an aqueous medium and then freeze-drying.

The term “prevention” comprises inhibiting and delaying the onset of a disease, and comprises not only prevention before the disease develops, but also prevention of its recurrence after treatment.

The term “treatment” means reversing, alleviating or eliminating a disorder or condition to which such term applies or the progression of one or more symptoms of such disorder or condition.

In certain embodiments, one or more compounds of the present invention may be used in combination with each other. The compounds of the present invention can also be optionally used in combination with any other active agent to prepare drugs or pharmaceutical compositions for regulating cell function or treating diseases. If a group of compounds is used, the compounds can be administered to a subject simultaneously, separately, or sequentially.

Obviously, according to the above contents of the present invention, according to the common technical knowledge and common means in the field, without departing from the above basic technical idea of the present invention, various other forms of modifications, replacements or changes can also be made.

The above contents of the present invention will be further described in detail below through examples. However, this should not be understood to mean that the scope of the above subject matter of the present invention is limited to the following examples. All technologies implemented based on the above contents of the present invention belong to the scope of the present invention.

### DETAILED DESCRIPTION OF THE INVENTION

The known starting materials of the present invention could be synthesized by methods known in the art, or could be purchased from companies such as Energy Chemical, Chengdu Kelong Chemical, Accela ChemBio Co., Ltd, J&K Scientific, etc.

When there was no special instructions in the examples, the reaction was carried out in nitrogen atmosphere. When there was no special explanation in the examples, the solution was an aqueous solution. When there was no special instructions in the examples, the reaction temperature was room temperature. The room temperature was the most suitable reaction temperature, which was 20° C. to 30° C. When there was no special explanation in the examples, M referred to mol per liter.

The structure of compound was determined by nuclear magnetic resonance (NMR) and mass spectrometry (MS). NMR shifts (6) were given in unit of 10⁻⁶ (ppm). NMR was measured using (Bruker AvanceIII 400 and Bruker Avance 600) nuclear magnetic instruments. The measurement solvents were deuterated dimethyl sulfoxide (DMSO-d6), deuterated chloroform (CDCl₃), and deuterated methanol (Methanol-d4), and the internal standard was tetramethylsilane (TMS). LC-MS measurement was performed using a Shimadzu liquid-chromatography-mass spectrometry instrument (Shimadzu LC-MS 2020 (ESI)). HPLC measurement was performed using a Shimadzu high-pressure liquid chromatograph (Shimadzu LC-20A). MPLC (medium-pressure preparative chromatography) was performed using a Gilson GX-281 reversed phase preparative chromatograph. Thin layer chromatography silica gel plates were Yantai Huanghai HSGF254 or Qingdao GF254 silica gel plates, and the specifications of thin layer chromatography for the separation and purification of products were 0.4 mm to 0.5 mm. Column chromatography generally used Yantai Huanghai Silica Gel 200-300 mesh silica gel as carrier.

Pd(dppf)Cl₂: [1,1′-bis(diphenylphosphino) ferrocene] palladium dichloride; TMSCl: trimethylchlorosilane; Xphos: 2-bicyclohexylphosphine-2′,4′,6′-triisopropylbiphenyl; Pd₂(dba)₃: tris(dibenzylideneacetone)dipalladium; EDCI: 1-ethyl-3-(3-dimethylaminopropyl)carbodiimide; HOBt: hydroxybenzotriazole; Found: MS value of product when measured by LC-MS.

### Synthesis of Intermediates: Synthesis of Intermediate Compounds R_{1A-1}, R_{1A-2}, R_{1A-3}, R_{1A-4}, and R_{1A-5}

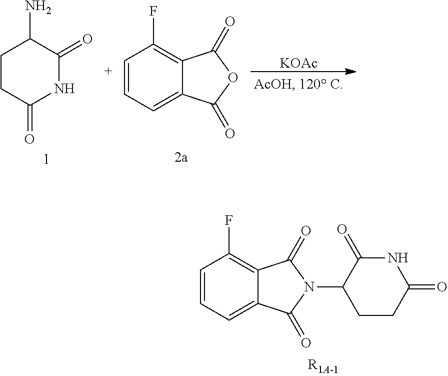

Compound 1 (1.00 mmol), Compound 2a (1.05 mmol), potassium acetate (1.20 mmol), and acetic acid (5 mL) were added to a 100 mL reaction bottle in sequence, heated to 120° C., stirred and reacted for 8 hours, and then water was added to quench the reaction (LC-MS monitoring). After extraction with saturated sodium chloride solution (25 mL) and ethyl acetate (3×25 mL), the organic phases were combined, the combined organic phase was dried over anhydrous sodium sulfate, and concentrated under reduced pressure to remove the solvent to obtain Compound R_{1A-1}. (C₁₃H₁₀FN₂O₄, [M+H]⁺ 277.1; found 277.2, yield 89.8%).

Referring to the synthesis method of Compound R_{1A-1}, the raw materials in the following table were used to replace Compound 2a, and other raw materials and operating methods were kept unchanged to obtain intermediate compounds R_{1A-2}, R_{1A-3}, R_{1A-4}, R_{1A-5}, R_{1A-6}.

| Compound | Structure and characterization | | |
|---|---|---|---|
| No. | data of compound | Raw material | Yield |
| R_{1A-2} | 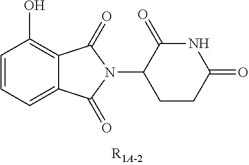 | 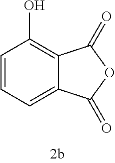 | 89.8% |
| | LC-MS: C₁₃H₁₁N₂O₅ [M + H]+ | | |
| | 275.1; found 275.2 | | |
| R_{1A-3} | 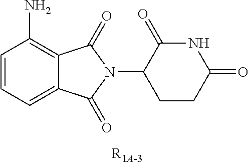 | 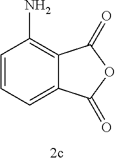 | 65.3% |
| | LC-MS: C₁₃H₁₂N₃O₄, [M + H]+ | | |
| | 274.1; found 274.3 | | |
| R_{1A-4} | 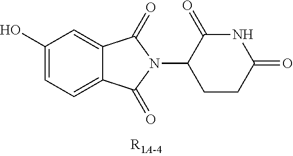 | 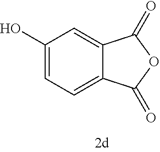 | 68.9% |
| | LC-MS: C₁₃H₁₁N₂O₅, [M + H]+ | | |
| | 275.1; found 275.2 | | |
| R_{1A-5} | 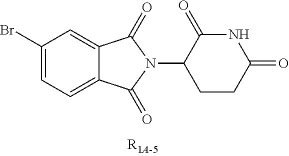 | 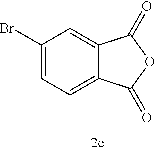 | 83.6% |
| | LC-MS: C₁₃H₁₀BrN₂O₄, [M + H]⁺ | | |
| | 337.0; found 337.1 | | |
| R_{1A-6} | 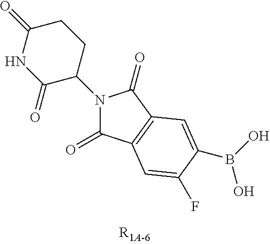 | 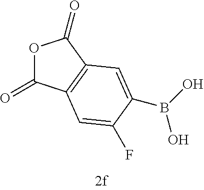 | 78.9% |
| | LC-MS: C₁₃H₁₁BFN₂O₆, [M + H]⁺ | | |
| | 321.2; found 321.1 | | |

### Synthesis of Intermediate: Synthesis of Intermediate R_{1A-7}

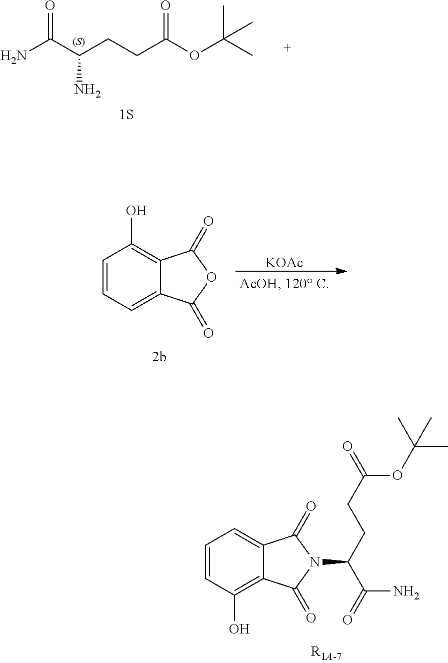

Compound 1S (1.00 mmol), Compound 2b (1.05 mmol), potassium acetate (1.20 mmol), and acetic acid (5 mL) were added to a 100 mL reaction bottle in sequence, heated to 120° C., stirred and reacted for 1 hour, and then water was added to quench the reaction (LC-MS monitoring). After extraction with saturated sodium chloride solution (25 mL) and ethyl acetate (3×25 mL), the organic phases were combined, the combined organic phase was dried over anhydrous sodium sulfate, and concentrated under reduced pressure to remove the solvent to obtain Compound R_{1A-7}. (C₁₇H₂₁N₂O₆, [M+H]⁺ 349.4; found 349.4, yield 49.6%).

### Synthesis of intermediates: Synthesis of intermediates R_{1B-1}, R_{1B-2}, R_{1B-3}, R_{1B-4}, R_{1B-5}, R_{1B-6}, R_{1B-7}

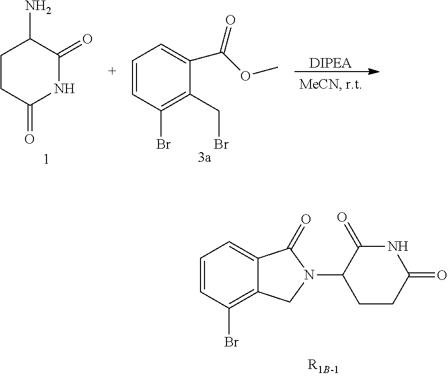

Compound 1 (1.00 mmol), Compound 3a (1.05 mmol), N,N-diisopropylethylamine (2.10 mmol), and acetonitrile (10 mL) were added in sequence to a 100 mL reaction bottle, reacted at room temperature for 8 hours, and then water was added to quench the reaction (LC-MS monitoring). After extraction with saturated sodium chloride solution (25 mL) and ethyl acetate (3×25 mL), the organic phases were combined, the combined organic phase was dried over anhydrous sodium sulfate, and concentrated under reduced pressure to remove the solvent to obtain Compound R_{1B-1} (C₁₃H₁₂BrN₂O₃, [M+H]⁺ 323.0; found 323.2, yield 90.3%).

Referring to the synthesis method of Compound R_{1B-1}, the raw materials in the following table were used to replace Compound 3a, and the other raw materials and operating methods remained unchanged, to obtain intermediate compounds R_{1B-2}, R_{1B-3}, R_{1B-4}, R_{1B-5}, R_{1B-6}, R_{1B-7}.

| Compound | Structure and characterization | | |
|---|---|---|---|
| No. | data of compound | Raw material | Yield |
| R_{1B-2} | 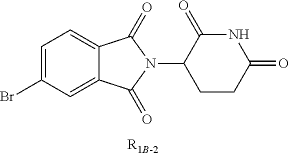 | 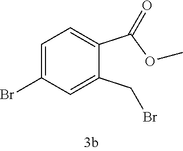 | 91.6% |
| | LC-MS: C₁₃H₁₂BrN₂O₃, [M + H]⁺ | | |
| | 323.0; found 323.1 | | |
| R_{1B-3} | 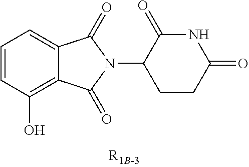 | 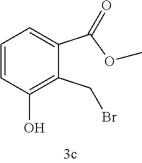 | 89.9% |
| | LC-MS: C₁₃H₁₃N₂O₄, [M + H]⁺ | | |
| | 261.1; found 261.3 | | |
| R_{1B-4} | 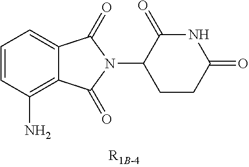 | 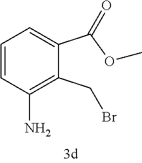 | 78.9% |
| | LC-MS: C₁₃H₁₄N₃O₃, [M + H]⁺ | | |
| | 260.1; found 260.2 | | |
| R_{1B-5} | 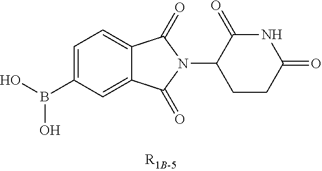 | 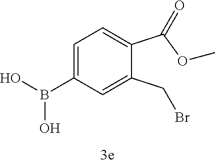 | 63.1% |
| | LC-MS: C₁₃H₁₄BN₂O₅, [M + H]⁺ | | |
| | 289.1; found 289.2 | | |
| R_{1B-6} | 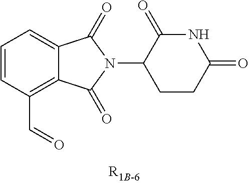 | 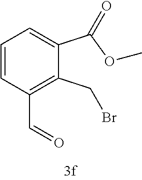 | 47.6% |
| | LC-MS: C₁₄H₁₃N₂O₄, [M + H]⁺ | | |
| | 273.1; found 273.3 | | |
| R_{1B-7} | 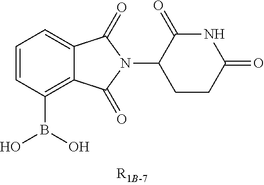 | 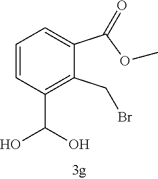 | 60.9% |
| | LC-MS: C₁₃H₁₄BN₂O₅, [M + H]⁺ | | |
| | 289.1; found 289.2 | | |

### Synthesis of Intermediates: Synthesis of Intermediate Compounds R_{2-A1}, R_{2-A2}, R_{2-B1}, R_{2-B1}

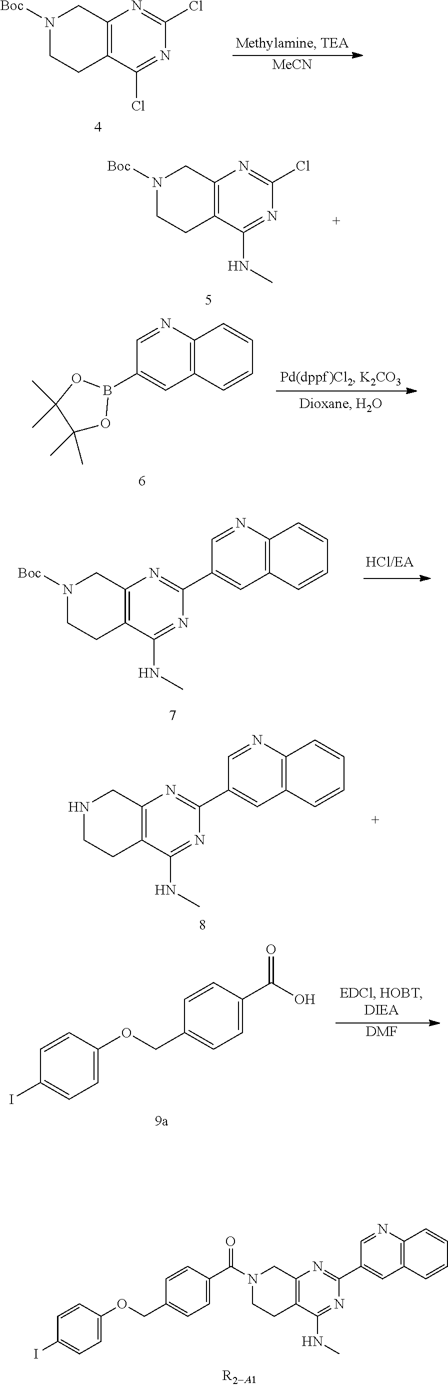

### Step 1. Synthesis of Compound 5

Compound 4 (1.00 mmol), methylamine (1.15 mmol), triethylamine (2 mL) and acetonitrile (10 mL) were added to a 50 mL reaction bottle, reacted at room temperature for 8 hours, then water was added to quench the reaction (LC-MS monitoring). After extraction with saturated sodium chloride solution (25 mL) and ethyl acetate (3×25 mL), the organic phases were combined, the combined organic phase was dried over anhydrous sodium sulfate, and concentrated under reduced pressure to remove the solvent to obtain Compound 5 (LC-MS: C₁₃H₂₀ClN₄O₂, [M+H]⁺ 299.1; found 299.3, crude product).

### Step 2. Synthesis of Compound 7

Under nitrogen protection, Compound 5 (1.00 mmol), Compound 6 (1.05 mmol), potassium carbonate (2.00 mmol), 1,4-dioxane and water (4 mL/1 mL) and Pd(dppf)Cl₂ (0.25 mol %) were added in sequence to a 50 mL reaction bottle. The reaction system was sealed, the reaction was carried out at 80° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound 7 (LC-MS: C₂₂H₂₆N₅O₂, [M+H]⁺ 392.2; found 392.4, yield 53.6%).

### Step 3. Synthesis of Compound 8

Compound 7 was dissolved in a mixed solution of 6N hydrochloric acid and ethyl acetate (5 mL) in a 50 mL reaction bottle, stirred and reacted at room temperature for 0.5 hour, and concentrated under reduced pressure to remove the solvent to obtain Compound 8 (LC-MS: C₁₇H₁₈N₅, [M+H]⁺ 292.2; found 292.1, crude product).

### Step 4. Synthesis of Compound R_{2-A1}

Compound 9a (1.00 mmol), EDCI (1.05 mmol), HOBt (1.05 mmol) and N,N-dimethylformamide (2 mL) were added in sequence to a 50 mL reaction bottle. After the temperature of the reaction system dropped to 0° C., Compound 8 (1.05 mmol) was added. The reaction was carried out under ice bath conditions and stirring for 1 hour, and then quenched (LC-MS monitoring). The extraction was carried out with saturated sodium chloride solution (10 mL) and ethyl acetate (3×25 mL). The organic phases were combined, and the combined organic phase was dried over anhydrous sodium sulfate, evaporated to remove the solvent, purified by medium-pressure preparative chromatography, and concentrated under reduced pressure to remove the solvent to obtain Compound R_{2-A1} (LC-MS: C₃₁H₂₇IN₅O₂, [M+H]⁺ 628.1; found 628.3, yield 86.5%).

Referring to the synthesis method of Compound R_{2-A1}, the raw materials in the following table were used to replace Compound 9a, and the other raw materials and operating methods remained unchanged, to obtain intermediate Compound R_{2-A2}.

| Com- | | | |
|---|---|---|---|
| pound | Structure and characeterization data of | | |
| No. | compound | Raw material | Yield |
| R_{2-A2} | 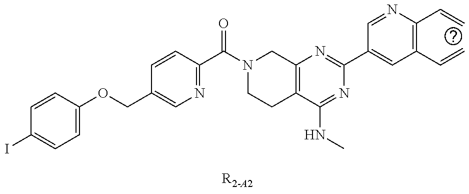 | 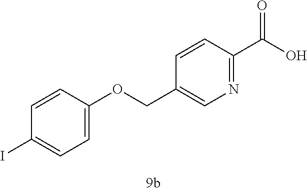 | 84.3% |
| | LC-MS: C₃₀H₂₆IN₆O₂, [M + H]⁺ 629.1; found | | |
| | 629.3 | | |

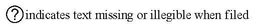

Referring to the synthesis method of Compound R_{2-A1}, the raw materials in the following table were used to replace Compound 9a, and the other raw materials and operating methods remained unchanged, to obtain intermediate compounds R_{2-B1} and R_{2-B2}.

| Com- | | | |
|---|---|---|---|
| pound | Structure and characterization data of | | |
| No. | compound | Raw material | Yield |
| R_{2-B1} | 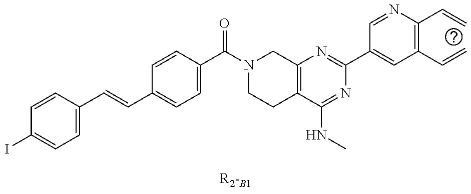 | 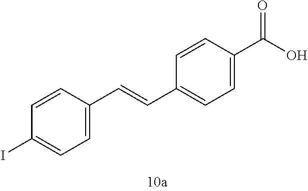 | 78.6% |
| | LC-MS: C₃₂H₂₇IN₅O, [M + H]⁺ 624.1; found | | |
| | 624.2 | | |
| R_{2-B2} | 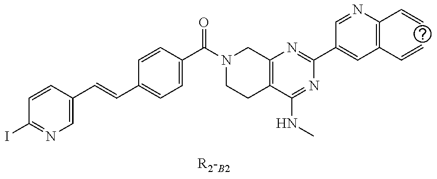 | 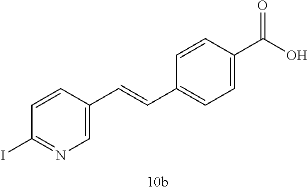 | 59.8% |
| | LC-MS: C₃₁H₂₆IN₆O, [M + H]⁺ 625.1; found | | |
| | 625.2 | | |

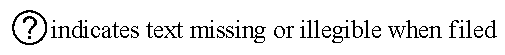

### Synthesis of Intermediates: Synthesis of Intermediate Compounds R_{2-C1}, R_{2-C2}, R_{2-C3}, R_{2-C4}, R_{2-C5}, R_{2-D1}, R_{2-D2}, R_{2-D3}, R_{2-D4}

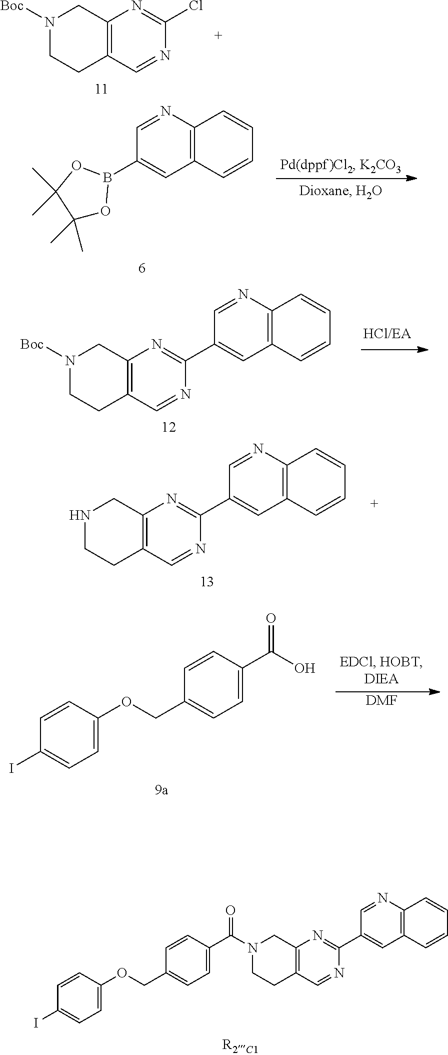

### Step 1. Synthesis of Compound 12

Under nitrogen protection, Compound 11 (1.00 mmol), Compound 6 (1.05 mmol), potassium carbonate (2.00 mmol), 1,4-dioxane and water (4 mL/1 mL) and Pd(dppf)Cl₂ (0.25 mol %) were added in sequence to a 50 mL reaction bottle. The reaction system was sealed, the reaction was carried out under stirring at 80° C. for 2 hours, and then the reaction was quenched (LC-MS monitoring). The extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed after concentration under reduced pressure to obtain Compound 12 (LC-MS: C₂₁H₂₃N₄O₂, [M+H]⁺ 363.2; found 363.3, yield 61.8%).

### Step 2. Synthesis of Compound 13

Compound 12 was dissolved in a mixed solution of 6N hydrochloric acid and ethyl acetate (5 mL) in a 50 mL reaction bottle, stirred and reacted at room temperature for 0.5 hour, and concentrated under reduced pressure to remove the solvent to obtain Compound 13 (LC-MS: C₁₆H₁₅N₄, [M+H]⁺ 263.1; found 263.2, crude product).

### Step 3. Synthesis of Compound R_{2-C1}

Compound 9a (1.00 mmol), EDCI (1.05 mmol), HOBT (1.05 mmol) and N,N-dimethylformamide (2 mL) were added in sequence to a 50 mL reaction bottle. After the temperature of the reaction system dropped to 0° C., Compound 13 (1.00 mmol) was added, the reaction was carried out under ice bath conditions and stirring for 1 hour, and then the reaction was quenched (LC-MS monitoring). The extraction was carried out with saturated sodium chloride solution (10 mL) and ethyl acetate (3×25 mL), the organic phases were combined, the combined organic phase was dried over anhydrous sodium sulfate, evaporated to remove the solvent, purified by medium-pressure preparative chromatography, and concentrated under reduced pressure to remove the solvent to obtain Compound R_{2-C1} (LC-MS: C₃₀H₂₄IN₄O₂, [M+H]⁺ 599.1; found 599.3, yield 73.2%).

Referring to the synthesis method of Compound R_{2-C1}, the raw materials in the following table were used to replace Compound 9a, and the other raw materials and operating methods remained unchanged to obtain intermediate compounds R_{2-C2}, R_{2-C3}, R_{2-C4} and R_{2-C5}.

| Com- | | | |
|---|---|---|---|
| pound | Structure and characterization data of | | |
| No. | compound | Raw material | Yield |
| R_{2-C2} | 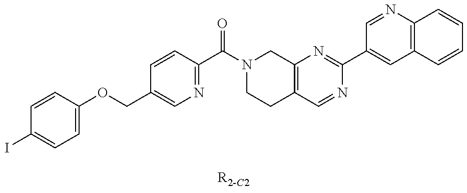 | 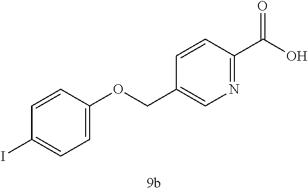 | 58.2% |
| | LC-MS: C₂₉H₂₃IN₅O₂, [M + H]⁺ 600.1; | | |
| | found 600.2 | | |
| R_{2-C3} | 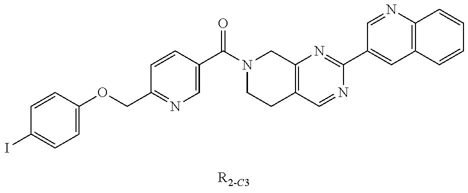 | 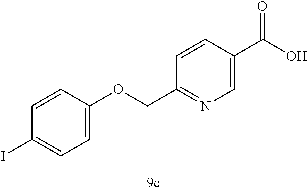 | 59.6% |
| | LC-MS: C₂₉H₂₃IN₅O₂, [M + H]⁺ 600.1; | | |
| | found 600.3 | | |
| R_{2-C4} | 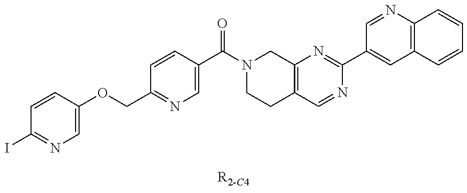 | 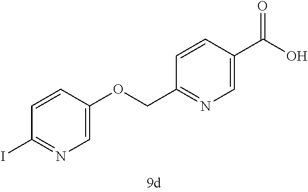 | 43.8% |
| | LC-MS: C₂₈H₂₂IN₆O₂, [M + H]⁺ 601.1; | | |
| | found 601.3 | | |
| R_{2-C5} | 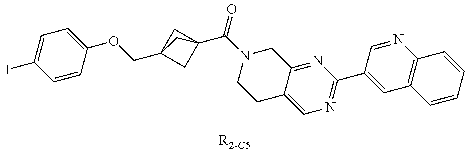 | 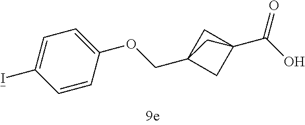 | 51.2% |
| | LC-MS: C₂₉H₂₆IN₄O₂, [M + H]⁺ 589.1; | | |
| | found 589.2 | | |

Referring to the synthesis method of Compound R_{2-C1}, the raw materials in the following table were used to replace Compound 9a, and the other raw materials and operating methods remained unchanged to obtain intermediate compounds R_{2-D1}, R_{2-D2}, R_{2-D3} and R_{2-D4}.

| Com- | | | |
|---|---|---|---|
| pound | Structure and characterization data of | | |
| No. | compound | Raw material | Yield |
| R_{2-D1} | 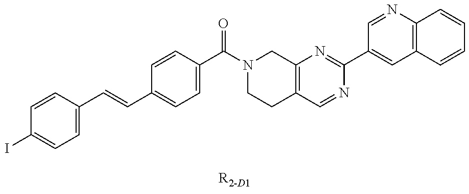 | 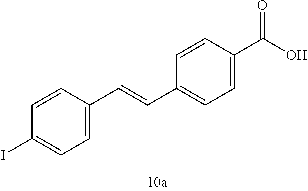 | 63.8% |
| | LC-MS: C₃₁H₂₄IN₄O, [M + H]⁺ 595.1; found | | |
| | 595.2 | | |
| R_{2-D2} | 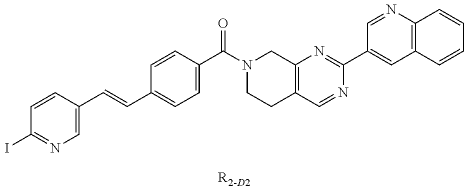 | 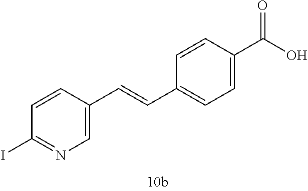 | 51.9% |
| | LC-MS: C₃₀H₂₃IN₅O, [M + H]⁺ 596.1; found | | |
| | 596.3 | | |
| R_{2-D3} | 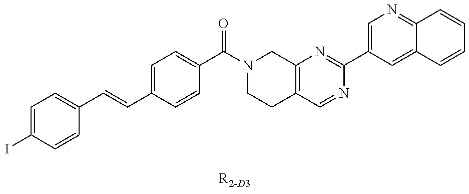 | 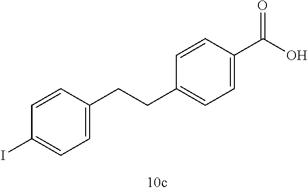 | 67.8% |
| | LC-MS: C₃₁H₂₆IN₄O, [M + H]⁺ 597.1; found | | |
| | 597.2 | | |
| R_{2-D4} | 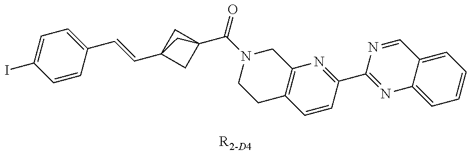 | 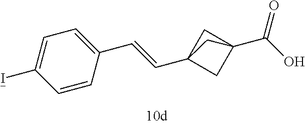 | 59.2% |
| | LC-MS: C₃₀H₂₆IN₄O, [M + H]⁺ 585.1; found | | |
| | 585.2 | | |

### Example 1. Synthesis of Compound A of the Present Invention

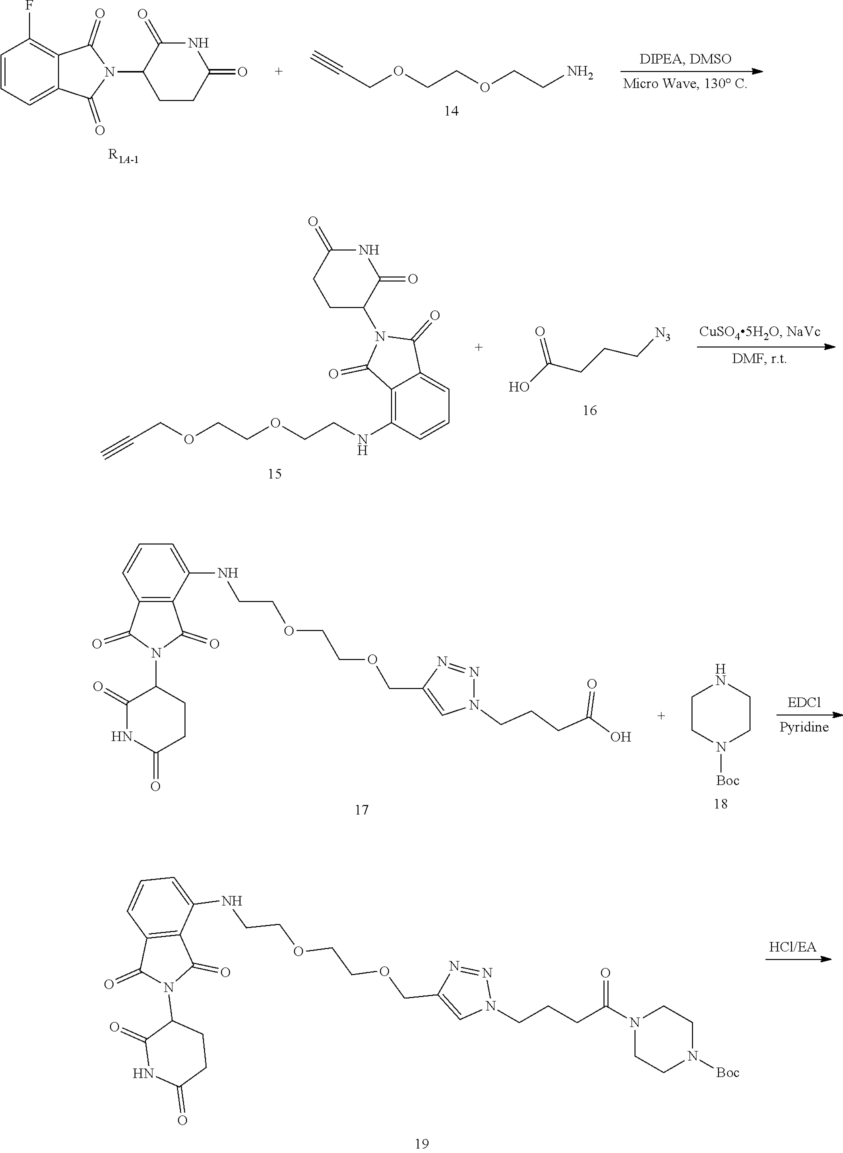

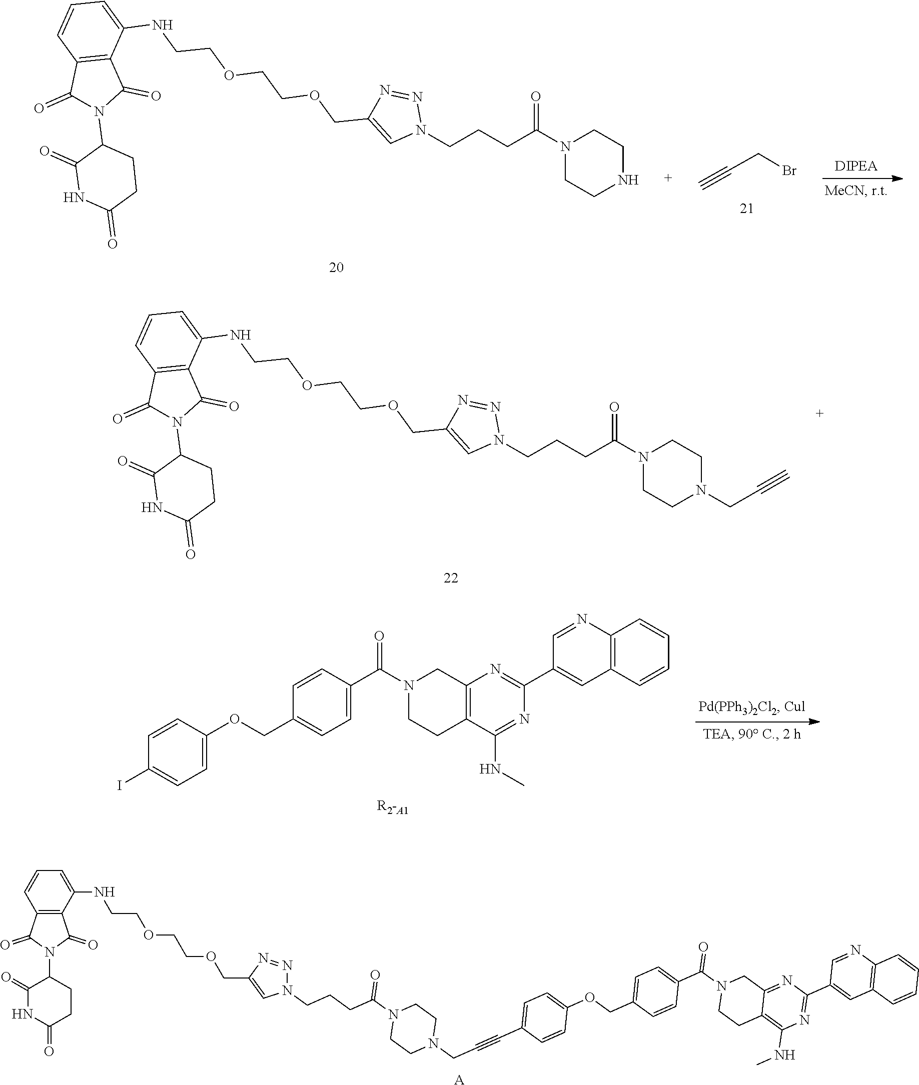

### Step 1. Synthesis of Compound 15

Compound R_{1A-1} (1.15 mmol), Compound 14 (1.15 mmol), N,N-diisopropylethylamine (2.80 mmol) and DMSO (5 mL) were added into a 50 mL reaction bottle, heated by a microwave reactor to 130° C. and reacted for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 15 (C₂₀H₂₂N₃O₆, [M+H]⁺ 400.2; found 400.3, crude product).

### Step 2. Synthesis of Compound 17

Compound 15 (0.85 mmol), Compound 16 (0.90 mmol) and N,N-dimethylformamide (2 mL) were added into a 50 mL reaction bottle. After the solution was clear, copper sulfate pentahydrate (1.75 mmol) and NaVc (0.90 mmol) were added in sequence, reacted at room temperature until the end of the reaction (LC-MS monitoring), the solid was filtered out, and the reaction solution was concentrated under reduced pressure to obtain Compound 17 (C₂₄H₂₉N₆O₈, [M+H]⁺ 529.2; found 529.1, yield 91.8%).

### Step 3. Synthesis of Compound 19

Compound 17 (0.75 mmol), EDCI (0.80 mmol) and pyridine (2 mL) were added in sequence to a 50 mL reaction bottle. After the temperature of the reaction system dropped to 0° C., Compound 18 (0.75 mmol) was added. The reaction was carried out under ice bath conditions and stirring for 0.5 hours, and then the reaction was quenched. Extraction was carried out with saturated sodium chloride solution (20 mL) and methylene chloride (3×15 mL). The organic phases were combined, and the combined organic phase was dried over anhydrous sodium sulfate and evaporated to dryness to remove the solvent, and purified by medium-pressure preparative chromatography, and the solvent was removed by concentration under reduced pressure to obtain Compound 19 (C₃₃H₄₅N₈O₉, [M+H]⁺ 697.3; found 697.4, yield 83.2%).

### Step 4. Synthesis of Compound 20

Compound 19 was dissolved in a mixed solution of 6N hydrochloric acid and ethyl acetate (5 mL) in a 50 mL reaction bottle, stirred and reacted at room temperature for 0.5 hours, and concentrated under reduced pressure to remove the solvent, to obtain Compound 20 (C₂₈H₃₇N₈O₇, [M+H]⁺597.3; found 597.2, crude product).

### Step 5. Synthesis of Compound 22

Compound 20 (0.55 mmol), Compound 21 (0.60 mmol), N,N-diisopropylethylamine (1.40 mmol) and acetonitrile (5 mL) were added into a 50 mL reaction bottle, stirred and reacted at room temperature for 2 hours, then added with water to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, and purified by medium-pressure preparative chromatography, and the solvent was removed by concentration under reduced pressure to obtain Compound 22 (C₃₁H₃₉N₈O₇, [M+H]⁺ 635.3; found 635.2, yield 68.2%).

### Step 6. Synthesis of Compound A

Under nitrogen protection, Compound 22 (0.25 mmol), Compound R_{2-A1} (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound A (42.8 mg, 37.76 μmol, purity 99.9%). LC-MS: C₆₂H₆₄N₁₃O₉, [M/2+H]⁺568.1; found 568.1. ¹H NMR (400 MHz, DMSO-d6) δ 11.10 (s, 1H), 9.82-9.74 (m, 1H), 9.31-9.21 (m, 1H), 8.22 (s, 1H), 8.12-8.05 (m, 2H), 7.88 (s, 1H), 7.72-7.70 (m, 1H), 7.67-7.58 (m, 5H), 7.53-7.49 (m, 3H), 7.15-7.11 (m, 1H), 7.09-7.04 (m, 2H), 7.01 (d, J=8.0 Hz, 1H), 6.60 (s, 1H), 5.23 (s, 2H), 5.08-5.03 (m, 1H), 4.76 (s, 2H), 4.52 (s, 2H), 4.45-4.34 (m, 4H), 3.98 (s, 2H), 3.72-3.58 (m, 8H), 3.47 (s, 3H), 3.32 (s, 3H), 3.10 (d, J=4.0 Hz, 3H), 2.93-2.85 (m, 1H), 2.67-2.57 (m, 3H), 2.53-2.50 (m, 4H), 2.39 (s, 2H), 2.06-2.03 (m, 2H). Purity >99%.

### Example 2. Synthesis of Compounds B1, B2 and B3 of the Present Invention

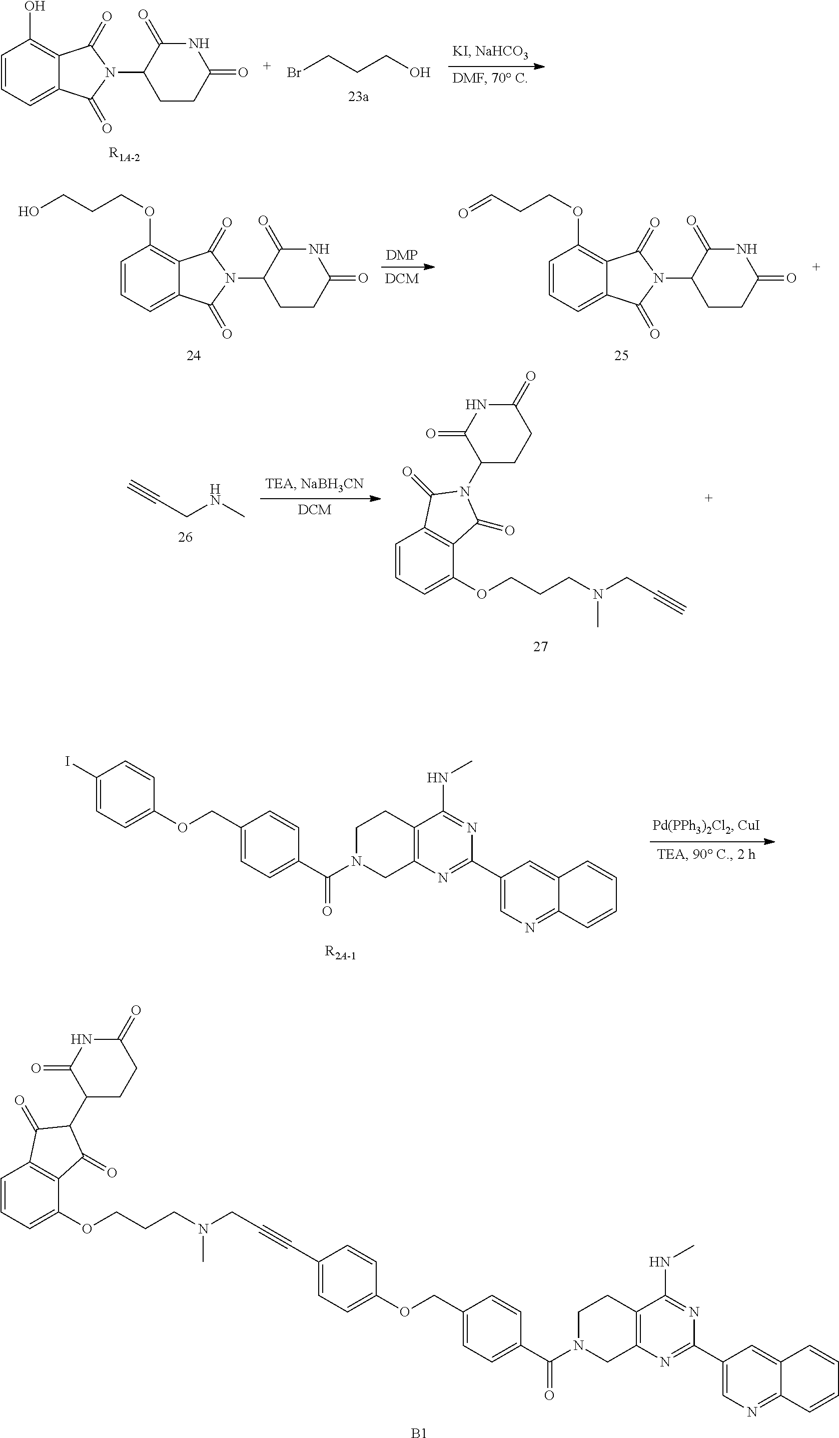

### Step 1. Synthesis of Compound 24

Compound R_{1A-2} (1.00 mmol), Compound 23a (2.25 mmol), potassium iodide (1.05 mmol), sodium bicarbonate (2.80 mmol) and N,N-dimethylformamide (5 mL) were added into a 50 mL reaction bottle, heated to 70° C., stirred and reacted for 8 hours, and then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 24 (C₁₆H₁₇N₂O₆, [M+H]⁺ 333.1; found 333.3, yield 59.8%).

### Step 2. Synthesis of Compound 25

Compound 24 (0.55 mmol), Dess-Martin Periodinane (1.20 mmol) and dichloroethane (5 mL) were added to a 50 mL reaction bottle, stirred and reacted at room temperature for 1 hour, and then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 25 (C₁₆H₁₅N₂O₆, [M+H]⁺ 331.1; found 331.2, yield 81.5%).

### Step 3. Synthesis of Compound 27

Compound 25 (0.50 mmol), Compound 26 (0.95 mmol), triethylamine (2 mL), sodium cyanoborohydride (1.02 mmol) and dichloroethane (5 mL) were added in sequence to a 50 mL reaction bottle, stirred and reacted at room temperature for 2 hours, and then the reaction was quenched (LC-MS monitoring). The solvent was evaporated to dryness, purification was carried out by medium-pressure preparative chromatography, and the solvent was removed by concentration under reduced pressure to obtain Compound 27 (C₂₀H₂₂N₃O₅, [M+H]⁺ 384.2; found 384.3, yield 68.9%).

### Step 4. Synthesis of Compound B1

Under nitrogen protection, Compound 27 (0.25 mmol), Compound R_{2-A1} (0.30 mmol), copper iodide (0.50 mmol), triethylamine (5 mL), and Pd(PPh₃)₂Cl₂ (0.25 mol %) were added in sequence to a 50 mL reaction bottle. The reaction system was sealed, the reaction was carried out at 90° C. under stirring for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to remove the solvent, separated by column chromatography (the volume ratio of the eluents used was petroleum ether/ethyl acetate=1:100 to 1:5), and the solvent was removed by concentration under reduced pressure to obtain Compound B1 (3.0 mg, 3.39 μmol, purity 89.7%). LC-MS: C₅₁H₄₇N₈O₇, [M/2+H]⁺442.4; found 422.6. ¹H NMR (400 MHz, Methanol-d4) δ 9.80-9.68 (m, 1H), 9.25-9.22 (m, 1H), 8.09-8.07 (m, 2H), 7.82-7.53 (m, 8H), 7.40-7.29 (m, 3H), 7.02-6.92 (m, 2H), 5.18 (s, 2H), 4.31 (s, 3H), 4.11 (s, 2H), 3.80-3.70 (m, 3H), 3.17 (s, 3H), 2.94 (s, 2H), 2.82-2.53 (m, 9H), 2.11-2.03 (m, 3H). Purity >85%.

According to the step 1 to step 4 of the synthesis method of Compound B1, the raw materials in the following table were used to replace Compound 23a, and the other raw materials and operating methods remained unchanged, to obtain Compounds B2 and B3.

| | | Raw | |
|---|---|---|---|
| Compound No. | Structure and characterization data of compound | material | Yield |
| B2 | 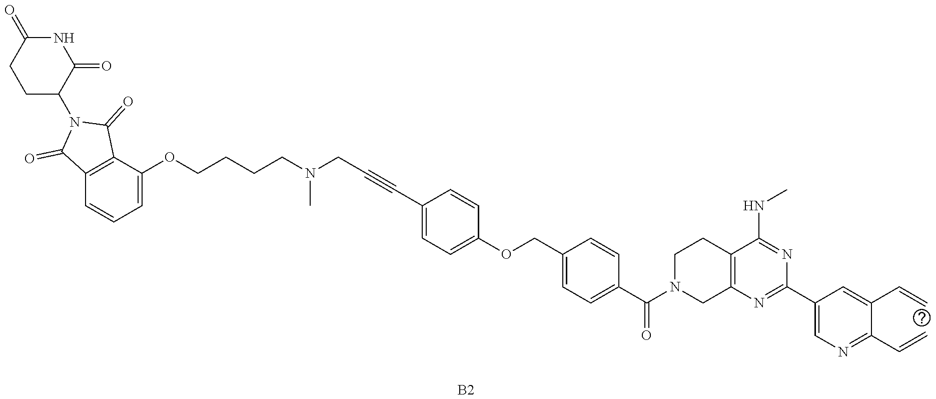 [SMILES: C=Cc1cc(-c2nc3c(c(NC)n2)CCN(C(=O)c2ccc(COc4ccc(C#CCN(C)CCCCOc5cccc6c5C(=O)N(C5CCC(=O)NC5=O)C6=O)cc4)cc2)C3)cnc1C=C ; "B2"] | 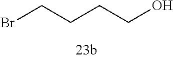 | 12.8% |
| | LC-MS: C₅₂H₄₉N₈O₇, [M/2 + H]⁺ 449.2; found 449.5. ¹H | | |
| | NMR (400 MHz, Methanol-d4) δ 9.78 (s, 1H), 9.54 (s, | | |
| | 1H), 8.29 (s, 1H), 8.23-8.21 (m, 1H), 8.07 (s, 1H), 7.88 (s, | | |
| | 1H), 7.74 (t, J = 8.0 Hz, 1H), 7.60 (q, J = 8.0 Hz, 4H), | | |
| | 7.43-7.39 (m, 2H), 7.37-7.32 (m, 2H), 6.91 (d, J = 8.3 Hz, | | |
| | 2H), 5.18 (s, 2H), 5.08-5.03 (m, 1H), 4.92 (s, 1H), 4.73 (s, | | |
| | 1H), 4.32-4.26 (m, 4H), 4.12 (s, 1H), 3.83 (s, 1H), 3.73- | | |
| | 3.67 (m, 1H), 3.47 (s, 1H), 3.25 (s, 3H), 3.03 (s, 3H), 2.83- | | |
| | 2.78 (m, 1H), 2.72-2.62 (m, 4H), 2.04 (s, 5H). | | |
| B3 | 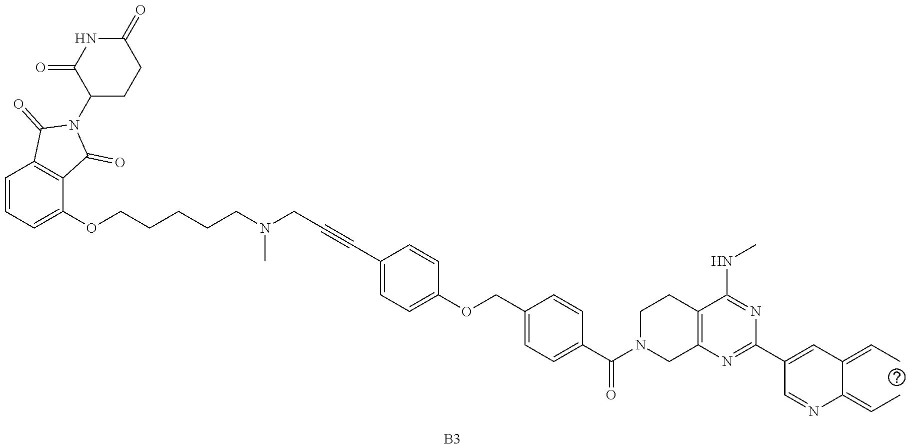 [SMILES: C/C=c1/cc(-c2nc3c(c(NC)n2)CCN(C(=O)c2ccc(COc4ccc(C#CCN(C)CCCCCOc5cccc6c5C(=O)N(C5CCC(=O)NC5=O)C6=O)cc4)cc2)C3)cn/c1=C/C ; "B3"] | 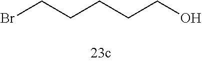 | 10.9% |
| | LC-MS: C₅₃H₅₁N₈O₇, [M/2 + H]⁺ 456.2; found 456.6. ¹H | | |
| | NMR (400 MHz, DMSO-d₆) δ 11.11 (s, 1H), 10.75-10.68 | | |
| | (m, 1H), 9.82-9.74 (m, 1H), 9.34-9.24 (m, 1H), 8.23 (s, | | |
| | 1H), 8.13 (s, 1H), 7.89 (s, 1H), 7.83-7.44 (m, 8H), 7.08 (d, | | |
| | J = 8.5 Hz, 2H), 5.23 (s, 2H), 5.09-5.06 (m, 1H), 4.76 (s, | | |
| | 1H), 4.56 (s, 1H), 4.32 (d, J = 4.8 Hz, 2H), 4.23 (t, J = 6.2 | | |
| | Hz, 2H), 3.84 (s, 1H), 3.21-3.16 (m, 2H), 3.10 (d, J = 3.8 | | |
| | Hz, 3H), 2.92-2.86 (m, 4H), 2.68-2.56 (m, 4H), 2.08-2.01 | | |
| | (m, 1H), 1.82-1.80 (m, 4H), 1.55-1.52 (m, 2H). | | |

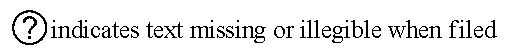

### Example 3. Synthesis of Compounds C1-1, C1-2 and C1-3 of the Present Invention

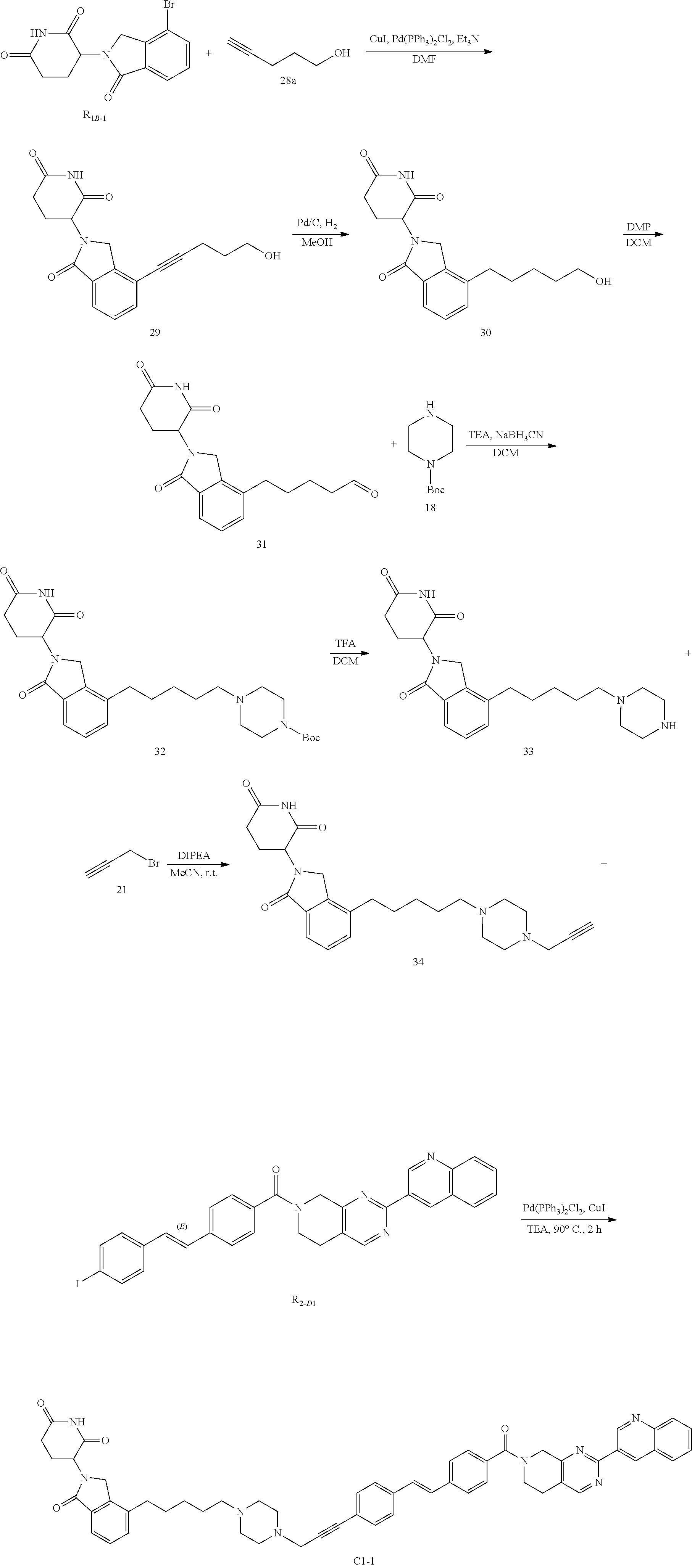

### Step 1. Synthesis of Compound 29

Under nitrogen protection, Compound R_{1B-1} (1.00 mmol), Compound 28a (1.05 mmol), copper iodide (2.00 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of eluents used was petroleum ether/ethyl acetate=1:100 to 1:5), and the solvent was removed by concentration under reduced pressure to obtain Compound 29 (C₁₈H₁₉N₂O₄, [M+H]⁺ 327.1; found 327.2, yield 68.5%).

### Step 2. Synthesis of Compound 30

Compound 29 (0.65 mmol), methanol (5 mL), and palladium-on-carbon (30.00 mg) were added in sequence to a 50 mL reaction bottle. The reaction system was replaced with hydrogen, maintained at an atmospheric pressure hydrogen atmosphere, and the reaction was carried out under stirring at room temperature for 3 hours (LC-MS monitoring). Filtration was carried to remove the palladium-on-carbon, washing was carried out with methanol (2×20 mL), the filtrate was collected, and concentrated under reduced pressure to remove the solvent to obtain Compound 30 (C₁₈H₂₃N₂O₄, [M+H]⁺ 331.2; found 331.1, crude product).

### Step 3. Synthesis of Compound 31

Compound 30 (0.55 mmol), Dess-Martin Periodinane (1.50 mmol) and dichloroethane (5 mL) were added to a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 31 (C₁₈H₂₁N₂O₄, [M+H]⁺ 329.2; found 329.1, yield 82.1%).

### Step 4. Synthesis of Compound 32

Compound 31 (0.45 mmol), Compound 18 (0.50 mmol), triethylamine (2 mL), sodium cyanoborohydride (0.55 mmol) and dichloroethane (5 mL) were added in sequence to a 50 mL reaction bottle. The reaction was carried out under stirring at room temperature for 2 hours, and then the reaction was quenched (LC-MS monitoring). The solvent was removed by evaporation to dryness, and purification was carried by medium-pressure preparative chromatography. The solvent was removed by concentration under reduced pressure to obtain Compound 32 (C₂₇H₃₉N₄O₅, [M+H]⁺ 499.3; found 499.2, yield 85.9%).

### Step 5. Synthesis of Compound 33

Compound 32 (0.35 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 33 (C₂₂H₃₁N₄O₃, [M+H]⁺ 399.2; found 399.1, crude product).

### Step 6. Synthesis of Compound 34

Compound 33 (0.35 mmol), Compound 21 (0.45 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 34 (C₂₅H₃₃N₄O₃, [M+H]⁺ 437.3; found 437.2, yield 71.4%).

### Step 7. Synthesis of Compound C1-1

Under nitrogen protection, Compound R_{2-D1} (0.25 mmol), Compound 34 (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound C1-1 (16.4 mg, 18.14 μmol). LC-MS: C₅₆H₅₅N₈O₄, [M/2+H]⁺452.2; found 452.6. ¹H NMR (400 MHz, DMSO-d6) δ 9.85-9.73 (m, 1H), 9.41-9.31 (m, 1H), 8.85 (s, 1H), 8.27-8.25 (m, 2H), 8.15-7.94 (m, 1H), 7.78-7.73 (m, 3H), 7.71-7.69 (m, 2H), 7.68 (d, J=8.0 Hz, 1H), 7.56 (d, J=4.0 Hz, 2H), 7.50-7.40 (m, 4H), 7.38-7.28 (m, 2H), 5.10-5.07 (m, 1H), 4.96-4.80 (m, 2H), 4.36-4.25 (m, 1H), 4.20-3.92 (m, 1H), 3.74 (s, 3H), 3.66-3.45 (m, 3H), 3.09-2.95 (m, 6H), 2.94-2.83 (m, 3H), 2.72-2.68 (m, 4H), 2.54-2.42 (m, 2H), 2.38-2.06 (m, 1H), 1.69-1.63 (m, 4H), 1.40-1.34 (m, 2H).

According to the step 1 to step 7 of the method for Compound C1-1, the raw material 1 in the following table was used to replace Compound R_{1B-1}, and the raw material 2 in the following table was used to replace Compound 28a, and the other raw materials and operating methods remained unchanged to obtain Compounds C1-2 and C1-3.

| Com- | | | Raw | |
|---|---|---|---|---|
| pound | Structure and characterization data of | Raw | material | |
| No. | compound | material 1 | 2 | Yield |
| C1-2 | 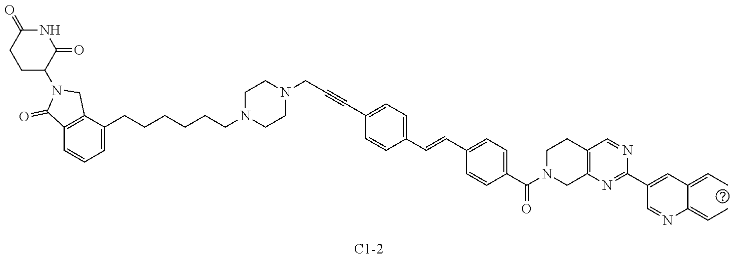 [SMILES: C/C=c1/cc(-c2ncc3c(n2)CN(C(=O)c2ccc(/C=C/c4ccc(C#CCN5CCN(CCCCCCc6cccc7c6CN(C6CCC(=O)NC6=O)C7=O)CC5)cc4)cc2)CC3)cn/c1=C/C ; "C1-2"] | 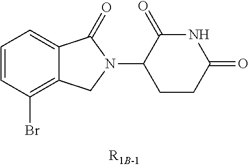 [SMILES: O=C1CCC(N2Cc3c(Br)cccc3C2=O)C(=O)N1] | 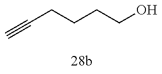 | 15.7% |
| | LC-MS: C₅₇H₅₇N₈O₄, [M/2 + H]⁺ 459.2; found | | | |
| | 459.6. ¹H NMR (400 MHz, DMSO-d₆) δ 9.86- | | | |
| | 9.76 (m, 1H), 9.48-9.36 (m, 1H), 8.86 (s, 1H), | | | |
| | 8.29-8.25 (m, 2H), 8.18-8.17 (m, 1H), 7.97- | | | |
| | 7.77 (m, 3H), 7.75-7.72 (m, 2H), 7.68 (d, J = | | | |
| | 8.0 Hz, 1H), 7.63 (d, J = 4.0 Hz, 2H), 7.60- | | | |
| | 7.50 (m, 4H), 7.49-7.38 (m, 2H), 5.10-5.07 | | | |
| | (m, 1H), 4.97-4.81 (m, 2H), 4.51-4.48 (m, | | | |
| | 1H), 4.36-3.93 (m, 1H), 3.79 (s, 3H), 3.12- | | | |
| | 3.01 (m, 7H), 2.94-2.86 (m, 3H), 2.72-2.65 | | | |
| | (m, 5H), 2.54-2.37 (m, 3H), 2.36-2.05 (m, | | | |
| | 1H), 1.63 (s, 4H). 1.35 (s, 4H). | | | |
| C1-3 | 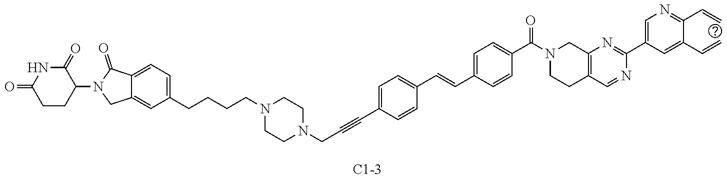 [SMILES: C=Cc1cc(-c2ncc3c(n2)CN(C(=O)c2ccc(/C=C/c4ccc(C#CCN5CCN(CCCCc6ccc7c(c6)CN(C6CCC(=O)NC6=O)C7=O)CC5)cc4)cc2)CC3)cnc1C=C] | 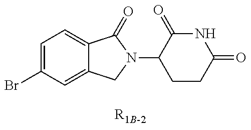 [SMILES: O=C1CCC(N2Cc3cc(Br)ccc3C2=O)C(=O)N1] | 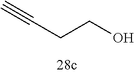 | 16.8% |
| | LC-MS: C₅₅H₅₃N₈O₄, [M/2 + H]⁺ 445.6; found | | | |
| | 445.4. ¹H NMR (400 MHz, DMSO-d₆) δ | | | |
| | 11.01 (s, 1H), 9.82-9.76 (m, 1H), 9.25-9.21 | | | |
| | (m, 1H), 8.85 (s, 1H), 8.22 (s, 1H), 8.10 (s, | | | |
| | 1H), 7.87-7.85 (m, 1H), 7.75-7.73 (m, 2H), | | | |
| | 7.68-7.66 (m, 4H), 7.57-7.55 (m, 2H), 7.48- | | | |
| | 7.46 (m, 3H), 7.40-7.37 (m, 3H), 5.05-5.03 | | | |
| | (m, 1H), 4.92-4.78 (m, 2H), 4.45-4.40 (m, | | | |
| | 1H), 4.32-4.27 (m, 1H), 3.71-3.69 (m, 4H), | | | |
| | 3.32-3.17 (m, 2H), 3.12-2.99 (m, 6H), 2.95- | | | |
| | 2.91 (m, 1H), 2.86-2.76 (m, 2H), 2.67-2.62 | | | |
| | (m, 4H), 2.44-2.41 (m, 1H), 2.38-2.32 (m, | | | |
| | 1H), 2.01-1.99 (m, 1H), 1.98-1.65 (m, 4H). | | | |

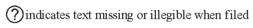

Compound C1-4 could be obtained by following the step 1 to step 7 of the method for Compound C1-1, in which the raw material 1 in the following table was used to replace Compound R_{2D-1}, and the other raw materials and operating methods remained unchanged.

| Com- | | | |
|---|---|---|---|
| pound | Structure and characterization data of | | |
| No. | compound | Raw material 1 | Yield |
| C1-4 | 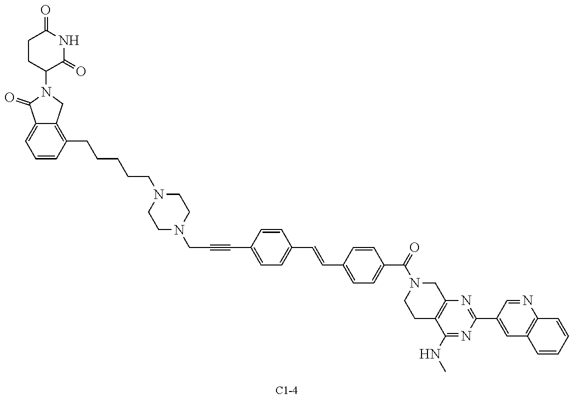 [SMILES: CNc1nc(-c2cnc3ccccc3c2)nc2c1CCN(C(=O)c1ccc(/C=C/c3ccc(C#CCN4CCN(CCCCCc5cccc6c5CN(C5CCC(=O)NC5=O)C6=O)CC4)cc3)cc1)C2] | 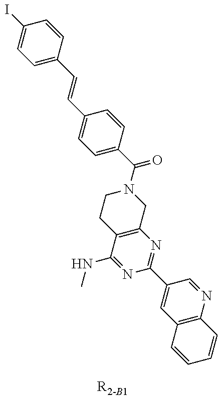 [SMILES: CNc1nc(-c2cnc3ccccc3c2)nc2c1CCN(C(=O)c1ccc(/C=C/c3ccc(I)cc3)cc1)C2] | 15.7% |
| | Compound C1-4 (25.7 mg, 37.76 μmol, 98.5% | | |
| | Purity). LC-MS: C₅₇H₅₈N₉O₄, [M/2 + H]⁺ 467.1; | | |
| | found 467.2. ¹H NMR (600 MHz, DMSO-d₆) δ 9.78- | | |
| | 9.72 (m, 1H), 9.41-9.33 (m, 1H), 8.28-8.18 (m, | | |
| | 2H), 7.99 (s, 1H), 7.76-7.39 (m, 14H), 5.13-5.12 | | |
| | (m, 1H), 4.82-4.64 (m, 2H), 4.50-4.31 (m, 2H), | | |
| | 3.99 (s, 1H), 3.84 (s, 1H), 3.14-3.11 (m, 7H), 2.92 | | |
| | (s, 2H), 2.67-2.43 (m, 13H), 2.06 (s, 1H), 1.69- | | |
| | 1.67 (m, 4H), 1.36 (s, 2H). Purity >95%. | | |

### Example 4. Synthesis of Compounds C2-1, C2-2, C2-3, C2-4, C2-5 and C2-6 of the Present Invention

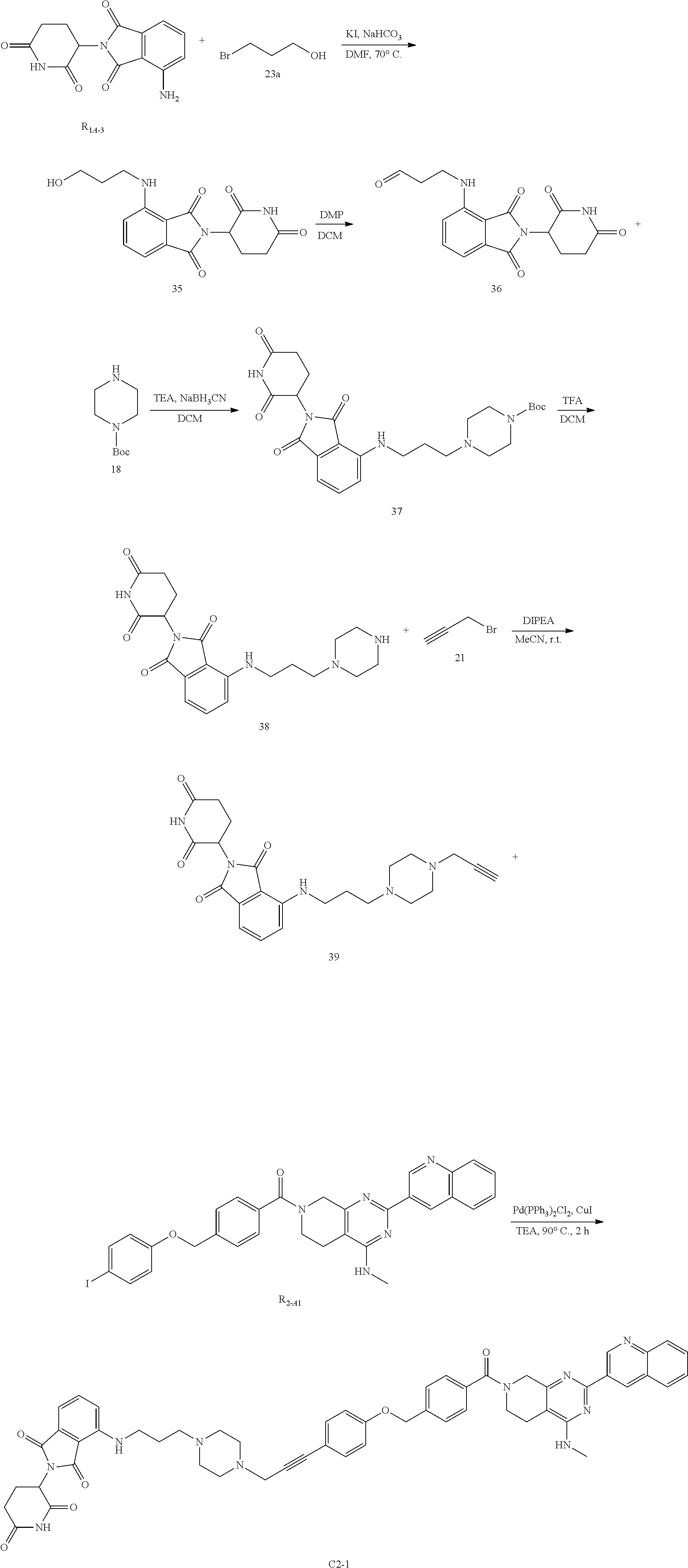

### Step 1. Synthesis of Compound 35

Compound R_{1A-3} (1.00 mmol), Compound 23a (1.80 mmol), potassium iodide (1.05 mmol), sodium bicarbonate (2.20 mmol) and N,N-dimethylformamide (5 mL) were added into a 50 mL reaction bottle, heated to 70° C., reacted under stirring for 8 hours, and then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 35 (C₁₆H₁₈N₃O₅, [M+H]⁺ 332.1; found 332.3, yield 61.2%).

### Step 2. Synthesis of Compound 36

Compound 35 (0.60 mmol), Dess-Martin Periodinane (1.80 mmol) and dichloroethane (5 mL) were added to a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 36 (C₁₆H₁₅N₂O₆, [M+H]⁺ 331.1; found 331.3, yield 91.3%).

### Step 3. Synthesis of Compound 37

Compound 36 (0.55 mmol), Compound 18 (0.75 mmol), triethylamine (2 mL), sodium cyanoborohydride (0.80 mmol) and dichloroethane (5 mL) were added in sequence to a 50 mL reaction bottle. The reaction was carried out under stirring at room temperature for 2 hours, and then the reaction was quenched (LC-MS monitoring), the solvent was removed by evaporation to dryness, purification was carried out by medium-pressure preparative chromatography, and the solvent was removed by concentration under reduced pressure to obtain Compound 37 (C₂₅H₃₄N₅O₆, [M+H]⁺ 500.2; found 500.4, yield 34.8%).

### Step 4. Synthesis of Compound 38

Compound 37 (0.30 mmol) was dissolved in dichloromethane (5 mL) in a 50 mL reaction bottle, then trifluoroacetic acid (1.5 mL) was added dropwise, the reaction was carried out under stirring at room temperature for 0.5 hour, the solvent was removed by concentration under reduced pressure to obtain Compound 38 (C₂₀H₂₆N₅O₄, [M+H]⁺ 400.2; found 400.1, crude product).

### Step 5. Synthesis of Compound 39

Compound 38 (0.30 mmol), Compound 21 (0.65 mmol), N,N-diisopropylethylamine (1.20 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the organic combined phase was dried over anhydrous sodium sulfate, evaporated to dryness to remove the solvent, purified by medium-pressure preparative chromatography, and concentrated under reduced pressure to remove the solvent to obtain Compound 39 (C₂₃H₂₈N₅O₄, [M+H]⁺ 438.2; found 438.3, yield 87.6%).

### Step 6. Synthesis of Compound C2-1

Under nitrogen protection, Compound 39 (0.25 mmol), Compound R_{2-A1} (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound C2-1 (5.9 mg, 6.38 μmol, 99.9% purity). LC-MS: C₅₄H₅₃N₁₀O₆, [M/2+H]⁺ 469.6; found 469.4. ¹H NMR (400 MHz, Methanol-d4) 9.79-9.69 (m, 1H), 9.24-9.13 (m, 1H), 8.22-8.07 (i, 2H), 7.82 (s, 1H), 7.73-7.52 (m, 6H), 7.36 (d, J=8.0 Hz, 2H), 7.07-6.96 (m, 4H), 5.19 (s, 2H), 5.05-5.00 (m, 1H), 4.58 (s, 4H), 4.28 (t, J=4.0 Hz, 1H), 4.27 (s, 1H), 3.76-3.74 (i, 2H), 3.51-3.48 (m, 2H), 3.38 (s, 2H), 3.16 (s, 3H), 2.79-2.51 (m, 12H), 1.87-1.85 (m, 2H).

Compounds C2-2 to C2-5 could be obtained by following the step 1 to step 6 of the method for Compound C2-1, in which Compound 23a was replaced with the raw materials in the below table, and the other raw materials and operating methods remained unchanged.

| Compound | | Raw | |
|---|---|---|---|
| No. | Structure and characterization data of compound | material | Yield |
| C2-2 | 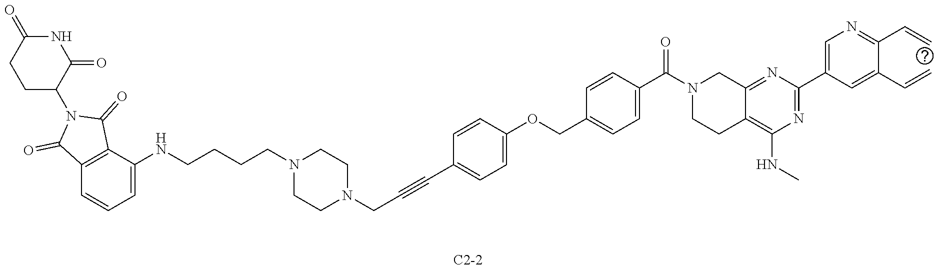 [SMILES: C=Cc1cc(-c2nc3c(c(NC)n2)CCN(C(=O)c2ccc(COc4ccc(C#CCN5CCN(CCCCNc6cccc7c6C(=O)N(C6CCC(=O)NC6=O)C7=O)CC5)cc4)cc2)C3)cnc1C=C ; "C2-2"] | 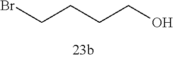 | 15.3% |
| | LC-MS: C₅₅H₅₅N₁₀O₆, [M/2 + H]⁺ 476.6; found 476.4. ¹H | | |
| | NMR (400 MHz, Methanol-d₄) δ 9.80-9.69 (m, 1H), | | |
| | 9.25-9.19 (m, 1H), 8.12 (s, 2H), 7.82 (s, 1H), 7.73-7.53 | | |
| | (m, 6H), 7.36 (d, J = 8.0 Hz, 2H), 7.07-6.93 (m, 4H), 5.19 | | |
| | (s, 2H), 5.05-5.00 (m, 1H), 4.58 (s, 5H), 4.28 (t, J = 4.0 | | |
| | Hz, 1H), 4.25 (s, 2H), 3.79-3.72 (m, 2H), 3.59-3.48 (m, | | |
| | 2H), 3.16 (s, 3H), 2.83-2.68 (m, 12H), 2.48-2.46 (m, 2H), | | |
| | 2.09-2.08 (m, 2H). | | |
| C2-3 | 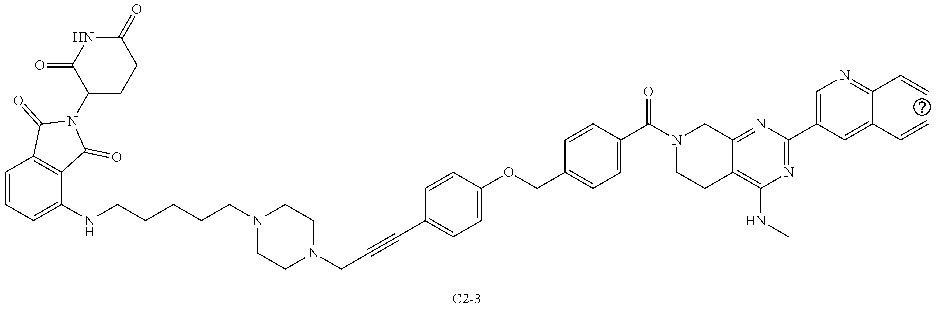 [SMILES: C=Cc1cc(-c2nc3c(c(NC)n2)CCN(C(=O)c2ccc(COc4ccc(C#CCN5CCN(CCCCCNc6cccc7c6C(=O)N(C6CCC(=O)NC6=O)C7=O)CC5)cc4)cc2)C3)cnc1C=C] | 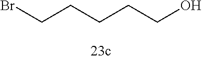 | 11.6% |
| | LC-MS: C₅₆H₅₇N₁₀O₆, [M/2 + H]⁺ 483.2; found 483.6. ¹H | | |
| | NMR (400 MHz, Methanol-d₄) δ 9.78-9.69 (m, 1H), | | |
| | 9.24-9.14 (m, 1H), 8.12 (s, 2H), 7.82 (s, 1H), 7.73-7.48 | | |
| | (m, 6H), 7.35 (d, J = 8.0 Hz, 2H), 7.06-6.96 (m, 4H), | | |
| | 5.19 (s, 2H), 5.07-5.01 (m, 1H), 4.58 (s, 4H), 4.28 (t, J = | | |
| | 4.0 Hz, 1H), 4.25 (s, 1H), 3.77-3.71 (m, 2H), 3.52-3.47 | | |
| | (m, 3H), 3.16 (s, 4H), 2.84-2.65 (m, 12H), 2.58-2.46 (m, | | |
| | 2H), 1.73-1.69 (m, 2H), 1.67-1.58 (m, 2H). | | |
| C2-4 | 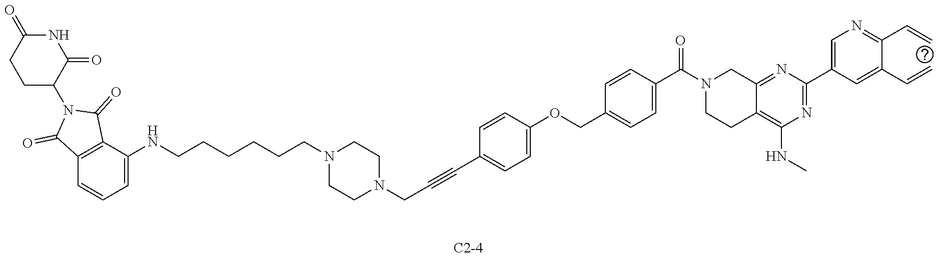 [SMILES: C=Cc1cc(-c2nc3c(c(NC)n2)CCN(C(=O)c2ccc(COc4ccc(C#CCN5CCN(CCCCCCNc6cccc7c6C(=O)N(C6CCC(=O)NC6=O)C7=O)CC5)cc4)cc2)C3)cnc1C=C] | 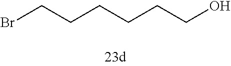 | 13.6% |
| | LC-MS: C₅₇H₅₉N₁₀O₆, [M/2 + H]⁺ 490.6; found 490.5. ¹H | | |
| | NMR (400 MHz, Methanol-d₄) δ 9.78-9.69 (m, 1H), | | |
| | 9.24-9.14 (m, 1H), 8.12 (s, 2H), 7.82 (s, 1H), 7.73-7.48 | | |
| | (m, 6H), 7.35 (d, J = 8.0 Hz, 2H), 7.06-6.96 (m, 4H), 5.19 | | |
| | (s, 3H), 4.58 (s, 4H), 4.30 (t, J = 4.0 Hz, 1H), 4.10 (s, | | |
| | 2H), 3.76-3.71 (m, 2H), 3.53 (s, 3H), 3.16 (s, 3H), 2.84- | | |
| | 2.65 (m, 12H), 2.48 (s, 2H), 2.10-2.08 (m, 2H), 1.70-1.56 | | |
| | (m, 4H). | | |
| C2-5 | 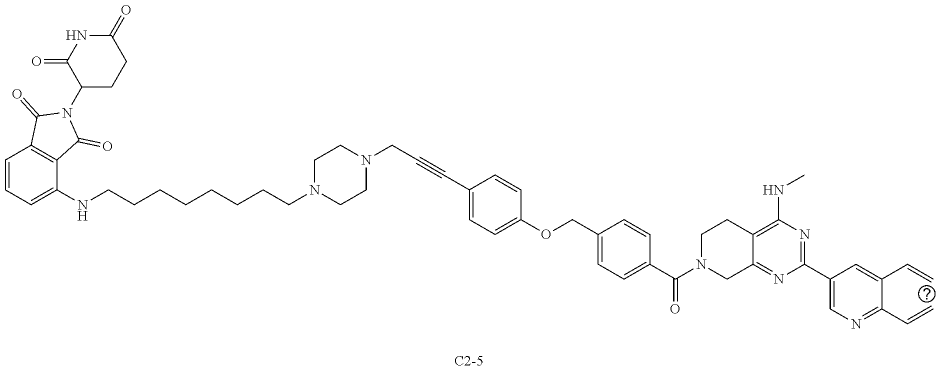 [SMILES: C=Cc1cc(-c2nc3c(c(NC)n2)CCN(C(=O)c2ccc(COc4ccc(C#CCN5CCN(CCCCCCCCNc6cccc7c6C(=O)N(C6CCC(=O)NC6=O)C7=O)CC5)cc4)cc2)C3)cnc1C=C] | 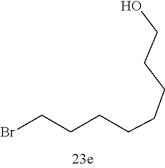 | 10.2% |
| | LC-MS: C₅₉H₆₃N₁₀O₆, [M/2 + H]⁺ 504.6; found 504.7. ¹H | | |
| | NMR (400 MHz, Methanol-d₄) δ 9.78-9.69 (m, 1H), | | |
| | 9.24-9.14 (m, 1H), 8.12 (s, 2H), 7.82 (s, 1H), 7.73-7.48 | | |
| | (m, 6H), 7.35 (d, J = 8.0 Hz, 2H), 7.06-6.96 (m, 4H), 5.19 | | |
| | (s, 2H), 5.07-5.01 (m, 1H), 4.58 (s, 4H), 4.30 (t, J = 4.0 | | |
| | Hz, 1H), 4.10 (s, 2H), 3.76-3.71 (m, 2H), 3.53 (s, 3H), | | |
| | 3.16 (s, 3H), 2.84-2.65 (m, 12H), 2.49-2.47 (m, 2H), | | |
| | 2.10-2.08 (m, 2H), 2.01-1.85 (m, 6H), 1.67-1.53 (m, 2H). | | |

Compound C2-6 could be obtained by following the step 1 to step 6 of the method for Compound C2-1, in which Compound R_{1A-3} was replaced with the raw materials in the following table, and the other raw materials and operating methods remained unchanged.

| Compound | | | |
|---|---|---|---|
| No. | Structure and characterization data of compound | Raw material | Yield |
| C2-6 | 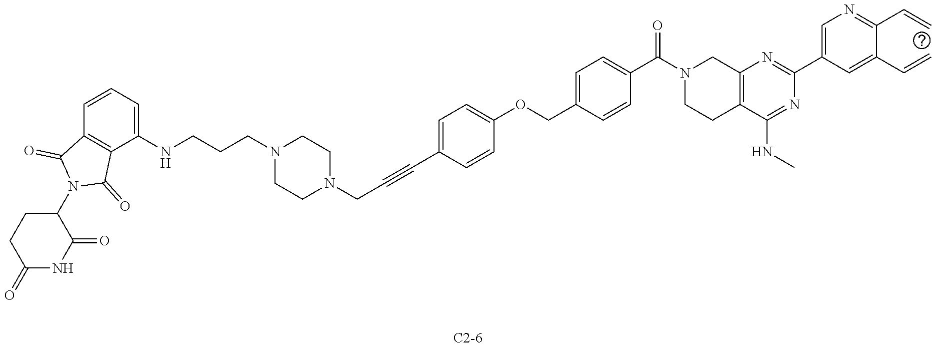 [SMILES: C=Cc1cc(-c2nc3c(c(NC)n2)CCN(C(=O)c2ccc(COc4ccc(C#CCN5CCN(CCCNc6cccc7c6C(=O)N(C6CCC(=O)NC6=O)C7=O)CC5)cc4)cc2)C3)cnc1C=C] | 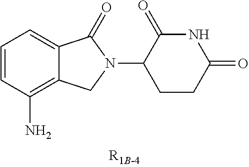 [SMILES: Nc1cccc2c1CN(C1CCC(=O)NC1=O)C2=O] | 20.1% |
| | LC-MS: C₅₄H₅₅N₁₀O₅, [M/2 + H]⁺ 462.2; found 462.6. ¹H | | |
| | NMR (400 MHz, Methanol-d₄) δ 9.78 (s, 1H), 9.51 (s, | | |
| | 1H), 8.26-8.20 (m, 2H), 8.05 (s, 1H), 7.86 (s, 1H), 7.61- | | |
| | 7.54 (m, 5H), 7.37-7.33 (m, 2H), 7.11 (d, J = 8.0 Hz, | | |
| | 1H), 7.05-6.97 (m, 2H), 6.87 (d, J = 8.0 Hz, 1H), 5.22- | | |
| | 5.13 (m, 5H), 4.73 (s, 2H), 4.29-4.23 (m, 2H), 4.15 (s, | | |
| | 1H), 3.81-3.77 (m, 2H), 3.71 (s, 2H), 3.57-3.36 (m, 6H), | | |
| | 3.26 (s, 5H), 3.19-2.91 (m, 6H), 2.88-2.76 (m, 1H), 2.66 | | |
| | (s, 1H), 2.19-2.17 (m, 1H), 2.15-2.03 (m, 2H). | | |

### Example 5. Synthesis of Compounds C3-1 to C3-18 of the Present Invention

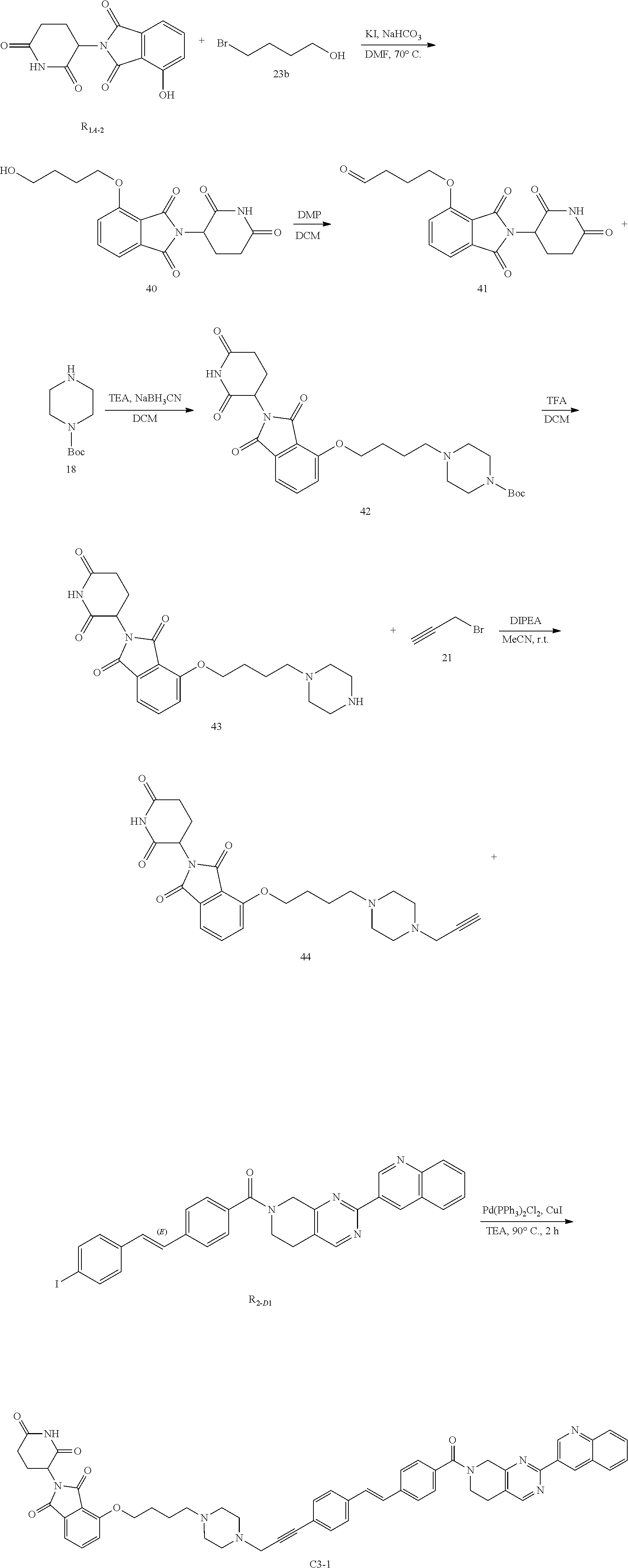

### Step 1. Synthesis of Compound 40

Compound R_{1A-2} (1.00 mmol), Compound 23b (1.12 mmol), potassium iodide (1.05 mmol), sodium bicarbonate (2.20 mmol) and N,N-dimethylformamide (5 mL) were added into a 50 mL reaction bottle, heated to 70° C., reacted under stirring for 8 hours, and then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 40 (C₁₇H₁₉N₂O₆, [M+H]⁺ 347.1; found 347.3, yield 63.8%).

### Step 2. Synthesis of Compound 41

Compound 40 (0.60 mmol), Dess-Martin Periodinane (1.80 mmol) and dichloroethane (5 mL) were added to a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 41 (C₁₇H₁₇N₂O₆, [M+H]⁺ 345.1; found 345.1, yield 91.6%).

### Step 3. Synthesis of Compound 42

Compound 41 (0.55 mmol), Compound 18 (0.65 mmol), triethylamine (2 mL), sodium cyanoborohydride (0.95 mmol) and dichloroethane (5 mL) were added in sequence to a 50 mL reaction bottle, reacted under stirring at room temperature for 2 hours, and then the reaction was quenched (LC-MS monitoring), the solvent was removed by evaporation to dryness, purification was carried out by medium-pressure preparative chromatography, and the solvent was removed by concentration under reduced pressure to obtain Compound 42 (C₂₆H₃₅N₄O₇, [M+H]⁺ 515.2; found 515.1, yield 63.6%).

### Step 4. Synthesis of Compound 43

Compound 42 (0.35 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 43 (C₂₁H₂₇N₄O₅, [M+H]⁺ 415.2; found 415.1, crude product).

### Step 5. Synthesis of Compound 44

Compound 43 (0.30 mmol), Compound 21 (0.65 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 44 (C₂₄H₂₉N₄O₅, [M+H]⁺ 453.2; found 453.3, yield 83.3%).

### Step 6. Synthesis of Compound C3-1

Under nitrogen protection, Compound R_{2-D1} (0.25 mmol), Compound 44 (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound C3-1 (9.0 mg, 9.78 μmol). LC-MS: C₅₅H₅₁N₈O₆, [M/2+H]⁺460.2; found 460.5. ¹H NMR (400 MHz, Methanol-d4) δ 10.10-9.80 (m, 2H), 8.83 (s, 1H), 8.39 (s, 1H), 8.24 (s, 1H), 8.13 (s, 1H), 7.94 (s, 1H), 7.80 (td, J=8.0, 4.0 Hz, 1H), 7.71 (d, J=4.0 Hz, 2H), 7.62-7.54 (m, 4H), 7.48-7.43 (m, 4H), 7.30 (s, 2H), 5.13-5.06 (m, 2H), 4.87 (s, 1H), 4.32-4.27 (m, 2H), 4.26-4.05 (m, 1H), 3.98 (s, 1H), 3.78 (s, 2H), 3.51-3.47 (m, 5H), 3.45-3.42 (m, 1H), 3.19-3.07 (m, 6H), 2.86-2.81 (m, 1H), 2.76-2.75 (m, 1H), 2.71-2.66 (m, 1H), 2.15-2.11 (m, 1H), 2.04-1.99 (m, 4H).

According to the step 1 to step 6 of the method for Compound C3-1, the raw materials in the following table were used to replace Compound 23b, and other raw materials and operating methods remained unchanged to obtain Compounds C3-2 and C3-3.

| Compound | | | |
|---|---|---|---|
| No. | Structure and characterization data of compound | Raw material | Yield |
| C3-2 | 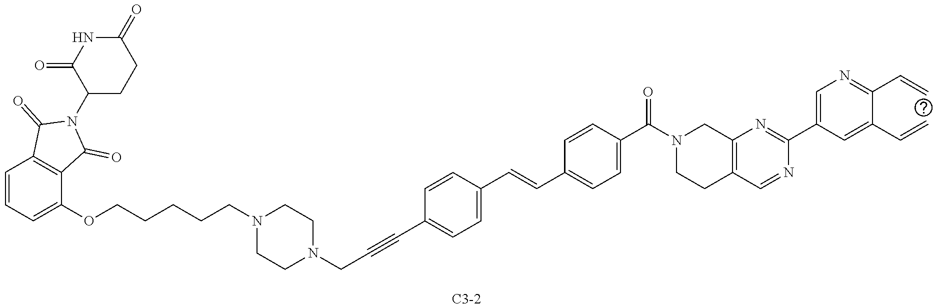 [SMILES: C=Cc1cc(-c2ncc3c(n2)CN(C(=O)c2ccc(/C=C/c4ccc(C#CCN5CCN(CCCCCOc6cccc7c6C(=O)N(C6CCC(=O)NC6=O)C7=O)CC5)cc4)cc2)CC3)cnc1C=C ; "C3-2"] | 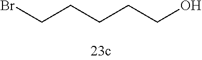 | 13.2% |
| | LC-MS: C₅₆H₅₃N₈O₆, [M + H]⁺ 933.4; found 933.2. ¹H | | |
| | NMR (400 MHz, Methanol-d₄) δ 9.93-9.62 (m, 2H), | | |
| | 8.80 (s, 1H), 8.23-8.17 (m, 2H), 8.16 (s, 1H), 7.99-7.70 | | |
| | (m, 4H), 7.62-7.54 (m, 4H), 7.47-7.43 (m, 4H), 7.30 (s, | | |
| | 2H), 5.12-5.07 (m, 2H), 4.30-4.26 (m, 3H), 4.23-3.81 | | |
| | (m, 3H), 3.73 (s, 2H), 3.67-3.42 (m, 3H), 3.31-3.21 (m, | | |
| | 3H), 3.19-3.05 (m, 4H), 2.85-2.70 (m, 5H), 2.00-1.92 | | |
| | (m, 4H), 1.72-1.67 (m, 2H). | | |
| C3-3 | 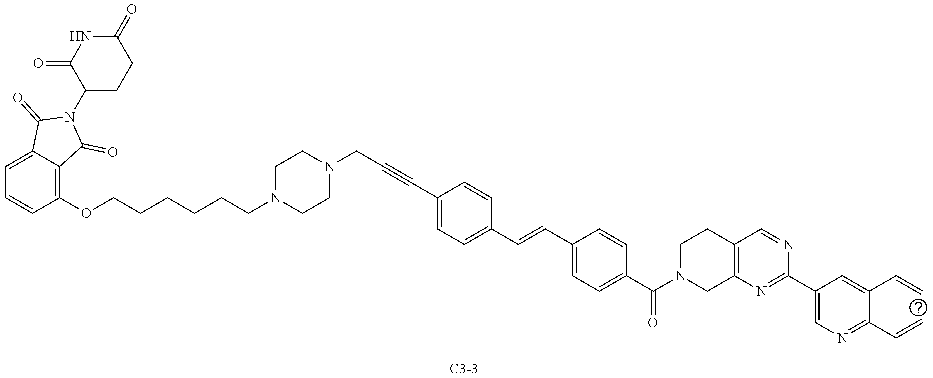 [SMILES: C=Cc1cc(-c2ncc3c(n2)CN(C(=O)c2ccc(/C=C/c4ccc(C#CCN5CCN(CCCCCCOc6cccc7c6C(=O)N(C6CCC(=O)NC6=O)C7=O)CC5)cc4)cc2)CC3)cnc1C=C ; "C3-3"] | 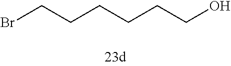 | 9.8% |
| | LC-MS: C₅₇H₅₅N₈O₆, [M/2 + H]⁺ 474.2; found 474.5. ¹H | | |
| | NMR (400 MHz, DMSO-d₆) δ 9.78-9.69 (m, 1H), 9.37- | | |
| | 9.25 (s, 1H), 8.78 (s, 1H), 8.21-8.17 (m, 2H), 8.10 (s, | | |
| | 1H), 7.91-7.77 (m, 4H), 7.72-7.70 (m, 2H), 7.62 (d, J = | | |
| | 8.0 Hz, 2H), 7.52-7.41 (m, 4H), 7.32 (s, 2H), 5.40-5.00 | | |
| | (m, 1H), 4.91-4.76 (m, 2H), 4.17 (s, 2H), 3.72 (s, 4H), | | |
| | 3.42-3.38 (m, 2H), 3.22-3.05 (m, 8H), 2.95-2.78 (m, | | |
| | 3H), 2.68-2.59 (m, 2H), 2.04-2.01 (m, 1H), 1.75 (s, 2H), | | |
| | 1.65 (s, 2H), 1.47 (s, 2H), 1.36 (d, J = 4.0 Hz, 2H). | | |

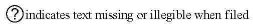

According to the step 1 to step 6 of the method for Compound C3-1, Compound 23c was used to replace Compound 23b, the raw material 1 in the following table was used to replace Compound R_{1A-2}, the raw material 2 was used to replace Compound R_{2-D1}, and the other raw materials and operating methods remained unchanged to obtain Compounds C3-4 to C3-13.

| Com- | | | | |
|---|---|---|---|---|
| pound | Structure and characterization | Raw | | |
| No. | data of compound | material 1 | Raw material 2 | Yield |
| C3-4 | 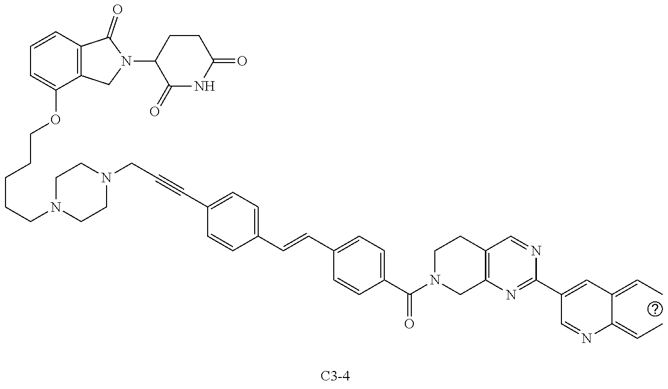 [SMILES: C/C=c1/cc(-c2ncc3c(n2)CN(C(=O)c2ccc(/C=C/c4ccc(C#CCN5CCN(CCCCCOc6cccc7c6CN(C6CCC(=O)NC6=O)C7=O)CC5)cc4)cc2)CC3)cn/c1=C/C] | 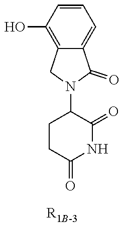 [SMILES: O=C1CCC(N2Cc3c(O)cccc3C2=O)C(=O)N1] | 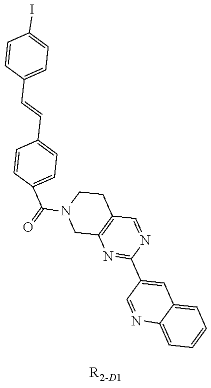 [SMILES: O=C(c1ccc(/C=C/c2ccc(I)cc2)cc1)N1CCc2cnc(-c3cnc4ccccc4c3)nc2C1] | 10.6% |
| | LC-MS: C₅₆H₅₅N₈O₅, [M/2 + H]⁺ | | | |
| | 460.2; found 460.6. ¹H NMR (400 | | | |
| | MHz, DMSO-d₆) δ 9.83-9.74 (m, | | | |
| | 1H), 9.37-9.26 (m, 1H), 8.85 (s, 1H), | | | |
| | 8.24-8.09 (m, 2H), 8.01-7.93 (m, | | | |
| | 1H), 7.78-7.72 (m, 3H), 7.72-7.70 | | | |
| | (m, 2H), 7.68 (d, J = 4.0 Hz, 2H), | | | |
| | 7.56 (d, J = 4.0 Hz, 2H), 7.50-7.44 | | | |
| | (m, 3H), 7.38-7.24 (m, 1H), 7.06 (d, | | | |
| | J = 8.0 Hz, 1H), 5.06 (s, 1H), 4.96- | | | |
| | 4.80 (m, 2H), 4.36-4.25 (m, 1H), | | | |
| | 4.20-3.92 (m, 1H), 3.72 (s, 2H), 3.66 | | | |
| | (d, J = 6.2 Hz, 2H), 3.50-3.45 (m, | | | |
| | 2H), 3.05-2.92 (m, 9H), 2.82 (s, 2H), | | | |
| | 2.78-2.71 (m, 2H), 2.54-2.40 (m, | | | |
| | 2H), 2.08 (t, J = 8.0 Hz, 1H), 1.66 (s, | | | |
| | 2H), 1.48 (d, J = 6.8 Hz, 2H), 1.28- | | | |
| | 1.17 (m, 2H). | | | |
| C3-5 | 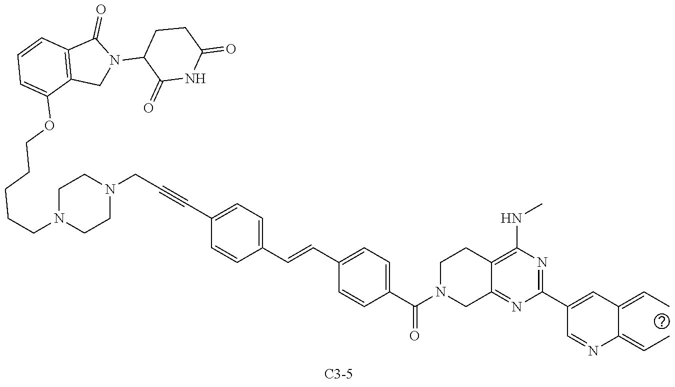 [SMILES: C/C=c1/cc(-c2nc3c(c(NC)n2)CCN(C(=O)c2ccc(/C=C/c4ccc(C#CCN5CCN(CCCCCOc6cccc7c6CN(C6CCC(=O)NC6=O)C7=O)CC5)cc4)cc2)C3)cn/c1=C/C] | 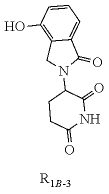 [SMILES: O=C1CCC(N2Cc3c(O)cccc3C2=O)C(=O)N1] | 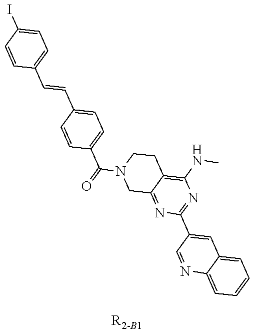 [SMILES: CNc1nc(-c2cnc3ccccc3c2)nc2c1CCN(C(=O)c1ccc(/C=C/c3ccc(I)cc3)cc1)C2] | 12.3% |
| | LC-MS: C₅₇H₅₈N₉O₅, [M/2 + H]⁺ | | | |
| | 474.7; found 475.1. ¹H NMR (400 | | | |
| | MHz, DMSO-d₆) δ 9.77-9.68 (m, | | | |
| | 1H), 9.39-9.25 (m, 1H), 8.24-8.15 | | | |
| | (m, 2H), 7.99 (s, 1H), 7.97-7.93 (m, | | | |
| | 3H), 7.77-7.75 (m, 2H), 7.71-7.48 | | | |
| | (m, 5H), 7.40-7.28 (m, 3H), 7.26- | | | |
| | 7.24 (m, 1H), 5.12-5.09 (m, 1H), | | | |
| | 4.97-4.81 (m, 1H), 4.69-4.52 (m, | | | |
| | 1H), 4.26-4.22 (m, 1H), 4.16-4.13 | | | |
| | (m, 1H), 3.75-3.54 (m, 6H), 3.48- | | | |
| | 3.40 (m, 4H), 3.12-2.92 (m, 12H), | | | |
| | 2.89-2.68 (m, 4H), 2.44-2.41 (m, | | | |
| | 2H), 2.09-2.06 (m, 1H), 1.81-1.69 | | | |
| | (m, 2H), 1.67-1.48 (m, 2H), 1.29- | | | |
| | 1.23 (m, 2H). | | | |
| C3-6 | 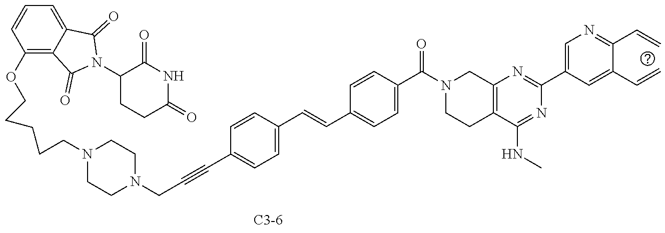 LC-MS: C₅₇H₅₆N₉O₆, [M/2 + H]⁺ 481.7; found 482.0. ¹H NMR (400 MHz, DMSO-d₆) δ 11.16 (s, 1H), 9.86-9.73 (m, 1H), 9.41-9.25 (m, 2H), 8.25-8.14 (m, 2H), 7.89-7.87 (m, 2H), 7.85-7.69 (m, 5H), 7.58- 7.43 (m, 6H), 7.39 (s, 2H), 5.14-5.10 (m, 2H), 4.78 (s, 2H), 4.64 (s, 1H), 4.23-4.21 (m, 2H), 4.01 (s, 1H), 3.89 (s, 3H), 3.56 (s, 2H), 3.19-3.07 (m, | 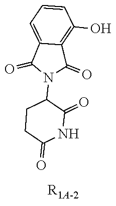 [SMILES: O=C1CCC(N2C(=O)c3cccc(O)c3C2=O)C(=O)N1] | 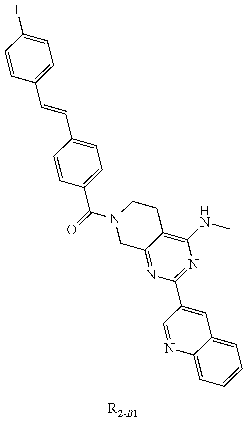 [SMILES: CNc1nc(-c2cnc3ccccc3c2)nc2c1CCN(C(=O)c1ccc(/C=C/c3ccc(I)cc3)cc1)C2] | 15.6% |
| | 8H), 2.97-2.94 (m, 1H), 2.93-2.88 | | | |
| | (m, 2H), 2.72-2.71 (m, 3H), 2.09- | | | |
| | 2.06 (m, 1H), 1.87-1.83 (m, 4H), | | | |
| | 1.57-1.54 (m, 2H). | | | |
| C3-7 | 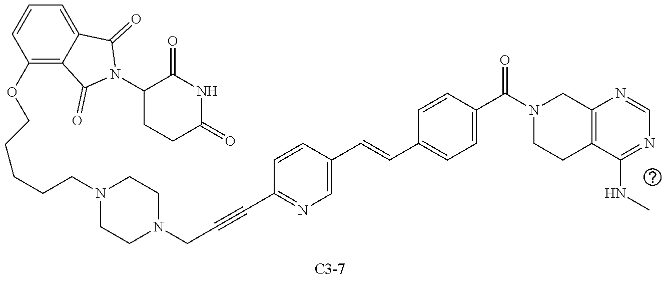 LC-MS: C₅₆H₅₅N₁₀O6, [M/2 + H]⁺ 482.2; found 482.4. ¹H NMR (400 | 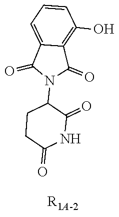 [SMILES: O=C1CCC(N2C(=O)c3cccc(O)c3C2=O)C(=O)N1] | 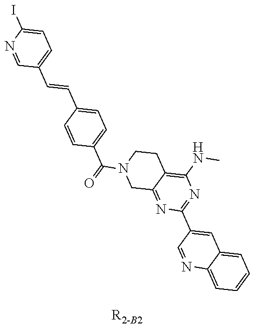 [SMILES: CNc1nc(-c2cnc3ccccc3c2)nc2c1CCN(C(=O)c1ccc(/C=C/c3ccc(I)nc3)cc1)C2] | 11.8% |
| | MHz, Methanol-d₄) δ 9.78 (s, 1H), | | | |
| | 9.51 (s, 1H), 8.71 (s, 1H), 8.28-8.20 | | | |
| | (m, 2H), 8.12 (dd, J = 4.0 Hz, 1H), | | | |
| | 8.07-8.04 (m, 1H), 7.86 (s, 1H), 7.80- | | | |
| | 7.76 (m, 3H), 7.59-7.53 (m, 3H), | | | |
| | 7.48-7.38 (m, 3H), 7.10 (s, 1H), 5.13- | | | |
| | 5.08 (m, 1H), 4.86 (s, 1H), 4.78 (s, | | | |
| | 1H), 4.28 (t, J = 8.8 Hz, 2H), 4.18 (s, | | | |
| | 1H), 3.95 (s, 1H), 3.78 (s, 1H), 3.59 | | | |
| | (s, 2H), 3.53 (s, 2H), 3.33-3.29 (m, | | | |
| | 6H), 3.23-3.13 (m, 4H), 2.86-2.82 | | | |
| | (m, 2H), 2.76-2.74 (m, 2H), 2.72- | | | |
| | 2.67 (m, 2H), 2.15-2.10 (m, 1H), | | | |
| | 1.99-1.93 (m, 4H), 1.72-1.67 (m, | | | |
| | 2H). | | | |
| C3-8 | 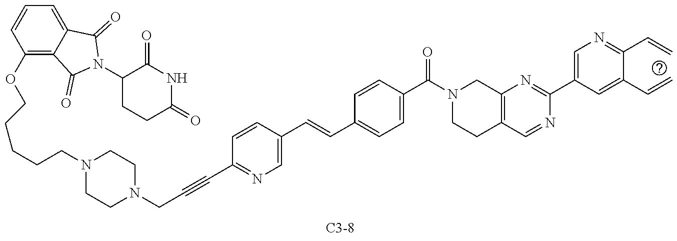 LC-MS: C₅₅H₅₂N₉O₆, [M/2 + H]⁺ 468.0; found 468.0. ¹H NMR (400 MHz, Methanol-d₄) δ 9.98-9.94 (m, 1H), 9.73-9.49 (m, 1H), 8.81 (s, 1H), | 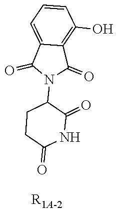 [SMILES: O=C1CCC(N2C(=O)c3cccc(O)c3C2=O)C(=O)N1] | 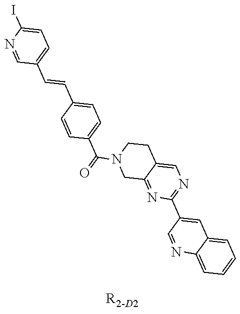 [SMILES: O=C(c1ccc(/C=C/c2ccc(I)nc2)cc1)N1CCc2cnc(-c3cnc4ccccc4c3)nc2C1] | 11.3% |
| | 8.72 (d, J = 0.8 Hz, 1H), 8.27-8.18 | | | |
| | (m, 2H), 8.12 (dd, J = 1.2 Hz, 1H), | | | |
| | 8.01 (s, 1H), 7.81-7.77 (m, 4H), 7.56 | | | |
| | (dd, J = 5.6 Hz, 3H), 7.48-7.38 (m, | | | |
| | 3H), 7.36-7.33 (m, 1H), 5.13-5.09 | | | |
| | (m, 1H), 4.86 (s, 1H), 4.78 (s, 1H), | | | |
| | 4.28 (t, J = 5.2 Hz, 2H), 4.18 (s, 1H), | | | |
| | 3.95 (s, 1H), 3.78 (s, 2H), 3.53-3.51 | | | |
| | (m, 2H), 3.30-3.23 (m, 5H), 3.16- | | | |
| | 3.06 (m, 3H), 2.91-2.66 (m, 5H), | | | |
| | 2.16-2.11 (m, 1H), 2.03-1.95 (m, | | | |
| | 4H), 1.73-1.88 (m, 2H). | | | |
| C3-9 | 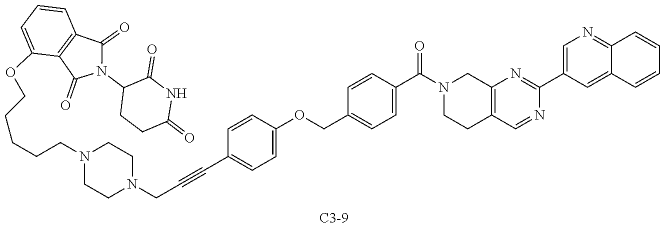 [SMILES: O=C1CCC(N2C(=O)c3cccc(OCCCCCN4CCN(CC#Cc5ccc(OCc6ccc(C(=O)N7CCc8cnc(-c9cnc%10ccccc%10c9)nc8C7)cc6)cc5)CC4)c3C2=O)C(=O)N1] LC-MS: C₅₅H₅₃N₈O₇, [M/2 + H]⁺ 469.5; found 469.5. ¹H NMR (400 MHz, DMSO-d₆) δ 9.82-9.72 (m, | 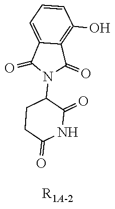 [SMILES: O=C1CCC(N2C(=O)c3cccc(O)c3C2=O)C(=O)N1] | 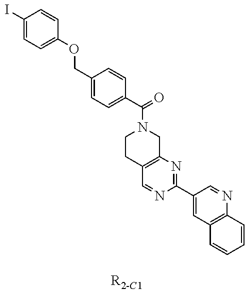 [SMILES: O=C(c1ccc(COc2ccc(I)cc2)cc1)N1CCc2cnc(-c3cnc4ccccc4c3)nc2C1] | 13.8% |
| | 1H), 9.36-9.25 (m, 1H), 8.85 (s, 1H), | | | |
| | 8.23-8.13 (m, 2H), 7.93-7.73 (m, | | | |
| | 3H), 7.64-7.41 (m, 8H), 7.13-7.06 | | | |
| | (m, 2H), 5.22 (s, 2H), 5.09-5.04 (m, | | | |
| | 1H), 4.97 (s, 1H), 4.76 (s, 1H), 4.22 | | | |
| | (s, 2H), 3.67 (s, 2H), 3.09-2.86 (m, | | | |
| | 9H), 2.71 (t, J = 2.0 Hz, 4H), 2.36 (t, | | | |
| | J = 2.0 Hz, 4H), 2.05 (t, J = 4.4 Hz, | | | |
| | 2H), 1.82 (s, 2H), 1.74 (s, 2H), 1.51 | | | |
| | (s, 2H). | | | |
| C3-10 | 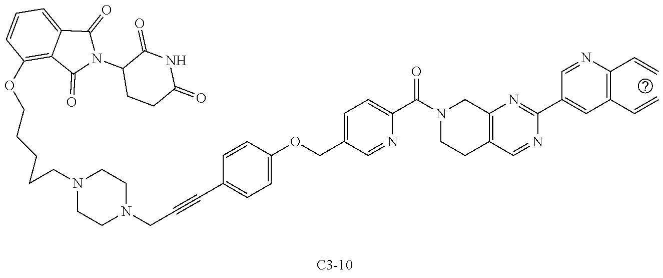 [SMILES: C=Cc1cc(-c2ncc3c(n2)CN(C(=O)c2ccc(COc4ccc(C#CCN5CCN(CCCCCOc6cccc7c6C(=O)N(C6CCC(=O)NC6=O)C7=O)CC5)cc4)cn2)CC3)cnc1C=C] | 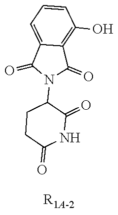 [SMILES: O=C1CCC(N2C(=O)c3cccc(O)c3C2=O)C(=O)N1] | 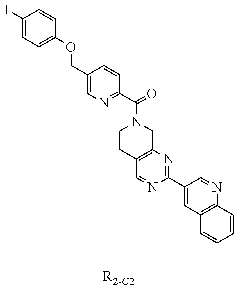 [SMILES: O=C(c1ccc(COc2ccc(I)cc2)cn1)N1CCc2cnc(-c3cnc4ccccc4c3)nc2C1] | 11.6% |
| | LC-MS: C₅₄H₅₂N₉O₇, [M/2 + H]⁺ | | | |
| | 469.7; found 469.6. ¹H NMR (400 | | | |
| | MHz, Methanol-d₄) δ 9.98-9.84 (m, | | | |
| | 1H), 9.73-9.55 (m, 1H), 8.82 (s, 1H), | | | |
| | 8.75 (s, 1H), 8.32-8.14 (m, 2H), 8.10- | | | |
| | 7.96 (m, 2H), 7.89-7.78 (m, 3H), | | | |
| | 7.46-7.39 (m, 4H), 7.03 (d, J = 8.0 | | | |
| | Hz, 2H), 5.27 (s, 2H), 5.09 (s, 2H), | | | |
| | 4.95 (s, 1H), 4.29-4.23 (m, 2H), 4.16 | | | |
| | (s, 1H), 3.88 (t, J = 4.0 Hz, 1H), 3.69 | | | |
| | (d, J = 4.0 Hz, 2H), 3.54-3.47 (m, | | | |
| | 2H), 3.24-3.08 (m, 7H), 2.90-2.63 | | | |
| | (m, 5H), 2.12-2.09 (m, 1H), 1.95- | | | |
| | 1.92 (m, 4H), 1.68 (s, 2H), 1.37-1.28 | | | |
| | (m, 1H). | | | |
| C3-11 | 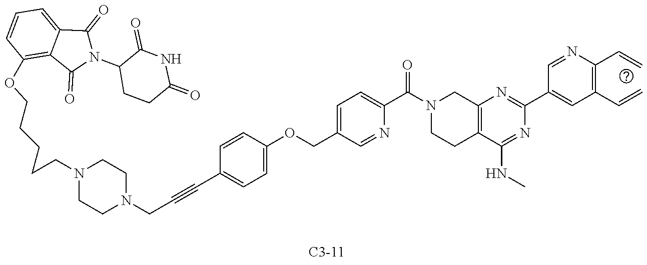 LC-MS: C₅₅H₅₅N₁₀O₇, [M/2 + H]⁺ 484.2; found 484.5. ¹H NMR (400 MHz, Methanol-d₄) δ 9.80-9.72 (m, 1H), 9.52-9.42 (m, 1H), 8.74 (s, 1H), 8.29-8.18 (m, 2H), 8.15-8.01 (m, 2H), 7.99-7.76 (m, 3H), 7.45-7.38 (m, 4H), 7.04-7.00 (m, 2H), 5.25 (s, 2H), 5.09-5.06 (m, 1H), 4.95 (s, 2H), | 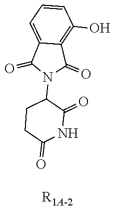 [SMILES: O=C1CCC(N2C(=O)c3cccc(O)c3C2=O)C(=O)N1] | 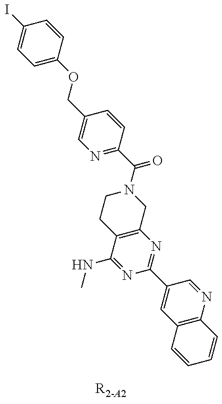 [SMILES: CNc1nc(-c2cnc3ccccc3c2)nc2c1CCN(C(=O)c1ccc(COc3ccc(I)cc3)cn1)C2] | 9.8% |
| | 4.28-4.25 (m, 2H), 4.26 (s, 1H), 3.90 | | | |
| | (t, J = 5.6 Hz, 1H), 3.68 (d, J = 5.2 | | | |
| | Hz, 2H), 3.48-3.47 (m, 2H), 3.25- | | | |
| | 3.17 (m, 6H), 3.14-2.87 (m, 4H), | | | |
| | 2.86-2.61 (m, 5H), 2.12-2.09 (m, | | | |
| | 1H), 1.94-1.93 (m, 4H), 1.69-1.65 | | | |
| | (m, 2H), 1.44-1.42 (m, 1H). | | | |
| C3-12 | 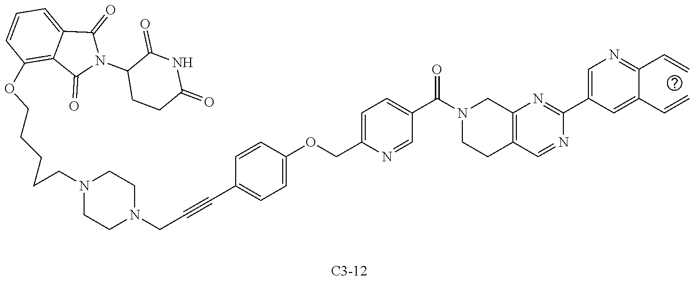 LC-MS: C₅₄H₅₂N₉O₇, [M/2 + H]⁺ 469.7; found 469.0. ¹H NMR (400 MHz, DMSO-d₆) δ 9.82-9.72 (m, 1H), 9.36-9.25 (m, 1H), 8.85 (s, 2H), 8.23-8.13 (m, 2H), 7.93-7.73 (m, 3H), 7.64-7.41 (m, 8H), 7.13-7.06 (m, 2H), 5.22 (s, 2H), 5.09-5.04 (m, 1H), 4.97 (s, 3H), 4.76 (s, 2H), 4.22 (s, 4H), 3.67 (s, 2H), 3.09-2.86 (m, 10H), 2.71 (t, J = 2.0 Hz, 4H), 2.36 (t, J = 2.0 Hz, 4H), 2.05 (t, J = 4.4 Hz, 2H), 1.82 (s, 2H), 1.74 (s, 2H), 1.51 (s, 2H). | 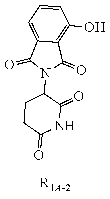 [SMILES: O=C1CCC(N2C(=O)c3cccc(O)c3C2=O)C(=O)N1] | 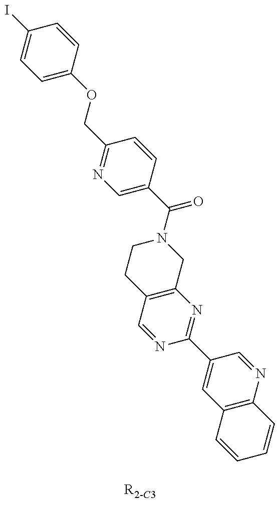 [SMILES: O=C(c1ccc(COc2ccc(I)cc2)nc1)N1CCc2cnc(-c3cnc4ccccc4c3)nc2C1] | 8.6% |
| C3-13 | 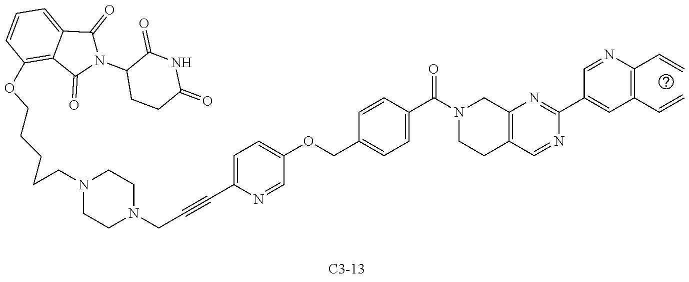 [SMILES: C=Cc1cc(-c2ncc3c(n2)CN(C(=O)c2ccc(COc4ccc(C#CCN5CCN(CCCCCOc6cccc7c6C(=O)N(C6CCC(=O)NC6=O)C7=O)CC5)nc4)cc2)CC3)cnc1C=C] LC-MS: C₅₄H₅₂N₉O₇, [M/2 + H]⁺ 469.7; found 470.2. ¹H NMR (400 MHz, DMSO-d₆) δ 9.91-9.87 (m, 1H), 9.62-9.50 (m, 1H), 8.85 (s, 1H), 8.38 (s, 2H), 8.22 (s, 1H), 8.06 (s, | 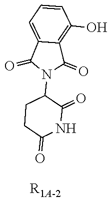 [SMILES: O=C1CCC(N2C(=O)c3cccc(O)c3C2=O)C(=O)N1] | 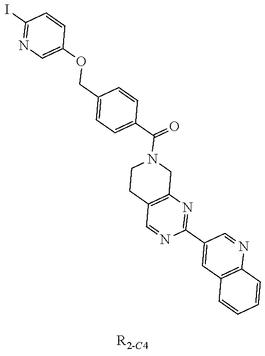 [SMILES: O=C(c1ccc(COc2ccc(I)nc2)cc1)N1CCc2cnc(-c3cnc4ccccc4c3)nc2C1] | 10.2% |
| | 1H), 7.84 (t, J = 8.0 Hz, 2H), 7.64- | | | |
| | 7.46 (m, 8H), 5.31 (s, 2H), 5.10-5.05 | | | |
| | (m, 1H), 4.98 (s, 1H), 4.86-4.77 (m, | | | |
| | 1H), 4.23-4.21 (m, 2H), 3.91 (s, 2H), | | | |
| | 3.72 (s, 2H), 3.44-3.38 (m, 4H), 3.23- | | | |
| | 3.09 (m, 7H), 2.99-2.90 (m, 1H), | | | |
| | 2.87-2.63 (m, 2H), 2.56 (s, 1H), 2.09- | | | |
| | 2.06 (m, 1H), 1.84-1.77 (m, 4H), | | | |
| | 1.60-1.50 (m, 2H). | | | |

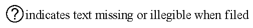

According to the step 1 to step 6 of the method for Compound C3-1, Compound R_{2-A1} was used to replace Compound R_{2-D1}, the raw materials in the following table were used to replace Compound 23b, and the other raw materials and operating methods remained unchanged to obtain Compounds C3-14 to C3-18.

| Com- | | | |
|---|---|---|---|
| pound | | Raw | |
| No. | Structure and characterization data of compound | material | Yield |
| C3-14 | 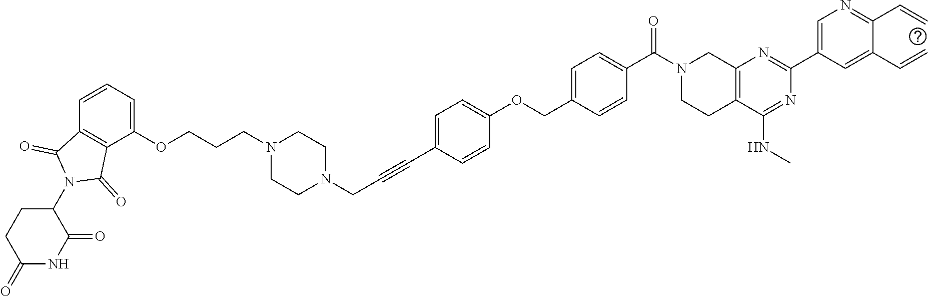 [SMILES: C=Cc1cc(-c2nc3c(c(NC)n2)CCN(C(=O)c2ccc(COc4ccc(C#CCN5CCN(CCCOc6cccc7c6C(=O)N(C6CCC(=O)NC6=O)C7=O)CC5)cc4)cc2)C3)cnc1C=C] | 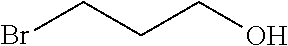 23a | 25.9% |
| | CF-14 | | |
| | LC-MS: C₅₄H₅₂N₉O₇, [M/2 + H]⁺ 470.0; found 469.9. ¹H | | |
| | NMR (400 MHz, Methanol-d₄) δ 9.78 (s, 1H), 9.54 (s, | | |
| | 1H), 8.29-8.21 (m, 2H), 8.07 (t J = 8.0 Hz, 1H), 7.89-7.79 | | |
| | (m, 2H), 7.69-7.37 (m, 8H), 6.96 (d, J = 8.0 Hz, 2H), 5.19 | | |
| | (s, 2H), 5.10-5.06 (m, 1H), 4.36-4.28 (m, 2H), 4.18-3.84 | | |
| | (m, 4H), 3.65-3.42 (m, 6H), 3.25-3.20 (m, 5H), 2.99-2.61 | | |
| | (m, 6H), 2.36 (t, J = 8.0 Hz, 2H), 2.05-2.03 (m, 2H), 1.86- | | |
| | 1.81 (m, 2H). | | |
| C3-15 | 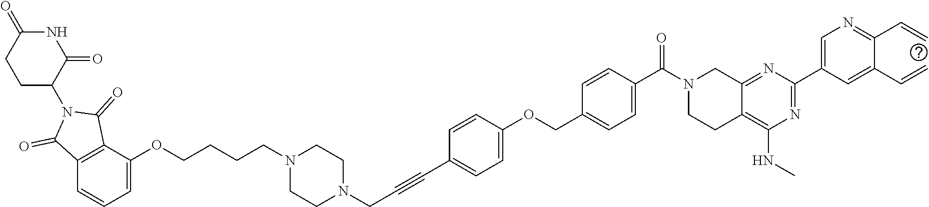 [SMILES: C=Cc1cc(-c2nc3c(c(NC)n2)CCN(C(=O)c2ccc(COc4ccc(C#CCN5CCN(CCCCOc6cccc7c6C(=O)N(C6CCC(=O)NC6=O)C7=O)CC5)cc4)cc2)C3)cnc1C=C] | 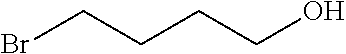 23b | 20.1% |
| | C3-15 | | |
| | LC-MS: C₅₅H₅₄N₉O₇, [M/2 + H]⁺ 477.0; found 477.0. ¹H | | |
| | NMR (400 MHz, Methanol-d₄) δ 9.78 (s, 1H), 9.51 (s, | | |
| | 1H), 8.27-8.20 (m, 2H), 8.03-7.97 (m, 1H), 7.85-7.70 (m, | | |
| | 2H), 7.69-7.36 (m, 8H), 6.97 (d, J = 8.0 Hz, 2H), 5.19 (s, | | |
| | 2H), 5.12-5.07 (m, 1H), 4.38-4.29 (m, 2H), 4.12 (s, 1H), | | |
| | 3.90 (s, 1H), 3.88 (s, 2H), 3.70-3.44 (m, 4H), 3.24 (s, 3H), | | |
| | 2.99-2.65 (m, 10H), 2.13-1.98 (m, 5H), 1.46-1.26 (m, | | |
| | 2H), 0.99-0.87 (m, 1H). | | |
| C3-16 | 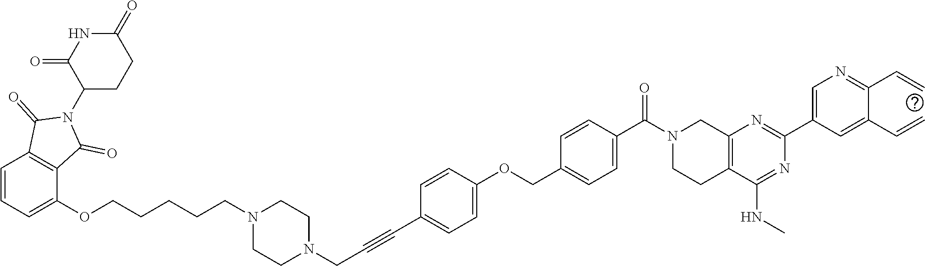 [SMILES: C=Cc1cc(-c2nc3c(c(NC)n2)CCN(C(=O)c2ccc(COc4ccc(C#CCN5CCN(CCCCCOc6cccc7c6C(=O)N(C6CCC(=O)NC6=O)C7=O)CC5)cc4)cc2)C3)cnc1C=C] | 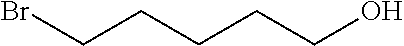 23c | 19.8% |
| | C3-16 | | |
| | LC-MS: C₅₆H₅₆N₉O₇, [M/2 + H]⁺ 483.7; found 484.0. ¹H | | |
| | NMR (400 MHz, Methanol-d₄) δ 9.78 (s, 1H), 9.52 (s, | | |
| | 1H), 8.28-8.22 (m, 2H), 8.20 (s, 1H), 8.06 (s, 1H), 7.87- | | |
| | 7.76 (m, 1H), 7.62-7.55 (m, 4H), 7.45 (t, J = 8.0 Hz, 2H), | | |
| | 7.38 (d, J = 8.0 Hz, 2H), 6.98 (d, J = 8.0 Hz, 2H), 5.19 | | |
| | (s, 2H), 5.10-5.06 (m, 1H), 4.86 (s, 1H), 4.56 (s, 1H), 4.26 | | |
| | (t, J = 5.6 Hz, 2H), 4.18-4.12 (m, 1H), 3.82 (s, 1H), 3.69 | | |
| | (s, 2H), 3.58-3.45 (m, 3H), 3.31-3.25 (m, 6H), 3.22-2.93 | | |
| | (m, 4H), 2.86-2.66 (m, 5H), 2.13-2.08 (m, 1H), 1.97-1.91 | | |
| | (m, 4H), 1.71-1.66 (m, 2H). | | |
| C3-17 | 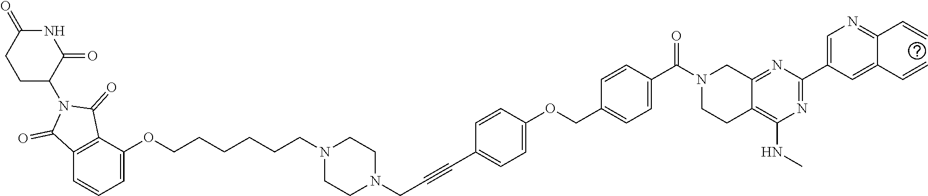 [SMILES: C=Cc1cc(-c2nc3c(c(NC)n2)CCN(C(=O)c2ccc(COc4ccc(C#CCN5CCN(CCCCCCOc6cccc7c6C(=O)N(C6CCC(=O)NC6=O)C7=O)CC5)cc4)cc2)C3)cnc1C=C] | 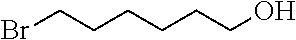 23d | 10.6% |
| | C3-17 | | |
| | LC-MS: C₅₇H₅₈N₉O₇, [M/2 + H]⁺ 491.0; found 491.0. ¹H | | |
| | NMR (400 MHz, Methanol-d₄) δ 9.82-9.79 (m, 1H), 9.28- | | |
| | 9.14 (m, 1H), 8.21-8.06 (m, 2H), 7.81-7.52 (m, 7H),7.43- | | |
| | 7.05 (m, 4H), 6.97 (d, J = 8.0 Hz, 2H), 5.19 (s, 2H), 5.12- | | |
| | 5.07 (m, 1H), 4.58 (s, 4H), 4.38-4.21 (m, 4H), 3.88 (s, | | |
| | 2H), 3.64 (s, 2H), 3.19 (s, 3H), 3.16-2.58 (m, 12H), 2.13- | | |
| | 1.98 (m, 2H), 1.85 (t, J = 8.0 Hz, 2H), 1.60-1.58 (m, 2H), | | |
| | 1.44-1.42 (m, 2H), 1.31-1.28 (m, 2H). | | |
| C3-18 | 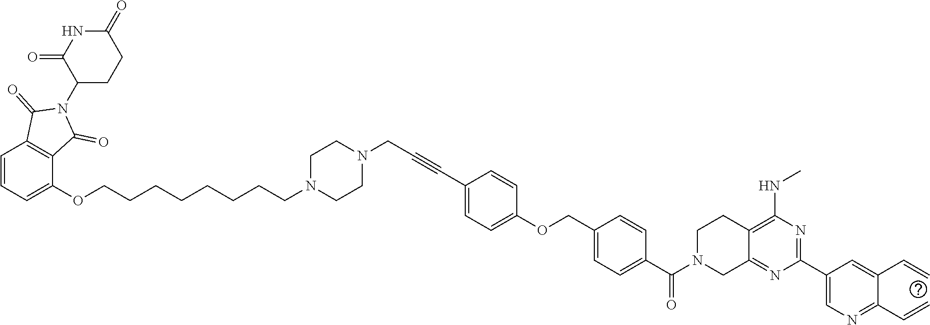 [SMILES: C=Cc1cc(-c2nc3c(c(NC)n2)CCN(C(=O)c2ccc(COc4ccc(C#CCN5CCN(CCCCCCCCOc6cccc7c6C(=O)N(C6CCC(=O)NC6=O)C7=O)CC5)cc4)cc2)C3)cnc1C=C] | 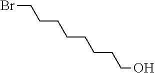 23e | 9.8% |
| | C3-18 | | |
| | LC-MS: C59H62N₉O₇, [M/2 + H]⁺ 505.1; found 505.0. 1H | | |
| | NMR (400 MHz, Methanol-d₄) δ 9.80-9.76 (m, 1H), 9.25- | | |
| | 9.14 (m, 1H), 8.06 (s, 2H), 7.97-7.52 (m, 7H),7.46-7.21 | | |
| | (m, 4H), 6.97 (d, J = 8.0 Hz, 2H), 5.19 (s, 2H), 5.12-5.07 | | |
| | (m, 1H), 4.58 (s, 4H), 4.28 (t, J = 8.0 Hz, 2H), 4.20 (t, | | |
| | J = 8.0 Hz, 2H), 3.54 (s, 2H), 3.16 (s, 3H), 2.99-2.65 (m, | | |
| | 12H), 2.51-2.49 (m, 4H), 2.20-2.09 (m, 2H), 1.84 (t, J = | | |
| | 8.0 Hz, 2H), 1.75-1.68 (m, 2H). | | |

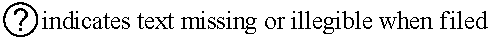

### Example 5B, Synthesis of Compound C3-19 of the Present Invention

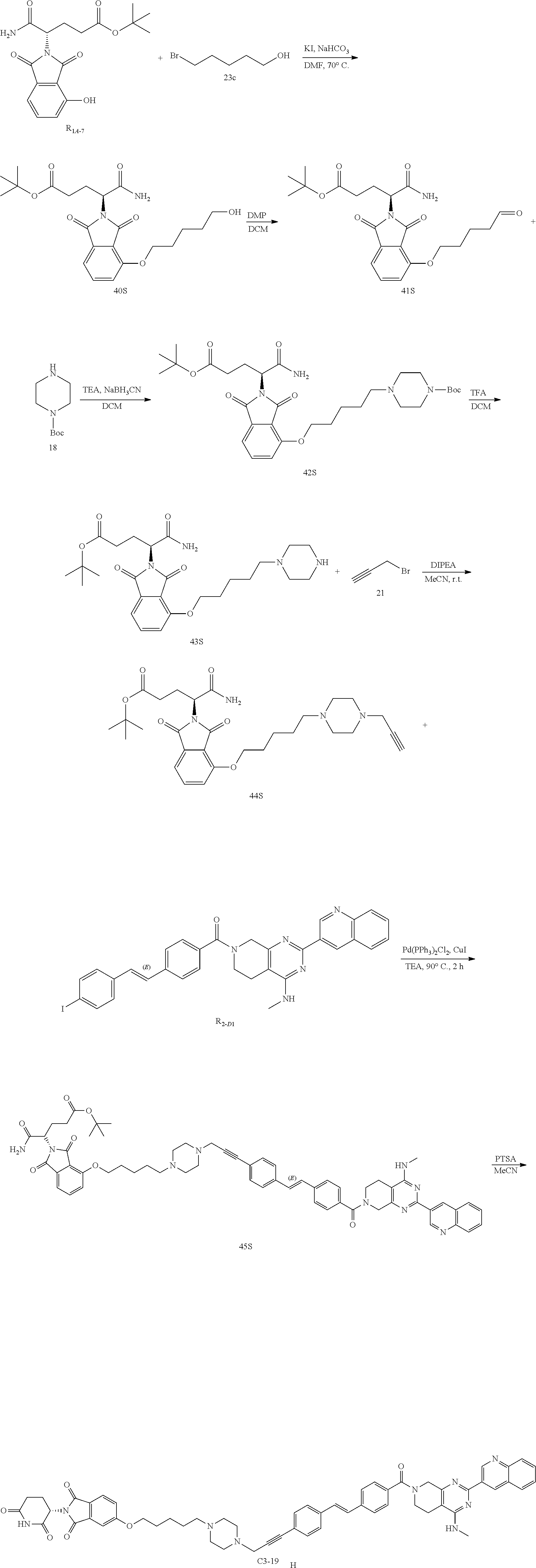

### Step 1. Synthesis of Compound 40S

Compound R_{1A-7} (1.00 mmol), Compound 23c (1.12 mmol), potassium iodide (1.05 mmol), sodium bicarbonate (2.20 mmol) and N,N-dimethylformamide (5 mL) were added into a 50 mL reaction bottle, heated to 70° C., reacted under stirring for 8 hours, and then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 40S (C₂₂H₃₁N₂O₇, [M+H]⁺ 435.5; found 435.5, yield 45.1%).

### Step 2. Synthesis of Compound 41S

Compound 40S (0.45 mmol), Dess-Martin Periodinane (1.20 mmol) and dichloromethane (5 mL) were added to a 50 mL reaction bottle, reacted under stirring at room temperature for 1 hour, and then added with water to quench the reaction (LC-MS monitoring). Extraction was carried out with saturated sodium chloride solution (25 mL) and ethyl acetate (3×25 mL), the organic phases were combined, the organic phase was dried over anhydrous sodium sulfate, evaporated to dryness to remove the solvent, purified by medium-pressure preparative chromatography, and concentrated under reduced pressure to remove the solvent to obtain Compound 41S (C₂₂H₂₉N₂O₇, [M+H]⁺ 433.5; found 433.2, yield 64.2%).

### Step 3. Synthesis of Compound 42S

Compound 41S (0.21 mmol), Compound 18 (0.42 mmol), triethylamine (2 mL), sodium cyanoborohydride (0.35 mmol) and dichloromethane (5 mL) were added in sequence to a 50 mL reaction bottle, and reacted under stirring at room temperature for 2 hours, then the reaction was quenched (LC-MS monitoring), the solvent was removed by evaporation to dryness, purification was carried out by medium-pressure preparative chromatography, and the solvent was removed by concentration under reduced pressure to obtain Compound 42S (C₃₁H₄₇N₄O₈, [M+H]⁺ 603.7; found 603.8, yield 68.9%).

### Step 4. Synthesis of Compound 43S

Compound 42S (0.10 mmol) was dissolved in dichloromethane (5 mL) in a 50 mL reaction bottle, then trifluoroacetic acid (1.5 mL) was added dropwise, the reaction was carried out under stirring at room temperature for 0.5 hour, and the solvent was removed by concentration under reduced pressure to obtain Compound 43S (C₂₁H₂₇N₄O₅, [M+H]⁺ 415.2; found 415.1, crude product).

### Step 5. Synthesis of Compound 44S

Compound 43S (crude product), Compound 21 (0.35 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 44S (C₂₉H₄₁N₄O₆, [M+H]⁺ 541.6; found 541.3, yield 83.3%).

### Step 6. Synthesis of Compound 45S

Under nitrogen protection, Compound R_{2-B1} (0.10 mmol), Compound 44S (0.09 mmol), copper iodide (0.25 mmol), triethylamine (5 mL), and Pd(PPh₃)₂Cl₂ (0.25 mol %) were added in sequence into a 50 mL reaction bottle. The reaction system was sealed, the reaction was carried out under stirring at 90° C. for 2 hours, and then the reaction was quenched (LC-MS monitoring).

Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound 45S (C₆₁H₆₆N₉O₇, [M/2+H]⁺518.6; found 518.3, yield 34.3%).

### Step 7. Synthesis of Compound C3-19

Compound 45S (27.00 μmol), p-toluenesulfonic acid (0.10 mmol) and acetonitrile (5 mL) were added to a 50 mL reaction bottle, reacted under stirring at room temperature for 1 hour, and then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound C3-19 (5.00 mg, 5.19 μmol). LC-MS: C₅₇H₅₆N₉O₆, [M/2+H]⁺482.0; found 482.0. ¹H NMR (600 MHz, DMSO-d6) δ 11.11 (s, 1H), 9.82-9.73 (m, 1H), 9.24-9.13 (m, 2H), 8.15-8.07 (m, 2H), 7.84-7.82 (m, 2H), 7.74-7.65 (m, 5H), 7.53 (dd, J=8.5, 2.4 Hz, 3H), 7.47 (dd, J=7.6, 3.8 Hz, 3H), 7.32 (s, 2H), 5.12-5.05 (m, 2H), 4.73 (s, 1H), 4.56 (s, 1H), 4.24 (t, J=6.2 Hz, 2H), 3.97 (s, 1H), 3.69 (s, 3H), 3.12-2.91 (m, 7H), 2.88-2.85 (m, 2H), 2.65-2.62 (m, 6H), 2.39 (s, 2H), 2.07-2.02 (m, 2H), 1.83-1.81 (m, 2H), 1.75 (s, 2H), 1.52-1.51 (m, 2H).

### Example 6. Synthesis of Compound D1-1 of the Present Invention

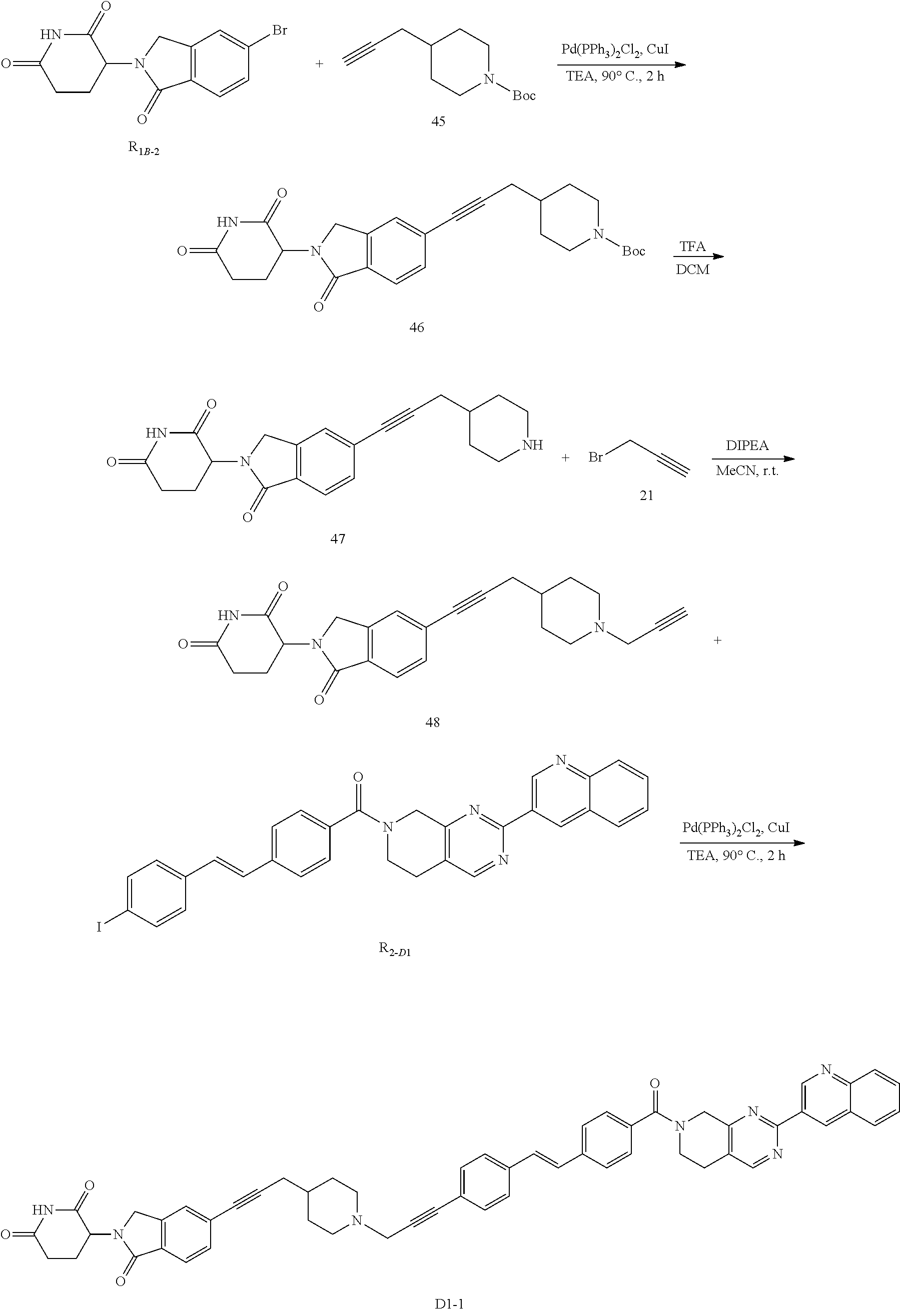

### Step 1. Synthesis of Compound 46

Under nitrogen protection, Compound R_{1B-2} (1.00 mmol), Compound 45 (1.05 mmol), copper iodide (2.05 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound 46 (C₂₆H₃₂N₃O₅, [M+H]⁺ 466.2; found 466.4, yield 46.8%).

### Step 2. Synthesis of Compound 47

Compound 46 (0.45 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 47 (C₂₁H₂₄N₃O₃, [M+H]⁺ 366.2; found 366.3, crude product).

### Step 3. Synthesis of Compound 48

Compound 47 (0.35 mmol), Compound 21 (0.65 mmol), N,N-diisopropylethylamine (1.25 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 48 (C₂₄H₂₆N₃O₃, [M+H]⁺ 366.2; found 366.3, crude product).

### Step 4. Synthesis of Compound D1-1

Under nitrogen protection, Compound R_{2-D1} (0.25 mmol), Compound 48 (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the organic combined phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound D1-1 (1.1 mg, 1.26 μmol). LC-MS: C₅₅H₄₈N₇O₄, [M+H]⁺ 435.7; found 436.2. ¹H NMR (400 MHz, DMSO-d6) δ 9.87-9.83 (m, 1H), 9.31-9.29 (m, 1H), 8.85 (s, 1H), 8.21 (s, 1H), 8.09 (s, 1H), 7.85 (s, 1H), 7.75-7.65 (m, 7H), 7.57-7.46 (m, 5H), 7.38 (s, 2H), 5.12-5.10 (m, 1H), 4.94-4.80 (m, 2H), 4.46-4.43 (m, 1H), 4.34-4.31 (m, 1H), 3.99 (s, 1H), 3.73 (s, 1H), 3.53 (s, 2H), 2.99 (s, 2H), 2.91 (s, 3H), 2.62-2.59 (m, 1H), 2.45-2.39 (m, 2H), 2.22 (s, 2H), 2.01 (s, 1H), 1.83-1.81 (m, 2H), 1.54 (s, 1H), 1.39-1.37 (m, 2H), 1.26-1.23 (m, 2H).

### Example 7. Synthesis of Compounds D2-1, D2-1(R), D2-2, D2-3, D2-4, D2-4-A, D2-5, D2-6, and D2-7 of the Present Invention

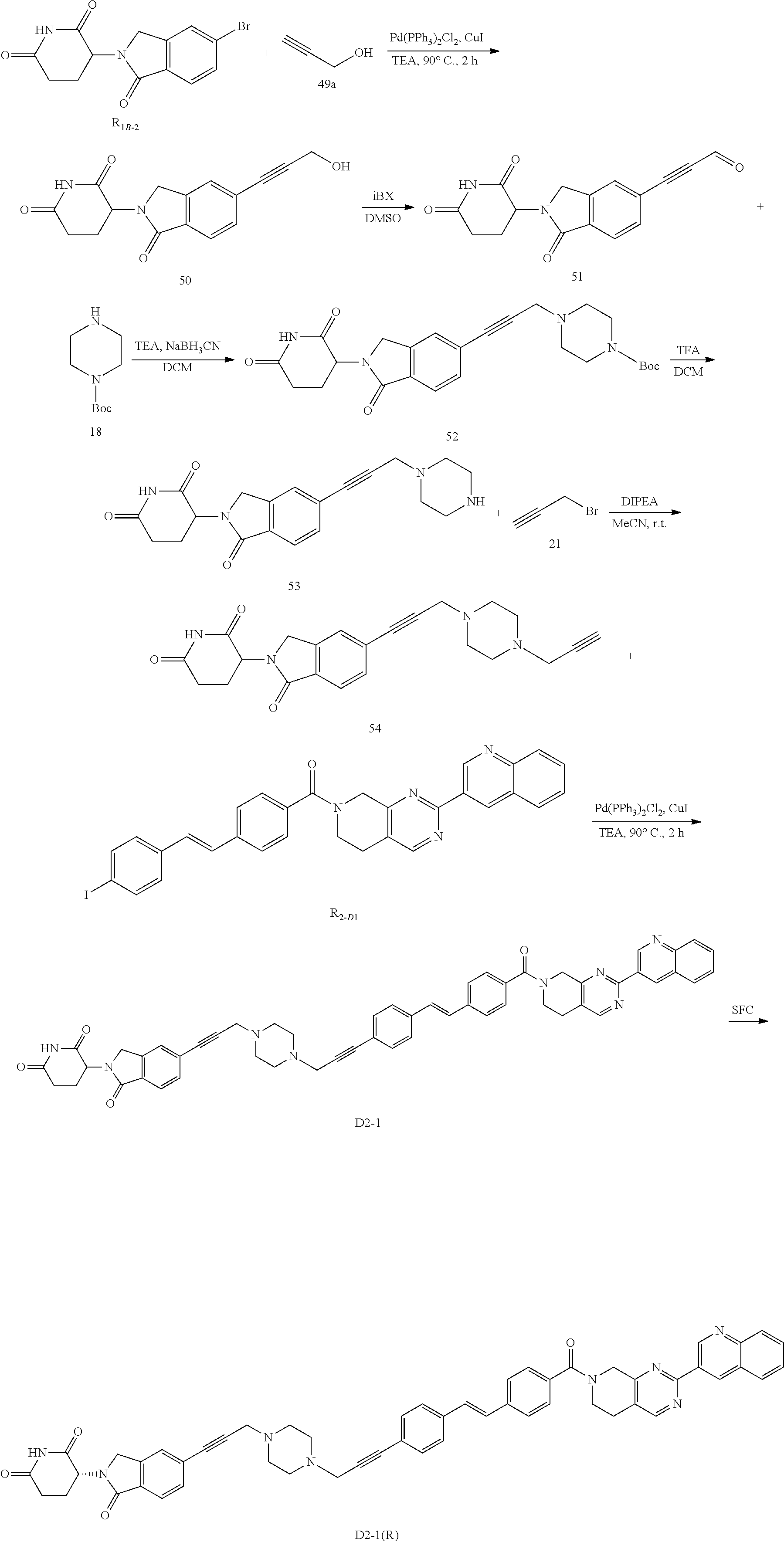

### Step 1. Synthesis of Compound 50

Under nitrogen protection, Compound R_{1B-2} (1.00 mmol), Compound 49a (1.02 mmol), copper iodide (2.00 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound 50 (C₁₆H₁₅N₂O₄, [M+H]⁺ 299.1; found 299.3, yield 48.9%).

### Step 2. Synthesis of Compound 51

Compound 50 (0.45 mmol), 2-iodoxybenzoic acid (1.02 mmol) and DMSO (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 51 (C₁₁H₁₃N₂O₄, [M+H]⁺ 297.1; found 297.2, 66.7%).

### Step 3. Synthesis of Compound 52

Compound 51 (0.30 mmol), Compound 18 (0.45 mmol), triethylamine (2 mL), sodium cyanoborohydride (0.55 mmol) and dichloroethane (5 mL) were added in sequence to a 50 mL reaction bottle, and reacted under stirring at room temperature for 2 hours, then the reaction was quenched (LC-MS monitoring), the solvent was removed by evaporation to dryness, purification was carried out by medium-pressure preparative chromatography, and the solvent was removed by concentration under reduced pressure to obtain Compound 52 (C₂₅H₃₁N₄O₅, [M+H]⁺ 467.2; found 467.1, yield 71.2%).

### Step 4. Synthesis of Compound 53

Compound 52 (0.21 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 53 (C₂₀H₂₃N₄O₃, [M+H]⁺ 367.2; found 367.1, crude product).

### Step 5. Synthesis of Compound 54

Compound 53 (0.20 mmol), Compound 21 (0.55 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 54 (C₂₃H₂₅N₄O₃, [M+H]⁺ 405.2; found 405.1, yield 76.9%).

### Step 6. Synthesis of Compound D2-1

Under nitrogen protection, Compound R_{2-D1} (0.15 mmol), Compound 54 (0.15 mmol), copper iodide (0.45 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, purified by column chromatography (the volume ratio of the eluents used was petroleum ether/ethyl acetate=1:100 to 1:5), and the solvent was removed by concentration under reduced pressure to obtain Compound D2-1 (19.0 mg, 21.8 μmol). LC-MS: C₅₄H₄₇N₈O₄, [M+H]⁺ 872.01; found 871.3. ¹H NMR (400 MHz, DMSO-d6) δ 11.01 (s, 1H), 9.87-9.83 (m, 1H), 9.31-9.29 (m, 1H), 8.86 (s, 1H), 8.21 (s, 1H), 8.07 (s, 1H), 7.95 (s, 1H), 7.77-7.73 (m, 2H), 7.72-7.70 (m, 3H), 7.66-7.63 (m, 2H), 7.58-7.54 (m, 3H), 7.48-7.46 (m, 2H), 7.40 (s, 2H), 5.14-5.09 (m, 1H), 4.95-4.81 (m, 1H), 4.48-4.43 (m, 1H), 4.35-4.31 (m, 1H), 4.06 (s, 1H), 3.59-3.56 (m, 3H), 3.17-3.16 (m, 1H), 3.00-2.97 (m, 2H), 2.92-2.86 (m, 2H), 2.68-2.67 (m, 3H), 2.66 (s, 4H), 2.23-2.02 (m, 3H), 2.00-1.97 (m, 2H).

### Step 7. Synthesis of Compound D2-1(R)

Compound D2-1 was separated by SFC (A:0.1% DEA/EtOH, B:0.1% DEA/DCM) to obtain Compound D2-1(R) (58.5 mg, found 871.4. ¹H NMR (600 MHz, DMSO-d6) δ 11.01 (s, 1H), 9.83-9.74 (m, 1H), 9.31-9.24 (m, 1H), 8.86 (s, 1H), 8.22 (d, J=9.0 Hz, 1H), 8.11 (s, 1H), 7.86 (s, 1H), 7.81-7.77 (m, 4H), 7.76-7.74 (m, 3H), 7.68 (t, J=8.4 Hz, 1H), 7.60-7.55 (m, 4H), 7.44-7.42 (m, 2H), 5.14-5.11 (m, 1H), 4.95-4.80 (m, 2H), 4.49-4.46 (m, 1H), 4.37-4.34 (m, 1H), 4.17-4.12 (m, 6H), 4.04-3.92 (m, 6H), 3.07-3.06 (m, 3H), 2.94-2.88 (m, 2H), 2.62-2.60 (m, 1H), 2.44-2.37 (m, 1H), 2.03-2.01 (m, 1H). Purity >90%.

According to the step 1 to step 6 of the method for Compound D2-1, the raw material 1 in the following table was used to replace Compound R_{1B-2}, and the raw material 2 in the following table was used to replace Compound 49a, and the other raw materials and operating methods remained unchanged to obtain Compounds D2-2 to D2-5.

| Com- | | | | |
|---|---|---|---|---|
| pound | | Raw | Raw | |
| No. | Structure and characterization data of compound | material 1 | material 2 | Yield |
| D2-2 | 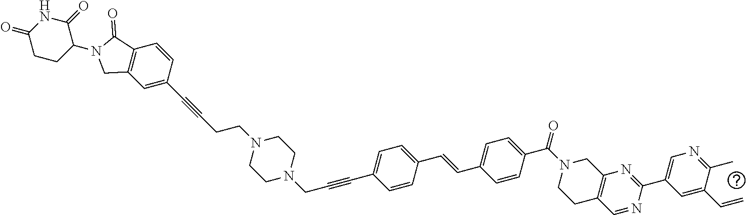 D2-2 LC-MS: C55H49N804, [M/2 + H]⁺ 443.5; found 443.7. ¹H NMR (400 MHz, DMSO-d₆) δ 11.01 (s, 1H), 9.84-9.78 (m, 1H), 9.34-9.28 (m, 1H), 8.86 (s, 1H), 8.23 (s, 1H), 8.11 (s, | 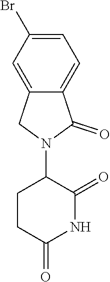 [SMILES: O=C1CCC(N2Cc3cc(Br)ccc3C2=O)C(=O)N1] R_{1B-2} | 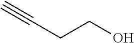 49b | 26.8% |
| | 1H), 7.87 (s, 1H), 7.74-7.63 (m, 7H), 7.56- | | | |
| | 7.49 (m, 5H), 7.40 (s, 2H), 5.12-5.10 (m, 1H), | | | |
| | 4.95-4.80 (m, 2H), 4.47-4.47 (m, 1H), 4.35- | | | |
| | 4.32 (m, 1H), 4.04 (s, 2H), 3.36 (s, 5H), 2.98- | | | |
| | 2.89 (m, 11H), 2.61-2.50 (m, 1H), 2.39 (s, | | | |
| | 2H), 2.01 (s, 1H). | | | |
| D2-3 | 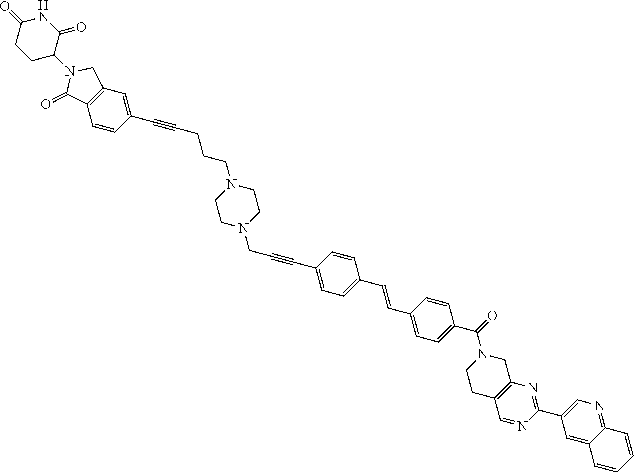 [SMILES: O=C1CCC(N2Cc3cc(C#CCCCN4CCN(CC#Cc5ccc(/C=C/c6ccc(C(=O)N7CCc8cnc(-c9cnc%10ccccc%10c9)nc8C7)cc6)cc5)CC4)ccc3C2=O)C(=O)N1] | 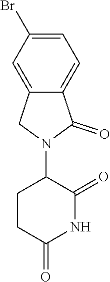 [SMILES: O=C1CCC(N2Cc3cc(Br)ccc3C2=O)C(=O)N1] R_{1B-2} | 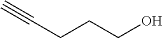 49c | 24.5% |
| | D2-3 | | | |
| | LC-MS: C₅₆H₅₁N₈O₄, [M/2 + H]⁺ 450.5; found | | | |
| | 450.5. ¹H NMR (400 MHz, Methanol-d₄) δ | | | |
| | 9.95-9.80 (m, 1H), 9.72-9.60 (m, 1H), 8.81 (s, | | | |
| | 1H), 8.30-8.25 (m, 2H), 8.18 (s, 1H), 8.04 (s, | | | |
| | 1H), 7.75-7.72 (m, 3H), 7.61-7.54 (m, 6H), | | | |
| | 7.43 (d, J = 8.0 Hz, 2H), 7.32-7.30 (m, 2H), | | | |
| | 5.16-5.12 (m, 2H), 4.46 (d, J = 8.0 Hz, 3H), | | | |
| | 3.97 (s, 2H), 3.77 (s, 2H), 3.72 (t, J = 4.0 Hz, | | | |
| | 1H), 3.48-3.39 (m, 3H), 3.13-2.85 (m, 6H), | | | |
| | 2.80-2.75 (m, 2H), 2.66-2.63 (m, 1H), 2.48 (t, | | | |
| | J = 8.0 Hz, 2H), 2.46-2.45 (m, 1H), 2.18-2.15 | | | |
| | (m, 1H), 2.14-2.07 (m, 2H), 1.88-1.85 (m, 1H). | | | |
| D2-4 | 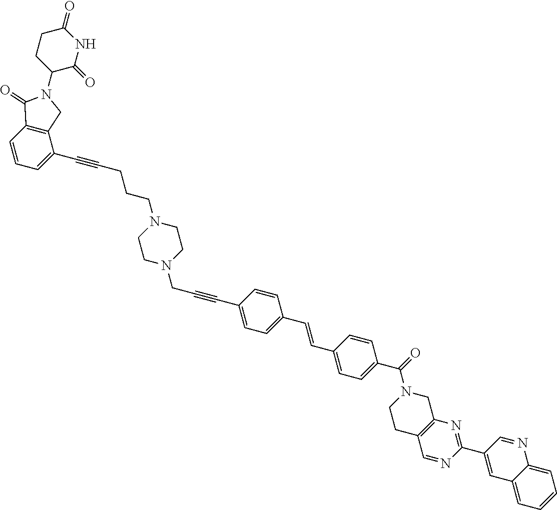 [SMILES: O=C1CCC(N2Cc3c(C#CCCCN4CCN(CC#Cc5ccc(/C=C/c6ccc(C(=O)N7CCc8cnc(-c9cnc%10ccccc%10c9)nc8C7)cc6)cc5)CC4)cccc3C2=O)C(=O)N1] | 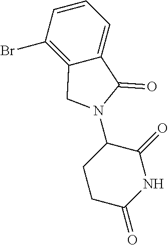 [SMILES: O=C1CCC(N2Cc3c(Br)cccc3C2=O)C(=O)N1] R1B-1 | 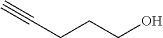 49c | 10.8% |
| | D2-4 | | | |
| | LC-MS: C₅₆H₅₁N₈O₄, [M/2 + H]⁺ 450.6; | | | |
| | found 450.7. ¹H NMR (400 MHz, DMSO- | | | |
| | d₆) δ 9.86-9.78 (m, 1H), 9.46-9.34 (m, 1H), | | | |
| | 8.86 (s, 1H), 8.29-8.21 (m, 2H), 8.16 (s, 1H), | | | |
| | 7.97-7.78 (m, 4H), 7.76-7.67 (m, 3H), 7.59- | | | |
| | 7.55 (m, 3H), 7.50 (d, J = 8.0 Hz, 2H), 7.41- | | | |
| | 7.38 (m, 2H), 5.13-5.10 (m, 1H), 4.97-4.81 | | | |
| | (m, 2H), 4.54-4.50 (m, 1H), 4.39-4.34 (m, | | | |
| | 1H), 3.79-3.61 (m, 4H), 3.45-3.22 (m, 6H), | | | |
| | 3.10-2.88 (m, 7H), 2.68-2.54 (m, 2H), 2.46- | | | |
| | 2.41 (m, 2H), 2.09-1.95 (m, 3H). | | | |
| D2-4-A | 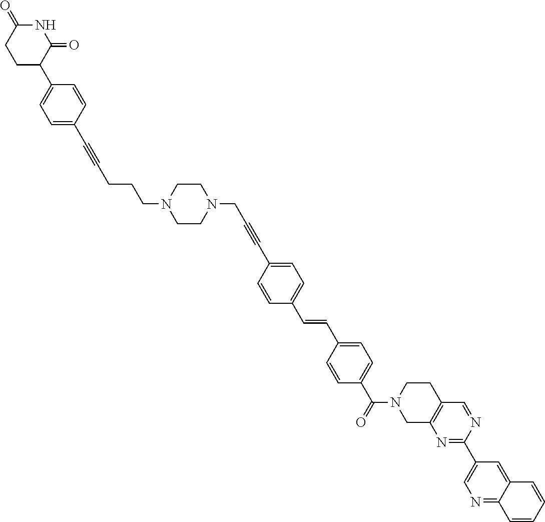 [SMILES: O=C1CCC(c2ccc(C#CCCCN3CCN(CC#Cc4ccc(/C=C/c5ccc(C(=O)N6CCc7cnc(-c8cnc9ccccc9c8)nc7C6)cc5)cc4)CC3)cc2)C(=O)N1] | 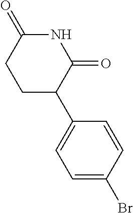 [SMILES: O=C1CCC(c2ccc(Br)cc2)C(=O)N1] R_{1C} | 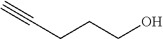 49c | 19.6% |
| | D2-4-A | | | |
| | LC-MS: C₅₄H₅₀N₇O₃, [M + H]⁺ 844.4; found | | | |
| | 844.3. ¹H NMR (600 MHz, Methanol-d₄) δ | | | |
| | 10.86 (s, 1H), 9.83-9.76 (m, 1H), 9.31-9.26 | | | |
| | (m, 1H), 8.86 (s, 1H), 8.23 (br, 1H), 8.11 (br, | | | |
| | 1H), 7.86 (s, 1H), 7.74 (d, J = 8.4 Hz, 2H), | | | |
| | 7.71 (d, J = 3.6 Hz, 2H), 7.67 (d, J = 8.4 Hz, | | | |
| | 2H), 7.56 (d, J = 8.4 Hz, 2H), 7.49-7.39 (m, | | | |
| | 5H), 7.22 (d, J = 8.4 Hz, 2H), 4.95-4.80 (m, | | | |
| | 2H), 4.02 (s, 1H), 3.98-3.81 (m, 2H), 3.39 (s, | | | |
| | 2H), 3.21 (s, 2H), 3.09-3.04 (m, 3H), 2.99- | | | |
| | 2.98 (m, 3H), 2.70-2.64 (m, 4H), 2.56-2.54 | | | |
| | (m, 2H), 2.21-2.18 (m, 2H), 2.03-1.93 (m, | | | |
| | 4H). Purity >99%. | | | |
| D2-5 | 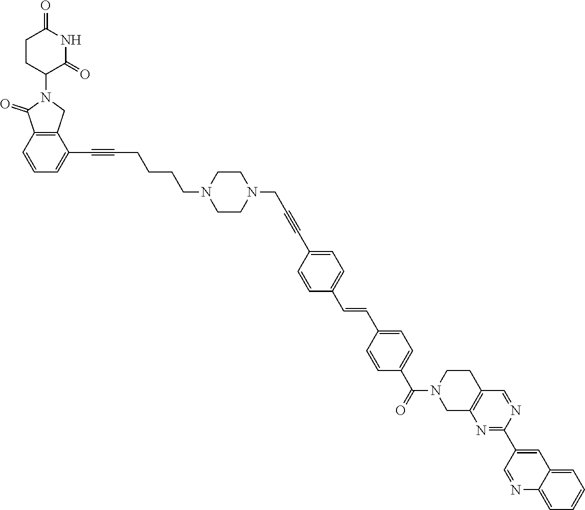 [SMILES: O=C1CCC(N2Cc3c(C#CCCCCN4CCN(CC#Cc5ccc(/C=C/c6ccc(C(=O)N7CCc8cnc(-c9cnc%10ccccc%10c9)nc8C7)cc6)cc5)CC4)cccc3C2=O)C(=O)N1] | 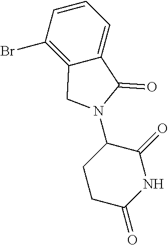 [SMILES: O=C1CCC(N2Cc3c(Br)cccc3C2=O)C(=O)N1] R1B-1 | 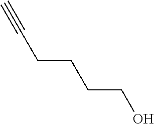 49d | 11.2% |
| | D2-5 | | | |
| | LC-MS: C₅₇H₅₃N₈O₄, [M/2 + H]⁺ 457.2; found | | | |
| | 457.7. ¹H NMR (400 MHz, DMSO-d₆) δ | | | |
| | 9.85-9.76 (m, 1H), 9.43-9.32 (m, 1H), 8.85 (s, | | | |
| | 1H), 8.27-8.14 (m, 2H), 7.95 (s, 1H), 7.77- | | | |
| | 7.73 (m, 4H), 7.72-7.66 (m, 3H), 7.60-7.55 | | | |
| | (m, 3H), 7.49 (d, J = 8.0 Hz, 2H), 7.41-7.38 | | | |
| | (m, 2H), 5.12-5.09 (m, 1H), 4.97-4.81 (m, | | | |
| | 2H), 4.52-4.48 (m, 1H), 4.37-4.33 (m, 1H), | | | |
| | 3.88-3.76 (m, 4H), 3.56-3.37 (m, 3H), 3.34- | | | |
| | 2.86 (m, 11H), 2.68-2.54 (m, 2H), 2.46-2.41 | | | |
| | (m, 2H), 2.09-2.06 (m, 1H), 1.82-1.78 (m, | | | |
| | 2H), 1.68-1.62 (m, 2H). | | | |

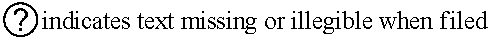

According to the step 1 to step 6 of the method for Compound D2-1, Compound R_{1B-1} was used to replace Compound R_{1B-2}, the raw material 1 in the following table was used to replace Compound R_{2-D1}, the raw material 2 in the following table was used to replace Compound 49a, and the other raw materials and operating methods remained unchanged to obtain Compounds D2-6, D2-7, D2-8, D2-9 and D2-10.

| Compound | | Raw | Raw | |
|---|---|---|---|---|
| No. | Structure and characterization data of compound | material 1 | material 2 | Yield |
| D2-6 | 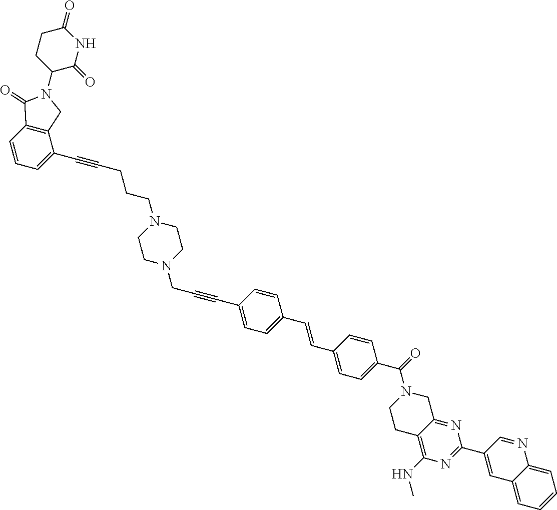 [SMILES: CNc1nc(-c2cnc3ccccc3c2)nc2c1CCN(C(=O)c1ccc(/C=C/c3ccc(C#CCN4CCN(CCCC#Cc5cccc6c5CN(C5CCC(=O)NC5=O)C6=O)CC4)cc3)cc1)C2] | 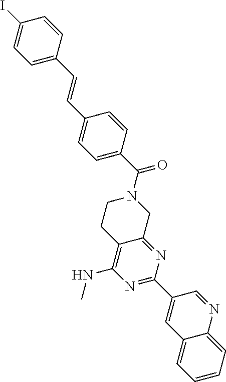 [SMILES: CNc1nc(-c2cnc3ccccc3c2)nc2c1CCN(C(=O)c1ccc(/C=C/c3ccc(I)cc3)cc1)C2] R2-B1 | 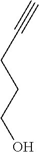 49c | 21.5% |
| | D2-6 | | | |
| | LC-MS: C₅₇H₅₄N₉O₄, [M/2 + H]⁺ 465.1; | | | |
| | found 465.1. ¹H NMR (400 MHz, DMSO- | | | |
| | d₆) δ 9.76-9.69 (m, 1H), 9.36-9.23 (m, 1H), | | | |
| | 8.24-8.15 (m, 2H), 8.14 (s, 1H), 7.77-7.75 | | | |
| | (m, 4H), 7.71-7.66 (m, 3H), 7.57-7.52 (m, | | | |
| | 3H), 7.50-7.48 (m, 2H), 7.37 (s, 2H), 5.14- | | | |
| | 5.09 (m, 1H), 4.79-4.61 (m, 2H), 4.54-4.50 | | | |
| | (m, 1H), 4.39-4.35 (m, 1H), 3.72 (s, 4H), | | | |
| | 3.52-3.23 (m, 6H), 3.20 (s, 4H), 3.12-2.87 | | | |
| | (m, 4H), 2.69-2.60 (m, 5H), 2.46-2.41 (m, | | | |
| | 2H), 2.11-1.95 (m, 3H). | | | |
| D2-7 | 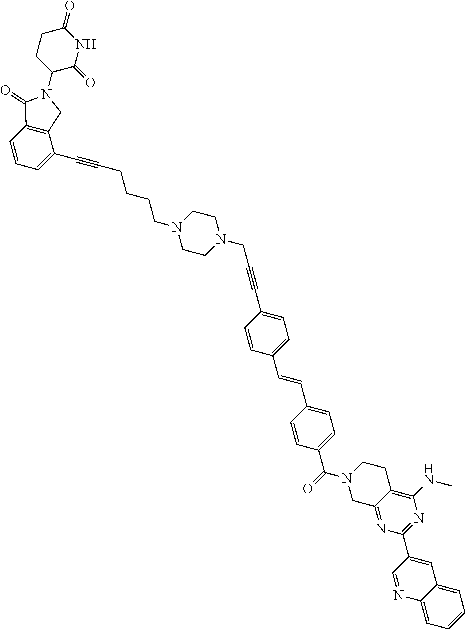 [SMILES: CNc1nc(-c2cnc3ccccc3c2)nc2c1CCN(C(=O)c1ccc(/C=C/c3ccc(C#CCN4CCN(CCCCC#Cc5cccc6c5CN(C5CCC(=O)NC5=O)C6=O)CC4)cc3)cc1)C2] | 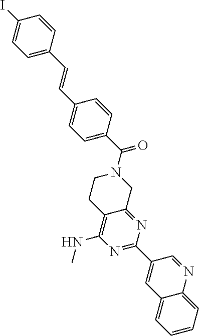 [SMILES: CNc1nc(-c2cnc3ccccc3c2)nc2c1CCN(C(=O)c1ccc(/C=C/c3ccc(I)cc3)cc1)C2] R2-B1 | 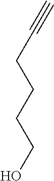 49d | 13.6% |
| | D2-7 | | | |
| | LC-MS: C₅₈H₅₆N₉O₄, [M + H]⁺943.1; found | | | |
| | 472.2. ¹H NMR (400 MHz, DMSO-d₆) δ | | | |
| | 9.76-9.68 (m, 1H), 9.37-9.24 (m, 1H), 8.29- | | | |
| | 8.24 (m, 2H), 8.17 (s, 1H), 7.97-7.75 (m, | | | |
| | 4H), 7.67 (d, J = 8.0 Hz, 3H), 7.57-7.53 (m, | | | |
| | 3H), 7.50-7.48 (m, 2H), 7.37 (s, 2H), 5.14- | | | |
| | 5.09 (m, 1H), 4.72-4.63 (m, 2H), 4.52-4.48 | | | |
| | (m, 1H), 4.37-4.33 (m, 1H), 3.73 (s, 4H), | | | |
| | 3.57-3.35 (m, 4H), 3.16 (s, 5H), 3.13-2.89 | | | |
| | (m, 4H), 2.68-2.58 (m, 5H), 2.46-2.43 (m, | | | |
| | 2H), 2.09-2.05 (m, 1H), 1.82-1.79 (m, 2H), | | | |
| | 1.69-1.59 (m, 2H). | | | |
| D2-8 | 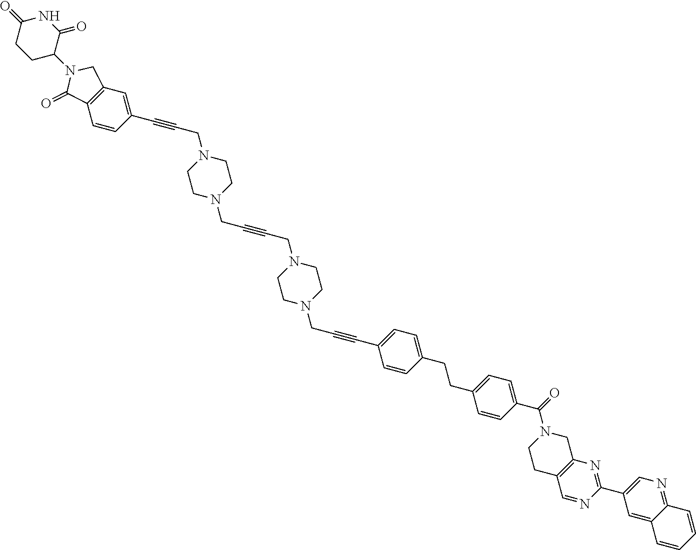 [SMILES: O=C1CCC(N2Cc3cc(C#CCN4CCN(CC#CCN5CCN(CC#Cc6ccc(CCc7ccc(C(=O)N8CCc9cnc(-c%10cnc%11ccccc%11c%10)nc9C8)cc7)cc6)CC5)CC4)ccc3C2=O)C(=O)N1] | 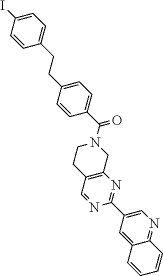 [SMILES: O=C(c1ccc(CCc2ccc(I)cc2)cc1)N1CCc2cnc(-c3cnc4ccccc4c3)nc2C1] R_{2-D3} | 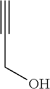 49a | 29.8% |
| | D2-8 | | | |
| | LC-MS: C₅₄H₄₉N₈O₄, [M/2 + H]⁺ 437.2; | | | |
| | found 437.6. ¹H NMR (600 MHz, DMSO- | | | |
| | d₆) δ11.01(s, 1H), 9.83-9.76 (m, 1H), 9.31- | | | |
| | 9.25 (m, 1H), 8.84 (m, 1H), 8.22 (br, 1H), | | | |
| | 8.10 (br, 1H), 7.86 (br, 1H), 7.77-7.75 (m, | | | |
| | 2H), 7.70-7.69 (m, 1H), 7.64-7.62 (m, 1H), | | | |
| | 7.44-7.42 (m, 4H), 7.34 (d, J = 7.8 Hz, 2H), | | | |
| | 7.29 (d, J = 7.8 Hz, 2H), 5.14-5.11 (m, 1H), | | | |
| | 4.93 (br, 1H), 4.75 (br, 1H), 4.47 (d, J = 17.4 | | | |
| | Hz, 1H), 4.35 (d, J = 17.4 Hz, 1H), 4.09 (s, | | | |
| | 3H), 3.99 (m, 6H), 3.74 (m, 10H), 2.92-2.88 | | | |
| | (m, 2H), 2.61 (d, J = 15.6 Hz, 1H), 2.44- | | | |
| | 2.36 (m, 1H), 2.03-2.01 (m, 1H). Purity >85%. | | | |
| D2-9 | 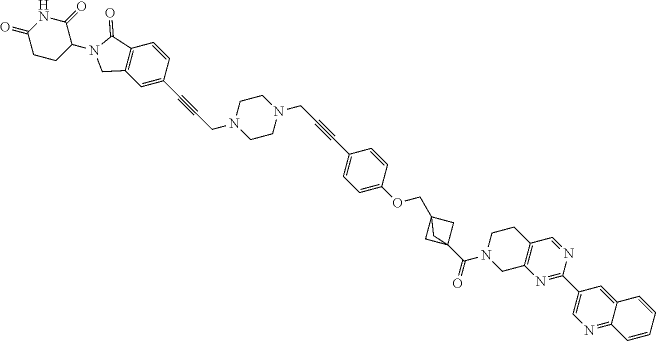 [SMILES: O=C1CCC(N2Cc3cc(C#CCN4CCN(CC#Cc5ccc(OCC67CC(C(=O)N8CCc9cnc(-c%10cnc%11ccccc%11c%10)nc9C8)(C6)C7)cc5)CC4)ccc3C2=O)C(=O)N1] D2-9 | 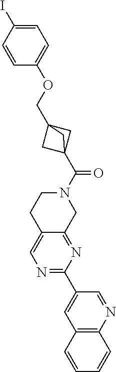 [SMILES: O=C(N1CCc2cnc(-c3cnc4ccccc4c3)nc2C1)C12CC(COc3ccc(I)cc3)(C1)C2] | 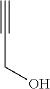 49a | 9.6% |
| | | R_{2-C5} | | |
| | LC-MS: C₅₂H₄₉N₈O₅, [M + H]⁺ 865.4; found | | | |
| | 865.1. ¹H NMR (600 MHz, DMSO-d₆) δ | | | |
| | 11.00 (s, 1H), 9.82-9.80 (m, 1H), 9.27 (s, | | | |
| | 1H), 8.81 (s, 1H), 8.21 (d, J = 7.8 Hz, 1H), | | | |
| | 8.10 (m, 1H), 7.85 (t, J = 7.8 Hz, 1H), 7.72- | | | |
| | 7.68 (m, 3H), 7.56 (d, J = 7.8 Hz, 1H), 7.37- | | | |
| | 7.36 (d, J = 8.4 Hz,, 2H), 6.95-6.94 (d, J = | | | |
| | 8.4 Hz, 2H), 5.13-5.10 (m, 1H), 4.95-4.78 | | | |
| | (m, 2H), 4.47-4.44 (d, J = 18.0 Hz 1H), 4.35- | | | |
| | 4.32 (d, J = 17.4 Hz, 1H), 4.08-4.04 (m, 3H), | | | |
| | 3.95-3.80 (m, 2H), 3.58 (s, 2H), 3.46 (s, 2H), | | | |
| | 3.34 (s, 2H), 2.94-2.86 (m, 3H), 2.61-2.59 | | | |
| | (m, 6H), 2.42-2.38 (qd, J = 13.2, 4.8 Hz 1H), | | | |
| | 2.27 (s, 6H), 2.02-2.00 (m, 1H). Purity >99%. | | | |
| D2-10 | 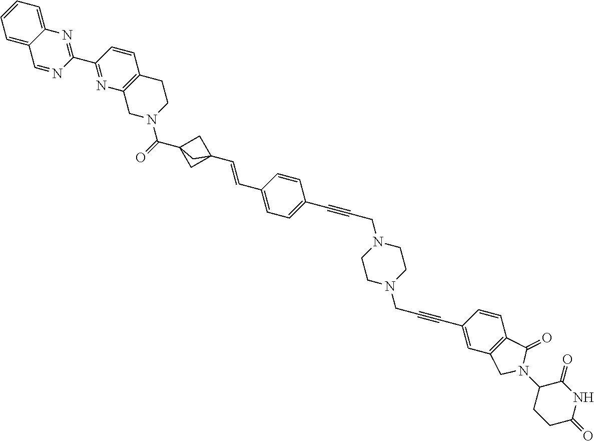 [SMILES: O=C1CCC(N2Cc3cc(C#CCN4CCN(CC#Cc5ccc(/C=C/C67CC(C(=O)N8CCc9ccc(-c%10ncc%11ccccc%11n%10)nc9C8)(C6)C7)cc5)CC4)ccc3C2=O)C(=O)N1] D2-10 | 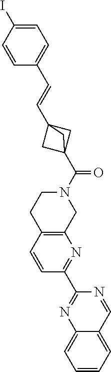 [SMILES: O=C(N1CCc2ccc(-c3ncc4ccccc4n3)nc2C1)C12CC(/C=C/c3ccc(I)cc3)(C1)C2] R_{2-D4} | 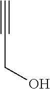 49a | 3.8% |
| | LC-MS: C₅₃H₄₉N₈O₄, [M/2 + H]⁺ 431.5; | | | |
| | found 431.7. ¹H NMR (600 MHz, DMSO- | | | |
| | d₆) δ 11.00 (s, 1H), 9.82-9.80 (m, 1H), 9.28- | | | |
| | 9.27 (m, 1H), 8.84-8.80 (m, 1H), 8.22 (t, J = | | | |
| | 8.4 Hz, 1H), 8.11-8.09 (m, 1H), 7.87-7.85 | | | |
| | (m, 1H), 7.84-7.82 (m, 2H), 7.81-7.78 (m, | | | |
| | 2H), 7.51-7.47 (m, 2H), 7.32-7.28 (m, 2H), | | | |
| | 6.60-6.53 (m, 1H), 5.81-5.76 (m, 1H), 5.13- | | | |
| | 5.11 (m, 1H), 4.87-4.80 (m, 1H), 4.74 (s, | | | |
| | 1H), 4.48-4.33 (m, 4H), 4.09-4.01 (m, 3H), | | | |
| | 3.88 (t, J = 6.0 Hz, 1H), 2.91-2.88 (m, 4H), | | | |
| | 2.62-2.59 (m, 2H), 2.41-2.39 (m, 2H), 2.28 | | | |
| | (s, 2H), 2.21-2.15 (m, 5H), 2.11-2.07 (m, | | | |
| | 2H), 2.04-2.01 (m, 3H). Purity >80%. | | | |

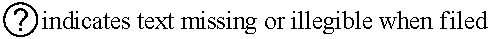

According to the step 1 to step 6 of the method for Compound D2-1, the raw material 1 in the following table was used to replace Compound R_{1B-2}, and the raw material 2 in the following table was used to replace Compound 49a, and the other raw materials and operating methods remained unchanged to obtain Compounds D2-11, D2-12, D2-13 and D2-14.

| Compound | | Raw | Raw | |
|---|---|---|---|---|
| No. | Structure and characterization data of compound | material 1 | material | Yield |
| D2-11 | 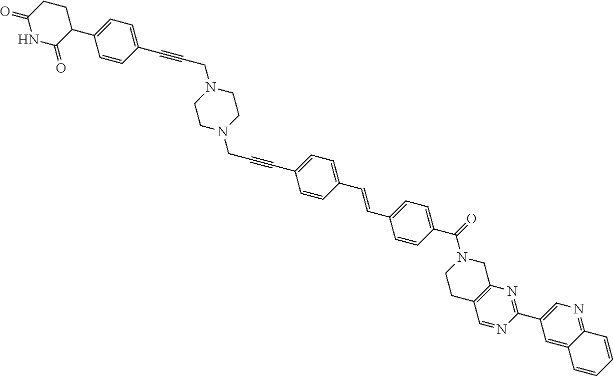 [SMILES: O=C1CCC(c2ccc(C#CCN3CCN(CC#Cc4ccc(/C=C/c5ccc(C(=O)N6CCc7cnc(-c8cnc9ccccc9c8)nc7C6)cc5)cc4)CC3)cc2)C(=O)N1] | 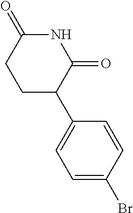 [SMILES: O=C1CCC(c2ccc(Br)cc2)C(=O)N1] R_{1C} | 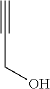 49a | 29.3% |
| | D2-11 | | | |
| | LC-MS: C₅₂H₄₆N₇O₃, [M/2 + H]⁺ 409.2; found | | | |
| | 409.1. ¹H NMR (600 MHz, DMSO-d₆) δ 10.88 | | | |
| | (s, 1H), 9.84-9.76 (m, 1H), 9.31-9.24 (m, 1H), | | | |
| | 8.86 (s, 1H), 8.23 (s, 1H), 8.10 (s, 1H), 7.86 (s, | | | |
| | 1H), 7.75 (d, J = 7.8 Hz, 2H), 7.69 (d, J = 8.4 | | | |
| | Hz, 3H), 7.61-7.53 (m, 5H), 7.48 (d, J = 7.8 | | | |
| | Hz, 2H), 7.43-7.42 (m, 2H), 7.29 (s, 1H), 4.95- | | | |
| | 4.80 (m, 2H), 4.06 (br, 4H), 3.94-3.91 (dd, J = | | | |
| | 12.0, 4.8 Hz 1H), 3.73 (br, 2H), 3.34-2.94 (m, | | | |
| | 10H), 2.71-2.65 (m, 1H), 2.50 (s, 1H), 2.23- | | | |
| | 2.21 (m, 1H), 2.08-2.01 (m, 1H). Purity >95%. | | | |
| D2-12 | 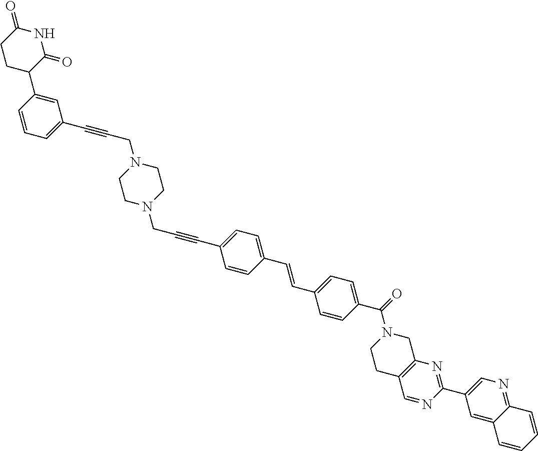 [SMILES: O=C1CCC(c2cccc(C#CCN3CCN(CC#Cc4ccc(/C=C/c5ccc(C(=O)N6CCc7cnc(-c8cnc9ccccc9c8)nc7C6)cc5)cc4)CC3)c2)C(=O)N1] | 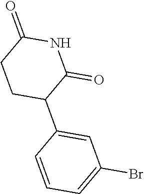 [SMILES: O=C1CCC(c2cccc(Br)c2)C(=O)N1] R_{2C} | 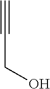 49a | 27.8% |
| | D2-12 | | | |
| | LC-MS: C₅₂H₄₆N₇O₃, [M/2 + H]⁺ 409.2; found | | | |
| | 409.0. ¹H NMR (600 MHz, DMSO-d₆) δ 10.88 | | | |
| | (s, 1H), 9.84-9.77 (m, 1H), 9.33-9.25 (m, 1H), | | | |
| | 8.85 (s, 1H), 8.23 (s, 1H), 8.11 (s, 1H), 7.87 (s, | | | |
| | 1H), 7.75 (d, J = 8.4 Hz, 2H), 7.69 (d, J = 8.4 | | | |
| | Hz, 3H), 7.57-7.53 (m, 4H), 7.43-7.38 (m, 5H), | | | |
| | 7.32-7.31 (d, J = 7.8 Hz, 1H), 4.95-4.80 (m, | | | |
| | 2H), 4.07 (s, 4H), 3.92-3.89 (dd, J = 12.0, 4.8 | | | |
| | Hz, 1H), 3.73 (br, 2H), 3.18-2.98 (m, 10H), | | | |
| | 2.72-2.66 (m, 1H), 2.55 (s, 1H), 2.27-2.21 (qd, | | | |
| | J = 12.6, 4.2 Hz, 1H), 2.06-2.02 (m, 1H). Purity | | | |
| | >99%. | | | |
| D2-13 | 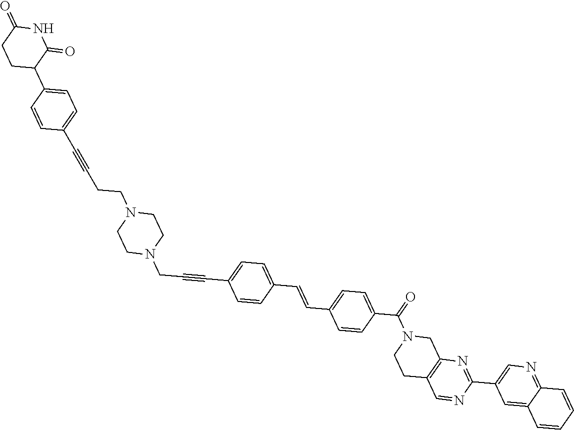 [SMILES: O=C1CCC(c2ccc(C#CCCN3CCN(CC#Cc4ccc(/C=C/c5ccc(C(=O)N6CCc7cnc(-c8cnc9ccccc9c8)nc7C6)cc5)cc4)CC3)cc2)C(=O)N1] | 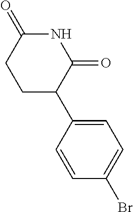 [SMILES: O=C1CCC(c2ccc(Br)cc2)C(=O)N1] R_{1C} | 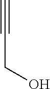 49b | 19.9% |
| | D2-13 | | | |
| | LC-MS: C₅₃H₄₈N₇O₃, [M + H]⁺ 830.4; found | | | |
| | 830.6. ¹H NMR (600 MHz, DMSO-d₆) δ 10.87 | | | |
| | (s, 1H), 9.86-9.78 (m, 1H), 9.37-9.29 (m, 1H), | | | |
| | 8.86 (s, 1H), 8.26 (s, 1H), 8.12 (s, 1H), 7.88- | | | |
| | 7.76 (s, 1H), 7.74-7.69 (m, 3H), 7.66-7.60 (m, | | | |
| | 2H), 7.57-7.55 (m, 2H), 7.54-7.53 (m, 2H), | | | |
| | 7.51-7.44 (m, 4H), 7.43-7.42 (d, J = 6.0 Hz, | | | |
| | 2H), 4.95 (br, 1H), 4.80 (br, 1H), 3.99-3.97 (m, | | | |
| | 1H), 3.91-3.88 (m, 1H), 3.82 (s, 2H), 3.74 (s, | | | |
| | 1H), 3.71-3.35 (m, 5H), 3.00-2.91 (m, 9H), | | | |
| | 2.70-2.65 (m, 1H), 2.49 (s, 1H), 2.22-2.19 (m, | | | |
| | 1H), 2.08-2.02 (m, 1H). Purity >95%. | | | |
| D2-14 | 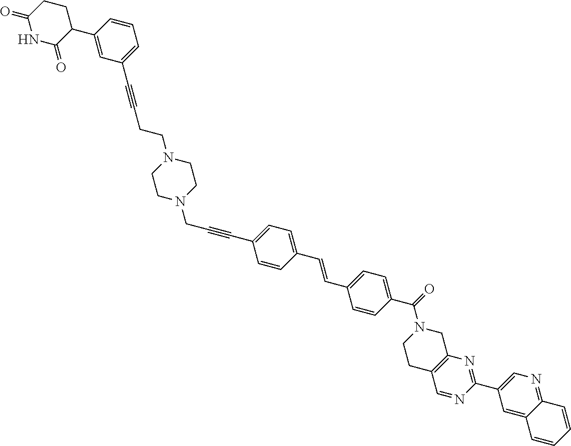 [SMILES: O=C1CCC(c2cccc(C#CCCN3CCN(CC#Cc4ccc(/C=C/c5ccc(C(=O)N6CCc7cnc(-c8cnc9ccccc9c8)nc7C6)cc5)cc4)CC3)c2)C(=O)N1] | 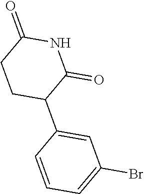 [SMILES: O=C1CCC(c2cccc(Br)c2)C(=O)N1] R_{2C} | 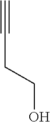 49b | 9.4% |
| | LC-MS: C₅₃H₄₈N₇O₃, [M + H]⁺ 830.4; found | | | |
| | 830.6. ¹H NMR (600 MHz, DMSO-d₆) δ 10.87 | | | |
| | (s, 1H), 9.85-9.77 (m, 1H), 9.32-9.25 (m, 1H), | | | |
| | 8.86 (s, 1H), 8.23 (s, 1H), 8.14 (s, 1H), 7.86 (s, | | | |
| | 1H), 7.77-7.75 (m, 2H), 7.74-7.73 (m, 3H), | | | |
| | 7.68-7.66 (d, J = 7.8 Hz, 2H), 7.56-7.55 (d, J = | | | |
| | 7.8 Hz, 2H), 7.50-7.49 (d, J = 8.4 Hz, 2H), | | | |
| | 7.43-7.40 (m, 2H), 7.37-7.28 (m, 2H), 4.95- | | | |
| | 4.76 (m, 2H), 4.00 (s, 1H), 3.90-3.88 (dd, J = | | | |
| | 12.0, 4.8 Hz, 1H), 3.78 (m, 3H), 3.60-2.91 (m, | | | |
| | 14H), 2.83-2.73 (m, 1H), 2.70-2.64 (m, 1H), | | | |
| | 2.23-2.21 (m, 1H), 2.04-2.02 (m, 1H). Purity | | | |
| | >99%. | | | |

According to the step 1 to step 6 of the method for Compound D2-1, Compound R_{2C} was used to replace Compound R_{1B-2}, the raw material 1 in the following table was used to replace Compound R_{2-D1}, the raw material 2 in the following table was used to replace Compound 49a, and the other raw materials and operations remained unchanged to obtain Compounds D2-15 and D2-16.

| Compound | | Raw | Raw | |
|---|---|---|---|---|
| No. | Structure and characterization data of compound | material 1 | material 2 | Yield |
| D2-15 | 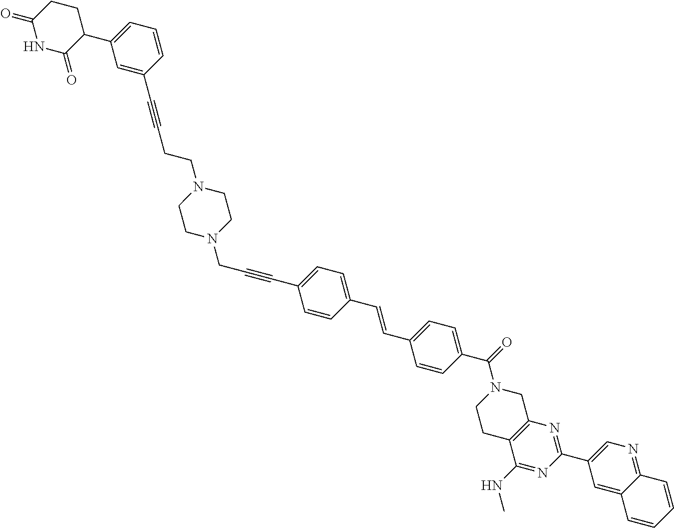 [SMILES: CNc1nc(-c2cnc3ccccc3c2)nc2c1CCN(C(=O)c1ccc(/C=C/c3ccc(C#CCN4CCN(CCC#Cc5cccc(C6CCC(=O)NC6=O)c5)CC4)cc3)cc1)C2] | 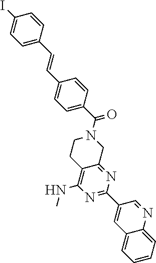 [SMILES: CNc1nc(-c2cnc3ccccc3c2)nc2c1CCN(C(=O)c1ccc(/C=C/c3ccc(I)cc3)cc1)C2] R2-B1 | 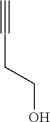 49b | 29.3% |
| | D2-15 | | | |
| | LC-MS: C₅₄H₅₁N₈O₃, [M + H]⁺ 859.4; found | | | |
| | 859.4. ¹H NMR (600 MHz, DMSO-d₆) δ 9.78- | | | |
| | 9.70 (m, 1H), 9.34-9.23 (m, 1H), 8.24-8.20 (m, | | | |
| | 1H), 8.13 (s, 1H), 7.91 (s, 1H), 7.74-7.73 (d, J = | | | |
| | 7.8 Hz, 3H), 7.66-7.65 (d, J = 7.8 Hz, 2H), | | | |
| | 7.53-7.48 (m, 4H), 7.37-7.27 (m, 4H), 7.25 (s, | | | |
| | 1H), 7.24-7.19 (m, 1H), 4.77 (br, 1H), 4.59 (br, | | | |
| | 1H), 3.95 (s, 1H), 3.88-3.85 (dd, J = 12.0, 4.8 | | | |
| | Hz, 1H), 3.80 (s, 2H), 3.53 (s, 2H), 3.33 (t, J = | | | |
| | 7.2 Hz, 6H), 3.10 (s, 3H), 2.96-2.89 (m, 6H), | | | |
| | 2.68-2.64 (m, 1H), 2.57-2.51 (m, 3H), 2.20-2.17 | | | |
| | (m, 1H), 2.03-2.01 (m, 1H). Purity >95%. | | | |
| D2-16 | 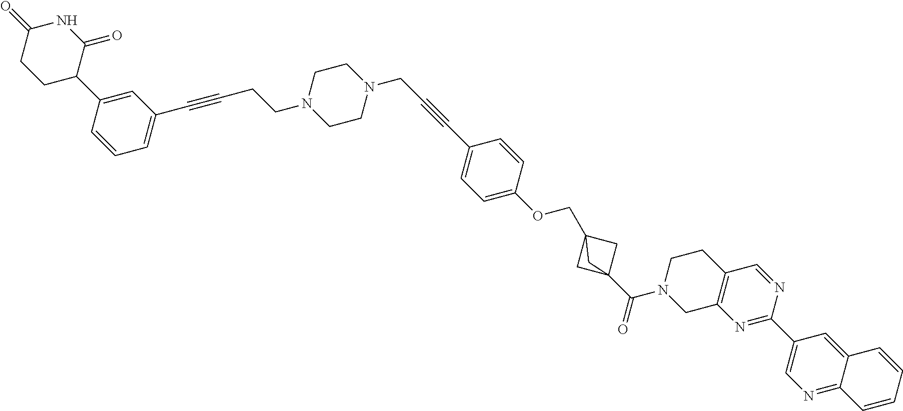 [SMILES: O=C1CCC(c2cccc(C#CCCN3CCN(CC#Cc4ccc(OCC56CC(C(=O)N7CCc8cnc(-c9cnc%10ccccc%10c9)nc8C7)(C5)C6)cc4)CC3)c2)C(=O)N1] D2-16 | 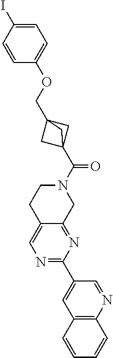 [SMILES: O=C(N1CCc2cnc(-c3cnc4ccccc4c3)nc2C1)C12CC(COc3ccc(I)cc3)(C1)C2] R_{2-C5} | 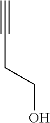 49b | 32.1% |
| | LC-MS: C₅₁H₅₀N₇O₄, [M + H]⁺ 824.4; found | | | |
| | 824.4. ¹H NMR (600 MHz, DMSO-d₆) δ 10.87 | | | |
| | (s, 1H), 9.82 (m, 1H), 9.31 (s, 1H), 8.83 (m, 1H), | | | |
| | 8.24 (d, J = 8.4 Hz, 1H), 8.11 (t, J = 8.4 Hz, | | | |
| | 1H), 7.88 (t, J = 7.2 Hz, 1H), 7.71 (t, J = 7.2 | | | |
| | Hz, 1H), 7.41-7.40 (m, 2H), 7.35-7.32 (m, 2H), | | | |
| | 7.28-7.25 (m, 2H), 6.97 (d, J = 9.0 Hz, 2H), | | | |
| | 4.95 (s. 1H), 4.79 (s, 2H), 4.10 (s, 2H), 3.96- | | | |
| | 3.94 (m, 1H), 3.89-3.87 (m, 1H), 3.80-3.78 (m, | | | |
| | 3H), 3.31-3.09 (m, 6H), 2.95-2.86 (m, 8H), | | | |
| | 2.70-2.64 (m, 1H), 2.23-2.22 (m, 1H), 2.20 (s, | | | |
| | 6H), 2.10-2.00 (m, 1H). Purity >99%. | | | |

### Example 8. Synthesis of Compounds E1-1 and E1-2 of the Present Invention

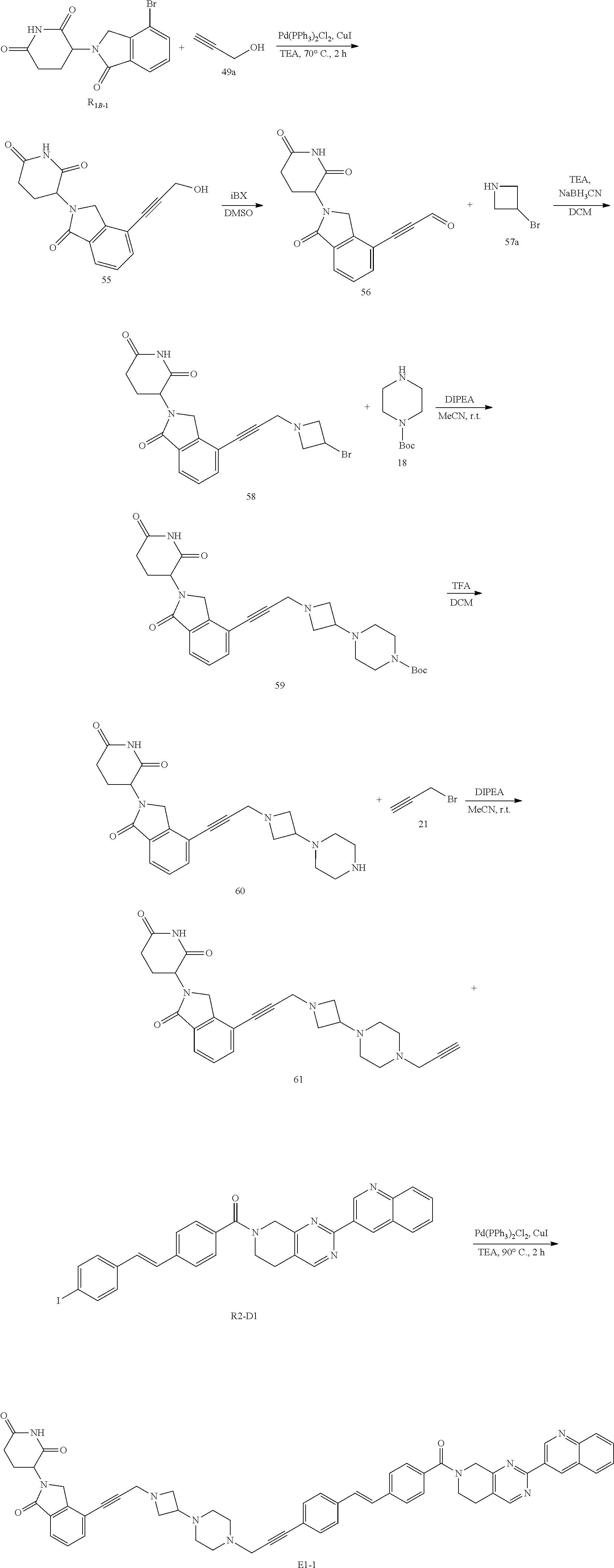

### Step 1. Synthesis of Compound 55

Under nitrogen protection, Compound R_{1B-1} (1.00 mmol), Compound 49a (1.02 mmol), copper iodide (2.00 mmol), triethylamine (5 mL), and Pd(PPh₃)₂Cl₂ (0.25 mol %) were added in sequence to a 50 mL reaction bottle. The reaction system was sealed, the reaction was carried out under stirring at 70° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound 55 (C₁₆H₁₅N₂O₄, [M+H]⁺ 299.1; found 299.3, yield 63.4%).

### Step 2. Synthesis of Compound 56

Compound 55 (0.60 mmol), 2-iodoxybenzoic acid (1.80 mmol) and DMSO (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 56 (C₁₆H₁₃N₂O₄, [M+H]⁺ 297.1; found 297.3, yield 78.2%).

### Step 3. Synthesis of Compound 58

Compound 56 (0.45 mmol), Compound 57a (0.65 mmol), triethylamine (2 mL), sodium cyanoborohydride (0.90 mmol) and dichloroethane (5 mL) were added in sequence to a 50 mL reaction bottle, reacted under stirring at room temperature for 2 hours, then the reaction was quenched (LC-MS monitoring), the solvent was removed by evaporation to dryness, purification was carried out by medium-pressure preparative chromatography, and the solvent was removed by concentration under reduced pressure to obtain Compound 58 (C₁₉H₁₉BrN₃O₃, [M+H]⁺ 416.1; found 416.2, yield 66.7%).

### Step 4. Synthesis of Compound 59

Compound 58 (0.30 mmol), Compound 18 (0.65 mmol), N,N-diisopropylethylamine (1.20 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 59 (C₂₈H₃₆N₅O₅, [M+H]⁺ 522.3; found 522.5, yield 63.8%).

### Step 5. Synthesis of Compound 60

Compound 59 (0.20 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 60 (C₂₃H₂₈N₅O₃, [M+H]⁺ 422.2; found 422.1, crude product).

### Step 6. Synthesis of Compound 61

Compound 60 (0.20 mmol), Compound 21 (0.50 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to remove the solvent, purified by medium-pressure preparative chromatography, and concentrated under reduced pressure to remove the solvent to obtain Compound 61 (C₂₆H₃₀N₅O₃, [M+H]⁺ 460.2; found 460.4, yield 75.2%).

### Step 7. Synthesis of Compound E1-1

Under nitrogen protection, Compound R_{2-D1} (0.15 mmol), Compound 61 (0.15 mmol), copper iodide (0.35 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound E1-1 (10.6 mg, 10.79 μmol). LC-MS: C₅₇H₅₂N₉O₄ [M/2+H]⁺464.1; found 464.2. ¹H NMR (400 MHz, Methanol-d4) δ 9.92 (s, 1H), 9.67 (s, 1H), 8.80 (s, 1H), 8.34-8.17 (m, 2H), 8.01 (s, 1H), 7.99 (s, 1H), 7.87-7.73 (m, 4H), 7.64-7.59 (m, 2H), 7.57-7.50 (m, 5H), 7.33 (s, 2H), 5.22-5.17 (m, 1H), 5.04-4.87 (m, 2H), 4.62-4.55 (m, 2H), 4.51-4.37 (m, 6H), 4.27-4.26 (m, 2H), 4.18-4.03 (m, 1H), 3.87-3.84 (m, 2H), 3.54-3.31 (m, 4H), 3.13-3.05 (m, 3H), 2.97-2.88 (m, 2H), 2.81-2.77 (m, 2H), 2.57-2.45 (m, 2H), 2.21-2.18 (m, 1H).

Compound E1-2 could be obtained by following the step 1 to step 7 of the method for Compound E1-1, replacing Compound 57a with the raw materials in the below table, and keeping other raw materials and operating methods unchanged.

| Compound | | Raw | |
|---|---|---|---|
| No. | Structure and characterization data of compound | material | Yield |
| E1-2 | 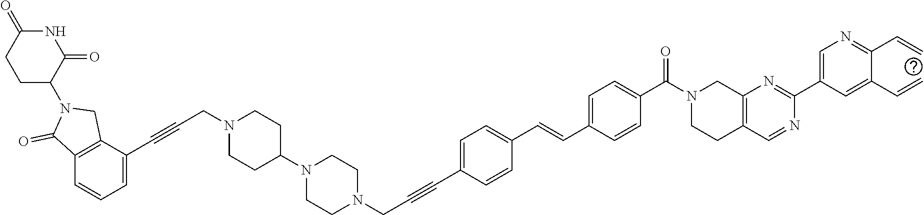 [SMILES: C=Cc1cc(-c2ncc3c(n2)CN(C(=O)c2ccc(/C=C/c4ccc(C#CCN5CCN(C6CCN(CC#Cc7cccc8c7CN(C7CCC(=O)NC7=O)C8=O)CC6)CC5)cc4)cc2)CC3)cnc1C=C] | 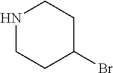 57b | 16.3% |
| | E1-2 | | |
| | LC-MS: C₅₉H₅₆N₉O₄ [M/2 + H]⁺ 478.5; found 478.1. ¹H | | |
| | NMR (400 MHz, Methanol-d₄) δ 9.95-9.88 (m, 1H), 9.72- | | |
| | 9.61 (m, 1H), 8.81 (s, 1H), 8.41-8.25 (m, 2H), 8.19-8.01 | | |
| | (m, 1H), 7.98-7.89 (m, 1H), 7.86-7.73 (m, 4H), 7.68-7.55 | | |
| | (m, 5H), 7.48-7.42 (m, 2H), 7.32 (s, 2H), 5.22-5.17 (m, | | |
| | 1H), 5.05-4.87 (m, 2H), 4.63-4.51 (m, 2H), 4.36 (s, 2H), | | |
| | 4.14 (s, 3H), 3.83-3.77 (m, 4H), 3.31-3.06 (m, 12H), 2.97- | | |
| | 2.92 (m, 1H), 2.89-2.77 (m, 1H), 2.53-2.49 (m, 1H), 2.48- | | |
| | 2.27 (m, 2H), 2.22-2.19 (m, 1H), 2.02-1.99 (m, 2H). | | |

### Example 9. Synthesis of Compounds F1A-1, F1A-2, F1A-3, F1A-4 and F1A-5 of the Present Invention

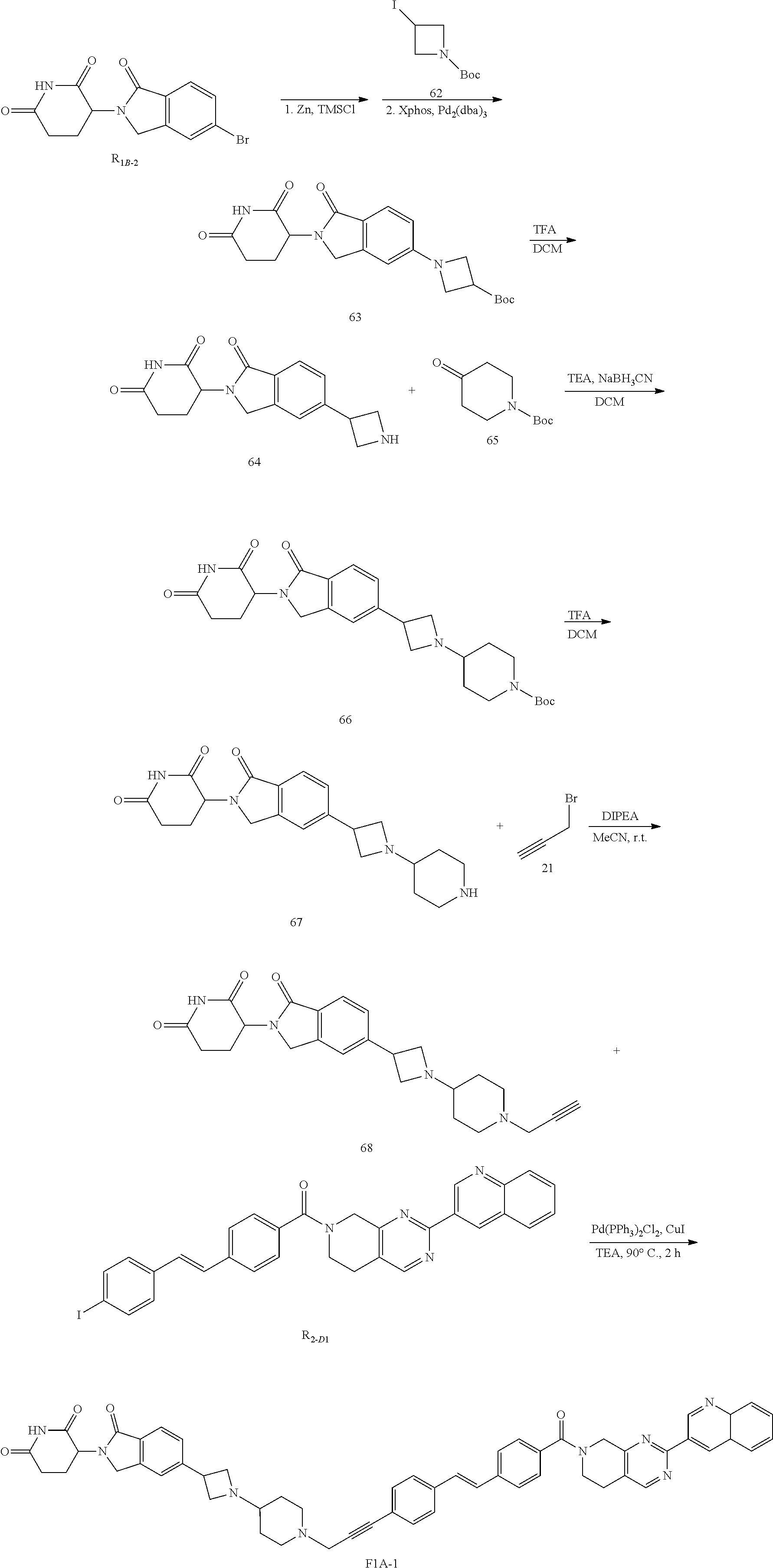

### Step 1. Synthesis of Compound 63

Compound R_{1B-2} (1.0 mmol), zinc powder and 1,4-dioxane (5 mL) were added in sequence to a 50 mL reaction bottle, TMSCl was added dropwise under ice bath conditions, slowly warmed to room temperature, reacted under stirring for 1 hour, and then the reaction was quenched (LC-MS monitoring). Extraction was carried out with saturated sodium chloride solution (20 mL) and ethyl acetate (3×20 mL), the organic phases were combined, the combined organic phase was dried over anhydrous sodium sulfate, and evaporated to dryness to remove the solvent. Under nitrogen protection, Compound 62 (1.0 mmol), Xphos (0.2 mmol), Pd₂(dba)₃ (0.25 mol %), and 1,4-dioxane (5 mL) were added into the reaction bottle. The reaction system was sealed, the reaction was carried out under stirring at 80° C. for 2 hours, and then the reaction was quenched (LC-MS monitoring). Extraction was carried out with saturated sodium chloride solution (20 mL) and ethyl acetate (3×20 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound 63 (C₂₁H₂₆N₃O₅, [M+H]⁺ 400.2; found 400.4, yield 56.8%).

### Step 2. Synthesis of Compound 64

Compound 63 (0.55 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 64 (C₁₆H₁₈N₃O₃, [M+H]⁺ 300.1; found 300.2, crude product).

### Step 3. Synthesis of Compound 66

Compound 64 (0.55 mmol), Compound 65 (0.65 mmol), triethylamine (2 mL), sodium cyanoborohydride (0.85 mmol) and dichloroethane (5 mL) were added in sequence to a 50 mL reaction bottle, reacted under stirring at room temperature for 2 hours, then the reaction was quenched (LC-MS monitoring), the solvent was removed by evaporation to dryness, purification was carried out by medium-pressure preparative chromatography, and the solvent was removed by concentration under reduced pressure to obtain Compound 66 (C₂₆H₃₅N₄O₅, [M+H]⁺ 483.3; found 483.4, yield 54.5%).

### Step 4. Synthesis of Compound 67

Compound 66 (0.30 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 67 (C₂₁H₂₇N₄O₃, [M+H]⁺ 383.2; found 383.2, crude product).

### Step 5. Synthesis of Compound 68

Compound 67 (0.30 mmol), Compound 21 (0.65 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to remove the solvent, purified by medium-pressure preparative chromatography, and concentrated under reduced pressure to remove the solvent to obtain Compound 68 (C₂₄H₂₉N₄O₃, [M+H]⁺ 421.2; found 421.1, yield 83.3%).

### Step 6. Synthesis of Compound F1A-1

Under nitrogen protection, Compound R_{2-D1} (0.25 mmol), Compound 68 (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound F1A-1 (11.9 mg, 13.40 μmol, 91.8% purity). LC-MS: C₅₅H₅₁N₈O₄, [M/2+H]⁺444.5; found 444.5. ¹H NMR (600 MHz, DMSO-d6) δ 9.82-9.78 (m, 1H), 9.29-9.23 (m, 1H), 8.85 (s, 1H), 8.21 (s, 1H), 8.09 (s, 1H), 7.84 (s, 1H), 7.75 (d, J=8.0 Hz, 2H), 7.67-7.64 (m, 4H), 7.58-7.55 (m, 3H), 7.49-7.45 (m, 3H), 7.39 (s, 2H), 5.10 (d, J=5.2 Hz, 1H), 4.94 (s, 1H), 4.80 (s, 1H), 4.44-4.41 (m, 1H), 4.32-4.29 (m, 1H), 3.99 (s, 1H), 3.69-3.63 (m, 3H), 3.52 (s, 2H), 3.11 (s, 2H), 2.99 (s, 2H), 2.91-2.89 (m, 1H), 2.80 (s, 2H), 2.61-2.58 (m, 1H), 2.26 (s, 2H), 2.09 (s, 1H), 1.99-1.94 (m, 1H), 1.75 (s, 2H), 1.24 (s, 4H).

Compounds F1A-4 and F1A-5 could be obtained by following the steps 1 to 6 of the method for Compound F1A-1, replacing R_{2-D1} with raw material 1 in the below table, and keeping other raw materials and operating methods unchanged.

| Compound | | | |
|---|---|---|---|
| No. | Structure and characterization data of compound | Raw material | Yield |
| F1A-4 | 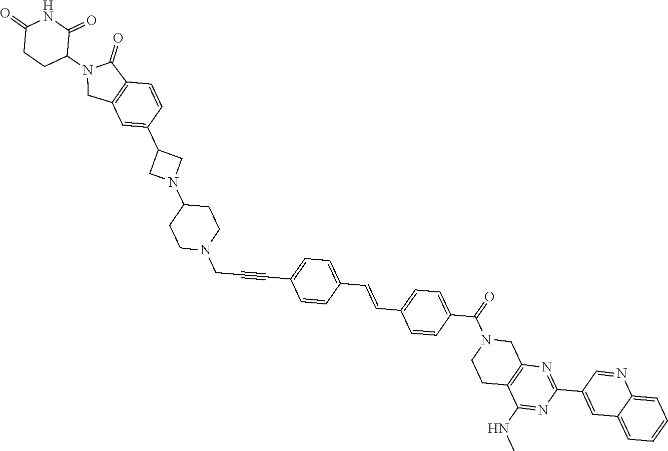 [SMILES: CNc1nc(-c2cnc3ccccc3c2)nc2c1CCN(C(=O)c1ccc(/C=C/c3ccc(C#CCN4CCC(N5CC(c6ccc7c(c6)CN(C6CCC(=O)NC6=O)C7=O)C5)CC4)cc3)cc1)C2] | 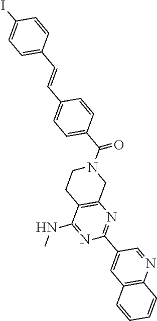 [SMILES: CNc1nc(-c2cnc3ccccc3c2)nc2c1CCN(C(=O)c1ccc(/C=C/c3ccc(I)cc3)cc1)C2] R2-B1 | 16.3% |
| | LC-MS: C₅₆H₅₄N₉O₄, [M + H]⁺ 916.4; found 916.8. ¹H | | |
| | NMR (600 MHz, DMSO-d₆) δ 9.82-9.73 (m, 1H), 9.34- | | |
| | 9.23 (m, 1H), 8.25-7.80 (m, 3H), 7.79-7.68 (m, 7H), | | |
| | 7.61-7.48 (m, 5H), 7.39 (s, 2H), 5.13-5.10 (m, 1H), 4.78- | | |
| | 4.25 (m, 12H), 3.99 (s, 1H), 3.74 (s, 3H), 3.11-2.89 (m, | | |
| | 6H), 2.64-2.59 (m, 3H), 2.46-2.40 (m, 1H), 2.28-2.27 | | |
| | (m, 2H), 2.04-2.02 (m, 1H), 1.72 (s, 2H). Purity >90%. | | |
| F1A-5 | 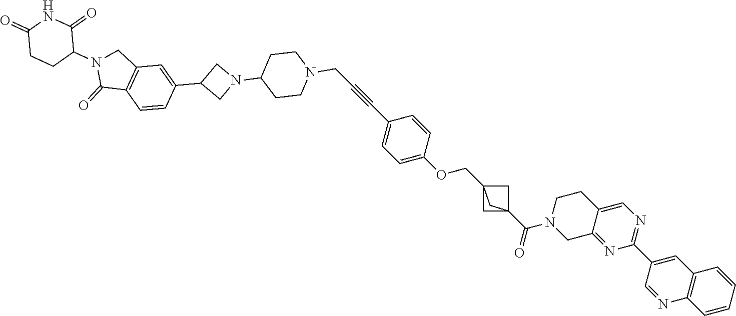 [SMILES: O=C1CCC(N2Cc3cc(C4CN(C5CCN(CC#Cc6ccc(OCC78CC(C(=O)N9CCc%10cnc(-c%11cnc%12ccccc%12c%11)nc%10C9)(C7)C8)cc6)CC5)C4)ccc3C2=O)C(=O)N1] F1A-5 | 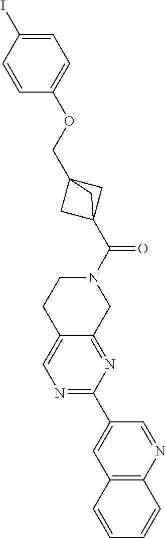 [SMILES: O=C(N1CCc2cnc(-c3cnc4ccccc4c3)nc2C1)C12CC(COc3ccc(I)cc3)(C1)C2] R_{2-C5} | 23.2% |
| | LC-MS: C₅₃H₅₃N₈O₅, [M/2 + H]⁺ 441.5; found 441.5. ¹H | | |
| | NMR (600 MHz, DMSO-d₆) δ 9.83 (s, 1H), 9.38-9.37 | | |
| | (m, 1H), 8.84-8.83 (m, 1H), 8.27 (d, J = 8.4 Hz, 1H), | | |
| | 8.15 (d, J = 8.4 Hz, 1H), 7.93 (t, J = 7.8 Hz, 1H), 7.80- | | |
| | 7.74 (m, 2H), 7.67 (s, 1H), 7.60 (d, J = 7.2 Hz, 1H), 7.50 | | |
| | (d, J = 1.8 Hz, 2H), 7.03 (d, J = 8.4 Hz, 2H), 5.72 (s, | | |
| | 1H), 5.12-5.09 (m, 1H), 4.96 (s, 1H), 4.80 (s, 1H), 4.56- | | |
| | 4.50 (m, 2H), 4.47 (s, 1H), 4.37 (s, 5H), 4.37 (s, 1H), | | |
| | 4.13 (s, 2H), 3.96 (s, 3H), 3.11 (s, 2H), 2.96-2.88 (m, | | |
| | 3H), 2.65-2.63 (m, 1H), 2.46-2.42 (m, 1H), 2.40-2.18 | | |
| | (m, 9H), 2.05-2.03 (m, 1H), 1.72-1.71 (m, 2H). Purity >90%. | | |

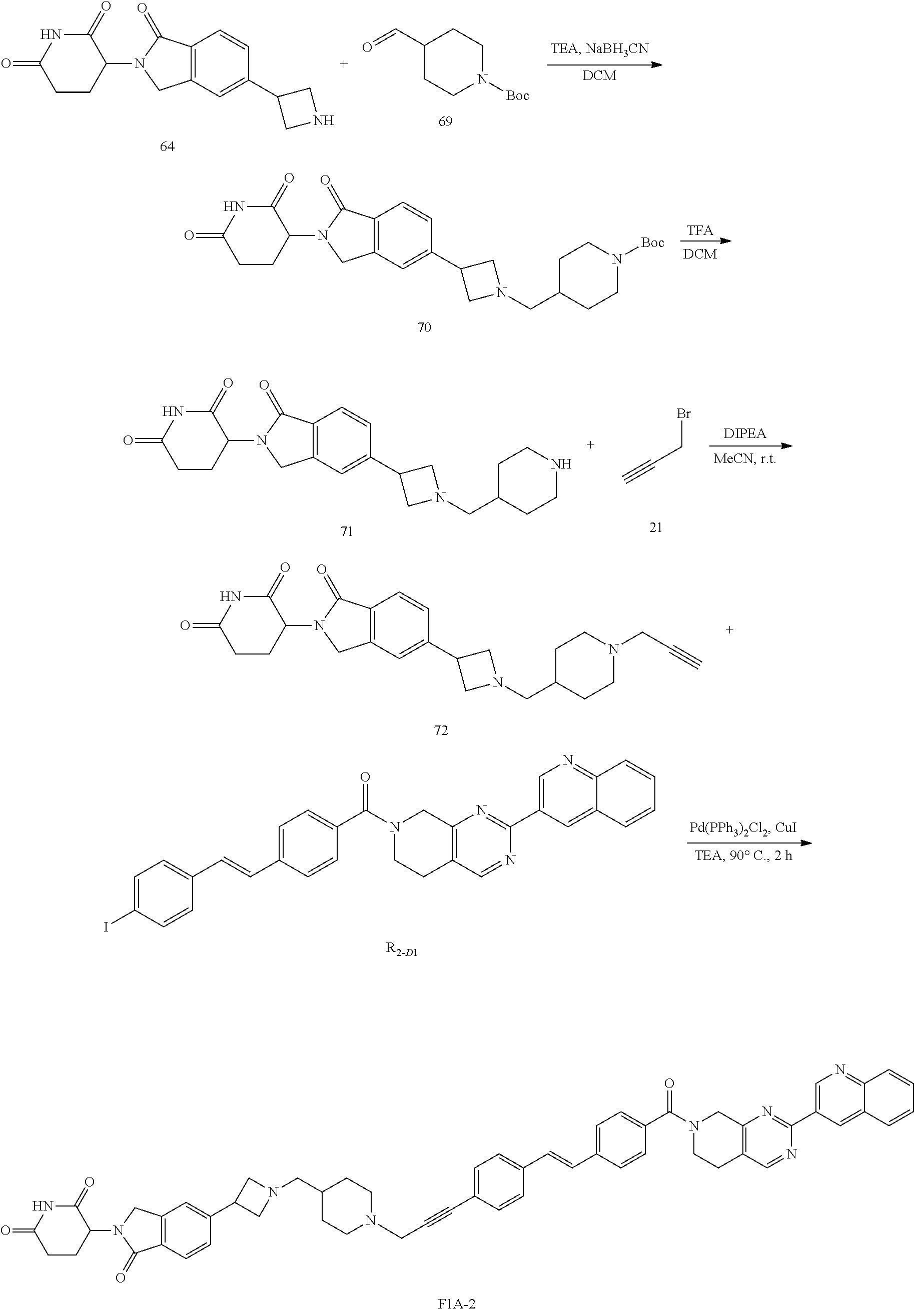

### Step 1. Synthesis of Compound 70

Compound 64 (0.55 mmol), Compound 69 (0.65 mmol), triethylamine (2 mL), sodium cyanoborohydride (0.85 mmol) and dichloroethane (5 mL) were added in sequence to a 50 mL reaction bottle, reacted under stirring at room temperature for 2 hours, then the reaction was quenched (LC-MS monitoring), the solvent was removed by evaporation to dryness, purification was carried out by medium-pressure preparative chromatography, and the solvent was removed by concentration under reduced pressure to obtain Compound 70 (C₂₇H₃₇N₄O₅, [M+H]⁺ 497.3; found 497.1, yield 63.6%).

### Step 2. Synthesis of Compound 71

Compound 70 (0.35 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 71 (C₂₂H₂₈N₄O₃, [M+H]⁺ 396.2; found 396.3, crude product).

### Step 3. Synthesis of Compound 72

Compound 71 (0.35 mmol), Compound 21 (0.65 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 72 (C₂₅H₃₁N₄O₃, [M+H]⁺ 435.2; found 435.1, yield 84.5%).

### Step 4. Synthesis of Compound F1A-2

Under nitrogen protection, Compound R_{2-D1} (0.25 mmol), Compound 72 (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound F1A-2 (9.4 mg, 10.42 μmol, 84.9% purity). LC-MS: C₅₆H₅₃N₈O₄, [M/2+H]⁺451.6; found 451.6. ¹H NMR (600 MHz, DMSO-d6) δ 11.00 (s, 1H), 10.27 (s, 1H), 9.83-9.75 (m, 1H), 9.31-9.24 (m, 1H), 8.86 (s, 1H), 8.22 (s, 1H), 8.10 (s, 1H), 7.85 (s, 1H), 7.78-7.67 (m, 6H), 7.60-7.56 (m, 5H), 7.43 (s, 2H), 5.12 (d, J=5.4 Hz, 1H), 4.95 (s, 1H), 4.79 (s, 1H), 4.55 (s, 2H), 4.45-4.33 (m, 6H), 4.24 (s, 3H), 3.26 (s, 2H), 3.09-2.96 (m, 4H), 2.95-2.90 (m, 1H), 2.61-2.60 (m, 2H), 2.46-2.39 (m, 2H), 2.02-2.01 (m, 3H), 2.00-1.91 (m, 1H), 1.47-1.45 (m, 2H).

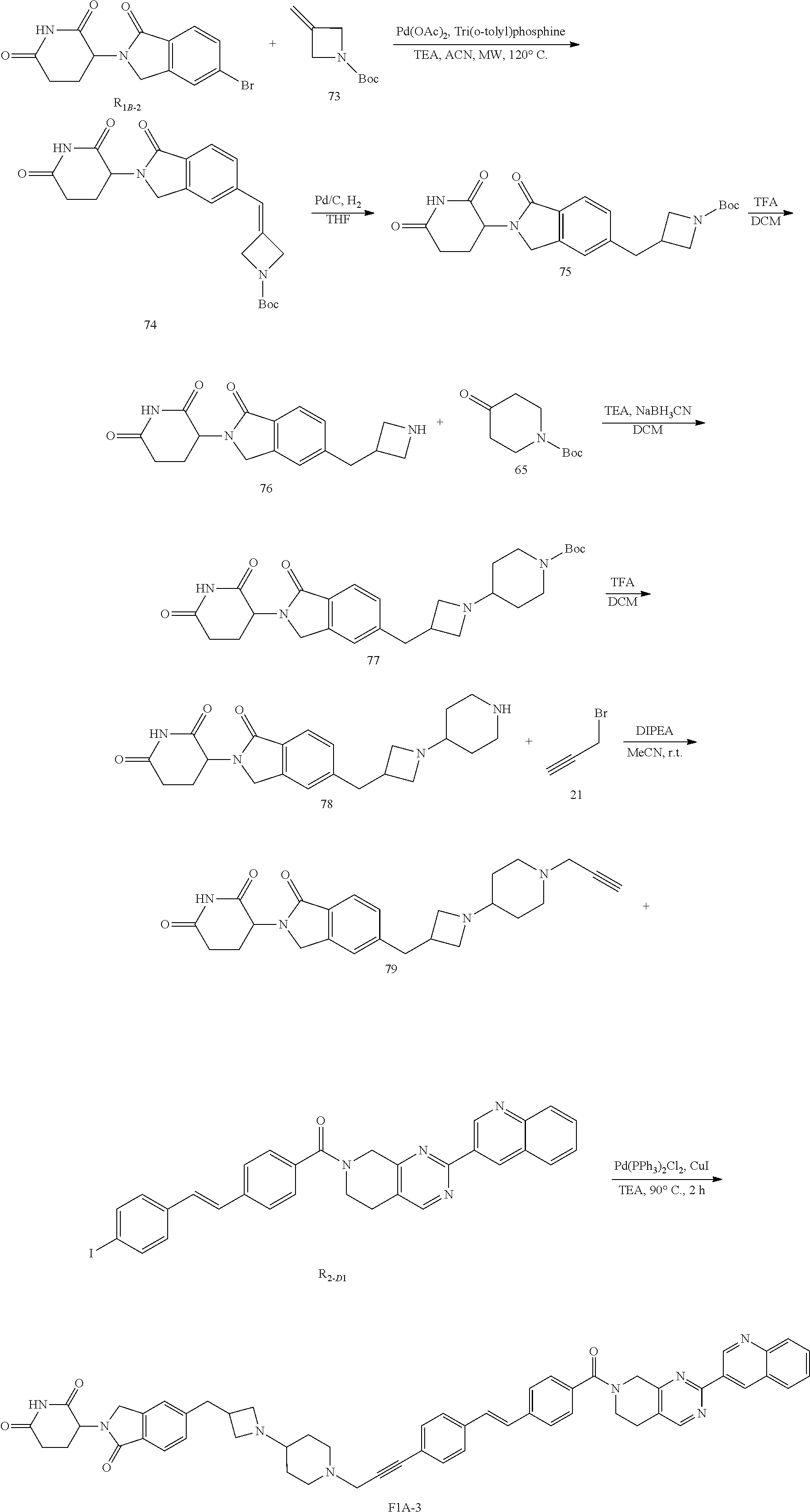

### Step 1. Synthesis of Compound 74

Under nitrogen protection, Compound R_{1B-2} (1.00 mmol), Compound 73 (1.20 mmol), tri(o-tolyl)phosphine (0.20 mmol), triethylamine (2 mL), and palladium acetate (0.25 mol %) were added in sequence to the 50 mL reaction bottle. The reaction system was sealed, the reaction was carried out under stirring in a microwave reactor at 120° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound 74 (C₂₂H₂₆N₃O₅, [M+H]⁺ 412.2; found 412.1, yield 63.2%).

### Step 2. Synthesis of Compound 75

Compound 74 (0.60 mmol), tetrahydrofuran (5 mL), and palladium-on-carbon (30.00 mg) were added in sequence to a 50 mL reaction bottle. The reaction system was replaced with hydrogen, maintained at an atmospheric pressure hydrogen atmosphere, and the reaction was carried out under stirring at room temperature for 3 hours (LC-MS monitoring). Filtration was carried out to remove palladium-on-carbon, washing was carried out with methanol (2×20 mL), the filtrate was collected, and concentrated under reduced pressure to remove the solvent to obtain Compound 75 (C₂₂H₂₈N₃O₅, [M+H]⁺ 414.2; found 414.1, crude product).

### Step 3. Synthesis of Compound 76

Compound 75 (0.55 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 76 (C₁₇H₂₀N₃O₃, [M+H]⁺ 314.2; found 314.3, crude product).

### Step 4. Synthesis of Compound 77

Compound 76 (0.55 mmol), Compound 65 (1.02 mmol), triethylamine (2 mL), sodium cyanoborohydride (64.10 mg, 1.02 mmol) and dichloroethane (5 mL) were added in sequence to a 50 mL reaction bottle, reacted under stirring at room temperature for 2 hours, then the reaction was quenched (LC-MS monitoring), the solvent was removed by evaporation to dryness, purification was carried out by medium-pressure preparative chromatography, and the solvent was removed by concentration under reduced pressure to obtain Compound 77 (C₂₇H₃₇N₄O₅, [M+H]⁺ 497.3; found 497.1, yield 63.6%).

### Step 5. Synthesis of Compound 78

Compound 77 (0.35 mmol) was dissolved in dichloromethane (5 mL) in a 50 mL reaction bottle, then trifluoroacetic acid (1.5 mL) was added dropwise, the reaction was carried out under stirring at room temperature for 0.5 hour, and the solvent was removed by concentration under reduced pressure to obtain Compound 78 (C₂₂H₂₉N₄O₃, [M+H]⁺ 397.2; found 397.3, crude product).

### Step 6. Synthesis of Compound 79

Compound 78 (0.35 mmol), Compound 21 (0.65 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, purified by medium-pressure preparative chromatography, and concentrated under reduced pressure to remove the solvent to obtain Compound 79 (C₂₅H₃₁N₄O₃, [M+H]⁺ 435.2; found 435.4, yield 71.4%).

### Step 7. Synthesis of Compound F1A-3

Under nitrogen protection, Compound R_{2-D1} (0.25 mmol), Compound 79 (0.25 mmol), copper iodide (0.55 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and the solvent was removed by concentration under reduced pressure to obtain Compound F1A-3 (20.3 mg, 22.50 μmol). LC-MS: C₅₆H₅₃N₈O₄, [M/2+H]⁺451.6; found 451.5. ¹H NMR (600 MHz, DMSO-d6) δ 9.82-9.77 (m, 1H), 9.29-9.22 (m, 1H), 8.85 (s, 1H), 8.21 (s, 1H), 8.09 (s, 1H), 7.84 (s, 1H), 7.74 (d, J=7.9 Hz, 2H), 7.63-7.54 (m, 4H), 7.45 (d, J=7.7 Hz, 2H), 7.40-7.32 (m, 5H), 7.31 (d, J=8.1 Hz, 1H), 5.10 (d, J=5.6 Hz, 1H), 4.94 (s, 1H), 4.80 (s, 1H), 4.42-4.40 (m, 1H), 4.29-4.27 (m, 1H), 3.99 (s, 1H), 3.73 (s, 1H), 3.49 (s, 2H), 3.24 (s, 2H), 2.98-2.91 (m, 6H), 2.78 (s, 4H), 2.21 (s, 2H), 1.98 (s, 2H), 1.63 (s, 2H), 1.23-1.17 (m, 3H).

### Example 10. Synthesis of Compounds F1B-1 and F1B-2 of the Present Invention

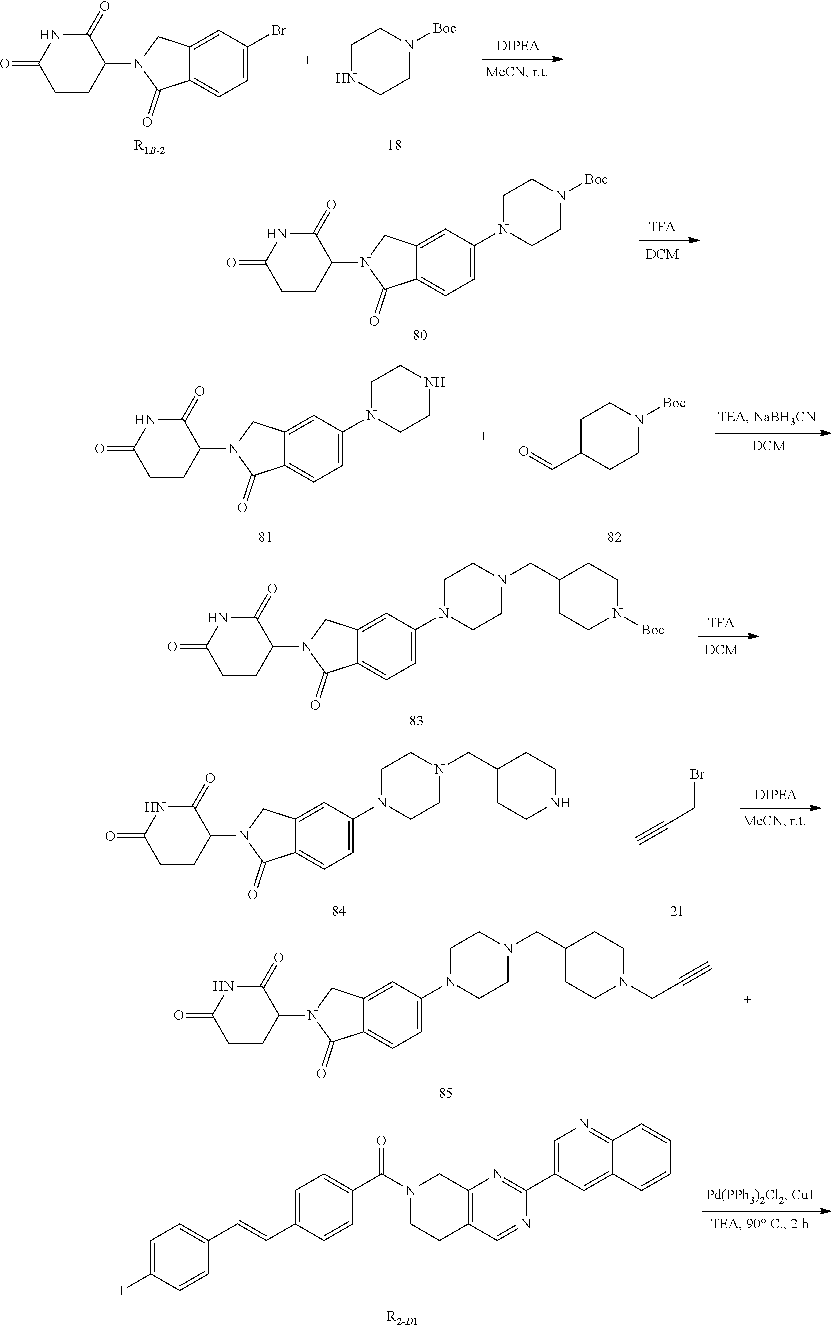

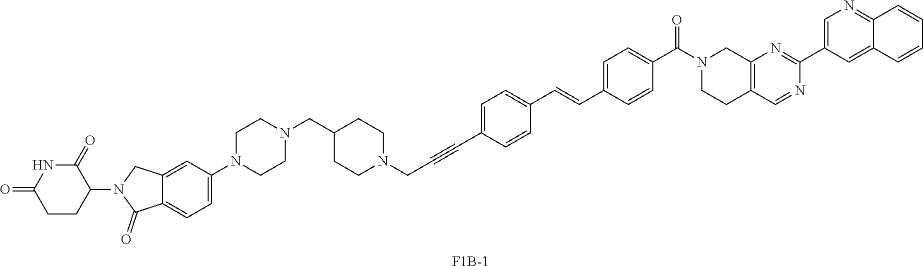 [SMILES: O=C1CCC(N2Cc3cc(N4CCN(CC5CCN(CC#Cc6ccc(/C=C/c7ccc(C(=O)N8CCc9cnc(-c%10cnc%11ccccc%11c%10)nc9C8)cc7)cc6)CC5)CC4)ccc3C2=O)C(=O)N1 ; "F1B-1"]

### Step 1. Synthesis of Compound 80

Compound R_{1B-2} (1.00 mmol), Compound 18 (1.15 mmol), N,N-diisopropylethylamine (2.10 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 80 (C₂₂H₂₉N₄O₅, [M+H]⁺ 429.1; found 429.3, yield 73.2%).

### Step 2. Synthesis of Compound 81

Compound 80 (0.35 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 81 (C₁₇H₂₁N₄O₃, [M+H]⁺ 329.2; found 329.3, crude product).

### Step 3. Synthesis of Compound 83

Compound 81 (0.25 mmol), Compound 82 (0.45 mmol), triethylamine (2 mL), sodium cyanoborohydride (0.55 mmol) and dichloromethane (5 mL) were added in sequence to a 50 mL reaction bottle, reacted under stirring at room temperature for 2 hours, then the reaction was quenched (LC-MS monitoring), the solvent was removed by evaporation to dryness, purification was carried out by medium-pressure preparative chromatography, and the solvent was removed by concentration under reduced pressure to obtain Compound 83 (C₂₈H₄₀N₅O₅, [M+H]⁺ 526.3; found 526.5, yield 81.6%).

### Step 4. Synthesis of Compound 84

Compound 83 (0.20 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 84 (C₂₃H₃₂N₅O₃, [M+H]⁺ 426.2; found 426.3, crude product).

### Step 5. Synthesis of Compound 85

Compound 84 (0.15 mmol), Compound 21 (0.35 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 85 (C₂₆H₃₄N₅O₃, [M+H]⁺ 464.3; found 464.5, yield 66.7%).

### Step 6. Synthesis of Compound F1B-1

Under nitrogen protection, Compound R_{2-D1} (0.10 mmol), Compound 85 (0.10 mmol), copper iodide (0.25 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound F1B-1 (7.2 mg, 7.73 μmol). LC-MS: C₅₇H₅₆N₉O₄, [M/2+H]⁺466.0; found 466.0. ¹H NMR (400 MHz, Methanol-d4) δ 9.99-9.89 (m, 1H), 9.53-9.44 (m, 1H), 8.80 (s, 1H), 8.25-8.14 (m, 2H), 8.09 (s, 1H), 7.94-7.75 (m, 2H), 7.73-7.71 (m, 1H), 7.66-7.64 (m, 2H), 7.57-7.53 (m, 5H), 7.34 (s, 2H), 7.18-7.16 (m, 2H), 5.22-5.10 (m, 2H), 4.49-4.42 (m, 2H), 4.42-4.38 (m, 2H), 3.87-3.47 (m, 6H), 3.34-3.22 (m, 4H), 3.13-3.05 (m, 2H), 2.93-2.90 (m, 1H), 2.89-2.86 (m, 1H), 2.79-2.75 (m, 1H), 2.52-2.47 (m, 1H), 2.45-2.03 (m, 4H), 1.73-1.67 (m, 2H).

According to the step 1 to step 6 of the method for Compound F1B-1, the raw material 1 in the following table was used to replace Compound R_{1B-2}, the raw material 2 was used to replace Compound R_{2-D1}, and other raw materials and operating methods remained unchanged, to obtain Compounds F1B-2, F1B-3 and F1B-4.

| Compound | Structure and characterization data | Raw | Raw | |
|---|---|---|---|---|
| No. | of compound | material 1 | material 2 | Yield |
| F1B-2 | 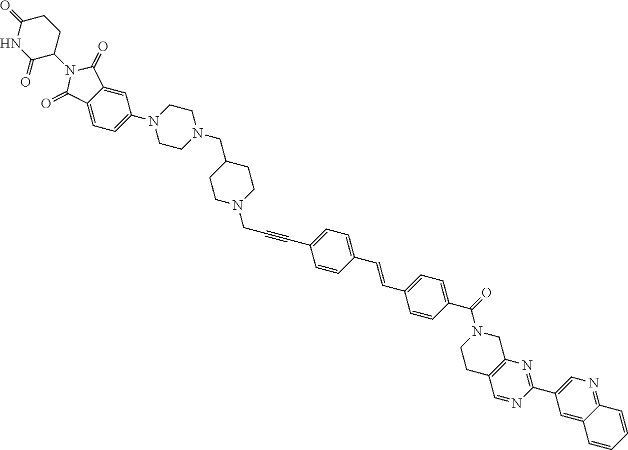 [SMILES: O=C1CCC(N2C(=O)c3ccc(N4CCN(CC5CCN(CC#Cc6ccc(/C=C/c7ccc(C(=O)N8CCc9cnc(-c%10cnc%11ccccc%11c%10)nc9C8)cc7)cc6)CC5)CC4)cc3C2=O)C(=O)N1] | 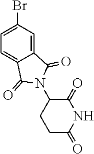 [SMILES: O=C1CCC(N2C(=O)c3ccc(Br)cc3C2=O)C(=O)N1] R_{1A-5} | 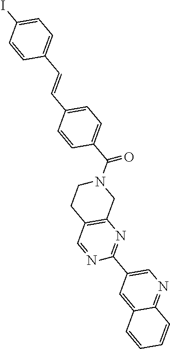 [SMILES: O=C(c1ccc(/C=C/c2ccc(I)cc2)cc1)N1CCc2cnc(-c3cnc4ccccc4c3)nc2C1] R_{2-D1} | 13.6% |
| | F1B-2 | | | |
| | LC-MS: C₅₇H₅₄N₉O₅, [M + H]⁺ 944.1; | | | |
| | found 944.3. ¹H NMR (400 MHz, | | | |
| | DMSO-d₆) δ 11.01 (s, 1H), 9.83-9.79 | | | |
| | (m, 1H), 9.31-9.28 (m, 1H), 8.85 (s, 1H), | | | |
| | 8.20 (s, 1H), 8.10 (s, 1H), 7.85 (s, 1H), | | | |
| | 7.76-7.68 (m, 2H), 7.66-7.54 (m, 4H), | | | |
| | 7.47-7.45 (m, 2H), 7.42-7.39 (m, 2H), | | | |
| | 7.34 (s, 2H), 7.33-7.26 (m, 1H), 7.24- | | | |
| | 7.23 (m, 1H), 5.05-5.03 (m, 1H), 4.95- | | | |
| | 4.81 (m, 2H), 4.10-4.01 (m, 1H), 3.99- | | | |
| | 3.73 (m, 1H), 3.52 (s, 2H), 3.43 (s, 4H), | | | |
| | 3.00-2.97 (m, 2H), 2.88-2.85 (m, 3H), | | | |
| | 2.67-2.52 (m, 3H),2.34-2.33 (m, 1H), | | | |
| | 2.29-2.17 (m, 5H), 2.01-2.00 (m, 1H), | | | |
| | 1.76-1.74 (m, 2H), 1.52 (s, 1H), 1.23- | | | |
| | 1.14 (m, 3H). | | | |
| F1B-3 | 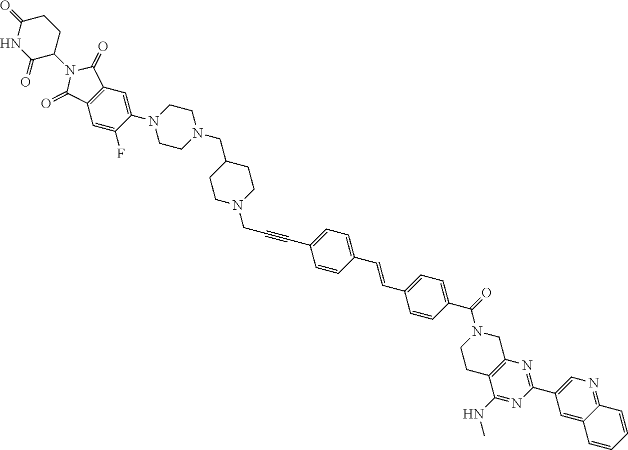 [SMILES: CNc1nc(-c2cnc3ccccc3c2)nc2c1CCN(C(=O)c1ccc(/C=C/c3ccc(C#CCN4CCC(CN5CCN(c6cc7c(cc6F)C(=O)N(C6CCC(=O)NC6=O)C7=O)CC5)CC4)cc3)cc1)C2] | 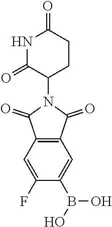 [SMILES: O=C1CCC(N2C(=O)c3cc(F)c(B(O)O)cc3C2=O)C(=O)N1] R_{1A-6} | 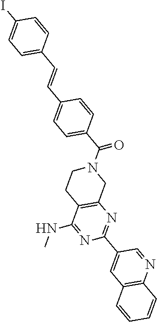 [SMILES: CNc1nc(-c2cnc3ccccc3c2)nc2c1CCN(C(=O)c1ccc(/C=C/c3ccc(I)cc3)cc1)C2] R2-B | 48.9% |
| | F1B-3 | | | |
| | LC-MS: C₅₈H₅₆N₁₀O₅F, [M + H]⁺ 991.4; | | | |
| | found 991.4. ¹H NMR (600 MHz, | | | |
| | DMSO-d₆) δ 11.12 (s, 1H), 9.94-9.75 | | | |
| | (m, 2H), 9.24-9.16 (m, 1H), 8.18-8.08 | | | |
| | (m, 2H), 7.85-7.82 (m, 2H), 7.76-7.71 | | | |
| | (m, 4H), 7.67-7.51 (m, 5H), 7.47 (s, 2H), | | | |
| | 7.22 (s, 1H), 5.14-5.11 (m, 1H), 4.56 (s, | | | |
| | 2H), 4.47 (s, 4H), 3.98-3.79 (m, 4H), | | | |
| | 3.66-3.64 (m, 4H), 3.48-3.09 (m, 10H), | | | |
| | 2.94-2.87 (m, 1H), 2.68-2.54 (m, 3H), | | | |
| | 2.16 (s, 1H), 2.08-2.03 (m, 3H), 1.51- | | | |
| | 1.48 (m, 2H). Purity >90%. | | | |
| F1B-4 | 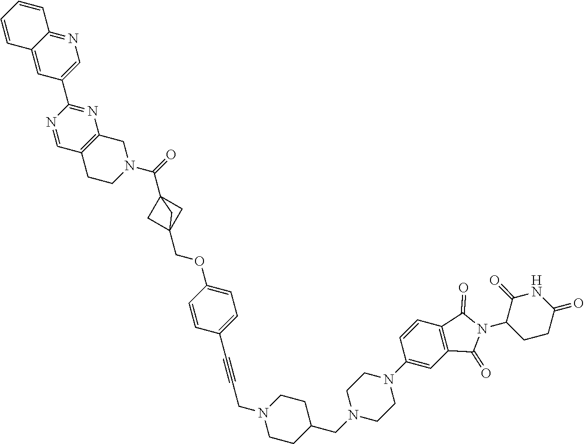 [SMILES: O=C1CCC(N2C(=O)c3ccc(N4CCN(CC5CCN(CC#Cc6ccc(OCC78CC(C(=O)N9CCc%10cnc(-c%11cnc%12ccccc%12c%11)nc%10C9)(C7)C8)cc6)CC5)CC4)cc3C2=O)C(=O)N1] | 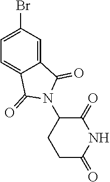 [SMILES: O=C1CCC(N2C(=O)c3ccc(Br)cc3C2=O)C(=O)N1] R_{1A-5} | 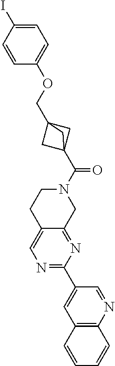 [SMILES: O=C(N1CCc2cnc(-c3cnc4ccccc4c3)nc2C1)C12CC(COc3ccc(I)cc3)(C1)C2] R_{2-C5} | 31.2% |
| | F1B-4 | | | |
| | LC-MS: C₅₅H₅₆N₉O₆, [M/2 + H]⁺ 470.0; | | | |
| | found 470.1. ¹H NMR (600 MHz, | | | |
| | DMSO-d₆) δ 11.10 (s, 1H), 9.83-9.82 | | | |
| | (m, 1H), 9.29 (s, 1H), 8.84 (s, 1H), 8.23 | | | |
| | (d, J = 7.8 Hz, 1H), 8.11 (d, J = 8.4 Hz, | | | |
| | 1H), 7.87 (t, J = 7.8 Hz, 1H), 7.76 (t, | | | |
| | J = 8.4 Hz, 1H), 7.70 (t, J = 7.2 Hz, 1H), | | | |
| | 7.51-7.48 (m, 3H), 7.36-7.34 (m, 1H), | | | |
| | 7.03-7.02 (m, 2H), 5.11-5.08 (m, 1H), | | | |
| | 4.95 (s, 1H), 4.79 (s, 2H), 4.34 (s, 2H), | | | |
| | 4.22 (s, 2H), 4.14 (s, 2H), 3.96-3.80 (m, | | | |
| | 2H), 3.64-3.62 (m, 4H), 3.47 (s, 3H), | | | |
| | 3.33-3.08 (m, 6H), 2.96-2.87 (m, 3H), | | | |
| | 2.61-2.50 (m, 2H), 2.20-2.17 (m, 7H), | | | |
| | 2.07-2.03 (m, 3H). Purity >99%. | | | |

### Example 11. Synthesis of Compounds F2-1, F2-2, F2-2-A, F2-3 and F2-4 of the Present Invention

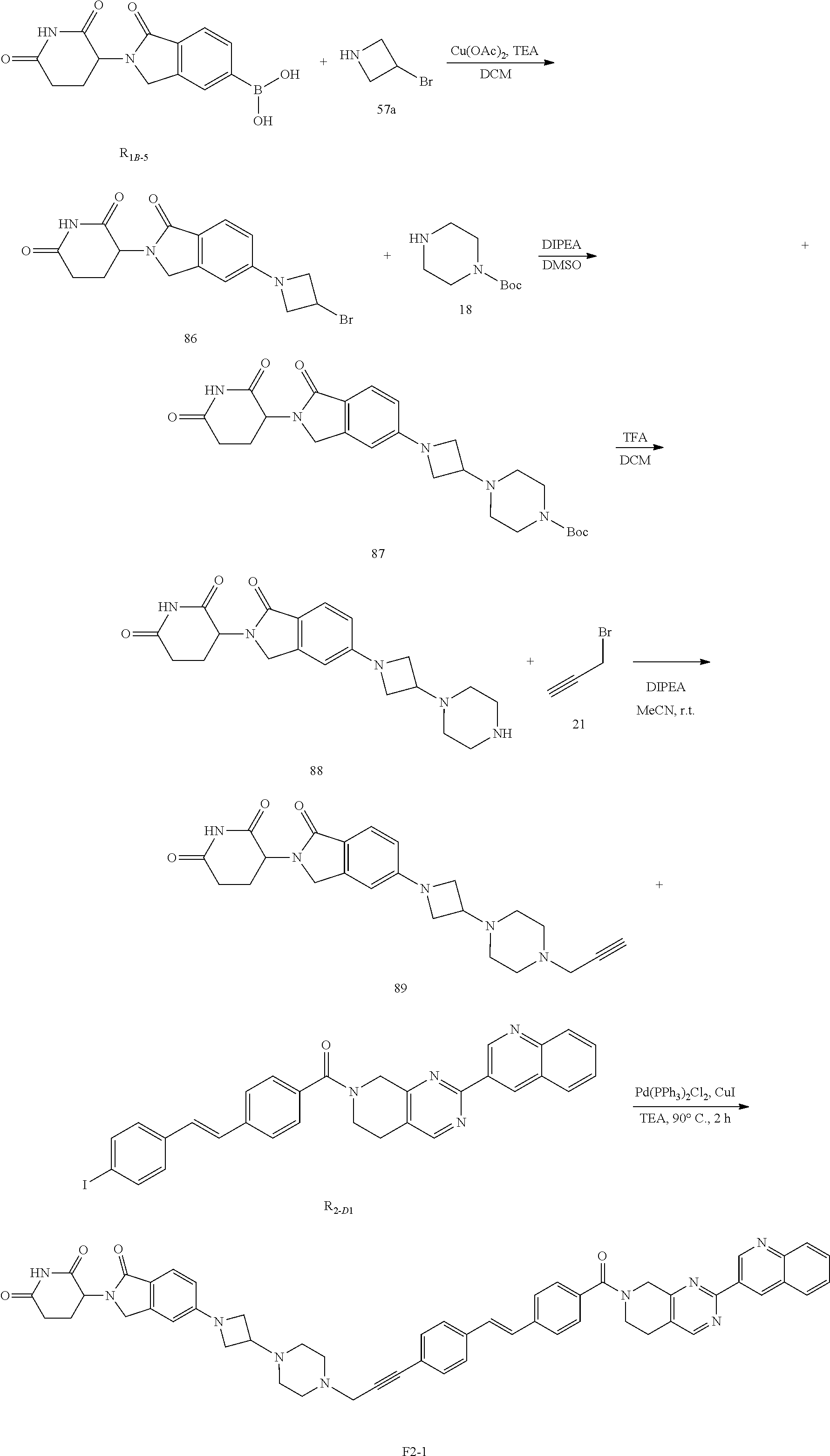

### Step 1. Synthesis of Compound 86

Under nitrogen protection, Compound R_{1B-5} (1.00 mmol), Compound 57a (1.05 mmol), Cu(OAc)₂ (2.00 mmol), and triethylamine (5 mL) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound 86 (C₁₆H₁₇BrN₃O₃, [M+H]⁺ 378.0; found 378.2, yield 48.2%).

### Step 2. Synthesis of Compound 87

Compound 86 (0.45 mmol), Compound 18 (0.75 mmol), N,N-diisopropylethylamine (1.02 mmol) and DMSO (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×5 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 87 (C₂₅H₃₄N₅O₅, [M+H]⁺ 484.3; found 484.2, yield 66.7%).

### Step 3. Synthesis of Compound 88

Compound 87 (0.30 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 88 (C₂₀H₂₆N₅O₃, [M+H]⁺ 384.2; found 384.2, crude product).

### Step 4. Synthesis of Compound 89

Compound 88 (0.30 mmol), Compound 21 (0.65 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 89 (C₂₃H₂₈N₅O₃, [M+H]⁺ 422.2; found 422.3, yield 33.3%).

### Step 5. Synthesis of Compound F2-1

Under nitrogen protection, Compound R_{2-D1} (0.10 mmol), Compound 89 (0.10 mmol), copper iodide (0.45 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound F2-1 (4.7 mg, 5.29 μmol). LC-MS: C₅₄H₅₀N₉O₄, [M/2+H]⁺ 445.0; found 445.1. ¹H NMR (400 MHz, DMSO-d6) δ 10.94 (s, 1H), 9.83-9.78 (m, 1H), 9.31-9.28 (m, 1H), 8.86 (s, 1H), 8.57-7.86 (m, 4H), 7.85-7.41 (m, 10H), 7.18-6.54 (m, 4H), 5.3-4.80 (m, 3H), 4.31-3.99 (m, 6H), 3.17-2.50 (m, 7H), 1.46-0.85 (m, 12H).

According to the step 1 to step 5 of the method for Compound F2-1, the raw material 1 in the following table was used to replace Compound R_{1B-5}, the raw material 2 in the following table was used to replace Compound 57a, and the other raw materials and operating methods remained unchanged to obtain Compounds F2-2, F2-2-A, F2-3, F2-4.

| Com- | | Raw | Raw | |
|---|---|---|---|---|
| pound | Structure and characterization data of | material | material | |
| No. | compound | 1 | 2 | Yield |
| F2-2 | 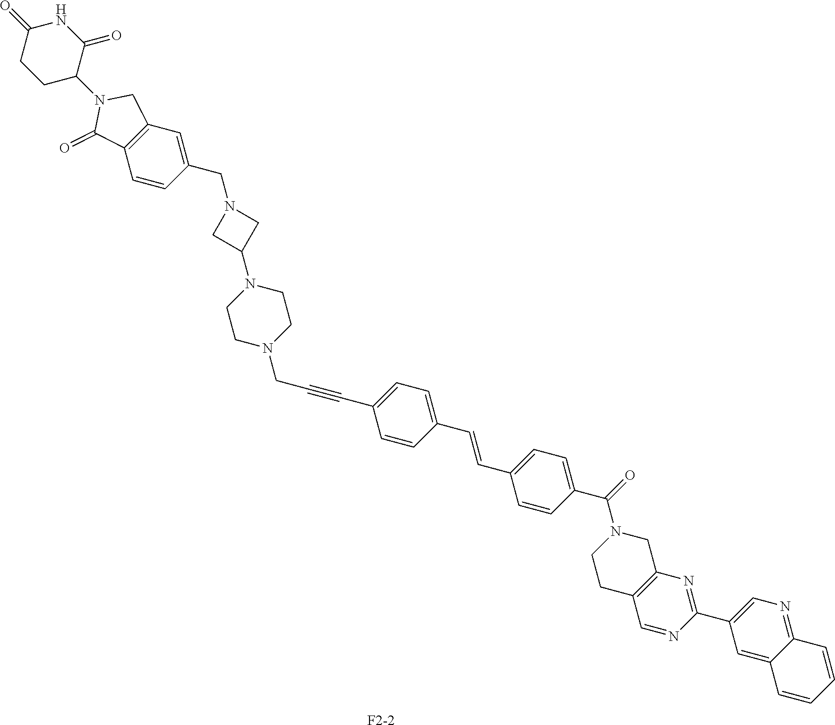 [SMILES: O=C1CCC(N2Cc3cc(CN4CC(N5CCN(CC#Cc6ccc(/C=C/c7ccc(C(=O)N8CCc9cnc(-c%10cnc%11ccccc%11c%10)nc9C8)cc7)cc6)CC5)C4)ccc3C2=O)C(=O)N1 ; "F2-2"] | 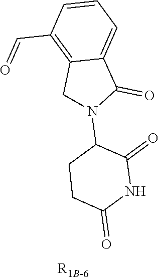 [SMILES: O=Cc1cccc2c1CN(C1CCC(=O)NC1=O)C2=O] | 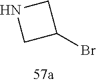 | 23.5% |
| | LC-MS: C₅₅H₅₂N₉O₄, [M/2 + H]⁺ 452.0; found | | | |
| | 452.2. ¹H NMR (400 MHz, DMSO-d₆) δ 9.85- | | | |
| | 9.78 (m, 1H), 9.40-9.31 (m, 1H), 8.86 (s, | | | |
| | 1H),8.23 (s, 1H), 8.10 (s, 1H), 7.86-7.82 (m, 2H), | | | |
| | 7.77-7.70 (m, 7H), 7.63 (d, J = 8.0 Hz, 1H), 7.57- | | | |
| | 7.55 (m, 4H), 7.44 (s, 2H), 5.16-5.12 (m, 1H), | | | |
| | 4.94-4.88 *m, 2H), 4.53 (s, 2H), 4.38-4.34 (m, | | | |
| | 4H), 4.16-4.13 (m, 2H), 4.05-4.04 (m, 2H), 3.43 | | | |
| | (t, J = 8.0 Hz, 2H), 3.29-3.17 (m, 3H), 3.10 (t, J = | | | |
| | 8.0 Hz, 3H), 2.99-2.94 (m, 1H), 2.89-2.81 (m, | | | |
| | 3H), 2.68-2.51 (m, 2H), 2.50-2.31 (m, 2H), 2.03- | | | |
| | 2.01 (m, 1H). | | | |
| F2- 2-A | 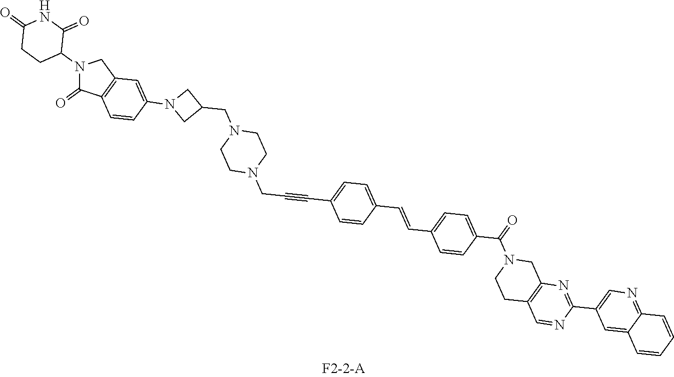 [SMILES: O=C1CCC(N2Cc3cc(N4CC(CN5CCN(CC#Cc6ccc(/C=C/c7ccc(C(=O)N8CCc9cnc(-c%10cnc%11ccccc%11c%10)nc9C8)cc7)cc6)CC5)C4)ccc3C2=O)C(=O)N1] | 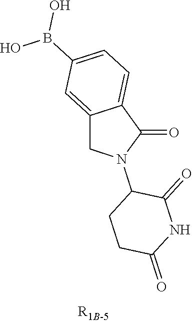 [SMILES: O=C1CCC(N2Cc3cc(B(O)O)ccc3C2=O)C(=O)N1] | 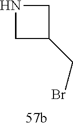 | 12.3% |
| | LC-MS: C₅₅H₅₂N₉O₄, [M/2 + H]⁺ 452.0 found | | | |
| | 451.9. ¹H NMR (600 MHz, CDCl₃) δ 9.81 (br, | | | |
| | 1H), 9.08 (br, 1H), 8.61 (s, 1H), 8.10 (d, J = 7.8 | | | |
| | Hz, 2H), 7.96 (d, J = 7.2 Hz, 1H), 7.88 (br, 1H), | | | |
| | 7.70 (t, J = 7.2 Hz, 1H), 7.61 (d, J = 8.4 Hz, 1H), | | | |
| | 7.54-7.50 (m, 3H), 7.48-7.45 (m, 2H), 7.41-7.37 | | | |
| | (m, 5H), 7.88 (m, 2H), 5.13-5.10 (m, 1H), 4.31- | | | |
| | 4.29 (m, 1H), 4,16-4.13 (m, 1H), 4.03-3.99 (m, | | | |
| | 3H), 3.85 (s, 1H), 3.57 (t, J = 6.0 Hz, 2H), 3.51- | | | |
| | 3.49 (m, 3H), 3.42 (s, 1H), 2.95 (br, 4H), 2.83- | | | |
| | 2.80 (m, 2H), 2.77-2.76 (m, 1H), 2.75 (s, 6H), | | | |
| | 2.27-2.22 (m, 2H), 2.15-2.10 (m, 2H). Purity | | | |
| | >75%. | | | |
| F2-3 | 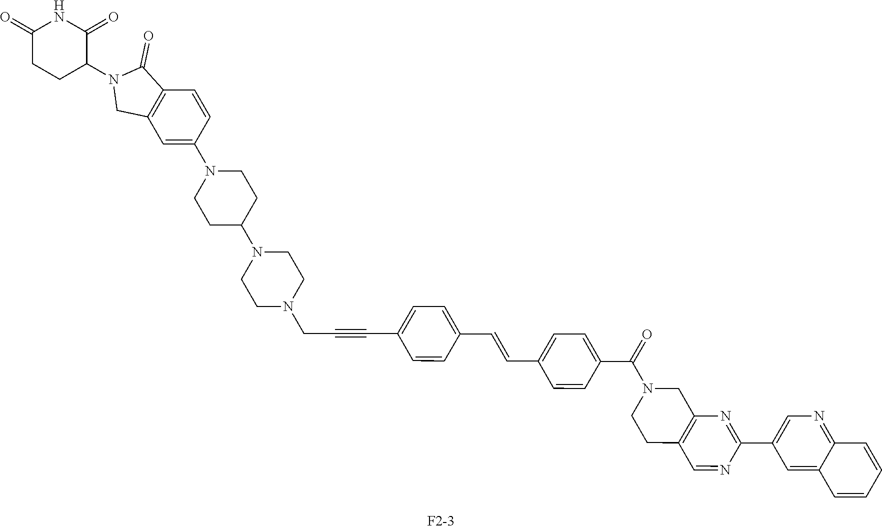 [SMILES: O=C1CCC(N2Cc3cc(N4CCC(N5CCN(CC#Cc6ccc(/C=C/c7ccc(C(=O)N8CCc9cnc(-c%10cnc%11ccccc%11c%10)nc9C8)cc7)cc6)CC5)CC4)ccc3C2=O)C(=O)N1] | 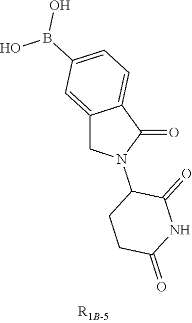 [SMILES: O=C1CCC(N2Cc3cc(B(O)O)ccc3C2=O)C(=O)N1] | 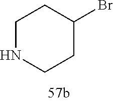 | 21.2% |
| | LC-MS: C₅₆H₅₄N₉O₄, [M/2 + H]⁺ 458.7; found | | | |
| | 458.7. ¹H NMR (600 MHz, DMSO-d₆) δ 9.79 (s, | | | |
| | 1H), 9.35 (s, 1H), 8.85 (s, 1H), 8.35-7.85 (m, | | | |
| | 2H), 7.88-7.29 (m, 11H), 7.16-7.11 (m, 2H), 5.33 | | | |
| | (s, 1H), 5.20-4.69 (m, 3H), 4.49-4.15 (m, 2H), | | | |
| | 3.74 (s, 3H), 3.65-3.40 (m, 5H), 3.16-2.80 (m, | | | |
| | 6H), 2.50 (s, 3H), 2.24-1.93 (m, 5H), 1.23 (s, | | | |
| | 6H). | | | |
| F2-4 | 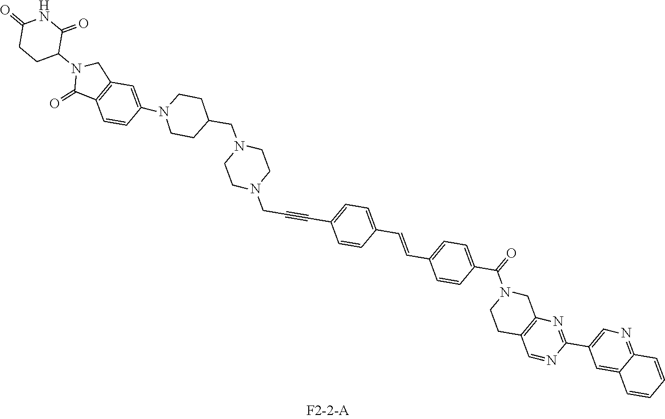 [SMILES: O=C1CCC(N2Cc3cc(N4CCC(CN5CCN(CC#Cc6ccc(/C=C/c7ccc(C(=O)N8CCc9cnc(-c%10cnc%11ccccc%11c%10)nc9C8)cc7)cc6)CC5)CC4)ccc3C2=O)C(=O)N1] | 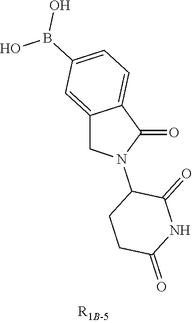 [SMILES: O=C1CCC(N2Cc3cc(B(O)O)ccc3C2=O)C(=O)N1] | 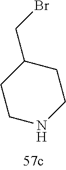 | 19.6% |
| | LC-MS: C₅₇H₅₆N₉O₄, [M/2 + H]⁺ 466.1; found | | | |
| | 466.0. ¹H NMR (400 MHz, DMSO-d₆) δ 10.95 | | | |
| | (s, 1H), 9.83-9.77 (m, 1H), 9.31-9.28 (m, 1H), | | | |
| | 8.86 (s, 1H), 8.21 (s, 1H), 8.10 (s, 1H), 7.85 (s, | | | |
| | 1H), 7.76-7.68 (m, 2H), 7.66-7.57 (m, 3H), 7.54- | | | |
| | 7.51 (m, 2H), 7.50-7.48 (m, 3H), 7.40 (s, 2H), | | | |
| | 7.08-7.06 (m, 2H), 5.03-5.02 (m, 1H), 4.95-4.81 | | | |
| | (m, 2H), 4.32-4.30 (m, 1H), 4.22-4.18 (m, 1H), | | | |
| | 3.92-3.90 (m, 2H), 3.74 (s, 4H), 3.12-3.07 (m, | | | |
| | 8H), 3.00-2.99 (m, 3H), 2.89-2.78 (m, 2H), 2.38- | | | |
| | 2.33 (m, 1H), 2.07-1.97 (m, 4H), 1.84-1.81 (m, | | | |
| | 2H), 1.33-1.16 (m, 3H). | | | |

According to the step 1 to step 5 of the method for Compound F2-1, the raw material 1 in the following table was used to replace compound R_{2D-1}, the raw material 2 in the following table was used to replace Compound 57a, and the other raw materials and operating methods remained unchanged, to obtain Compounds F2-7 to F2-10.

| Com- | | Raw | Raw | |
|---|---|---|---|---|
| pound | Structure and characterization data of | material | material | |
| No. | compound | 1 | 2 | Yield |
| F2-7 | 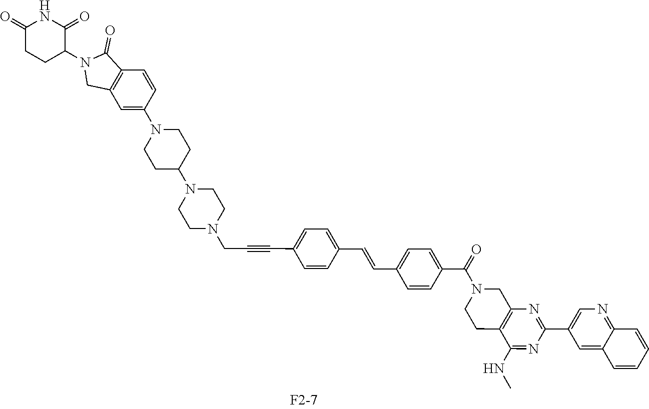 [SMILES: CNc1nc(-c2cnc3ccccc3c2)nc2c1CCN(C(=O)c1ccc(/C=C/c3ccc(C#CCN4CCN(C5CCN(c6ccc7c(c6)CN(C6CCC(=O)NC6=O)C7=O)CC5)CC4)cc3)cc1)C2] | 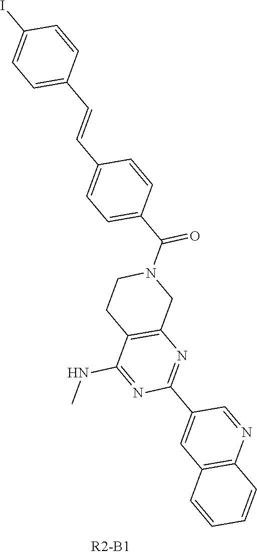 [SMILES: CNc1nc(-c2cnc3ccccc3c2)nc2c1CCN(C(=O)c1ccc(/C=C/c3ccc(I)cc3)cc1)C2] | 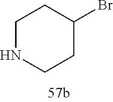 | 15.8% |
| | LC-MS: C₅₇H₅₇N₁₀O₄, [M/2 + H]⁺ 473.6; found | | | |
| | 473.7. ¹H NMR (600 MHz, DMSO-d₆) δ 10.97- | | | |
| | 10.95 (m, 1H), 9.82-9.73 (m, 1H), 9.24-9.13 (m, | | | |
| | 2H), 8.16-8.08 (m, 2H), 7.82 (s, 1H), 7.23 (d, J = | | | |
| | 8.4 Hz, 2H), 7.65 (t, J = 7.2 Hz, 3H), 7.55-7.52 | | | |
| | (m, 3H), 7.48-7.45 (m, 2H), 7.39 (s, 2H), 7.22- | | | |
| | 7.19 (m, 1H), 7.13-7.11 (m, 1H), 5.10-5.04 (m, | | | |
| | 1H), 4.73 (s, 1H), 4.57 (s, 1H), 4.36-4.31 (m, 1H), | | | |
| | 4.22-4.20 (m, 1H), 4.08-3.96 (m, 4H), 3.71-3.68 | | | |
| | (m, 2H), 3.09-3.08 (m, 8H), 2.89-2.77 (m, 4H), | | | |
| | 2.65-2.58 (m, 6H), 2.39-2.37 (m, 1H), 2.12 (s, | | | |
| | 2H), 1.99-1.98 (m, 1H), 1.68 (s, 2H). Purity | | | |
| | >90%. | | | |
| F2-8 | 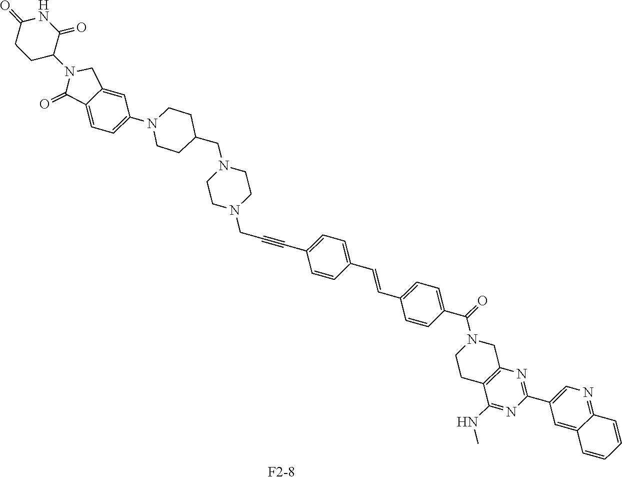 [SMILES: CNc1nc(-c2cnc3ccccc3c2)nc2c1CCN(C(=O)c1ccc(/C=C/c3ccc(C#CCN4CCN(CC5CCN(c6ccc7c(c6)CN(C6CCC(=O)NC6=O)C7=O)CC5)CC4)cc3)cc1)C2] | 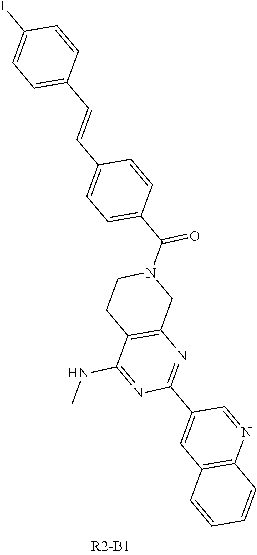 [SMILES: CNc1nc(-c2cnc3ccccc3c2)nc2c1CCN(C(=O)c1ccc(/C=C/c3ccc(I)cc3)cc1)C2] | 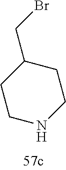 | 16.7% |
| | LC-MS: C₅₈H₅₉N₁₀O₄, [M + H]⁺ 959.5; found | | | |
| | 958.1. ¹H NMR (600 MHz, DMSO-d₆) δ 11.00 (s, | | | |
| | 1H), 9.82-9.80 (m, 1H), 9.27 (s, 1H), 8.81 (s, 1H), | | | |
| | 8.21 (d, J = 8.4 Hz, 1H), 8.10 (d, J = 3.0 Hz, 1H), | | | |
| | 7.85 (t, J = 8.4 Hz, 1H), 7.72-7.68 (m, 3H), 7.56 | | | |
| | (d, J = 5.8 Hz, 1H), 7.37-7.36 (m, 2H), 6.95-6.94 | | | |
| | (m, 2H), 5.13-5.10 (m, 1H), 4.95-4.78 (m, 2H), | | | |
| | 4.47-4.44 (m, 1H), 4.35-4.32 (m, 1H), 4.08-4.04 | | | |
| | (m, 3H), 3.95-3.80 (m, 2H), 3.65 (s, 2H), 3.58 (s, | | | |
| | 2H), 3.34 (s, 2H), 2.94-2.86 (m, 3H), 2.61-2.59 | | | |
| | (m, 6H), 2.42-2.38 (m, 1H), 2.27 (s, 6H), 2.02- | | | |
| | 2.00 (m, 1H). Purity >90%. | | | |
| F2-9 | 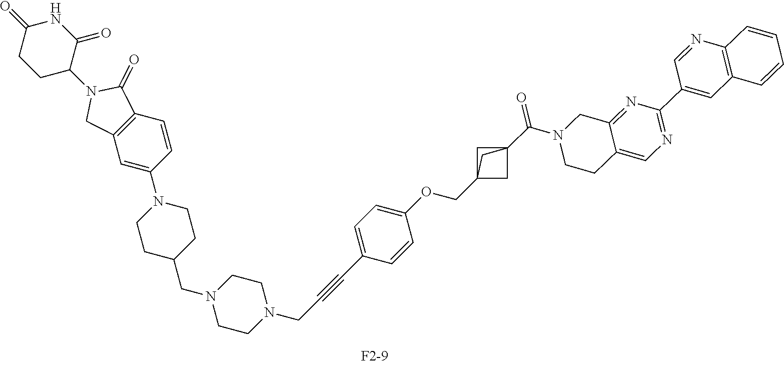 [SMILES: O=C1CCC(N2Cc3cc(N4CCC(CN5CCN(CC#Cc6ccc(OCC78CC(C(=O)N9CCc%10cnc(-c%11cnc%12ccccc%12c%11)nc%10C9)(C7)C8)cc6)CC5)CC4)ccc3C2=O)C(=O)N1] | 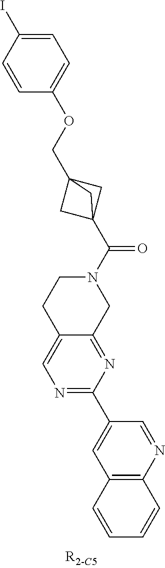 [SMILES: O=C(N1CCc2cnc(-c3cnc4ccccc4c3)nc2C1)C12CC(COc3ccc(I)cc3)(C1)C2] | 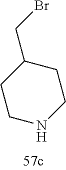 | 35.2% |
| | LC-MS: C₅₅H₅₈N₉O₅, [M/2 + H]⁺ 463.1; found | | | |
| | 462.8. ¹H NMR (600 MHz, DMSO-d₆) δ 9.87 (s, | | | |
| | 1H), 9.46-9.44 (m, 1H), 8.84 (s, 1H), 8.32-8.31 | | | |
| | (m, 1H), 8.18-8.17 (m, 1H), 7.97-7.96 (m, 1H), | | | |
| | 7.80-7.78 (m, 1H), 7.56-7.55 (m, 1H), 7.44-7.43 | | | |
| | (m, 2H), 7.10-7.09 (m, 2H), 7.01-6.99 (m, 2H), | | | |
| | 5.05-4.80 (m, 3H), 4.36-4.33 (m, 1H), 4.24-4.21 | | | |
| | (m, 1H), 4.11 (s, 2H), 3.96-3.90 (m, 6H), 3.38 (s, | | | |
| | 3H), 3.14-2.88 (m, 12H), 2.63-2.56 (m, 1H), | | | |
| | 2.41-2.34 (m, 1H), 2.18 (s, 6H), 2.07-1.98 (m, | | | |
| | 2H), 1.84-1.83 (m, 2H), 1.31-1.30 (m, 2H). Purity | | | |
| | >99%. | | | |
| F2-10 | 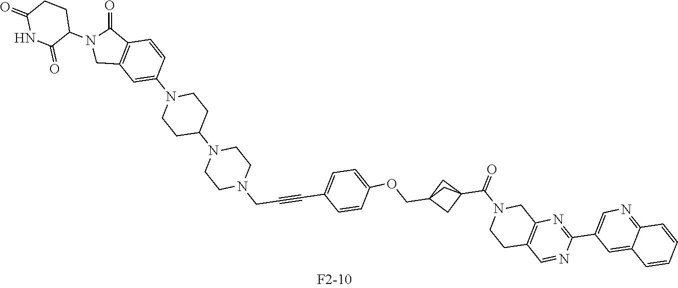 [SMILES: O=C1CCC(N2Cc3cc(N4CCC(N5CCN(CC#Cc6ccc(OCC78CC(C(=O)N9CCc%10cnc(-c%11cnc%12ccccc%12c%11)nc%10C9)(C7)C8)cc6)CC5)CC4)ccc3C2=O)C(=O)N1] | 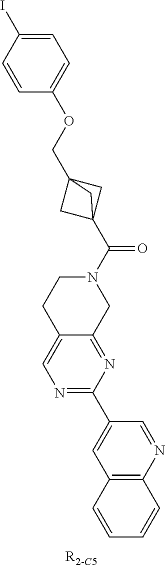 [SMILES: O=C(N1CCc2cnc(-c3cnc4ccccc4c3)nc2C1)C12CC(COc3ccc(I)cc3)(C1)C2] | 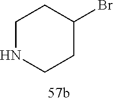 | 21.2% |
| | LC-MS: C₅₄H₅₆N₉O₅, [M/2 + H]⁺ 456.0; found | | | |
| | 455.8. ¹H NMR (600 MHz, DMSO-d₆) δ 10.97- | | | |
| | 10.95 (m, 1H), 9.82 (s, 1H), 9.29 (s, 1H), 8.84 (s, | | | |
| | 1H), 8.23 (d, J = 6.6 Hz, 1H), 8.11 (d, J = 8.4 Hz, | | | |
| | 1H), 7.87 (t, J = 7.2 Hz, 1H), 7.70 (t, J = 7.2 Hz, | | | |
| | 1H), 7.55-7.54 (m, 1H), 7.46-7.45 (m, 2H), 7.38- | | | |
| | 7.37 (m, 2H), 7.02-6.96 (m, 2H), 5.08-5.04 (m, | | | |
| | 1H), 4.95-4.79 (m, 2H), 4.36-4.20 (m, 2H), 4.09- | | | |
| | 3.95 (m, 6H), 3.80 (s, 1H), 3.68-3.64 (m, 3H), | | | |
| | 3.17 (s, 4H), 3.06-2.77 (m, 5H), 2.64-2.62 (m, | | | |
| | 3H), 2.39-2.36 (m, 1H), 2.16-2.03 (m, 9H), 2.00- | | | |
| | 1.95 (m, 1H), 1.67 (s, 2H). Purity >95%. | | | |

According to the step 1 to step 5 of the method for Compound F2-1, Compound R_{1B-5} was replaced with Compound R_{1A-5}, Compound R_{2-D1} was replaced with the raw material 1 in the following table, Compound 57a was replaced with the raw material 2 in the following table, and the other raw materials and operating method remained unchanged to obtain Compound F2-11.

| | | Raw | Raw | |
|---|---|---|---|---|
| Compound | Structure and characterization data of | material | material | |
| No. | compound | 1 | 2 | Yield |
| F2-11 | 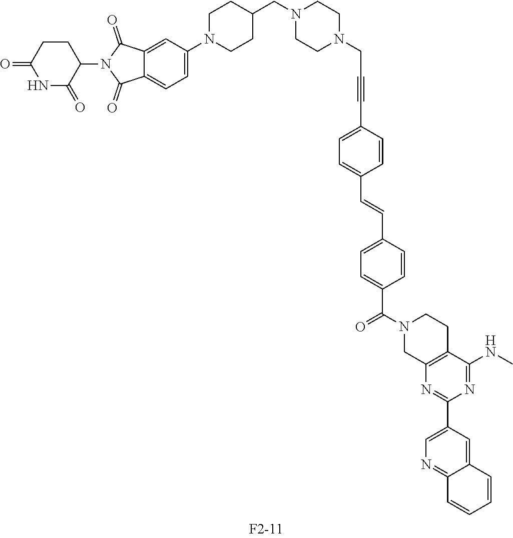 [SMILES: CNc1nc(-c2cnc3ccccc3c2)nc2c1CCN(C(=O)c1ccc(/C=C/c3ccc(C#CCN4CCN(CC5CCN(c6ccc7c(c6)C(=O)N(C6CCC(=O)NC6=O)C7=O)CC5)CC4)cc3)cc1)C2] | 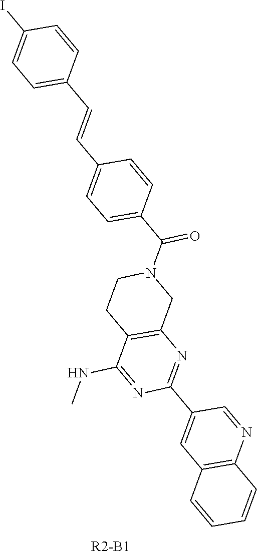 [SMILES: CNc1nc(-c2cnc3ccccc3c2)nc2c1CCN(C(=O)c1ccc(/C=C/c3ccc(I)cc3)cc1)C2] | 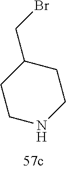 | |
| | LC-MS: C₅₈H₅₇N₁₀O₅, [M + H]⁺ 973.44; found | | | |
| | 973.5. ¹H NMR (400 MHz, DMSO-d₆) δ 11.01 (s, | | | |
| | 1H), 9.87-9.75 (m, 1H), 9.19-9.12 (m, 1H), 8.15- | | | |
| | 8.06 (m, 2H), 7.80 (m, 1H), 7.73 (d, J =12.6 Hz, | | | |
| | 2H), 7.68-7.63 (m, 4H), 7.54-7.52 (m, 2H), 7.47- | | | |
| | 7.45 (m, 2H), 7.38 (s, 2H), 7.33 (m, 1H), 7.24 (m, | | | |
| | 1H), 7.17 (s, 1H), 5.09-5.06 (m, 1H), 4.71-4.57 | | | |
| | (m, 2H), 3.95-3.70 (m, 2H), 3.53 (s, 2H), 3.42 (s, | | | |
| | 4H), 3.34 (m, 2H), 3.18-3.17 (m, 3H), 2.94-2.86 | | | |
| | (m, 3H), 2.64-2.53 (m, 4H), 2.49-2.42 (m, 4H), | | | |
| | 2.23-2.17 (m, 4H), 2.03-1.99 (m, 1H), 1.74 (m, | | | |
| | 2H), 1.52 (s, 1H). Purity >99%. | | | |

### Example 12. Synthesis of Compounds F2-5 and F2-6 of the Present Invention

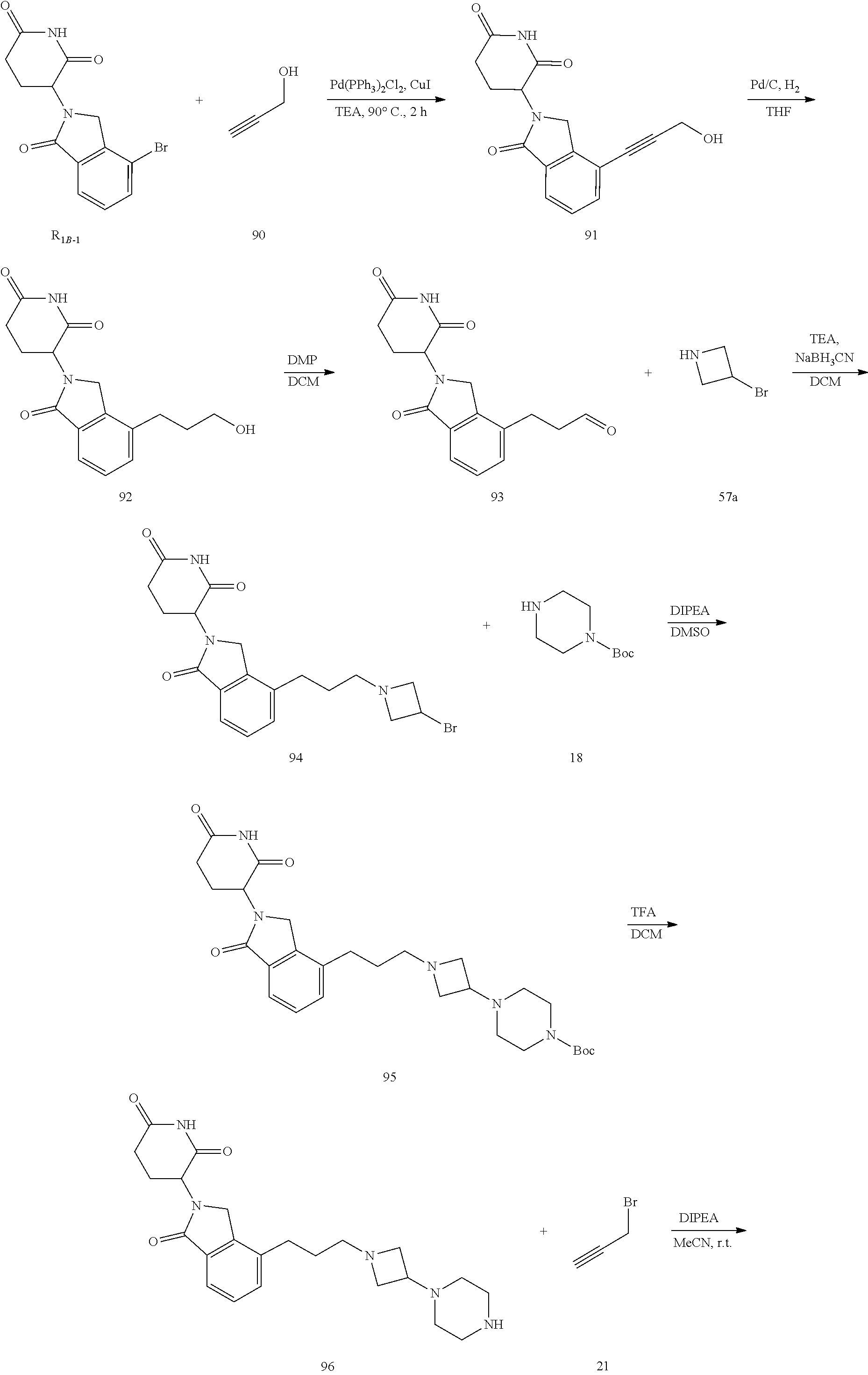

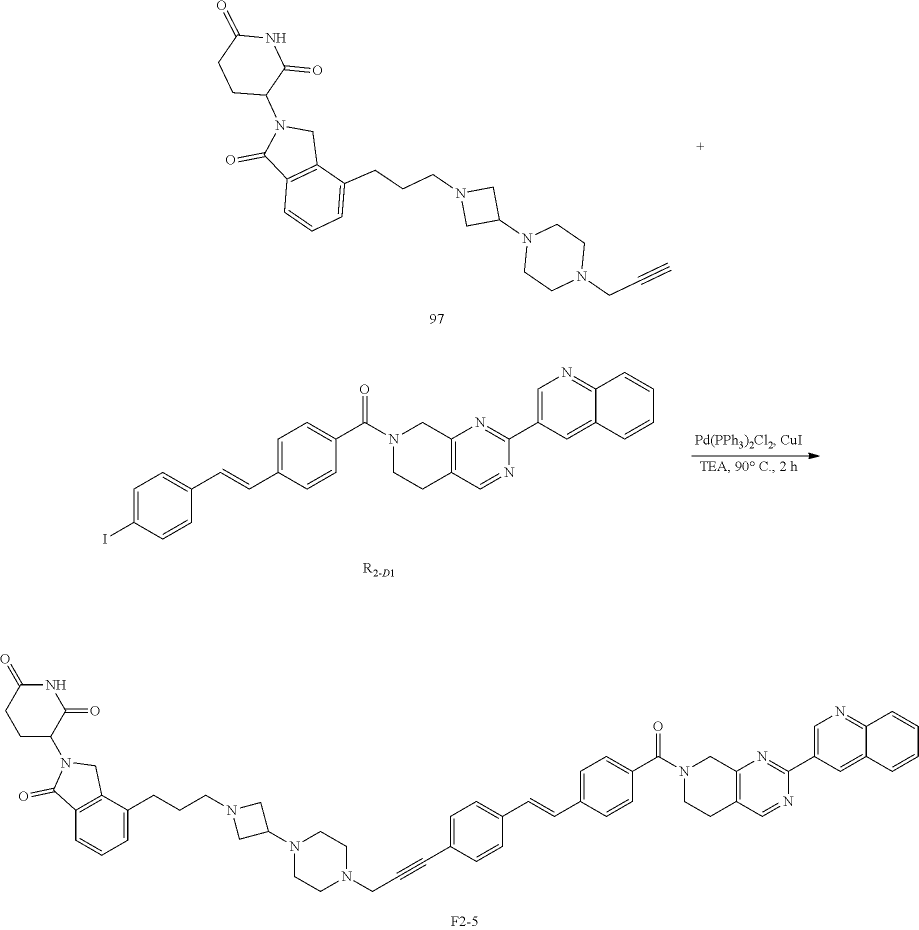 [SMILES: C#CCN1CCN(C2CN(CCCc3cccc4c3CN(C3CCC(=O)NC3=O)C4=O)C2)CC1 ; O=C(c1ccc(/C=C/c2ccc(I)cc2)cc1)N1CCc2cnc(-c3cnc4ccccc4c3)nc2C1 ; "TEA, 90° C., 2 h" ; O=C1CCC(N2Cc3c(CCCN4CC(N5CCN(CC#Cc6ccc(/C=C/c7ccc(C(=O)N8CCc9cnc(-c%10cnc%11ccccc%11c%10)nc9C8)cc7)cc6)CC5)C4)cccc3C2=O)C(=O)N1]

### Step 1. Synthesis of Compound 91

Under nitrogen protection, Compound R_{1B-1} (1.00 mmol), compound 90 (1.05 mmol), copper iodide (2.00 mmol), triethylamine (5 mL), and Pd(PPh₃)₂Cl₂ (1.12 mg, 0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound 91 (C₁₆H₁₅N₂O₄, [M+H]⁺ 299.1; found 299.2, yield 58.6%).

### Step 2. Synthesis of Compound 92

Compound 91 (0.55 mmol), tetrahydrofuran (5 mL), and palladium-on-carbon (30.00 mg) were added in sequence to a 50 mL reaction bottle. The reaction system was replaced with hydrogen, maintained at an atmospheric pressure hydrogen atmosphere, and the reaction was carried out under stirring at room temperature for 3 hours (LC-MS monitoring). Filtration was carried out to remove palladium-on-carbon, washing was carried out with methanol (2×20 mL), the filtrate was collected, and concentrated under reduced pressure to remove the solvent to obtain Compound 92 (C₁₆H₁₉N₂O₄, [M+H]⁺ 303.1; found 303.2, crude product).

### Step 3. Synthesis of Compound 93

Compound 92 (0.55 mmol), Dess-Martin Periodinane (1.02 mmol) and dichloromethane (5 mL) were added to a 50 mL reaction bottle, reacted under stirring at room temperature for 1 hour, and then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 93 (C₁₆H₁₇N₂O₄, [M+H]⁺ 301.1; found 301.2, yield 81.8%).

### Step 4. Synthesis of Compound 94

Compound 93 (0.45 mmol), Compound 57a (0.65 mmol), triethylamine (2 mL), sodium cyanoborohydride (0.85 mmol) and dichloroethane (5 mL) were added in sequence to a 50 mL reaction bottle, reacted under stirring at room temperature for 2 hours, then the reaction was quenched (LC-MS monitoring), the solvent was removed by evaporation to dryness, purification was carried out by medium-pressure preparative chromatography, and concentrated under reduced pressure to remove the solvent to obtain Compound 94 (C₁₉H₂₃BrN₃O₃, [M+H]⁺ 420.1; found 420.2, yield 77.8%).

### Step 5. Synthesis of Compound 95

Compound 94 (0.35 mmol), Compound 18 (0.65 mmol), N,N-diisopropylethylamine (1.02 mmol) and DMSO (5 mL) were added into a 50 mL reaction bottle, stirred and reacted at room temperature for 1 hour, then water was added to quench the reaction (LC-MS monitoring).

Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 95 (C₂₈H₄₀N₅O₅, [M+H]⁺ 526.3; found 526.5, yield 85.7%).

### Step 6. Synthesis of Compound 96

Compound 95 (0.30 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 96 (C₂₃H₃₂N₅O₃, [M+H]⁺ 426.2; found 426.4, crude product).

### Step 7. Synthesis of Compound 97

Compound 96 (0.20 mmol), Compound 21 (0.55 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 97 (C₂₆H₃₄N₅O₃, [M+H]⁺ 464.3; found 464.5, yield 51.8%).

### Step 8. Synthesis of Compound F2-5

Under nitrogen protection, Compound R_{2-D1} (0.10 mmol), Compound 97 (0.09 mmol), copper iodide (0.25 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound F2-5 (7.2 mg, 7.73 μmol). LC-MS: C₅₇H₅₆N₉O₄, [M/2+H]⁺465.7; found 466.2. ¹H NMR (400 MHz, Methanol-d4) δ 9.96-9.90 (m, 1H), 9.58-9.51 (m, 1H), 8.79 (s, 1H), 8.22-8.15 (m, 2H), 7.98 (s, 1H), 7.97-7.73 (m, 3H), 7.71-7.70 (m, 1H), 7.68 (d, J=4.0 Hz, 2H), 7.63-7.45 (m, 6H), 7.33 (s, 2H), 5.21-5.16 (m, 1H), 5.09-4.98 (m, 2H), 4.57-4.44 (m, 2H), 4.37 (s, 2H), 4.32-3.68 (m, 6H), 3.59-3.30 (m, 5H), 3.29-3.05 (m, 4H), 2.96-2.91 (m, 2H), 2.89-2.78 (m, 3H), 2.68-2.66 (m, 1H), 2.55-2.44 (m, 2H), 2.21-2.16 (m, 2H), 1.96-1.71 (m, 2H).

Compound F2-6 could be obtained by following the step 1 to step 8 of the method for Compound F2-5, replacing Compound 57a with the raw materials in the following table, and keeping other raw materials and operating methods unchanged.

| Compound | | Raw | |
|---|---|---|---|
| No. | Structure and characterization data of compound | material | Yield |
| F2-6 | 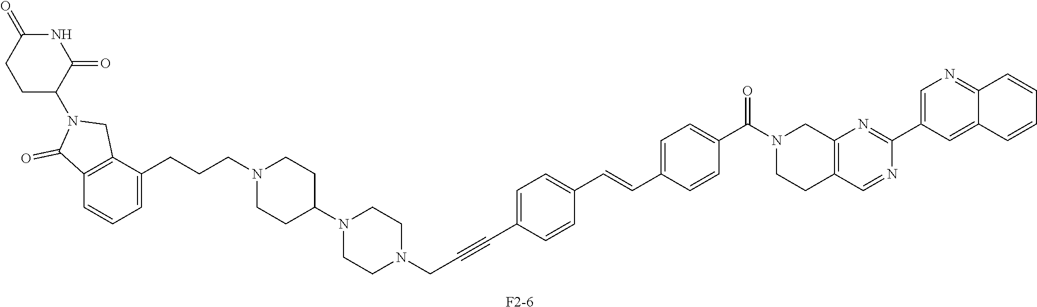 [SMILES: O=C1CCC(N2Cc3c(CCCN4CCC(N5CCN(CC#Cc6ccc(/C=C/c7ccc(C(=O)N8CCc9cnc(-c%10cnc%11ccccc%11c%10)nc9C8)cc7)cc6)CC5)CC4)cccc3C2=O)C(=O)N1 ; "F2-6"] | 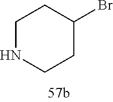 | 20.8% |
| | LC-MS: C₅₉H₆₀N₉O₄, [M/2 + H]⁺ 479.8; found 480.1. ¹H | | |
| | NMR (400 MHz, Methanol-d₄) δ 10.01-9.91 (m, 1H), 9.80- | | |
| | 9.74 (m, 1H), 8.80 (d, J = 4.0 Hz, 1H), 8.34-8.33 (m, 2H), | | |
| | 8.08 (s, 1H), 7.89 (s, 1H), 7.74-7.69 (m, 3H), 7.62-7.59 (m, | | |
| | 2H), 7.56-7.47 (m, 6H), 7.30 (d, J = 4.0 Hz, 2H), 5.21-5.16 | | |
| | (m, 1H), 5.03-4.98 (m, 2H), 4.57-4.38 (m, 3H), 4.12-4.08 (m, | | |
| | 3H), 3.85-3.71 (m, 3H), 3.31-2.97 (m, 12H), 2.95-2.88 (m, | | |
| | 2H), 2.80-2.78 (m, 3H), 2.57-2.46 (m, 2H), 2.26-1.95 (m, | | |
| | 7H). | | |

### Example 13. Synthesis of Compounds F3A-1, F3A-2, F3A-3, and F3A-4 of the Present Invention

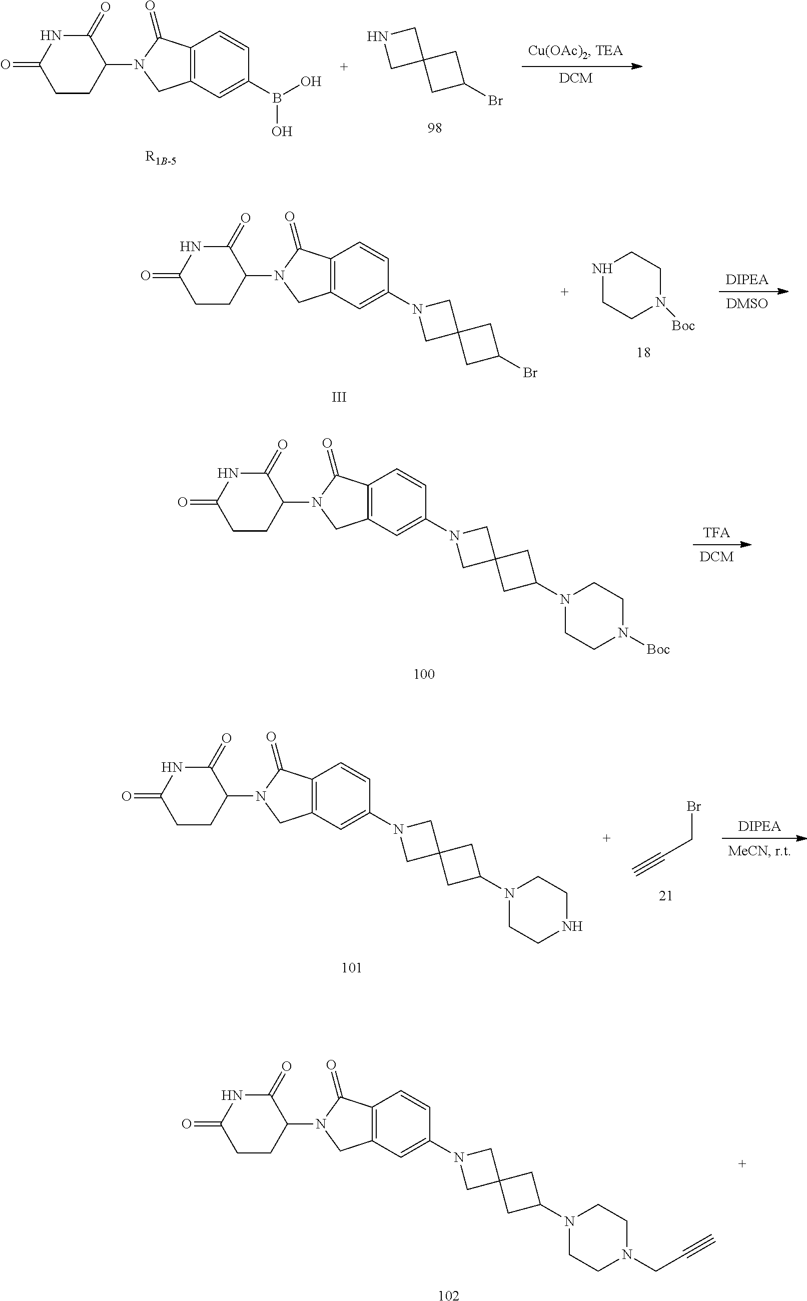

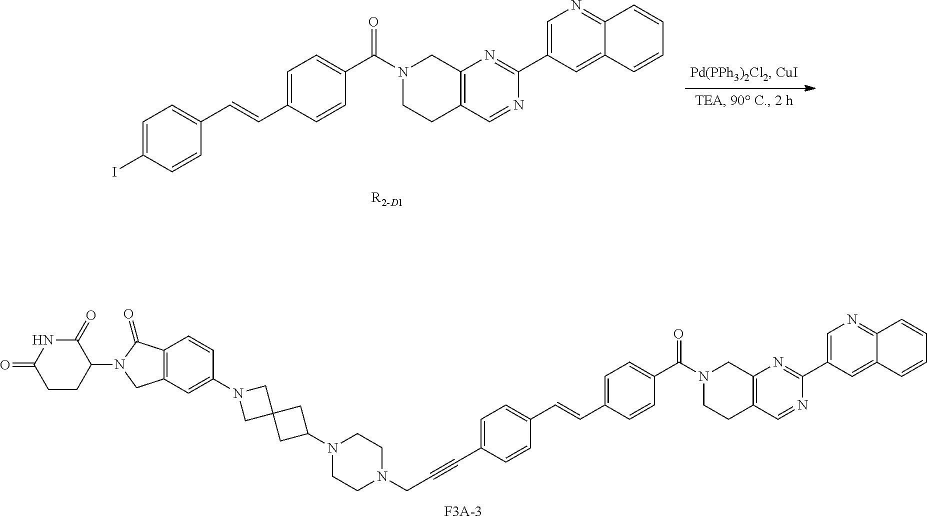

### Step 1. Synthesis of Compound III

Under nitrogen protection, Compound R_{1B-5} (1.0 mmol), Compound 98 (1.0 mmol), Cu(OAc)₂ (2.0 mmol), and triethylamine (5 mL) were added to a 50 mL reaction bottle in sequence. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound III (C₁₉H₂₁BrN₃O₃, [M+H]⁺ 418.1; found 418.2, yield 78.9%).

### Step 2. Synthesis of Compound 100

Compound III (0.60 mmol), Compound 18 (0.85 mmol), N,N-diisopropylethylamine (1.02 mmol) and DMSO (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 100 (C₂₈H₃₈N₅O₅, [M+H]⁺ 524.3; found 524.5, yield 76.2%).

### Step 3. Synthesis of Compound 101

Compound 100 (0.45 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 101 (C₂₃H₃₀N₅O₃, [M+H]⁺ 424.2; found 424.1, crude product).

### Step 4. Synthesis of Compound 102

Compound 101 (0.40 mmol), Compound 21 (0.85 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 102 (C₂₆H₃₂N₅O₃, [M+H]⁺ 462.2; found 462.3, yield 37.5%).

### Step 5. Synthesis of Compound F3A-3

Under nitrogen protection, Compound R_{2-D1} (0.15 mmol), Compound 102 (0.15 mmol), copper iodide (0.45 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound F3A-3 (2.4 mg, 2.70 μmol). LC-MS: C₅₄H₅₀N₉O₄, [M/2+H]⁺464.7; found 465.0. ¹H NMR (400 MHz, DMSO-d6) δ 10.93 (s, 1H), 9.83-9.61 (m, 1H), 9.30-9.29 (m, 1H), 8.86 (s, 1H), 8.21-7.40 (m, 13H), 7.28-7.02 (m, 2H), 6.77-6.48 (m, 2H), 5.32-4.80 (m, 3H), 4.31-3.72 (m, 8H), 3.10-2.99 (m, 8H), 1.46-0.85 (m, 13H).

Compounds F3A-1, F3A-2 and F3A-4 could be obtained by following the step 2 to step 5 of the method for Compound F3A-3, replacing Compound III with the raw materials in the following table, and keeping the other raw materials and operating methods unchanged.

The preparation of Compounds IA, IB and II was as follows:

### Synthesis of Compound I

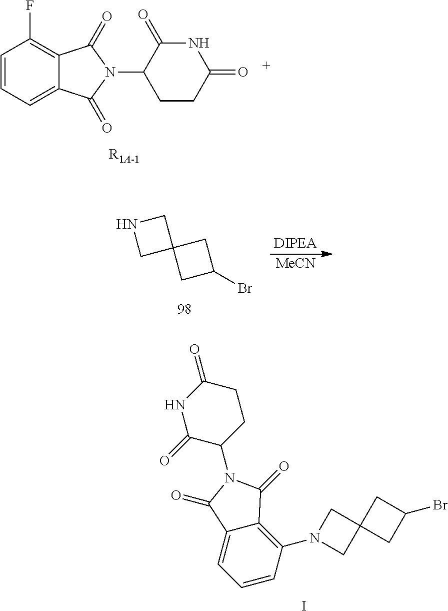

Compound R_{1A-1} (1.0 mmol), Compound 98 (1.15 mmol), N,N-diisopropylethylamine (2.80 mmol) and acetonitrile (10 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Intermediate I (C₁₉H₁₉BrN₃O₄, [M+H]⁺ 432.1; found 432.3, yield 86.9%).

### Synthesis of Compound II

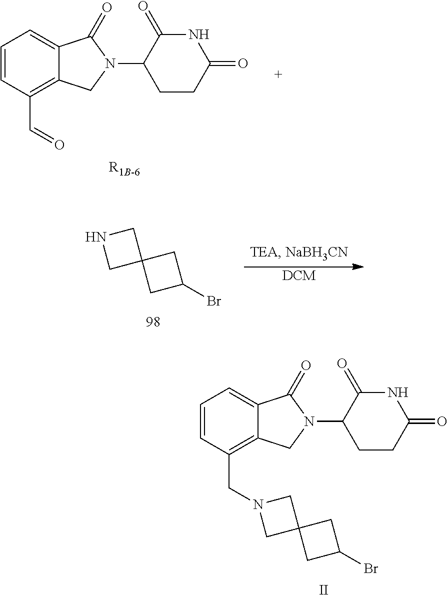

Compound R_{1B-6} (1.00 mmol), Compound 98 (1.05 mmol), triethylamine (2 mL), sodium cyanoborohydride (1.55 mmol) and dichloromethane (5 mL) were added in sequence to a 50 mL reaction bottle, reacted under stirring at room temperature for 2 hours, then the reaction was quenched (LC-MS monitoring), the solvent was removed by evaporation to dryness, purified by medium-pressure preparative chromatography, and concentrated under reduced pressure to remove the solvent to obtain Intermediate II (C₂₀H₂₃BrN₃O₃, [M+H]⁺ 432.1; found 432.3, yield 85.2%).

### Synthesis of Compound IB

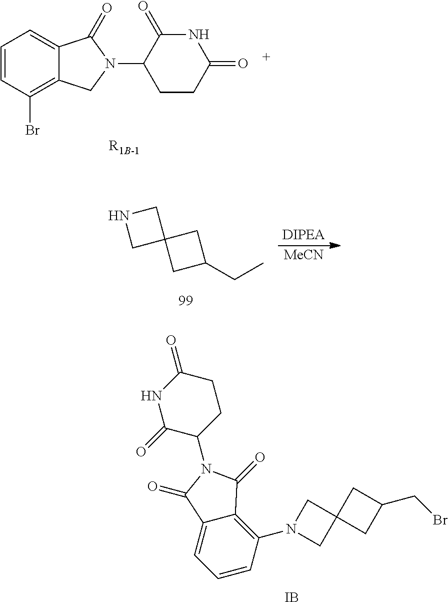

Compound R_{1B-1} (1.0 mmol), Compound 99 (1.15 mmol), N,N-diisopropylethylamine (2.80 mmol) and acetonitrile (10 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Intermediate TB (C₂₀H₂₃BrN₃O₃, [M+H]⁺433.3; found 433.3, yield 79.2%).

| Compound | Structure and characterization data of | | |
|---|---|---|---|
| No. | compound | Raw material | Yield |
| F3A-1 | 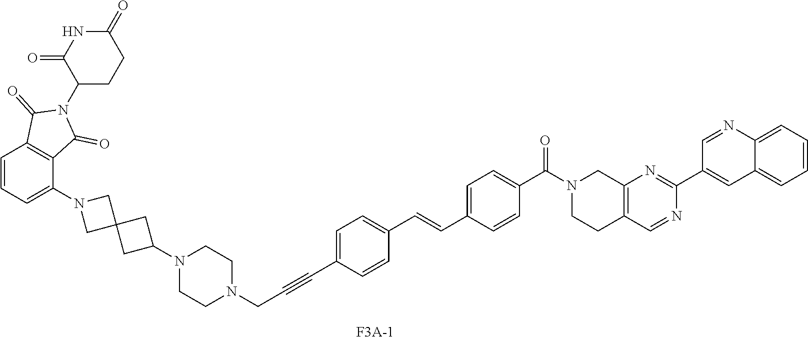 [SMILES: O=C1CCC(N2C(=O)c3cccc(N4CC5(CC(N6CCN(CC#Cc7ccc(/C=C/c8ccc(C(=O)N9CCc%10cnc(-c%11cnc%12ccccc%12c%11)nc%10C9)cc8)cc7)CC6)C5)C4)c3C2=O)C(=O)N1 ; "F3A-1"] | 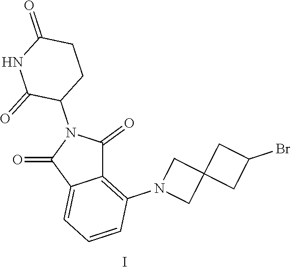 | 20.1% |
| | LC-MS: C₅₇H₅₂N₉O₅, [M/2 + H]⁺ 472.0; found 472.1. | | |
| | ¹H NMR (400 MHz, DMSO-d₆) δ 9.85-9.75 (m, 1H), | | |
| | 9.45-9.34 (m, 1H), 8.85 (s, 1H), 8.29-8.24 (m, 2H), | | |
| | 8.16 (s, 1H), 7.97-7.79 (m, 3H), 7.78-7.76 (m, 2H), | | |
| | 7.68 (d, J = 8.0 Hz, 1H), 7.61 (d, J = 4.0 Hz, 2H), | | |
| | 7.58-7.52 (m, 2H), 7.38 (s, 2H), 7.17 (d, J = 7.2 Hz, | | |
| | 1H), 6.80 (d, J = 8.0 Hz, 1H), 5.10-4.97 (m, 3H), | | |
| | 4.25-4.18 (m, 3H), 3.99-3.65 (m, 5H), 3.16-2.81 (m, | | |
| | 12H), 2.73-2.62 (m, 3H), 2.58-2.41 (m, 3H), 2.05- | | |
| | 2.04 (m, 1H). | | |
| F3A-2 | 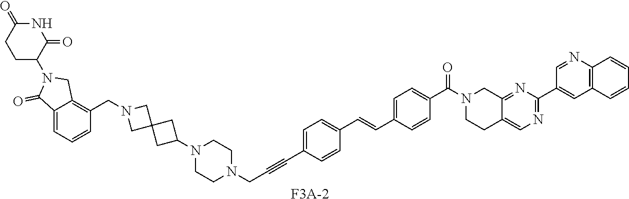 | 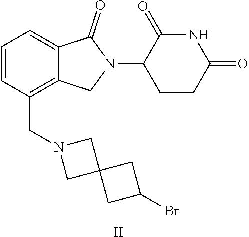 | 18.6% |
| | LC-MS: C₅₈H₅₆N₉O₄, [M/2 + H]⁺ 472.0; found 471.9. | | |
| | ¹H NMR (400 MHz, DMSO-d₆) δ 9.79-9.73 (m, 1H), | | |
| | 9.33-9.31 (m, 1H), 8.85 (s, 1H), 8.21-8.10 (m, 2H), | | |
| | 7.86-7.84 (m, 2H), 7.77-7.65 (m, 7H), 7.57-7.47 (m, | | |
| | 4H), 7.38 (s, 2H), 5.33-5.14 (m, 2H), 4.95-4.92 (m, | | |
| | 1H), 4.80-4.77 (m, 1H), 4.62-4.58 (2H), 4.49-4.45 | | |
| | (m, 3H), 4.28-4.21 (m, 3H), 4.08-4.02 (m, 3H), 3.58- | | |
| | 3.52 (m, 2H), 3.19-2.89 (m, 7H), 2.03-1.97 (m, 2H), | | |
| | 1.23 (s, 6H). | | |
| F3A-4 | 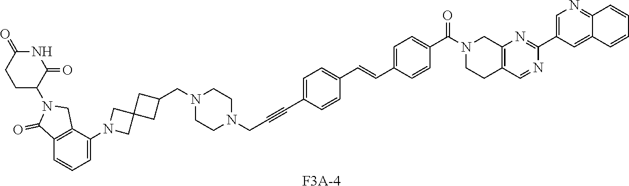 [SMILES: O=C1CCC(N2Cc3c(cccc3N3CC4(CC(CN5CCN(CC#Cc6ccc(/C=C/c7ccc(C(=O)N8CCc9cnc(-c%10cnc%11ccccc%11c%10)nc9C8)cc7)cc6)CC5)C4)C3)C2=O)C(=O)N1 ; "F3A-4"] | 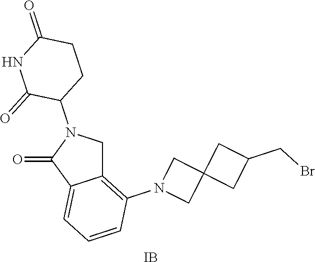 | 12.7% |
| | LC-MS: C₅₈H₅₆N₉O₄, [M + H]⁺ 472.0; found 472.0. | | |
| | ¹H NMR (400 MHz, DMSO-d₆) δ 9.84-9.77 (m, 1H), | | |
| | 9.40-9.33 (m, 1H), 8.86 (s, 1H), 8.25-8.13 (m, 2H), | | |
| | 7.93 (s, 1H), 7.77-7.69 (m, 3H), 7.67-7.63 (m, 2H), | | |
| | 7.60-7.48 (m, 4H), 7.38 (s, 2H), 7.34-7.30 (m, 1H), | | |
| | 7.09 (d, J = 8.0 Hz, 1H), 6.57 (d, J = 7.8 Hz, 1H), | | |
| | 5.12-5.06 (m, 1H), 4.96-4.81 (m, 2H), 4.47-4.42 (m, | | |
| | 1H), 4.31-4.27 (m, 1H), 4.03-4.01 (m, 2H), 3.91-3.87 | | |
| | (m, 2H), 3.72 (s, 4H), 3.58-3.49 (m, 1H), 3.38-3.32 | | |
| | (m, 2H), 3.19-3.17 (m, 3H), 3.12-3.08 (m, 3H), 3.00- | | |
| | 2.88 (m, 5H), 2.71-2.68 (m, 2H), 2.44-2.35 (m, 3H), | | |
| | 2.08-2.06 (m, 2H). | | |

### Example 14. Synthesis of Compounds F31B-1, F31B-2, and F31B-3 of the Present Invention

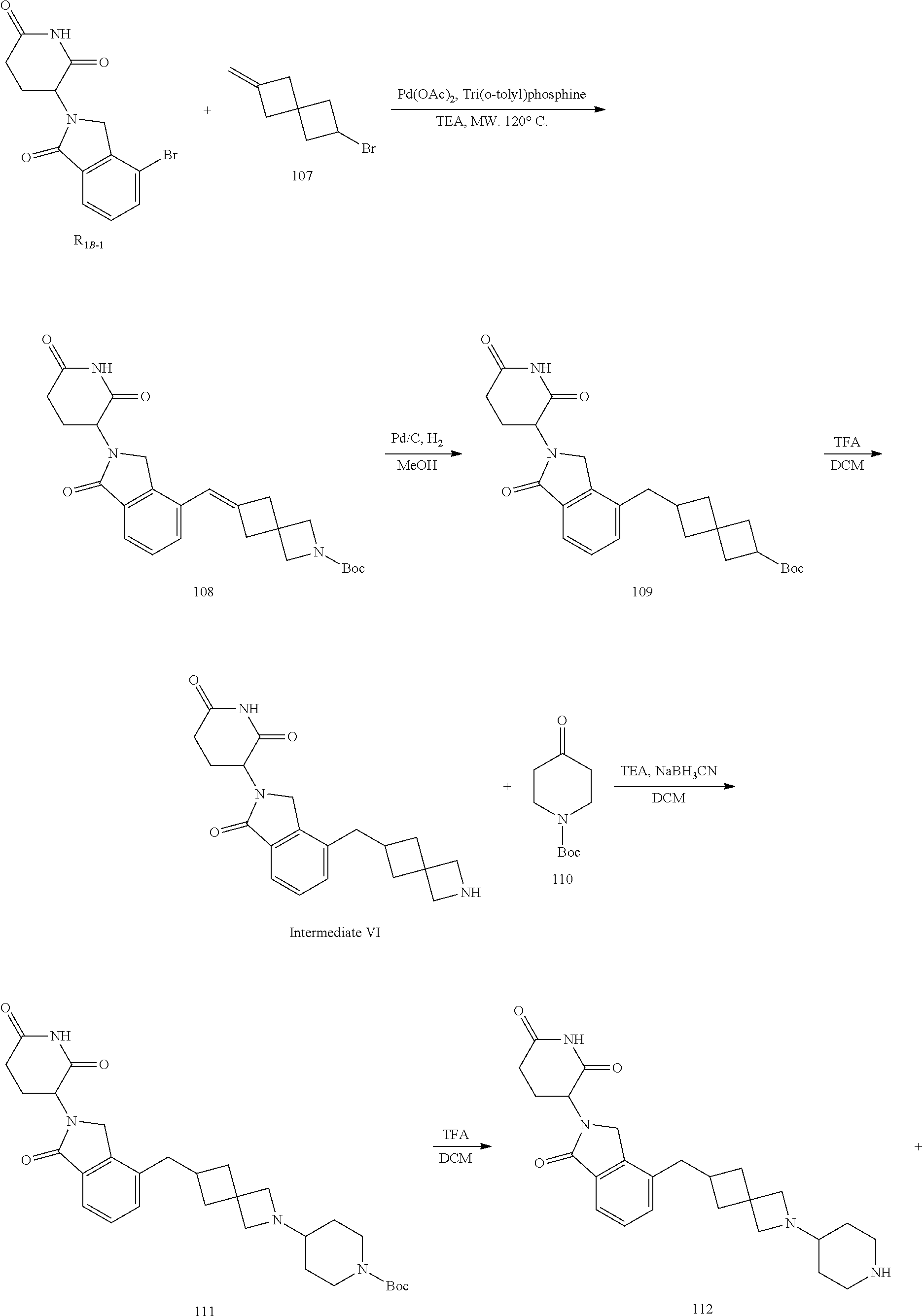

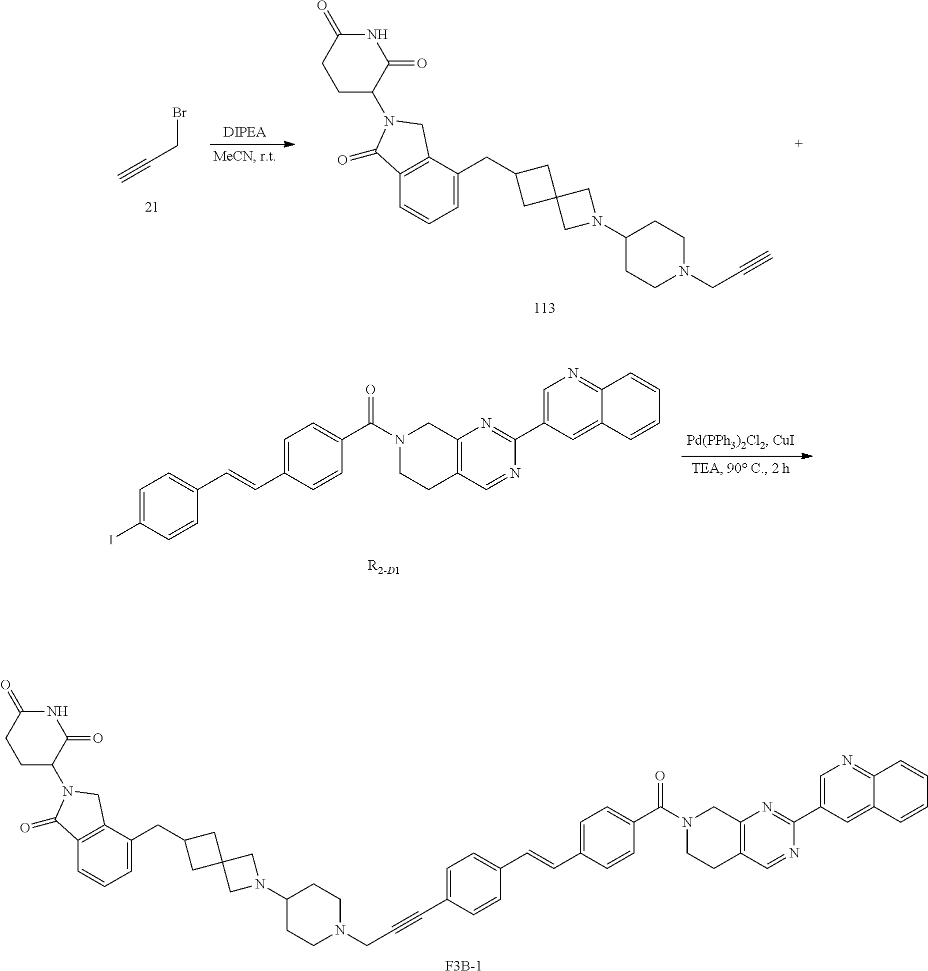

### Step 1-3, Synthesis of Intermediate VI

Under nitrogen protection, Compound R_{1B-1} (1.00 mmol), Compound 107 (1.05 mmol), tri(o-tolyl)phosphine (1.05 mmol), triethylamine (2 mL), and palladium acetate (0.25 mol %) were added in sequence to a 50 mL reaction bottle. The reaction system was sealed, the reaction was carried out under stirring in a microwave reactor at 120° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound 108 (C₂₅H₃₀N₃O₅, [M+H]⁺ 452.2; found 452.3, yield 48.2%).

Compound 108 (0.45 mmol), methanol (5 mL), and palladium-on-carbon (30.00 mg) were added in sequence to a 50 mL reaction bottle. The reaction system was replaced with hydrogen, maintained at an atmospheric pressure hydrogen atmosphere, and the reaction was carried out under stirring at room temperature for 3 hours (LC-MS monitoring). The palladium-on-carbon was removed by filtration, and washing was carried out with methanol (2×20 mL). The filtrate was collected and concentrated under reduced pressure to remove the solvent to obtain Compound 109 (C₂₅H₃₂N₃O₅, [M+H]⁺ 454.2; found 454.1, crude product).

Compound 109 (0.30 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound Intermediate VI (C₂₀H₂₄N₃O₃, [M+H]⁺ 354.2; found 354.1, crude product).

### Step 4. Synthesis of Compound 111

Intermediate VI (0.30 mmol), Compound 110 (0.55 mmol), triethylamine (2 mL), sodium cyanoborohydride (0.75 mmol) and dichloromethane (5 mL) were added in sequence to a 50 mL reaction bottle, reacted under stirring at room temperature for 2 hours, then the reaction was quenched (LC-MS monitoring), the solvent was removed by evaporation to dryness, purified by medium-pressure preparative chromatography, and concentrated under reduced pressure to remove the solvent to obtain Compound 111 (C₃₀H₄₁N₄O₅, [M+H]⁺ 537.3; found 537.5, yield 71.8%).

### Step 5. Synthesis of Compound 112

Compound 111 (0.21 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound 112 (C₂₅H₃₃N₄O₃, [M+H]⁺ 437.3; found 437.5, crude product).

### Step 6. Synthesis of Compound 113

Compound 112 (0.21 mmol), Compound 21 (0.44 mmol), N,N-diisopropylethylamine (0.95 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 113 (C₂₈H₃₅N₄O₃, [M+H]⁺ 457.3; found 457.5, yield 71.8%).

### Step 7. Synthesis of Compound F3B-1

Under nitrogen protection, Compound R_{2-D1} (0.15 mmol), Compound 113 (0.15 mmol), copper iodide (0.35 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound F3B-1 (9.1 mg, 9.66 μmol,). LC-MS: C₅₉H₅₇N₈O₄, [M/2+H]⁺471.6; found 471.7. ¹H NMR (400 MHz, DMSO-d6) δ 9.87-9.77 (m, 1H), 9.36-9.24 (m, 1H), 8.86 (s, 1H), 8.22-8.12 (m, 2H), 7.90 (s, 1H), 7.78-7.72 (m, 5H), 7.62-7.51 (m, 6H), 7.49-7.47 (m, 1H), 7.44-7.42 (m, 3H), 5.10-5.09 (m, 1H), 4.97 (s, 1H), 4.80-4.77 (m, 1H), 4.48-4.44 (m, 1H), 4.36-4.28 (m, 3H), 4.18-4.15 (m, 2H), 4.09 (s, 2H), 3.51 (s, 2H), 3.44-3.41 (m, 1H), 3.10-2.95 (m, 4H), 2.92-2.89 (m, 1H), 2.75-2.73 (m, 2H), 2.68-2.63 (m, 1H), 2.44-2.41 (m, 3H), 2.20-2.16 (m, 4H), 2.06-1.96 (m, 4H), 1.62-1.55 (m, 2H).

According to the step 4 to step 7 of the method for Compound F3B-1, the raw materials in the following table were used to replace Intermediate VI, and the other raw materials and operating methods remained unchanged to obtain Compounds F3B-2 and F3B-3.

The synthesis of Compounds IV and V was as follows:

### Synthesis of Intermediate IV

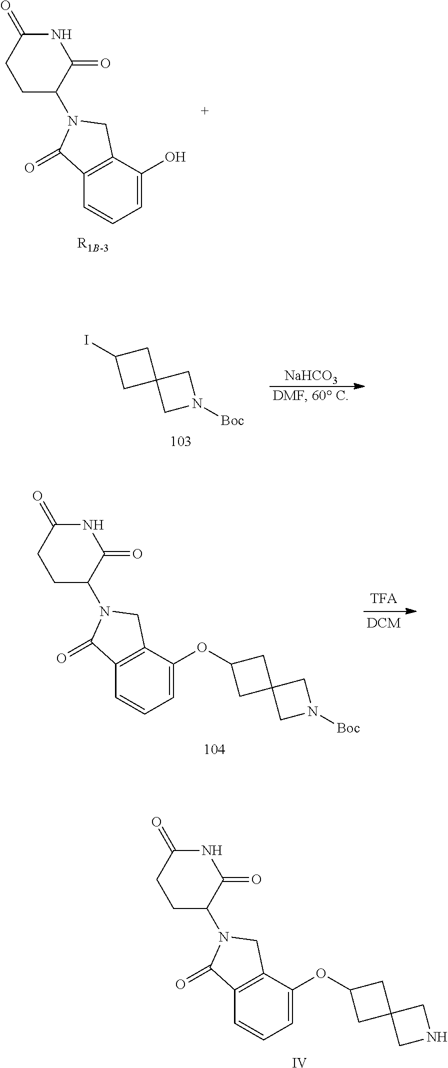

Compound R_{1B-3} (1.00 mmol), Compound 103 (1.05 mmol), sodium bicarbonate (1.15 mmol) and N,N-dimethylformamide (5 mL) were added into a 50 mL reaction bottle, heated and reacted at 60° C. After 1 hour, the reaction was quenched by adding water (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 104 (C₂₄H₃₀N₃O₆, [M+H]⁺ 456.2; found 456.4, yield 37.5%).

Compound 104 (0.35 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound Intermediate IV (C₁₉H₂₂N₃O₄, [M+H]⁺ 356.2; found 356.3, crude product).

### Synthesis of Intermediate V

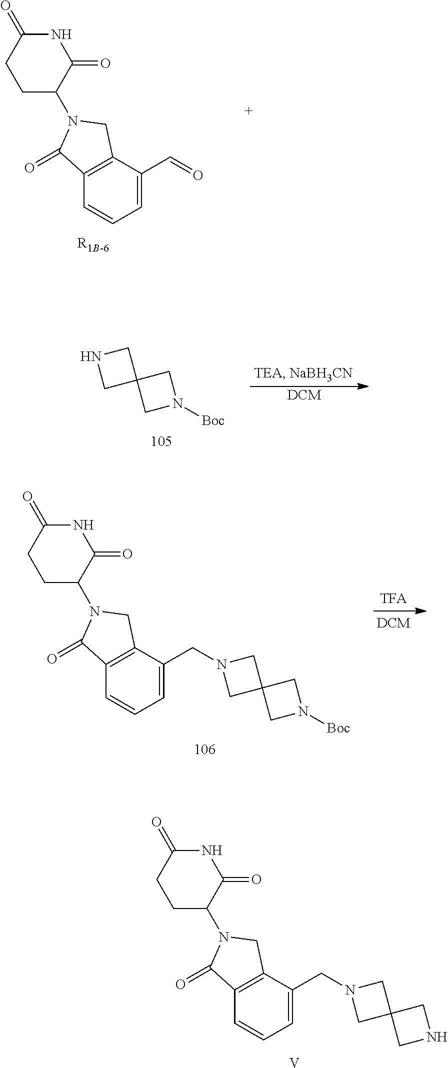

Compound R_{1B-6} (1.00 mmol), Compound 105 (1.05 mmol), triethylamine (2 mL), sodium cyanoborohydride (1.15 mmol) and dichloromethane (5 mL) were added in sequence to a 50 mL reaction bottle, reacted under stirring at room temperature for 2 hours, and then the reaction was quenched (LC-MS monitoring). The solvent was removed by evaporation to dryness, purified by medium-pressure preparative chromatography, and concentrated under reduced pressure to remove the solvent to obtain Compound 106 (C₂₄H₃₁N₄O₅, [M+H]⁺ 455.2; found 455.5, yield 58.9%).

Compound 106 (0.55 mmol) was dissolved in dichloromethane (5 mL) in a 50 mL reaction bottle, then trifluoroacetic acid (1.5 mL) was added dropwise, and the reaction was carried out under stirring at room temperature for 0.5 hour. The solvent was removed by concentration under reduced pressure to obtain Compound Intermediate V (C₁₉H₂₃N₄O₃, [M+H]⁺ 355.2; found 355.4, crude product).

| Compound | | | |
|---|---|---|---|
| No. | Structure and characterization data of compound | Raw material | Yield |
| F3B-2 | 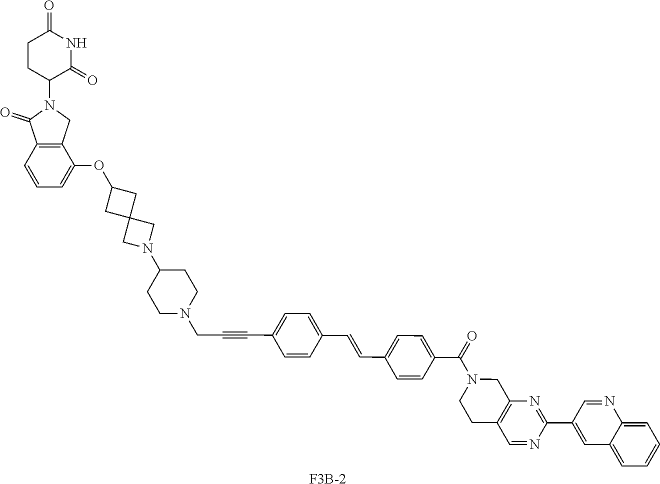 [SMILES: O=C1CCC(N2Cc3c(OC4CC5(C4)CN(C4CCN(CC#Cc6ccc(/C=C/c7ccc(C(=O)N8CCc9cnc(-c%10cnc%11ccccc%11c%10)nc9C8)cc7)cc6)CC4)C5)cccc3C2=O)C(=O)N1 ; "F3B-2"] | 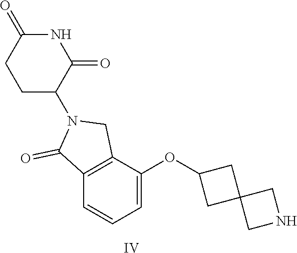 | 18.6% |
| | LC-MS: C₅₈H₅₅N₈O₅, [M/2 + H]⁺ 472.2; found 472.5. | | |
| | ¹H NMR (400 MHz, DMSO-d₆) δ 9.85-9.77 (m, 1H), | | |
| | 9.44-9.33 (m, 1H), 8.86 (s, 1H), 8.27-8.15 (m, 2H), | | |
| | 7.95 (s, 1H), 7.78-7.72 (m, 5H), 7.60-7.53 (m, 4H), | | |
| | 7.51-7.49 (m, 1H), 7.42-7.38 (m, 2H), 7.36-7.34 (m, | | |
| | 1H), 7.07 (d, J = 4.0 Hz, 1H), 5.07-5.06 (m, 1H), 4.86 | | |
| | (s, 1H), 4.84-4.82 (m, 2H), 4.41-4.36 (m, 3H), 4.26- | | |
| | 4.21 (m, 4H), 3.73 (s, 2H), 3.50-3.45 (m, 2H), 3.16 (s, | | |
| | 5H), 3.14-3.08 (m, 2H), 3.01 (s, 2H), 2.93-2.89 (m, | | |
| | 3H), 2.71-2.66 (m, 2H), 2.48-2.47 (m, 2H), 2.44-2.41 | | |
| | (m, 1H), 2.37-2.36 (m, 2H), 2.23-2.03 (m, 1H), 1.66- | | |
| | 1.64 (m, 2H), 1.28-1.25 (m, 1H). | | |
| F3B-3 | 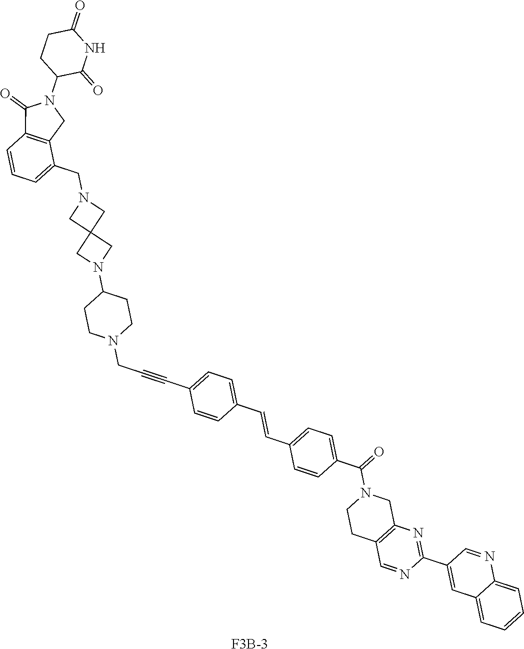 [SMILES: O=C1CCC(N2Cc3c(CN4CC5(C4)CN(C4CCN(CC#Cc6ccc(/C=C/c7ccc(C(=O)N8CCc9cnc(-c%10cnc%11ccccc%11c%10)nc9C8)cc7)cc6)CC4)C5)cccc3C2=O)C(=O)N1 ; "F3B-3"] | 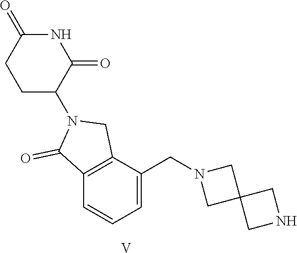 | 19.5% |
| | LC-MS: C₅₈H₅₆N₉O₄, [M/2 + H]⁺ 472.0; found 472.2. | | |
| | ¹H NMR (400 MHz, DMSO-d₆) δ 9.83-9.75 (m, 1H), | | |
| | 9.34-9.25 (m, 1H), 8.86 (s, 1H), 8.22-8.14 (m, 2H), | | |
| | 8.12-7.89 (s, 2H), 7.87-7.66 (m, 7H), 7.58-7.55 (m, | | |
| | 4H), 7.42 (s, 2H), 5.19-5.15 (m, 2H), 4.96-4.92 (m, | | |
| | 1H), 4.80-4.77 (m, 1H), 4.62-4.57 (2H), 4.48-4.36 (m, | | |
| | 10H), 4.03-3.91 (m, 2H), 3.44-3.38 (m, 2H), 3.19- | | |
| | 3.00 (m, 5H), 2.65-2.55 (m, 3H), 2.22-2.07 (m, 4H), | | |
| | 1.62 (s, 2H). | | |

### Example 15. Synthesis of Compounds G1-1, G1-2, G1-3, G1-4, and G1-5 of the Present Invention

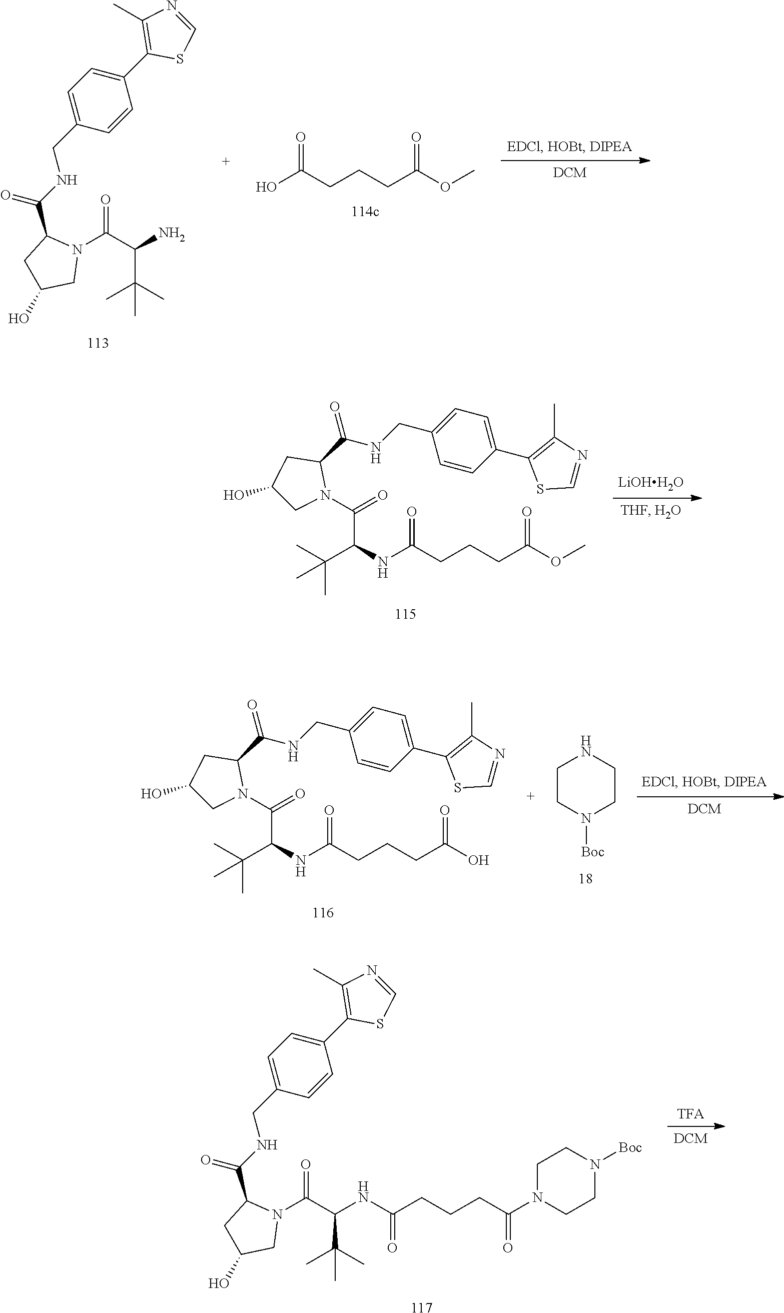

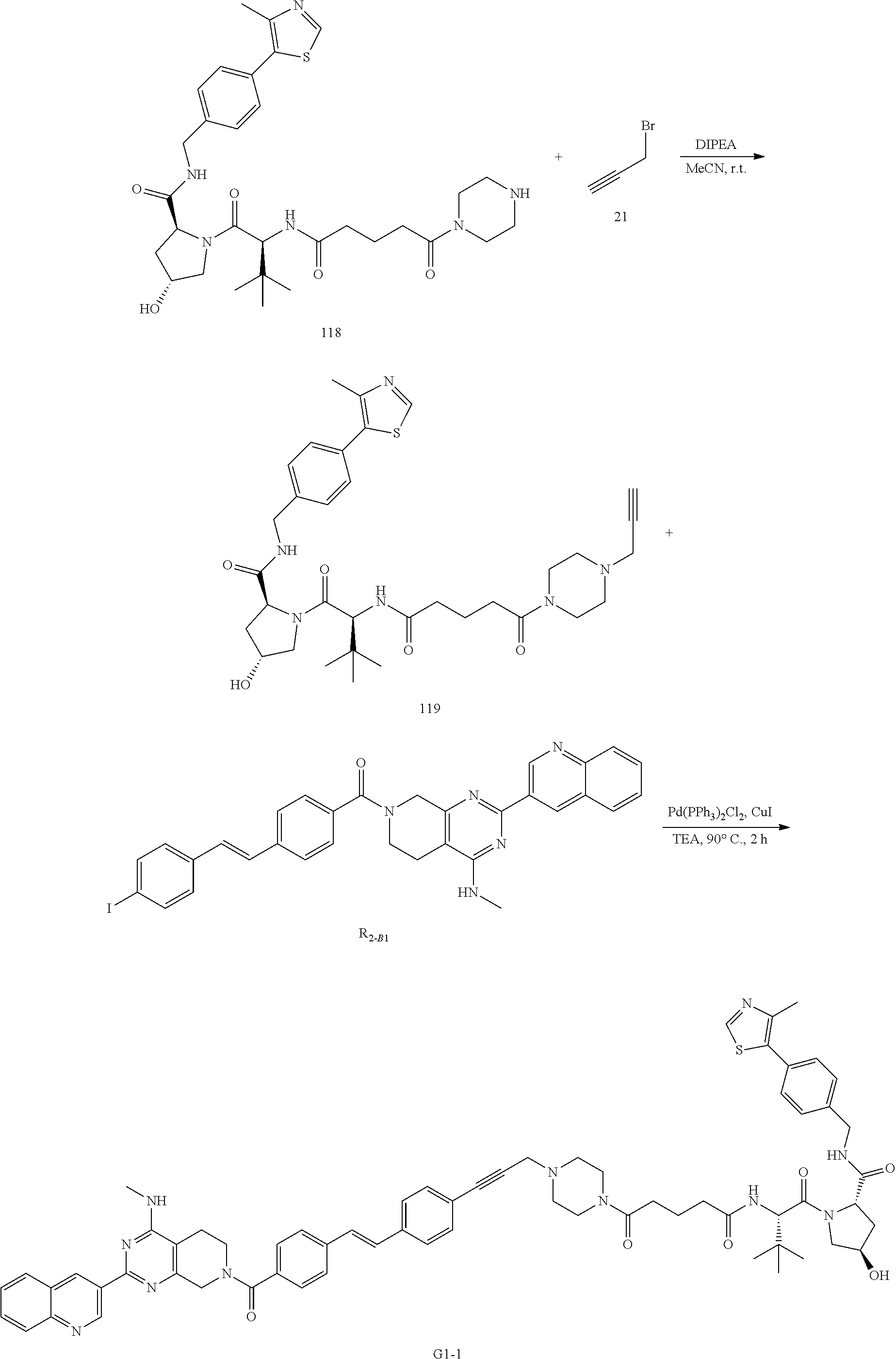

### Step 1. Synthesis of Compound 115

Compound 114a (1.00 mmol), EDCI (1.05 mmol), HOBt (1.05 mmol) and dichloromethane (2 mL) were added in sequence to a 50 mL reaction bottle. After the temperature of the reaction system dropped to 0° C., Compound 113 (1.05 mmol) was added. The reaction was carried out under stirring and ice bath conditions for 1 hour, and then the reaction was quenched (LC-MS monitoring). Extraction was carried out with saturated sodium chloride solution (10 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 115 (C₂₈H₃₉N₄O₆S, [M+H]⁺ 559.3; found 559.5, yield 78.2%).

### Step 2. Synthesis of Compound 116

Compound 115 (0.75 mmol), lithium hydroxide monohydrate (8.10 mmol), tetrahydrofuran (4 mL) and water (2 mL) were added in sequence to a 50 mL reaction bottle, reacted under stirring at room temperature for 8 hours, and then the reaction was quenched (LC-MS monitoring). 1N hydrochloric acid solution was used to adjust the pH value of the system to 6-7, and the reaction solution was concentrated to obtain Compound 116 (C₂₇H₃₇N₄O₆S, [M+H]⁺ 545.2; found 545.5, crude product).

### Step 3. Synthesis of Compound 117

Compound 116 (0.70 mmol), EDCI (0.75 mmol), HOBt (0.75 mmol) and dichloroethane (2 mL) were added in sequence to a 50 mL reaction bottle. After the temperature of the reaction system dropped to 0° C., Compound 18 (0.95 mmol) was added, reacted under stirring and ice bath conditions for 1 hour, and then the reaction was quenched (LC-MS monitoring). Extraction was carried out with saturated sodium chloride solution (10 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 117 (C₃₆H₅₃N₆O₇S, [M+H]⁺ 713.4; found 713.2, yield 64.3%).

### Step 4. Synthesis of Compound 118

Compound 117 (0.45 mmol) was dissolved in dichloromethane (5 mL) in a 50 mL reaction bottle, then trifluoroacetic acid (1.5 mL) was added dropwise, and reacted under stirring at room temperature for 0.5 hour. The solvent was removed by concentration under reduced pressure to obtain Compound 118 (C₃₁H₄₅N₆O₅S, [M+H]⁺ 613.3; found 613.1, crude product).

### Step 5. Synthesis of Compound 119

Compound 118 (0.40 mmol), Compound 21 (0.80 mmol), N,N-diisopropylethylamine (1.02 mmol) and acetonitrile (5 mL) were added into a 50 mL reaction bottle, stirred and reacted at room temperature for 1 hour, then water was added to quench the reaction (LC-MS monitoring). Extraction was carried out with saturated sodium chloride solution (25 mL) and ethyl acetate (3×25 mL), the organic phases were combined, the combined organic phase was dried over anhydrous sodium sulfate, evaporated to dryness to remove the solvent, purified by medium-pressure preparative chromatography, and concentrated under reduced pressure to remove the solvent to obtain Compound 119 (C₃₄H₄₇N₆O₅S, [M+H]⁺ 651.3; found 651.5, yield 51.2%).

### Step 6. Synthesis of Compound G1

Under nitrogen protection, Compound R_{2-B1} (0.20 mmol), Compound 119 (0.20 mmol), copper iodide (0.60 mmol), triethylamine (5 mL), and Pd(PPh₃)₂Cl₂ (0.25 mol %) were added in sequence to a 50 mL reaction bottle. The reaction system was sealed, the reaction was carried out under stirring at 90° C. for 2 hours, and then the reaction was quenched (LC-MS monitoring). Extraction was carried out with saturated sodium chloride solution (20 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound G1 (3.8 mg, 3.31 μmol, 97.2% purity). LC-MS: C₆₆H₇₂N₁₁O₆S, [M/2+H]⁺574.2; found 574.2. ¹H NMR (600 MHz, Methanol-d4) δ 9.78 (s, 1H), 9.51 (s, 1H), 8.89 (d, J=6.3 Hz, 1H), 8.27-8.20 (m, 2H), 8.17 (s, 1H), 7.86 (s, 1H), 7.72 (t, J=6.9 Hz, 2H), 7.63 (dd, J=8.0, 5.7 Hz, 2H), 7.55-7.48 (m, 4H), 7.45-7.33 (m, 4H), 7.32 (s, 2H), 4.86 (s, 1H), 4.61-4.51 (m, 4H), 4.48 (s, 1H), 4.38-4.33 (m, 2H), 4.12 (s, 1H), 3.93-3.79 (m, 7H), 3.49-3.42 (m, 5H), 3.25 (s, 3H), 2.67 (s, 2H), 2.54-2.46 (m, 5H), 2.36 (t, J=7.2 Hz, 2H), 2.22-2.13 (m, 1H), 2.10-2.03 (m, 1H), 1.93-1.90 (m, 2H), 1.03-1.01 (m, 9H).

Compounds G1-2, G1-3, G1-4, and G1-5 could be obtained by following the step 1 to step 6 of the method for Compound G1-1, replacing Compound 114a with the raw materials in the following table, and keeping the other raw materials and operating methods unchanged.

| Compound | Structure and characterization data of | | |
|---|---|---|---|
| No. | compound | Raw material | Yield |
| G1-2 | 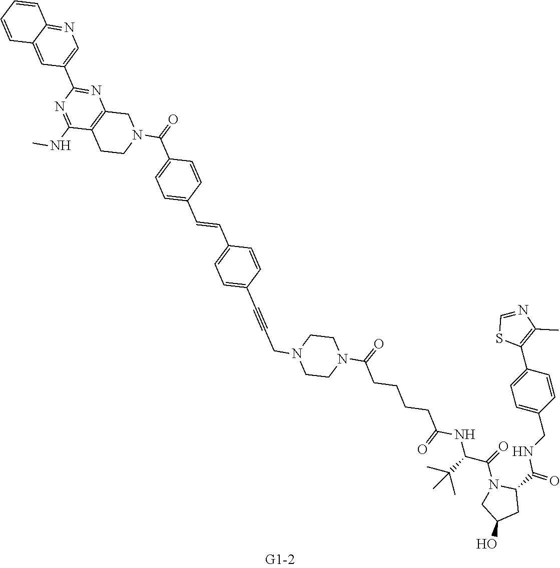 [SMILES: CNc1nc(-c2cnc3ccccc3c2)nc2c1CCN(C(=O)c1ccc(/C=C/c3ccc(C#CCN4CCN(C(=O)CCCCC(=O)N[C@H](C(=O)N5C[C@H](O)C[C@H]5C(=O)NCc5ccc(-c6scnc6C)cc5)C(C)(C)C)CC4)cc3)cc1)C2] | 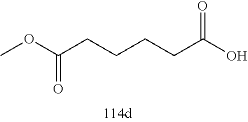 | 30.6% |
| | LC-MS: C₆₇H₇₄N₁₁O₆S, [M/2 + H]⁺ 581.2; found | | |
| | 581.4. ¹H NMR (600 MHz, Methanol-d₄) δ 9.77 (s, | | |
| | 1H), 9.54 (s, 1H), 8.96 (s, 1H), 8.29 (s, 1H), 8.28- | | |
| | 8.21 (m, 1H), 8.07 (s, 1H), 7.88 (s, 1H), 7.72 (d, J = | | |
| | 7.9 Hz, 2H), 7.61 (d, J = 7.9 Hz, 2H), 7.55-7.45 | | |
| | (m, 4H), 7.44-7.30 (m, 4H), 7.28 (s, 2H), 4.89 (s, | | |
| | 1H), 4.63 (s, 1H), 4.57-4.49 (m, 3H), 4.38-4.35 (m, | | |
| | 3H), 4.33 (s, 1H), 3.91-3.81 (m, 4H), 3.80-3.79 (m, | | |
| | 2H), 3.51-3.31 (m, 5H), 3.25 (s, 3H), 2.66 (s, 2H), | | |
| | 2.49 (s, 5H), 2.48-2.47 (m, 1H), 2.34-2.31 (m, 1H), | | |
| | 2.21-2.20 (m, 1H), 2.10-2.08 (m, 1H), 1.67-1.62 | | |
| | (m, 4H), 1.06-1.01 (m, 9H). | | |
| G1-3 | 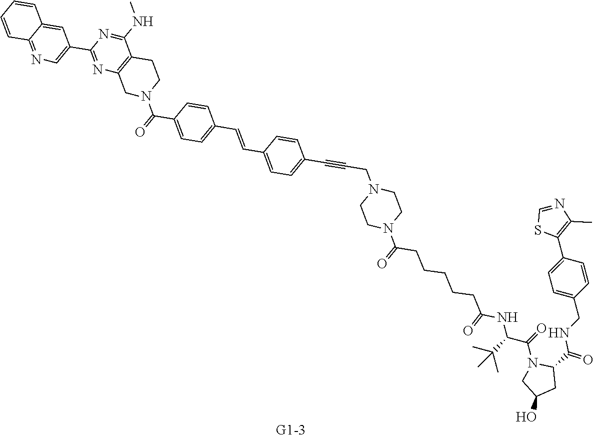 [SMILES: CNc1nc(-c2cnc3ccccc3c2)nc2c1CCN(C(=O)c1ccc(/C=C/c3ccc(C#CCN4CCN(C(=O)CCCCCC(=O)N[C@H](C(=O)N5C[C@H](O)C[C@H]5C(=O)NCc5ccc(-c6scnc6C)cc5)C(C)(C)C)CC4)cc3)cc1)C2] | 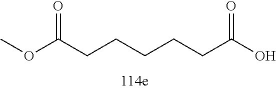 | 29.8% |
| | LC-MS: C₆₈H₇₆N₁₁O₆S, [M/2 + H]⁺ 588.2; found | | |
| | 588.2. ¹H NMR (600 MHz, Methanol-d₄) δ 9.87- | | |
| | 9.76 (m, 1H), 9.42-9.35 (m, 1H), 8.87 (s, 1H), 8.31- | | |
| | 8.15 (m, 2H), 8.00 (m, 1H), 7.97-7.80 (m, 1H), 7.71 | | |
| | (d, J = 8.0 Hz, 2H), 7.61 (d, J = 8.0 Hz, 2H), 7.54- | | |
| | 7.49 (m, 4H), 7.49-7.30 (m, 4H), 7.28 (s, 2H), 4.86 | | |
| | (s, 1H), 4.62-4.49 (m, 4H), 4.37-4.34 (m, 1H), 4.29 | | |
| | (s, 2H), 4.11 (s, 1H), 3.90-3.78 (m, 7H), 3.40-3.31 | | |
| | (m, 5H), 3.21 (s, 3H), 2.63 (s, 2H), 2.46-2.44 (m, | | |
| | 5H), 2.31-2.25 (m, 2H), 2.23-2.20 (m, 1H), 2.10- | | |
| | 2.03 (m, 1H), 1.66-1.61 (m, 4H), 1.41-1.37 (m, 2H), | | |
| | 1.03 (s, 9H). | | |
| G1-4 | 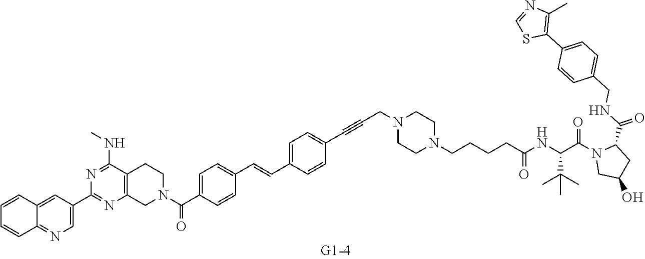 [SMILES: CNc1nc(-c2cnc3ccccc3c2)nc2c1CCN(C(=O)c1ccc(/C=C/c3ccc(C#CCN4CCN(CCCCC(=O)N[C@H](C(=O)N5C[C@H](O)C[C@H]5C(=O)NCc5ccc(-c6scnc6C)cc5)C(C)(C)C)CC4)cc3)cc1)C2] | 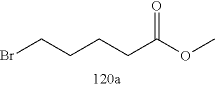 | 39.1% |
| | LC-MS: C₆₆H₇₄N₁₁O₅S, [M/2 + H]⁺ 567.3; found | | |
| | 567.1. ¹H NMR (600 MHz, DMSO-d₆) δ 9.83-9.74 | | |
| | (m, 1H), 9.28-9.13 (m, 1H), 8.98 (s, 1H), 8.55 (d, | | |
| | J = 6.0 Hz, 1H), 8.20-8.07 (m, 2H), 7.94 (d, J = 9.6 | | |
| | Hz, 1H), 7.83 (s, 1H), 7.72 (d, J = 4.8 Hz, 3H), 7.66 | | |
| | (d, J = 4.8 Hz, 3H), 7.53 (d, J = 4.8 Hz, 2H), 7.47 | | |
| | (d, J = 4.8 Hz, 2H), 7.41 (d, J = 4.8 Hz, 2H), 7.39- | | |
| | 7.37 (m, 6H), 7.26 (s, 1H), 4.73 (s, 1H), 4.54 (d, | | |
| | J = 9.3 Hz, 2H), 4.45-4.41 (m, 2H), 4.35 (s, 1H), | | |
| | 4.24-4.21 (m, 2H), 3.97 (s, 2H), 3.68-3.62 (m, 5H), | | |
| | 3.57 (s, 3H), 3.01 (s, 4H), 2.63-2.57 (m, 5H), 2.44 | | |
| | (s, 3H), 2.34-2.29 (m, 1H), 2.23-2.18 (m, 2H), 2.17- | | |
| | 2.07 (m, 1H), 1.9-1.89 (m, 1H), 1.62-1.56 (m, 2H), | | |
| | 1.53 (t, J = 4.8 Hz, 2H), 0.95 (s, 9H). | | |
| G1-5 | 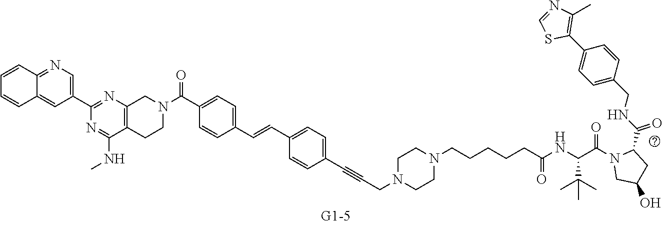 [SMILES: CNc1nc(-c2cnc3ccccc3c2)nc2c1CCN(C(=O)c1ccc(/C=C/c3ccc(C#CCN4CCN(CCCCCC(=O)N[C@H](C(=O)N5C[C@H](O)C[C@H]5C(=O)NCc5ccc(-c6scnc6C)cc5)C(C)(C)C)CC4)cc3)cc1)C2] | 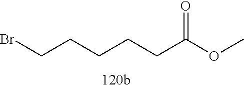 | 37.2% |
| | LC-MS: C₆₇H₇₆N₁₁O₅S, [M + H]⁺ 1146.6; found | | |
| | 1146.6. ¹H NMR (600 MHz, DMSO-d₆) δ 9.82- | | |
| | 9.73 (m, 1H), 9.20-9.10 (m, 1H), 8.98 (s, 1H), 8.55 | | |
| | (t, J = 6.0 Hz, 1H), 8.17-8.06 (m, 2H), 7.84 (d, J = | | |
| | 9.6 Hz, 1H), 7.73 (s, 1H), 7.72 (d, J = 4.8 Hz, 2H), | | |
| | 7.63 (d, J = 4.8 Hz, 3H), 7.52 (d, J = 4.8 Hz, 3H), | | |
| | 7.43 (d, J = 4.8 Hz, 2H), 7.36 (d, J = 4.8 Hz, 2H), | | |
| | 7.34-7.32 (m, 4H), 7.18-7.16 (m, 1H), 5.14 (s, 1H), | | |
| | 4.89-4.75 (m, 1H), 4.52 (d, J = 9.3 Hz, 1H), 4.44- | | |
| | 4.41 (m, 2H), 4.35 (s, 1H), 4.25-4.18 (m, 2H), 3.97 | | |
| | (s, 1H), 3.68-3.62 (m, 5H), 3.57 (s, 3H), 3.08 (s, | | |
| | 3H), 2.61 (s, 2H), 2.44 (d, J = 4.8 Hz, 3H), 2.39 (s, | | |
| | 1H), 2.29-2.25 (m, 2H), 2.11 (t, J = 6.0 Hz, 1H), | | |
| | 2.07 (t, J = 9.9 Hz, 1H), 1.52-1.42 (m, 5H), 1.40- | | |
| | 1.23 (s, 3H), 0.93 (s, 9H). | | |

According to the step 1 to step 6 of the method for Compound G1-1, the raw material 1 in the following table was used to replace Compound 113, the raw material 2 in the following table was used to replace Compound 114a, and the other raw materials and operating methods remained unchanged to obtain Compounds G2-1, G2-2, G2-3, G2-4, G2-5, G2-6, G2-7.

| Com- | | Raw | Raw | |
|---|---|---|---|---|
| pound | Structure and characterization data of | material | material | |
| No. | compound | 1 | 2 | Yield |
| G2-1 | 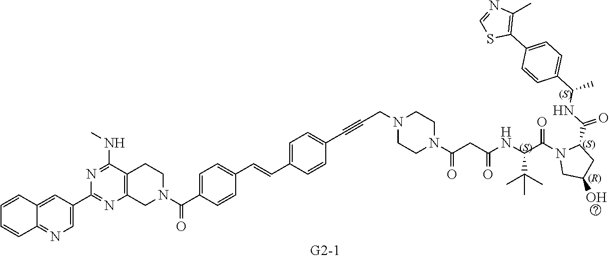 | 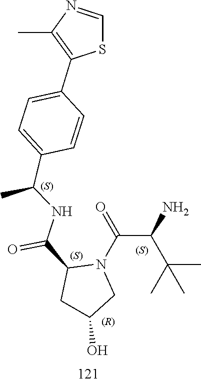 | 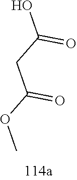 | 42.1% |
| | LC-MS: C₆₅H₇₀N₁₁O₆S, [M/2 + H]⁺ 567.3; found | | | |
| | 567.2. ¹H NMR (600 MHz, DMSO-d₆) δ 9.82- | | | |
| | 9.73 (m, 1H), 9.20-9.10 (m, 1H), 8.97 (d, J = 6.6 | | | |
| | Hz, 1H), 8.39 (d, J = 7.8 Hz, 1H), 8.17 (d, J = 9.4 | | | |
| | Hz, 1H), 8.06 (s, 2H), 7.80 (s, 1H), 7.73 (d, J = | | | |
| | 8.0 Hz, 2H), 7.64 (d, J = 8.1 Hz, 3H), 7.52 (d, J = | | | |
| | 7.9 Hz, 2H), 7.46-7.42 (m, 4H), 7.38-7.20 (m, | | | |
| | 4H), 7.18-1.76 (m, 2H), 5.11 (d, J = 3.5 Hz, 1H), | | | |
| | 4.91 (t, J = 7.4 Hz, 1H), 4.71 (s, 1H), 4.52 (d, J = | | | |
| | 9.3 Hz, 1H), 4.43 (t, J = 8.1 Hz, 1H), 4.28 (s, 1H), | | | |
| | 3.97 (s, 1H), 3.69 (s, 1H), 3.50-3.50 (m, 4H), | | | |
| | 3.48-3.46 (m, 4H), 3.08 (d, J = 4.4 Hz, 3H), 2.61 | | | |
| | (s, 4H), 2.45 (s, 4H), 2.23 (s, 3H), 2.07-2.02 (m, | | | |
| | 1H), 1.81-1.78 (m, 1H), 1.37 (d, J = 7.0 Hz, 3H), | | | |
| | 0.94 (s, 9H). | | | |
| G2-2 | 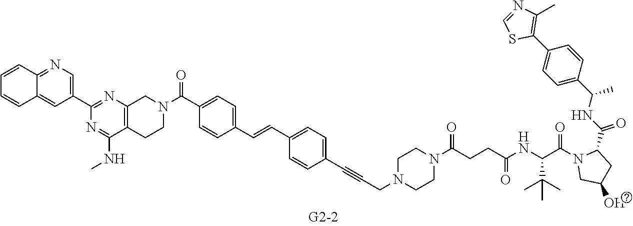 [SMILES: CNc1nc(-c2cnc3ccccc3c2)nc2c1CCN(C(=O)c1ccc(/C=C/c3ccc(C#CCN4CCN(C(=O)CCC(=O)N[C@H](C(=O)N5C[C@H](O)C[C@H]5C(=O)N[C@@H](C)c5ccc(-c6scnc6C)cc5)C(C)(C)C)CC4)cc3)cc1)C2] | 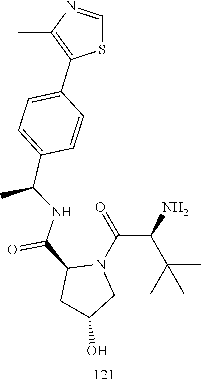 [SMILES: Cc1ncsc1-c1ccc([C@H](C)NC(=O)[C@@H]2C[C@@H](O)CN2C(=O)[C@@H](N)C(C)(C)C)cc1] | 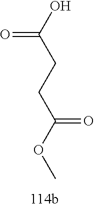 | 40.9% |
| | C₆₆H₇₂N₁₁O₆S LC-MS 1146.5; [M/2 + H]⁺ found | | | |
| | 574.2. ¹H NMR (600 MHz, DMSO-d₆) δ 9.82- | | | |
| | 9.73 (m, 1H), 9.25-9.14 (m, 1H), 8.98 (s, 1H), | | | |
| | 8.37 (d, J = 7.8 Hz, 1H), 8.15-8.08 (m, 2H), 7.88 | | | |
| | (d, J = 9.0 Hz, 1H), 7.87 (s, 1H), 7.78 (d, J = 8.0 | | | |
| | Hz, 2H), 7.74 (d, J = 8.1 Hz, 2H), 7.71 (d, J = 7.9 | | | |
| | Hz, 1H), 7.69-7.61 (m, 4H), 7.56 (d, J = 8.4 Hz, | | | |
| | 1H), 7.54-7.44 (m, 4H), 7.42-7.34 (m, 2H), 7.29 | | | |
| | (s, 1H), 5.11 (d, J = 3.5 Hz, 1H), 4.93-4.90 (m, | | | |
| | 1H), 4.50 (d, J = 2.4 Hz, 1H), 4.43 (t, J = 8.1 Hz, | | | |
| | 1H), 4.40-4.35 (m, 1H), 4.28 (s, 2H), 3.97 (s, 1H), | | | |
| | 3.60(s, 1H), 3.57-3.55 (m, 4H), 3.16-3.08 (m, | | | |
| | 6H), 2.79 (s, 1H), 2.61-2.55 (m, 5H), 2.53 (s, 3H), | | | |
| | 2.45-2.35 (m, 2H), 2.01-1.99 (m, 1H), 1.79-1.77 | | | |
| | (m, 1H), 1.37 (d, J = 7.0 Hz, 3H), 1.27-1.23 (m, | | | |
| | 1H), 0.93 (s, 9H). | | | |
| G2-3 | 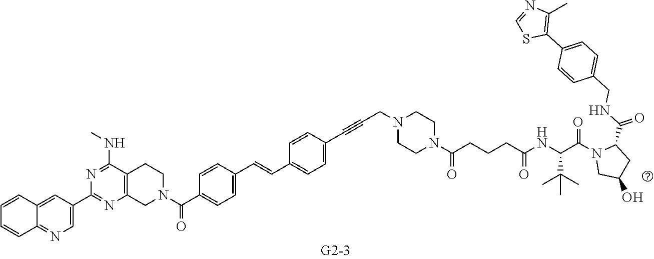 [SMILES: CNc1nc(-c2cnc3ccccc3c2)nc2c1CCN(C(=O)c1ccc(/C=C/c3ccc(C#CCN4CCN(C(=O)CCCC(=O)N[C@H](C(=O)N5C[C@H](O)C[C@H]5C(=O)NCc5ccc(-c6scnc6C)cc5)C(C)(C)C)CC4)cc3)cc1)C2] | 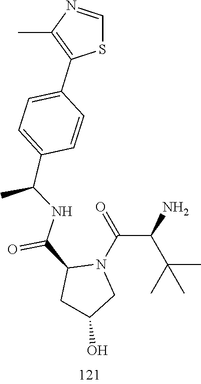 [SMILES: Cc1ncsc1-c1ccc([C@H](C)NC(=O)[C@@H]2C[C@@H](O)CN2C(=O)[C@@H](N)C(C)(C)C)cc1] | 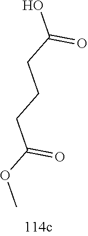 | 38.5% |
| | LC-MS: C₆₇H₇₄N₁₁O₆S, [M/2 + H]⁺ 581.3; found | | | |
| | 581.2. ¹H NMR (600 MHz, DMSO-d₆) δ 9.83- | | | |
| | 9.81 (m, 1H), 9.28-9.17 (m, 1H), 8.98 (s, 1H), | | | |
| | 8.36 (d, J = 7.7 Hz, 1H), 8.22-8.10 (m, 2H), 7.84 | | | |
| | (d, J = 9.0 Hz, 2H), 7.74 (d, J = 8.4 Hz, 2H), 7.70 | | | |
| | (d, J = 2.4 Hz, 2H), 7.68-7.67 (m, 3H), 7.57-7.53 | | | |
| | (m, 3H), 7.44-7.40 (m, 4H), 7.38-7.33 (m, 3H), | | | |
| | 4.93-4.90 (m, 1H), 4.74-4.69 (m, 1H), 4.57 (s, | | | |
| | 1H), 4.51 (d, J = 9.3 Hz, 1H), 4.42 (t, J = 8.1 Hz, | | | |
| | 1H), 4.40-4.35 (m, 4H), 3.97 (s, 3H), 3.69 (s, 3H), | | | |
| | 3.09 (d, J = 3.0 Hz, 4H), 2.62-2.58 (m, 5H), 2.45 | | | |
| | (s, 3H), 2.37 (t, J = 7.5 Hz, 3H), 2.26-2.20 (m, | | | |
| | 1H), 2.13 (t, J = 6.0 Hz, 1H), 2.02 (t, J = 9.9 Hz, | | | |
| | 1H), 1.84-1.73 (m, 1H), 1.75-1.71 (m, 2H), 1.37 | | | |
| | (d, J = 7.0 Hz, 3H), 0.93 (s, 9H). | | | |
| G2-4 | 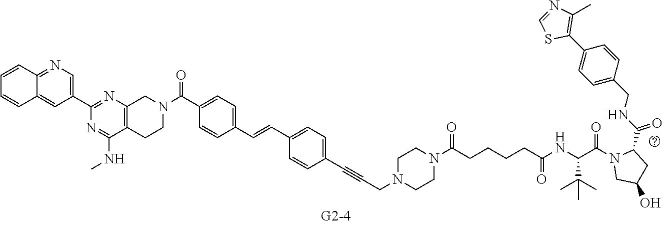 [SMILES: CNc1nc(-c2cnc3ccccc3c2)nc2c1CCN(C(=O)c1ccc(/C=C/c3ccc(C#CCN4CCN(C(=O)CCCCC(=O)N[C@H](C(=O)N5C[C@H](O)C[C@H]5C(=O)NCc5ccc(-c6scnc6C)cc5)C(C)(C)C)CC4)cc3)cc1)C2] | 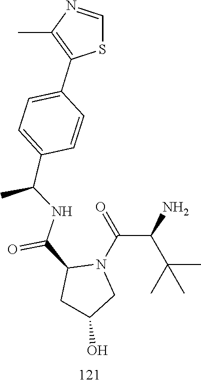 [SMILES: Cc1ncsc1-c1ccc([C@H](C)NC(=O)[C@@H]2C[C@@H](O)CN2C(=O)[C@@H](N)C(C)(C)C)cc1] | 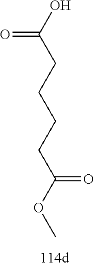 | 35.7% |
| | LC-MS: C₆₈H₇₆N₁₁O₆S, [M/2 + H]⁺ 588.3; found | | | |
| | 588.5. ¹H NMR (600 MHz, DMSO-d₆) δ 9.83- | | | |
| | 9.73 (m, 1H), 9.27-9.16 (m, 1H), 8.98 (s, 1H), | | | |
| | 8.36 (d, J = 7.7 Hz, 1H), 8.20-8.09 (m, 2H), 7.84 | | | |
| | (s, 1H), 7.80 (d, J = 3.6 Hz, 1H), 7.79-7.69 (m, | | | |
| | 6H), 7.57-7.53 (m, 4H), 7.46-7.42 (m, 4H), 7.36- | | | |
| | 7.35 (m, 2H), 7.34 (s, 1H), 4.98-4.90 (m, 1H), | | | |
| | 4.89-4.75 (m, 1H), 4.57 (s, 1H), 4.52 (d, J = 9.3 | | | |
| | Hz, 1H), 4.42 (t, J = 8.1 Hz, 1H), 4.40 - 4.35 (m, | | | |
| | 3H), 3.98 (s, 2H), 3.09 (s, 3H), 2.61-2.57 (m, 9H), | | | |
| | 2.45 (s, 3H), 2.36 (t, J = 7.5 Hz, 3H), 2.26-2.20 | | | |
| | (m, 1H), 2.13 (t, J = 6.0 Hz, 1H), 2.02 (t, J = 9.9 | | | |
| | Hz, 1H), 1.84-1.73 (m, 1H), 1.75-1.70 (m, 5H), | | | |
| | 1.37 (d, J = 7.0 Hz, 3H), 0.94 (s, 9H). | | | |
| G2-5 | 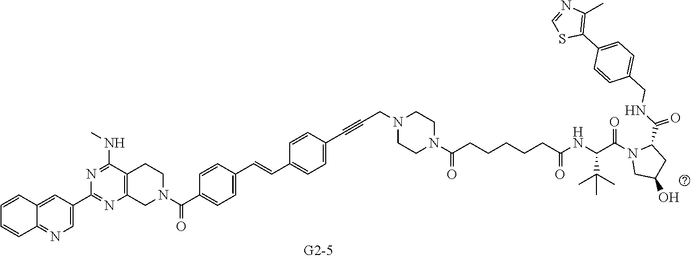 [SMILES: CNc1nc(-c2cnc3ccccc3c2)nc2c1CCN(C(=O)c1ccc(/C=C/c3ccc(C#CCN4CCN(C(=O)CCCCCC(=O)N[C@H](C(=O)N5C[C@H](O)C[C@H]5C(=O)NCc5ccc(-c6scnc6C)cc5)C(C)(C)C)CC4)cc3)cc1)C2] | 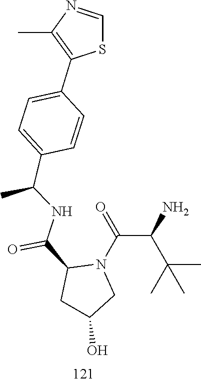 [SMILES: Cc1ncsc1-c1ccc([C@H](C)NC(=O)[C@@H]2C[C@@H](O)CN2C(=O)[C@@H](N)C(C)(C)C)cc1] | 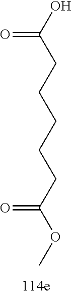 | 19.2% |
| | LC-MS: C₆₉H₇₈N₁₁O₆S, [M + H]⁺ 1188.6; found | | | |
| | 1188.2. ¹H NMR (600 MHz, DMSO-d₆) δ 9.86- | | | |
| | 9.73 (m, 1H), 9.20-9.10 (m, 1H), 8.97 (s, 1H), | | | |
| | 8.36 (d, J = 7.7 Hz, 1H), 8.16-8.06 (m, 2H), 7.78 | | | |
| | (d, J = 6.6 Hz, 2H), 7.72 (d, J = 3.6 Hz, 2H), 7.64 | | | |
| | (d, J = 9.8 Hz, 3H), 7.52 (d, J = 9.8 Hz, 2H), 7.46- | | | |
| | 7.42 (m, 4H), 7.38-7.33 (m, 4H), 7.19-7.14 (m, | | | |
| | 2H), 5.10 (s, 1H), 4.98-4.90 (m, 1H), 4.89-4.75 | | | |
| | (m, 1H), 4.62-4.50 (m, 2H), 4.42 (t, J = 8.1 Hz, | | | |
| | 1H), 4.40 (s, 1H), 3.98 (s, 1H), 3.85-3.62 (m, 1H), | | | |
| | 3.62-3.55 (m, 4H), 3.48 (s, 4H), 3.09 (s, 3H), | | | |
| | 2.56-2.53 (m, 2H), 2.47-2.45 (m, 5H), 2.36 (t, J = | | | |
| | 7.5 Hz, 3H), 2.26-2.20 (m, 1H), 2.13 (t, J = 6.0 | | | |
| | Hz, 1H), 2.02 (t, J = 9.9 Hz, 1H), 1.81-1.77 (m, | | | |
| | 1H), 1.52-1.47 (m, 4H), 1.37 (d, J = 7.0 Hz, 3H), | | | |
| | 1.28-1.24 (m, 3H), 0.94 (s, 9H). | | | |
| G2-6 | 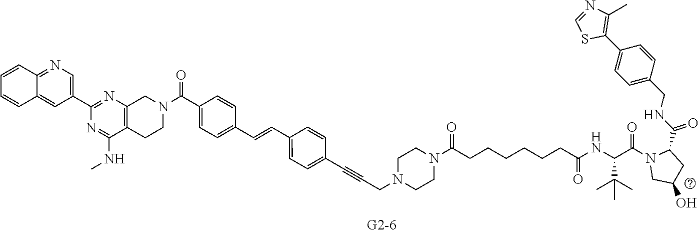 [SMILES: CNc1nc(-c2cnc3ccccc3c2)nc2c1CCN(C(=O)c1ccc(/C=C/c3ccc(C#CCN4CCN(C(=O)CCCCCCC(=O)N[C@H](C(=O)N5C[C@H](O)C[C@H]5C(=O)NCc5ccc(-c6scnc6C)cc5)C(C)(C)C)CC4)cc3)cc1)C2] | 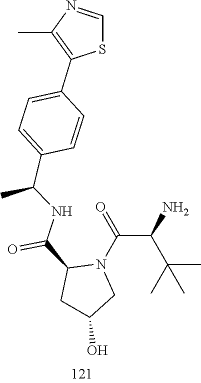 [SMILES: Cc1ncsc1-c1ccc([C@H](C)NC(=O)[C@@H]2C[C@@H](O)CN2C(=O)[C@@H](N)C(C)(C)C)cc1] | 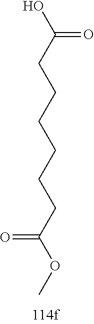 | 33.7% |
| | LC-MS: C₇₀H₃₀N₁₁O₆S, [M/2 + H]⁺ 602.3; found | | | |
| | 602.3. ¹H NMR (600 MHz, DMSO-d₆) δ 9.82- | | | |
| | 9.73 (m, 1H), 9.21-9.10 (m, 1H), 8.97 (s, 1H), | | | |
| | 8.36 (d, J = 7.7 Hz, 1H), 8.17-8.06 (m, 2H), 7.78 | | | |
| | (d, J = 6.6 Hz, 2H), 7.72 (d, J = 3.6 Hz, 2H), 7.64 | | | |
| | (d, J = 9.8 Hz, 3H), 7.52 (d, J = 9.8 Hz, 2H), 7.45- | | | |
| | 7.42 (m, 4H), 7.38-7.32 (m, 5H), 7.19-7.15 (m, | | | |
| | 1H), 5.10 (s, 1H), 4.98-4.90 (m, 1H), 4.89-4.75 | | | |
| | (m, 1H), 4.57 (s, 1H), 4.52 (d, J = 9.3 Hz, 1H), | | | |
| | 4.42 (t, J = 8.1 Hz, 1H), 4.27 (s, 1H), 3.97 (s, 1H), | | | |
| | 3.85-3.68 (m, 1H), 3.62-3.58 (m, 4H), 3.48 (s, | | | |
| | 4H), 3.07 (s, 3H), 2.61 (s, 1H), 2.56-2.45 (m, | | | |
| | 5H), 2.36 (t, J = 7.5 Hz, 2H), 2.26-2.20 (m, 1H), | | | |
| | 2.11 (t, J = 6.0 Hz, 1H), 2.07 (t, J = 9.9 Hz, 1H), | | | |
| | 1.81-1.77 (m, 1H), 1.52-1.47 (m, 4H), 1.37 (d, | | | |
| | J = 7.0 Hz, 3H), 1.37-1.24 (m, 6H), 0.93 (s, 9H). | | | |
| G2-7 | 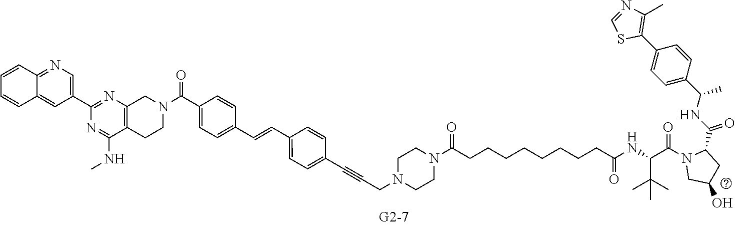 [SMILES: CNc1nc(-c2cnc3ccccc3c2)nc2c1CCN(C(=O)c1ccc(/C=C/c3ccc(C#CCN4CCN(C(=O)CCCCCCCCC(=O)N[C@H](C(=O)N5C[C@H](O)C[C@H]5C(=O)N[C@@H](C)c5ccc(-c6scnc6C)cc5)C(C)(C)C)CC4)cc3)cc1)C2] | 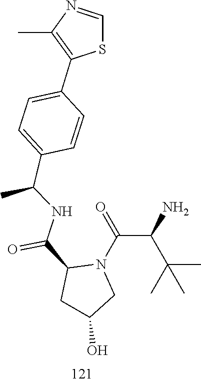 [SMILES: Cc1ncsc1-c1ccc([C@H](C)NC(=O)[C@@H]2C[C@@H](O)CN2C(=O)[C@@H](N)C(C)(C)C)cc1] | 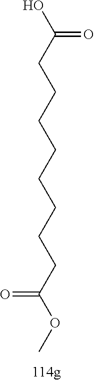 | 19.8% |
| | LC-MS: C₇₂H₈₄N₁₁O₆S, [M/2 + H]⁺ 616.3; found | | | |
| | 616.3. ¹H NMR (600 MHz, DMSO-d₆) δ 9.83- | | | |
| | 9.73 (m, 1H), 9.20-9.15 (m, 1H), 8.97 (s, 1H), | | | |
| | 8.36 (d, J = 7.7 Hz, 1H), 8.17-8.07 (m, 2H), 7.78 | | | |
| | (d, J = 6.6 Hz, 2H), 7.72 (d, J = 3.6 Hz, 2H), 7.64 | | | |
| | (d, J = 9.8 Hz, 3H), 7.52 (d, J = 9.8 Hz, 2H), 7.45- | | | |
| | 7.42 (m, 4H), 7.38-7.32 (m, 4H), 7.19-7.15 (m, | | | |
| | 2H), 5.10 (s, 1H), 4.98-4.90 (m, 1H), 4.89-4.75 | | | |
| | (m, 1H), 4.57 (s, 1H), 4.52 (d, J = 9.3 Hz, 1H), | | | |
| | 4.42 (t, J = 8.1 Hz, 1H), 4.40 (s, 1H), 3.97 (s, 1H), | | | |
| | 3.85-3.68 (m, 1H), 3.62-3.58 (m, 4H), 3.48 (s, | | | |
| | 4H), 3.08 (s, 3H), 2.57-2.53 (m, 3H), 2.47-2.45 | | | |
| | (m, 5H), 2.36 (t, J = 7.5 Hz, 3H), 2.11 (t, J = 6.0 | | | |
| | Hz, 1H), 2.07 (t, J = 9.9 Hz, 1H), 1.81-1.78 (m, | | | |
| | 1H), 1.52-1.47 (m, 4H), 1.37 (d, J = 7.0 Hz, 3H), | | | |
| | 1.28-1.24 (m, 8H), 0.93 (s, 9H). | | | |

### Example 16. Synthesis of Compound G3-1 of the Present Invention

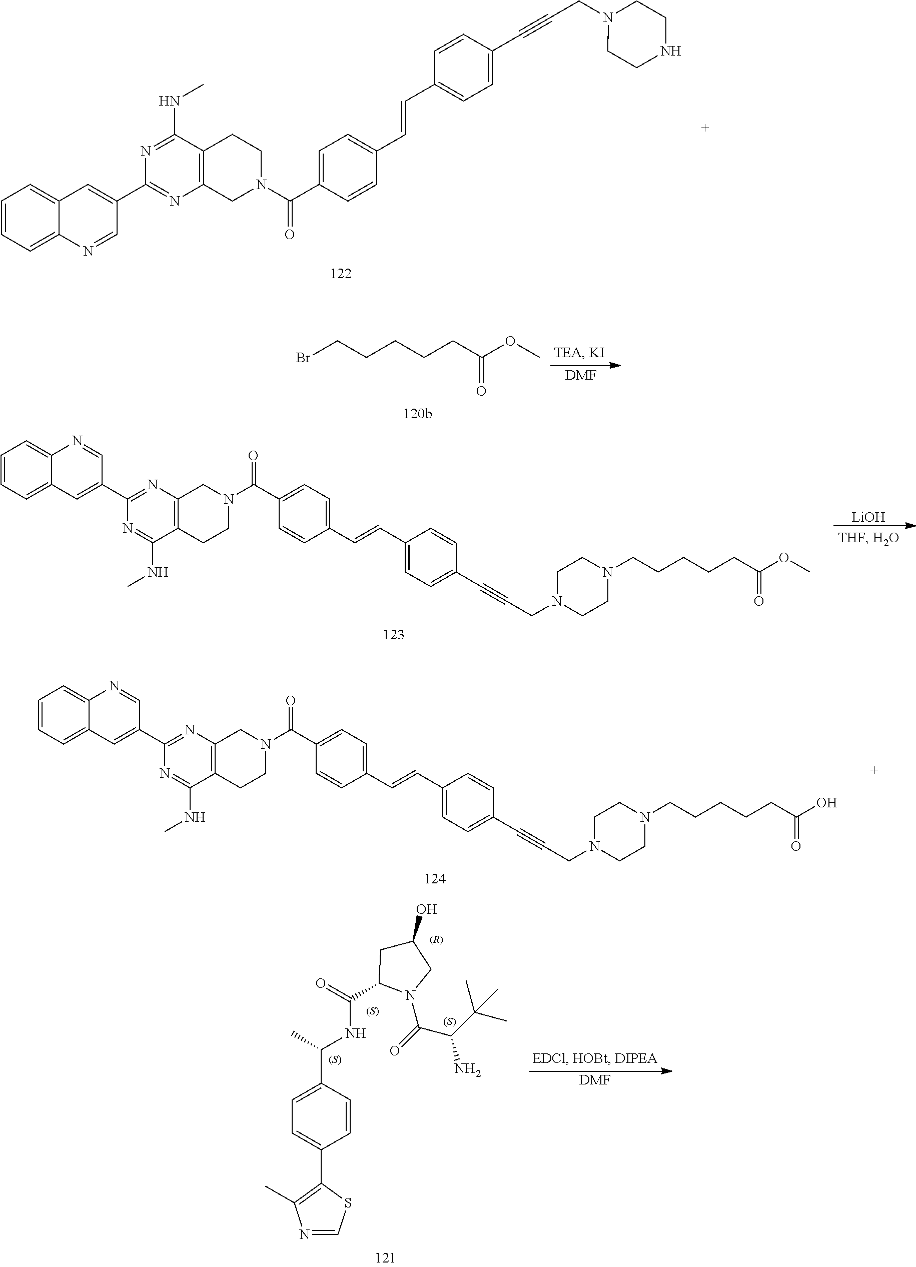

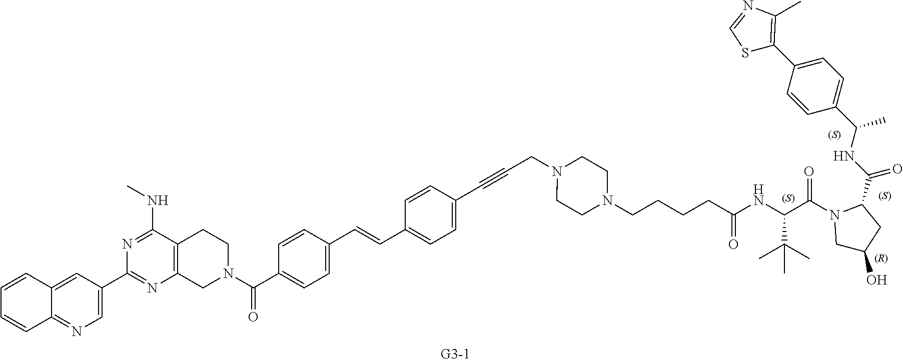

### Step 1. Synthesis of Compound 123

Compound 122 (1.00 mmol), Compound 120b (2.25 mmol), potassium iodide (1.05 mmol), triethylamine (2 mL) and NN-dimethylformamide (5 mL) were added into a 50 mL reaction bottle, and reacted at room temperature. After the reaction was completed, water was added to quench the reaction (LC-MS monitoring). Extraction was carried out with saturated sodium chloride solution (25 mL) and ethyl acetate (3×25 mL), the organic phases were combined, the organic phase was dried over anhydrous sodium sulfate, evaporated to dryness to remove the solvent, purified by medium-pressure preparative chromatography, and concentrated under reduced pressure to remove the solvent to obtain Compound 123 (C₄₂H₄₂N₇O₃, [M+H]⁺ 692.3; found 692.2, yield 59.8%).

### Step 2. Synthesis of Compound 124

Compound 123 (0.75 mmol), lithium hydroxide monohydrate (8.10 mmol), tetrahydrofuran (4 mL) and water (2 mL) were added in sequence to a 50 mL reaction bottle, stirred and reacted at room temperature for 8 hours, and then the reaction was quenched (LC-MS monitoring). 1N hydrochloric acid solution was used to adjust the pH value of the system to 6-7, and the reaction solution was concentrated to obtain Compound 124 (C₄₁H₄₀N₇O₃, [M+H]⁺ 678.3; found 678.5).

### Step 3. Synthesis of Compound G3-1

Compound 124 (crude product), EDCI (0.75 mmol), HOBt (0.75 mmol) and dichloromethane (2 mL) were added in sequence to a 50 mL reaction bottle. After the temperature of the reaction system dropped to 0° C., Compound 121 (0.95 mmol) was added, reacted under stirring and ice bath conditions for 1 hour, and then the reaction was quenched (LC-MS monitoring). Extraction was carried out with saturated sodium chloride solution (10 mL) and ethyl acetate (3×25 mL), the organic phases were combined, the combined organic phase was dried over anhydrous sodium sulfate, evaporated to dryness to remove the solvent, and separated by column chromatography (the volume ratio of the eluents used was petroleum ether/ethyl acetate=1:100 to 1:5), and concentrated under reduced pressure to remove the solvent to obtain Compound G3-1 (6.5 mg, 5.67 μmol, 90.9% purity). LC-MS: C₆₇H₇₆N₁₁O₅S, [M/2+H]⁺574.2; found 574.2. ¹H NMR (600 MHz, DMSO-d6) δ 9.82-9.74 (m, 1H), 9.21-9.11 (m, 1H), 8.98 (s, 1H), 8.36 (d, J=6.0 Hz, 1H), 8.17-8.07 (m, 2H), 7.95 (s, 1H), 7.78 (d, J=4.8 Hz, 2H), 7.72 (d, J=4.8 Hz, 2H), 7.66 (d, J=4.8 Hz, 3H), 7.52 (d, J=4.8 Hz, 2H), 7.46-7.42 (m, 4H), 7.38-7.36 (m, 4H), 7.19-7.15 (m, 1H), 5.10 (s, 1H), 4.91 (t, J=5.4 Hz, 1H), 4.73 (s, 1H), 4.50 (d, J=9.3 Hz, 2H), 4.42 (t, J=4.8 Hz, 1H), 4.31-4.28 (m, 1H), 3.97 (s, 1H), 3.69 (s, 1H), 3.62 (t, J=6.6 Hz, 2H), 3.58 (s, 2H), 3.07 (s, 3H), 2.89 (s, 4H), 2.73 (s, 4H), 2.57 (s, 3H), 2.45 (s, 3H), 2.25 (s, 3H), 2.18-2.10 (m, 1H), 2.02-1.99 (m, 1H), 1.80-1.77 (m, 1H), 1.49-1.47 (m, 2H), 1.38-1.36 (m, 4H), 0.93 (s, 9H).

### Synthesis of Intermediate 129

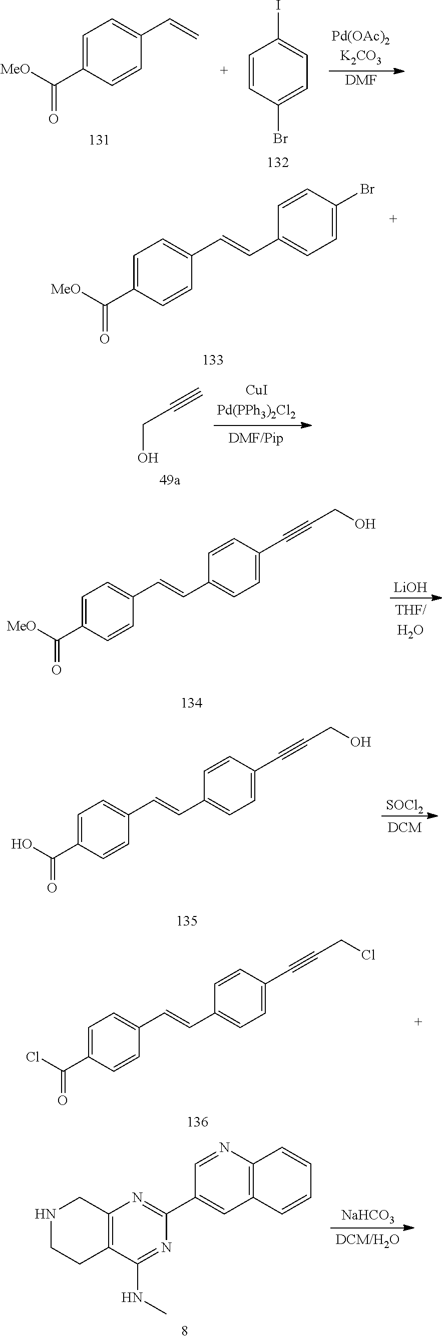

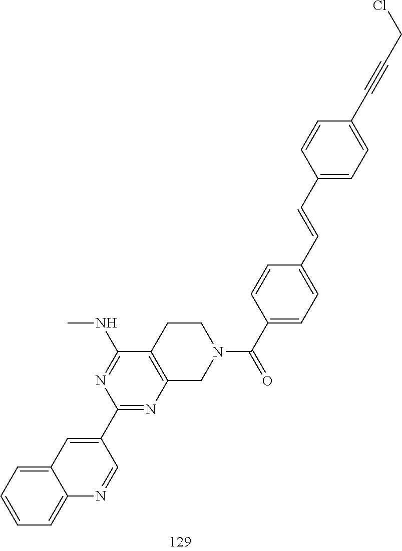 [SMILES: CNc1nc(-c2cnc3ccccc3c2)nc2c1CCN(C(=O)c1ccc(/C=C/c3ccc(C#CCCl)cc3)cc1)C2]

### Step 1. Synthesis of Compound 133

Compound 131 (1.00 g, 6.16 mmol, 1.0 eq) and Compound 132 (2.09 g, 7.41 mmol, 1.2 eq) were added into a 50 mL reaction bottle, then added with N,N-dimethylformamide (26 mL), potassium carbonate (1.71 g, 12.34 mmol, 2.0 eq), and palladium acetate (0.05 g, 0.31 mmol, 0.05 eq) under the protection of nitrogen gas, heated and reacted at 80° C. overnight, and purified by MPLC to obtain Compound 133 (0.78 g, 2.46 mmol, yield 40.00%).

### Step 2. Synthesis of Compound 134

Compound 133 (0.78 g, 2.46 mmol, 1.0 eq) and Compound 49a (0.43 g, 7.37 mmol, 3.0 eq) were added into a 50 mL reaction bottle, then added with N,N-dimethylformamide (10 mL), copper iodide (46.80 mg, 0.25 mmol, 0.1 eq.), and Pd(PPh₃)₂Cl₂ (0.086 g, 0.12 mmol, 0.05 eq.) under the protection of nitrogen gas, heated and reacted at 80° C. overnight, and purified by MPLC to obtain Compound 134 (0.57 g, 1.95 mmol, yield 80.00%).

### Step 3. Synthesis of Compound 135

Compound 134 (0.57 g, 1.95 mmol, 1 eq.), LiOH (0.17 g, 3.9 mmol, 2.0 eq.), and THF/H₂O (10 mL, 2:1) were added in sequence to a 50 mL reaction bottle, and reacted under stirring at room temperature for 4 hours, and then the reaction was quenched (LC-MS monitoring), the pH value of the system was adjusted to 6-7 with 1M hydrochloric acid solution, and the reaction solution was concentrated to obtain Compound 135 (0.57 g, crude product).

### Step 4. Synthesis of Compound 136

Compound 135 (0.57 g, 2.03 mmol, 1 eq.), DCM (10 mL), and SOCl₂ (2 mL) were added in sequence to a 50 mL reaction bottle, heated and reacted at 50° C. for 2 hours, and the reaction solution was concentrated to obtain Compound 136 (0.66 g, crude product).

### Step 5. Synthesis of Intermediate 129

Compound 136 (0.66 g, 2.10 mmol, 1 eq.), Compound 8 (0.55 g, 2.10 mmol, 1 eq.), NaHCO₃ (0.35 g, 4.20 mmol, 2 eq.) and DCM/H₂O (9 mL, 2:1) were added into a 50 ML reaction bottle, heated and reacted at 50° C. for half an hour, then the reaction solution was filtered and concentrated to obtain Compound 129 (1.10 g, crude product).

Referring to the synthesis method of intermediate Compound 129, Compound 12 in the following table was used to replace Compound 8, and the other raw materials and operating methods remained unchanged to obtain intermediate Compound 130.

| Inter- | | |
|---|---|---|
| mediate | | |
| Com- | | |
| pound | | |
| No. | Intermediate compound structure | Raw material |
| 130 | 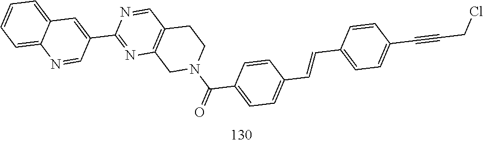 [SMILES: O=C(c1ccc(/C=C/c2ccc(C#CCCl)cc2)cc1)N1CCc2cnc(-c3cnc4ccccc4c3)nc2C1] | 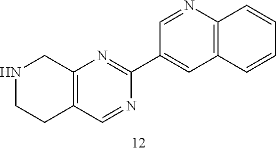 [SMILES: c1ccc2ncc(-c3ncc4c(n3)CNCC4)cc2c1] |

### Example 17. Synthesis of Compound H1-1 of the Present Invention

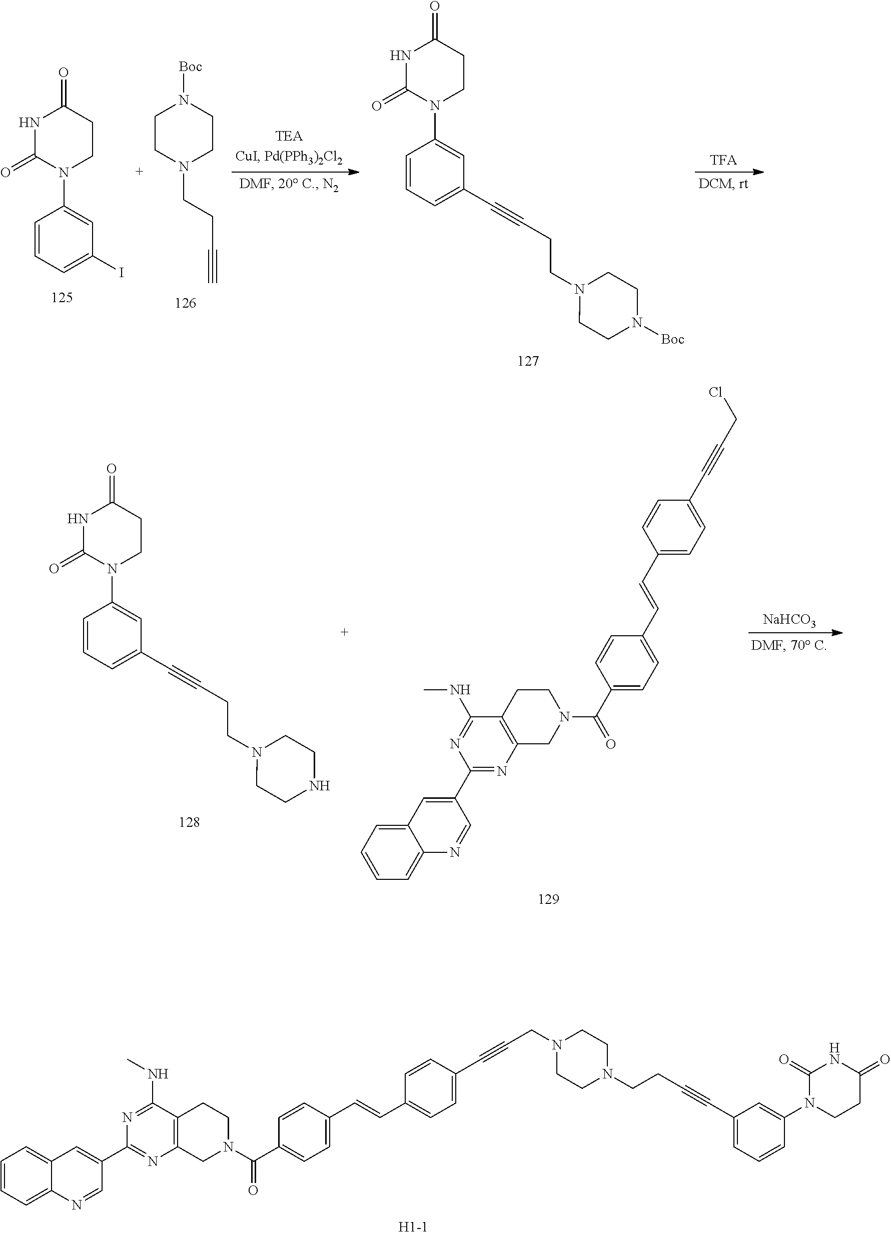

### Step 1. Synthesis of Compound 127

Compound 125 (317.00 mg, 1.00 mmol), Compound 126 (286.81 mg, 1.20 mmol), copper iodide (19.10 mg, 100.29 μmol), and bis(triphenylphosphine)palladium dichloride (35.15 mg, 50.14 μmol) and triethylamine (725.50 mg, 7.17 mmol, 1 mL) were added into a 25 mL round-bottom flask, then added with DMF (4 mL) under nitrogen protection, stirred at room temperature for 2 hours, water was added to quench the reaction, and extracted with ethyl acetate. The obtained organic phase was washed with saturated brine, dried over anhydrous sodium sulfate, and then the solvent was rotary evaporated to obtain Compound 127 (600.00 mg, 1.41 mmol).

### Step 2. Synthesis of Compound 128

Compound 127 (600 mg, 1.41 mmol), DCM (5 mL), and triethylamine (5 mL) were added into a 25 mL round-bottom flask. After stirring at room temperature for half an hour, the solvent was evaporated under reduced pressure, and purification was carried out by MPLC to obtain Compound 128 (220.00 mg, 674.04 μmol, yield 47.91%).

### Step 3. Synthesis of Compound H1-1

Compound 128 (50.00 mg, 153.19 μmol), NaHCO₃ (64.34 mg, 765.95 μmol), Compound 129 (87.33 mg, 153.19 μmol) and DMF (3 mL) were added into a 25 mL round-bottomed flask, reacted under stirring at 70° C. for half an hour, then water was added to quench the reaction, and extraction was carried out with ethyl acetate. The obtained organic phase was washed with saturated brine, dried over Na₂SO₄, evaporated under reduced pressure to remove the solvent, and purified by MPLC to obtain Compound H1-1 (15.00 mg, 17.44 μmol, yield 11.39%, purity 95.9%). LC-MS: C₅₃H₅₀N₉O₃, [M+H]⁺: 861.4, found: 860.8. ¹H NMR (600 MHz, DMSO-d6) δ 10.42 (s, 1H), 9.85-9.72 (m, 1H), 9.24-9.21 (m, 1H), 8.15-8.13 (m, 2H), 7.85 (s, 1H), 7.76-7.73 (m, 2H), 7.68-7.65 (m, 3H), 7.54-7.52 (m, 4H), 7.43-7.38 (m, 4H), 7.36-7.34 (m, 2H), 7.31-7.30 (m, 1H), 4.74 (s, 1H), 4.57 (s, 1H), 4.10-4.05 (m, 2H), 3.64-3.59 (m, 2H), 3.37-3.33 (m, 4H), 3.20-3.03 (m, 7H), 2.96 (s, 3H), 2.81 (s, 2H), 2.70 (t, J=6.6 Hz, 2H), 2.58 (s, 3H). HPLC>95%.

According to the synthesis method of Compound H1-1, the raw material 130 in the following table was used to replace Compound 129, and the other raw materials and operating methods remained unchanged to obtain Compound H1-2.

| Compound | Structure and characterization data of | | |
|---|---|---|---|
| No. | compound | Raw material | Yield |
| H1-2 | 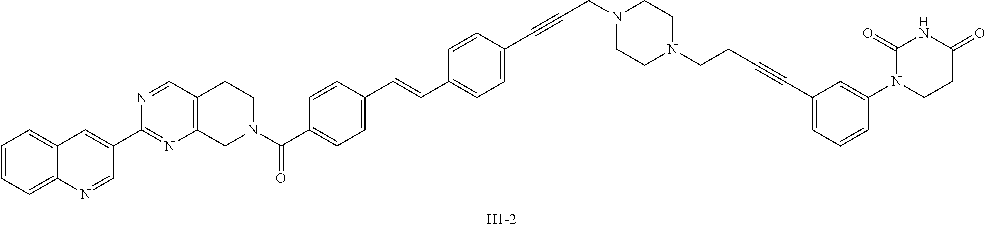 [SMILES: O=C1CCN(c2cccc(C#CCCN3CCN(CC#Cc4ccc(/C=C/c5ccc(C(=O)N6CCc7cnc(-c8cnc9ccccc9c8)nc7C6)cc5)cc4)CC3)c2)C(=O)N1] | 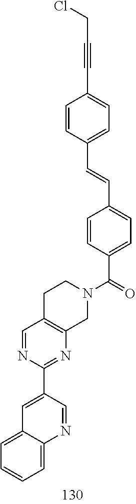 [SMILES: O=C(c1ccc(/C=C/c2ccc(C#CCCl)cc2)cc1)N1CCc2cnc(-c3cnc4ccccc4c3)nc2C1] | 21.64% |
| | H1-2 (9.30 mg, 10.81 umol, yield 21.64%, purity | | |
| | 97.6%). LC-MS: C₅₂H₄₇N₈0₃, [M + 1]⁺ 831.4, | | |
| | found: 831.6. | | |
| | ¹H NMR (600 MHz, DMSO-d₆) δ 10.43 (s, 1H), | | |
| | 9.80-9.76 (m, 1H), 9.26-9.23 (m, 1H), 8.17-8.14 | | |
| | (m, 2H), 7.85 (s, 1H), 7.81-7.79 (m, 2H), 7.68-7.64 | | |
| | (m, 3H), 7.50-7.48 (m, 4H), 7.43-7.38 (m, 4H), | | |
| | 7.36-7.34 (m, 2H), 7.30-7.28 (m, 1H), 4.74 (s, 1H), | | |
| | 4.57 (s, 1H), 4.10-4.05 (m, 2H), 3.63-3.60 (m, 2H), | | |
| | 3.36-3.30 (m, 4H), 3.18-3.03 (m, 7H), 2.96 (s, 3H), | | |
| | 2.81 (s, 2H), 2.70 (t, J = 6.6 Hz, 2H). HPLC >95%. | | |

The technical effects of the compounds of the present invention were illustrated below through experimental examples:

NaVc: sodium vitamin C; His-Tag Labeling Kit-RED-tris-NTA: RED-Tris-NTA protein labeling kit; Dianthus 384 well plates: Dianthus 384-well plate; Zeba™ Spin Desalting Columns 7K: Zeba desalting spin column; DMSO/Dimethyl Sulfoxide: dimethyl sulfoxide; BCL-XL/BAK Binding Assay Kit: BCLXL/BAK binding kit; S series NTA sensor chip: S series NTA chip; NaOH 50: sodium hydroxide; EDTA: ethylenediaminetetraacetic acid; NiCl₂: nickel chloride; EDC: 1-ethyl-(3-dimethylaminopropyl)carbodiimide; NHS: N-Hydroxysulfosuccinimide; Ethanolamine-HCl (pH 8.5): ethanolamine-hydrochloride (pH 8.5); HuMan BCL2L1/BCL-XL Protein: human BCL2L1/BCL-XL protein; AlphaLISA®Nikel Chelate Acceptor beads: AlphaLISA nickel receptor magnetic beads; Strep-Tactin® Alpha Donor beads: Strep-Tactin Alpha donor magnetic beads; RPMI medium modified: RPMI 1640 medium; FBS: fetal bovine serum; P/S: penicillin-streptomycin double antibody; CellTiter-Glo Luminescent Cell Viability Assay: CellTiter-Glo® luminescent viable cell detection system; Diluent buffer: dilution buffer salt; Glycine: glycine; Imidazole: imidazole.

### Experimental Example 1, BCL-XL TRIC (Temperature-Dependent Fluorescence Intensity Change Method) Binding Detection

### 1. Experimental Materials and Reagents

Na₂HPO₄ (Sigma), NaH₂PO₄ (Sigma), sodium chloride (Sigma), Tween 20 (Sigma), His-Tag Labeling Kit-RED-tris-NTA (NanoTemper), Dianthus 384 well plates (NanoTemper), deionized water (NanoTemper), BCL-XL protein (Sino Biological), Zeba™ Spin Desalting Columns 7K (Thermo scientific).

### 2. Experimental Methods

### 2.1 RED-Tris-NTA Protein Labeling

### 2.1.1 Protein Stock Buffer Replacement:

Zeba™ Spin Desalting Columns 7K MWCO desalting column was used to replace the stock buffer of BCL-XL with 10 mM NaH₂PO₄, 40 mM Na₂HPO₄, 150 mM sodium chloride, 0.03% Tween 20, 10% glycerol, pH7.4, so as to remove the imidazole and hydroxymethylaminomethane ingredients in the stock buffer.

### 2.1.2 Testing of Affinity Between Dye and Protein

To a 5×PBST (from RED-tris-NTA protein labeling kit) bottle, 8.0 mL of ddH₂O was added for dilution to obtain 1×PBST, 25.0 μL of PBST was added to dilute the dye to 5 μM, 2.0 μL of 5 μM dye and 198.0 μL of PBST were mixed to obtain 200.0 μL of 50 nM dye; 30.0 μL of 4 μM histidine-tagged BCL-XL protein diluted with PBST was prepared and subjected to a 2-fold gradient dilution with 16 points. 10 μL of protein with different concentrations was taken and thoroughly mixed with 10.0 μL of 50 nM dye, incubated at room temperature for 30 minutes, and read with DI. Kd was calculated by DI.SA, and the calculated Kd was <10 nM.

### 2.1.3 Affinity of Dye and Protein

2.0 μL of 5 μM dye was taken and mixed with 98.0 μL of PBST to obtain 100.0 μL of 100 nM dye. Histidine-tagged BCL-XL protein was diluted to 200 nM. 90.0 μL of 200 nM histidine-tagged BCL-XL protein was taken and thoroughly mixed with 90.0 μL of 100 nM dye, and incubate at room temperature for 30 minutes. After centrifugation at 15000 g for 10 minutes at 4° C., the supernatant was taken and placed in a new tube.

### 2.2. Testing of Compound Affinity

The compound was diluted with buffer PBST by a dilution with a total of 16 concentration gradient. 10.0 μL of the compound of each concentration was taken and thoroughly mixed with 10.0 μL of the labeled protein (the final concentration of the RED-tris-NTA-labeled protein used was not less than 20 nM.), and read by DI. Kd was calculated by DI.SA.

### 3. Data Analysis

DI.SA was used to determine the binding of ligand/compound to target and the specific Kd.

### Experimental Example 2. Detection of BCL-XL Inhibitory Activity of Compound

### 1. Experimental Instruments and Reagents:

Microplate reader (Tecan Spark), ECHO (LABCYTE Echo 665), microplate constant temperature oscillator (Hangzhou Ruicheng Instrument Co., Ltd.), BCL-XL/BAK Binding Assay Kit (CISbIO), 384-well plates

### 2. Experimental Method:

DMSO was used to dissolve the dry powder of compound into a 10.00 mM solution. The instrument ECHO was used to perform gradient dilution of the compound, which was added to a 384-well reaction plate, so that the final concentration of DMSO in the entire reaction system (12.0 μL) was less than 0.5%. At the same time, an equal amount of DMSO was added as a control.

The Diluent buffer (Lot 06A) in BCL-XL/BAK Binding Assay Kit was used to dilute BCL-XL and Tag2-BAK to 4 times the required final concentration, and 3.0 μL of each was pipetted and added to the 384-well reaction plate where the compound had been added. After centrifugation at 1000 rpm for 1 minute, the plate was placed on a microplate constant-temperature shaker and pre-incubated at 25° C., 280 rpm for 15 minutes. Then the detection Buffer (Lot 10A) in BCL-XL/BAK Binding Assay Kit was used to dilute 100× Anti-tag1-Eu³⁺ (Lot 06A) and Anti-tag2-XL665 (Lot 104A) to 1x, respectively, and prepare Anti-tag1-Eu³⁺/Anti-tag2-XL665 mixture at a ratio of 1:1. 6.0 μL of Anti-tag1-Eu³⁺/Anti-tag2-XL665 mixture was pipetted and added to the 384-well reaction plate, centrifuged at 1000 rpm for 1 minute and placed on a microplate constant-temperature shaker, and incubated at 25° C., 280 rpm for 2 hours. After the reaction, the microplate reader was used to read the fluorescence signal value in the 384-well reaction plate (Ex=320 nm, Em=665/620 nm).

### 3. Data Analysis

The vehicle group (containing 1×Tag1-Bcl-XL, 1×Tag2-BAK, 1× Anti-Tag1-Eu³⁺ and 1× Anti-Tag2-XL665, 0.5% DMSO) was used as a negative control, and the reaction buffer group (containing 1× Anti-Tag1-Eu³⁺ and 1× Anti-Tag2-XL665, 0.5% DMSO) was used as a blank control;

The remaining activity percentage of each concentration was calculated by the following formula:

Remaining activity (%)=100%×(Flu_{compound group}−Flu_{blank control})/(Flu_{negative control}−Flu_{blank control})

Then GraphPad 6.0 was used to fit the dose-effect curve to calculate IC₅₀ value.

### Experimental Example 3, Binding Detection of BCL-XL by Surface Plasmon Resonance (SPR)

### 1. Experimental Instruments and Reagents:

Biacore T200 (GE Healthcare), S series NTA sensor chip (GE Healthcare), NaOH 50 (GE Healthcare), DMSO (MP), Na₂HPO₄ (Sigma), NaH₂PO₄ (Sigma), EDTA (Sigma), NiCl₂ (Sigma), sodium chloride (Sigma), Tween 20 (Sigma), EDC (GE Healthcare), NHS (GE Healthcare), Ethanolamine-hydrochloric acid (pH 8.5) (GE Healthcare), 96-well plate, HuMan BCL2L1/BCL-XL Protein (Sino Biological), Zeba™ Spin Desalting Columns 7K (Thermo scientific).

### 2. Test Method of SPR

Preparation of running buffers: The protein fixation buffer had the same ingredients as running buffer A, in which the concentration of NaH₂PO₄ was 10 mM, the concentration of Na₂HPO₄ was 40 mM, the concentration of sodium chloride was 150 mM, the content of Tween 20 was 0.03%, and the pH was adjusted to 7.4; in the running buffer B, the concentration of NaH₂PO₄ was 10 mM, the concentration of Na₂HPO₄ was 40 mM, the concentration of sodium chloride was 150 mM, the content of Tween 20 was 0.03%, the content of DMSO was 5.00%, and the pH was adjusted to 7.4. After the running buffers were prepared, they were filtered with a 0.22 μm filter.

Replacement of protein stock buffer: Zeba™ Spin Desalting Columns 7K MWCO desalting column was used to replace the BCL-XL stock buffer with 10 mM NaH₂PO₄, 40 mM Na₂HPO₄, 150 mM sodium chloride, 0.03% Tween 20, 10% glycerol, pH 7.4, so as to remove the imidazole and Tris ingredients in the stock buffer.

Immobilization of BCL-XL protein: Protein fixation buffer was used to immobilize BCL-XL on the S series NTA chip through His capture and amino coupling. The surface of the NTA chip was cleaned with 50 mM NaOH and 350 mM EDTA respectively, with a flow rate of 60.0 μL/min, 60 seconds each time; then activated with 10 mM NiCl₂ for 1100 seconds, and activated with a mixture of EDC (75.00 mg/mL) and NHS (11.50 mg/mL) at a volume ratio of 1:1 for 650 seconds, with an activation flow rate of 10.0 μL/min; then, BCL-XL (0.04 mg/mL) was injected at 4.0 μL/min for 850 seconds. After injection of BCL-XL, a mixture of EDC (75.00 mg/mL) and NHS (11.50 mg/mL) with a volume ratio of 1:1 was cross-linked at a speed of 10.0 μL/min for 200 seconds, and finally 1M ethanolamine (pH 8.5) was injected at a speed of 6.0 μL/min for 7 minutes to block the chip surface. The final fixation amount of BCL-XL was 4690.00 RU.

Dilution of compound: The test compound was diluted with 100% DMSO to 100 times the required final concentration; after evenly mixing, 4.0 μL was taken and added to 396 μL of running buffer A, and centrifuged at 15000 rpm for 5 minutes to obtain 1× compound solution containing 1% DMSO for subsequent dilution. The compound was serially diluted by 3-fold to 8 concentrations starting from 100 μM with the running buffer B. The diluted compound was transferred to a 96-well plate for sample injection.

Running program: The experiment was run at 25° C. When running the program, the running buffer B was used at a flow rate of 30.0 μL/min. After running buffer B was injected 8 times to complete the equilibrium, the compound was injected sequentially from the lowest concentration to the highest concentration. The binding time and dissociation time were both 120 seconds. After each injection, the injection needle was cleaned with 50% DMSO. The solvent differences caused by DMSO were corrected with 0.50%, 0.75%, 1.00%, 1.25% and 1.50% DMSO.

### 3. Data Analysis

The response value of the compound binding to BCL-XL was analyzed after subtracting the reference channel and 0 concentration. The affinity Kd was fitted by Biacore T200 Evaluation Software using a steady state affinity model (1:1 binding model).

### Experimental Example 4, Detection of Compound-Induced Formation of Ternary Complex Between BCL-XL and CRBN-DDB1 by AlphaLISA Method

### 1. Experimental Instruments and Reagents:

Na₂HPO₄(Sigma), NaH₂PO₄(Sigma), sodium chloride (Sigma), Tween-20 (Sigma), AlphaLISA®Nikel Chelate Acceptor beads (PerkinElmer), Strep-Tactin® Alpha Donor beads (PerkinElmer), Assay plate (Corning), Strep-BCl-XL (HitGen), His-CRBN/DDB1 (HitGen).

### 2. Experimental Methods

DMSO was used to dissolve the dry powder of the test compound to obtain a 10.00 mM solution. The instrument ECHO was used to perform gradient dilution of the compound, which was then added to a 384-well reaction plate (Corning, Cat #3824), so that the final concentration of DMSO in the entire reaction system (10.0 μL) was less than 1%, and the same amount of DMSO was added and used as a control.

Strep-BCl-XL and His-CRBN/DDB1 proteins were dissolved in buffer (10 mM NaH₂PO₄, 40 mM Na₂HPO₄, 150 mM sodium chloride, 0.03% Tween20, pH=7.4) to obtain a protein mixture of 60 nM Strep-BCL-XL and 60 nM His-CRBN/DDB1, the protein mixture (5ul/well) was added to the 384-well plate in which the compound had been added, and incubated at room temperature for 30 minutes, then 5 μl of a detection mixture with 50 μg/ml AlphaLISA®Nikel Chelate Acceptor beads and 50 μg/ml Strep-Tactin® Alpha Donor beads was added, and incubated at room temperature for 2 hours; and then a microplate reader was used to detect AlphaLISA signal.

### 3. Data Analysis

Fifth order polynomial or Sixth order polynomial in Polynomial of Graphpad 6.0 was used for data fitting.

TABLE 1
PROTAC ternary complex signal intensity table

| | AlphaLISA | | AlphaLISA | | AlphaLISA |
|---|---|---|---|---|---|
| No. | signal | No. | signal | No. | signal |
| A | ++ | C3-1 | + | F3B-3 | ++ |
| B3 | + | C3-2 | + | F2-4 | ++ |
| C2-1 | ++ | C3-14 | + | F1B-2 | + |
| C2-2 | ++++ | C3-13 | + | G2-1 | ++ |
| C2-3 | +++++ | C3-10 | ++ | G2-2 | + |
| C2-4 | ++++ | C3-6 | + | G2-3 | + |
| C2-5 | +++ | C3-4 | + | G2-4 | + |
| C3-17 | ++++ | C1-1 | + | G2-5 | + |
| B2 | + | D2-4 | + | G2-7 | + |
| B1 | + | C3-5 | ++ | G2-6 | + |
| C3-15 | + | D2-6 | ++ | G1-5 | ++ |
| C3-16 | ++++ | F3B-2 | ++ | G1-4 | + |
| C3-18 | +++ | E1-1 | + | G3-1 | + |
| C3-19 | +++ | E1-2 | ++ | | |
| C3-3 | + | D2-1 | + | | |

Wherein, the number of + indicates the intensity of AlphaLISA peak signal.

### Experimental Example 5, Evaluation Experiment of Inhibitory Activity of Compound on MOLT4 Cell Proliferation by Cell Titer-Glo Method

### 1. Experimental Instruments and Reagents:

RPMI medium modified (Hyclone), FBS (Corning), Dimethyl Sulfoxide, P/S, CellTiter-Gbo Luminescent Cell Viability Assay (Promega), MOLT-4 cell (ATCC).

### 2. Experimental Methods

MOLT-4 cells were cultured in monolayer in vitro in an incubator containing 5% CO₂ air at 37° C. The culture medium was RPMI 1640 with 10% FBS and 1% P/S. 15 μL of MOLT4 cells, containing 5×10⁴ cells per ml, was inoculated to a 384-well plate (Corning, 3707), 750 cells/well, and incubated at 37° C. and 5% CO₂ for 30 minutes. Then the compound that had been diluted in advance was added, with a total of 9 concentration points, and 2 parallel repetitions for each concentration point. The cell growing group without adding the compound was used as a negative control (maximum signal control), and the culture medium group was used as a blank control (minimum signal control). At the same time, it was ensured that the final DMSO content in each reaction well was 0.1%. The compound and the cells were incubated in a cell culture incubator at 37° C., 5% CO₂ for 3 days (72 hours).

The 384-well plate was taken out from the cell culture incubator and allowed to stand at room temperature, equilibrated for 20 to 30 minutes, and then 25 μl of Cell Titer-Glo detection reagent was added to each reaction well, lysed on a shaker for 2 minutes, incubated for 10 minutes, and BMG PHERAStar (Luminescence) was used for reading.

### 3. Data Analysis

The inhibition rate was calculated based on the luminescence signals: the average value of the negative control (maximum signal control) and the blank control (minimum signal control) was first calculated, then the inhibition rates of the compound at different concentrations for the cells were calculated as follows:

$$Inhibition \% = 1 - \frac{S ( compound signal value ) - S ( blank control signal value )}{S ( negative control signal value ) - S ( blank control signal value )} ) \times 100 \% .$$

The IC₅₀ of the compound to inhibit cell activity was calculated by fitting with GraphPad Prism 6 using log(inhibitor) vs. response−Variable slope mode. The fitting equation was: Y=Bottom+(Top−Bottom)/(1+10\hat{ }((LogIC₅₀−X)*HillSlope)), wherein Y represented the inhibition rate and X represented the known compound concentration after Log.

TABLE 2
IC₅₀ concentrations for inhibiting MOLT-4 cells

| | IC₅₀ | | IC₅₀ | | IC₅₀ |
|---|---|---|---|---|---|
| No. | concentration | No. | concentration | No. | concentration |
| A | +++ | D2-2 | +++ | F3A-4 | ++ |
| C1-1 | ++ | D2-3 | + | F3B-1 | ++ |
| C1-2 | + | D2-4 | ++ | F3B-2 | +++ |
| C1-3 | ++++ | D2-5 | ++ | F3B-3 | ++++ |
| C1-4 | ++++ | D2-6 | ++ | F2-1 | ++ |
| C2-1 | +++ | D2-7 | ++ | F2-2 | +++ |
| C2-2 | +++ | D2-15 | +++++ | F2-3 | ++++ |
| C2-3 | ++ | D2-16 | ++++ | F2-4 | ++++ |
| C2-6 | ++++ | E1-1 | ++ | F2-5 | + |
| C3-2 | ++ | E1-2 | +++ | F2-6 | ++ |
| C3-4 | ++ | F1A-1 | +++++ | F2-7 | ++++ |
| C3-5 | ++ | F1A-2 | +++++ | F2-8 | ++++ |
| C3-6 | +++ | F1A-3 | ++++ | F2-9 | +++ |
| C3-7 | + | F1A-5 | ++++ | F2-10 | ++++ |
| C3-8 | + | F1B-1 | +++ | F2-11 | +++ |
| C3-11 | ++ | F1B-2 | +++ | G1-3 | + |
| C3-12 | +++ | F1B-3 | +++ | G2-1 | + |
| C3-16 | +++ | F1B-4 | ++++ | G1-5 | + |
| C3-17 | +++ | F3A-1 | + | H1-1 | +++++ |
| D1-1 | ++ | F3A-2 | ++ | | |
| D2-1 | ++ | F3A-3 | + | | |

Wherein, + represented IC₅₀ > 200 μM, ++ represented 200 μM > IC₅₀ > 100 μM, +++ represented 100 μM > IC₅₀ > 50 μM, ++++ represented 10 μM > IC₅₀ > 1 μM, +++++ represented IC₅₀ < 1 μM.

### Experimental Example 6. Experiment to Evaluate Degradation Effect of Compound on BCL-XL Protein in MOLT-4 Cells by Western Blot Method

### 1. Experimental Instruments and Reagents:

SDS-PAGE gel (4-12%) (Genscript), PMSF 100 mM (Biyotime), Cocktail 100× (Biyotime), P/S, RPMI medium modified (Hyclone), FBS (Corning), 4× loading buffer (Thermo), Bcl-xL (54H6) Rabbit mAb (CST), Anti-rabbit IgG-HRP (CST), Anti-β-actin-HRP (Abcam), RIPA (Biyotime), Tween-20, MOLT-4 cell (ATCC), BCA Protein Assay Kit (TIANGEN), Immobilon Western Chemilunescent HRP Substrate (Millipore).

### 2. Experimental Methods

1) Incubation of compound and cells: 1 mL of 10⁶ MOLT-4 cells (culture medium: 89% RPMI, 10% FBS, 1% P/S) was inoculated in a 6-well cell culture plate, the compound was subjected to gradient dilution with DMSO, then the compound diluted in DMSO in gradient was diluted again into the MOLT-4 medium, and 1 mL of the diluted compound was directly added to the 6-well plate with the cultured cells and incubated for 24 hours.

2) Protein extraction: After the compound and the cells were co-incubated for 24 hours, the cells were collected and placed in a 1.5 mL centrifuge tube, washed twice with ice-cold PBS, the supernatant was discarded, then 150 μL of RIPA lysis buffer was added and lysis was carried out on ice for 30 minutes; then the cell lysate was centrifuged in a low-temperature centrifuge at 15,000 rpm for 10 minutes, and the supernatant was collected; and BCA Protein Assay Kit was used to quantify the protein in the resulting cell lysate supernatant. 4× loading buffer was added to the cell lysate supernatant and incubated at 85° C. for 10 minutes to obtain a sample. The obtained sample was stored in −80° C. refrigerator for the use on the next day. 20 μg of the sample was taken to perform SDS-Page and transmembrane, 5% skim milk was used to block the membrane for 1 hour, and then Bcl-xL (54H6) rabbit mAb, Anti-rabbit IgG-HRP, Anti-β-actin-HRP were used for incubation. After incubation, Immobilon Western Chemiluminescent HRP Substrate was used for color development, and a gel analyzer was used for analysis.

### 3. Data Analysis

Analysis was performed with a grayscale analysis software, the amount of BCL-XL protein was corrected by the amount of β-actin, and then Graphpad 6 was used to calculate DC₅₀.

TABLE 3
DC₅₀ concentration for BCL-XL protein degradation

| | DC₅₀ | | DC₅₀ | | DC₅₀ |
|---|---|---|---|---|---|
| No. | concentration | No. | concentration | No. | concentration |
| C2-2 | +++ | C3-13 | ++ | F1B-3 | ++++ |
| C2-3 | +++ | C3-10 | +++ | F2-4 | ++++ |
| C3-17 | ++++ | C3-6 | +++++ | F2-8 | +++++ |
| C3-16 | ++++ | E1-2 | ++++ | F2-9 | ++++ |
| C3-1 | ++ | D2-1 | +++++ | F2-11 | ++++ |
| C3-2 | ++++ | D2-15 | ++++ | F3B-3 | +++++ |
| C3-14 | ++ | F1B-2 | +++ | H1-1 | +++++ |

Wherein, + represented DC₅₀ > 200 μM, ++ represented 200 μM > DC₅₀ > 100 μM, +++ represented 100 μM > DC₅₀ > 50 μM, ++++ represented 10 μM > DC₅₀ > 1 μM, +++++ represented DC₅₀ < 1 μM.

1. A compound represented by Formula I, or deuterated compound thereof, or stereoisomer thereof, or tautomer thereof, or polymorph thereof, or solvate thereof, or N-oxide thereof, or isotopically labeled compound thereof, or metabolite thereof, or prodrug thereof, or pharmaceutically acceptable salt thereof:
X-Y-Z Formula I
wherein,
X represents a group binding to a BCL-XL protein;
Y represents a connecting group;
Z represents a group binding to an E3 ubiquitin-protein ligase.

2. The compound according to claim 1, wherein X is selected from 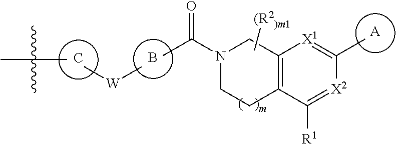
wherein,
Ring A is selected from C_{6˜10} aromatic ring or 6- to 10-membered aromatic heterocycle; wherein the aromatic ring and the aromatic heterocycle can be further substituted by one, two or three R^{A1} groups;
each R^{A1} is independently selected from hydrogen, halogen, cyano, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{A2} or —C_{0˜4} alkylene-NR^{A2}R^{A3};
R^{A2} and R^{A3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
X¹ and X² are each independently selected from N or CR^{X1};
each R^{X1} is independently selected from hydrogen, halogen, cyano, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{X2} or —C_{0˜4} alkylene-NR^{X2}R^{X3};
R^{X2} and R^{X3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
R¹ is selected from hydrogen, halogen, cyano, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR¹¹ or —C_{0˜4} alkylene-NR¹¹R¹²;
R¹¹ and R¹² are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
each R² is independently selected from hydrogen, halogen, cyano, ═O, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR²¹ or —C_{0˜4} alkylene-NR²¹R²²;
R²¹ and R²² are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
m1 is selected from 0, 1, 2 or 3;
m is selected from 0, 1 or 2;
Ring B is selected from C_{3˜10} cycloalkane, 3- to 10-membered heterocycloalkane, benzene ring, 5- to 6-membered aromatic heterocycle or 5- to 12-membered bridged ring; wherein, the cycloalkane, heterocycloalkane, benzene ring, aromatic heterocycle and bridged ring can be further substituted by one, two or three R^{B1};
each R^{B1} is independently selected from hydrogen, halogen, cyano, ═O, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{B2} or —C_{0˜4} alkylene-NR^{B2}R^{B3};
R^{B2} and R^{B3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl;
W is selected from —C_{1˜6} alkylene-, —C_{1˜6} alkylene-O—, —O—C_{1˜6} alkylene-, —C_{2˜6} alkenylene-, —C_{2˜6} alkenylene-O—, —O—C_{2˜6} alkenylene-, —C_{2˜6} alkynylene-, —C_{2˜6} alkynylene-O— or —O—C_{2˜6} alkynylene-;
Ring C is selected from benzene ring or 5- to 6-membered aromatic heterocycle; wherein, benzene ring and aromatic heterocycle can be further substituted by one, two or three R^{C1};
each R^{C1} is independently selected from hydrogen, halogen, cyano, —C_{1˜6} alkyl, —C_{2˜6} alkenyl, —C_{2˜6} alkynyl, halogen-substituted C_{1˜6} alkyl, halogen-substituted —C_{2˜6} alkenyl, halogen-substituted —C_{2˜6} alkynyl, —C_{0˜4} alkylene-OR^{C2} or —C_{0˜4} alkylene-NR^{C2}R^{C3};
R^{C2} and R^{C3} are each independently selected from hydrogen, —C_{1˜6} alkyl, —C_{2˜6} alkenyl, —C_{2˜6} alkynyl, halogen-substituted —C_{1˜6} alkyl, halogen-substituted —C_{2˜6} alkenyl or halogen-substituted —C_{2˜6} alkynyl.

3. The compound according to claim 2, wherein Ring A is selected from naphthalene ring or 8- to 10-membered aromatic heterocycle; wherein, the naphthalene ring and aromatic heterocycle can be further substituted by one, two or three R^{A1}, each R^{A1} is independently selected from hydrogen, halogen, cyano or —C_{1˜6} alkyl; preferably, Ring A is selected from naphthalene ring, benzopyrimidine ring or quinoline ring; more preferably, Ring A is selected from quinoline ring.

4. The compound according to claim 2, wherein X¹ and X² are each independently selected from N or CH; preferably, X¹ and X² are selected from N.

5. The compound according to claim 2, wherein R¹ is selected from hydrogen, halogen, cyano, —C_{1˜6} alkyl or —NR¹¹R¹², and R¹¹ and R¹² are each independently selected from hydrogen or —C_{1˜6} alkyl; preferably, R¹ is selected from hydrogen or —NR¹¹R¹², and R¹¹ and R¹² are each independently selected from hydrogen or —C_{1˜6} alkyl; more preferably, R¹ is selected from hydrogen

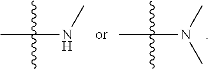

6. The compound according to claim 2, wherein m is selected from 1.

7. The compound according to claim 2, wherein Ring B is selected from benzene ring or 6-membered nitrogen-containing heterocycloalkane, 6-membered nitrogen-containing aromatic heterocycle, 5- to 6-membered cycloalkane or 5- to 8-membered bridged ring; wherein, the benzene ring, heterocycloalkane, aromatic heterocycle, cycloalkane and bridged ring can be further substituted by one, two or three R^{B1}; each R^{B1} is independently selected from hydrogen, halogen, cyano or —C_{1˜6} alkyl; preferably, Ring B is selected from benzene ring, pyridine ring or 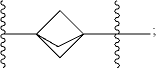 [SMILES: *C12CC(*)(C1)C2]
more preferably, Ring B is selected from benzene ring.

8. The compound according to claim 2, wherein W is selected from —C_{1˜4} alkylene-, —C_{1˜4} alkylene-O—, —O—C_{1˜4} alkylene-, —C_{2˜4} alkenylene-, —C_{2˜4} alkenylene-O—, —O—C_{2˜4} alkenylene-, —C_{2˜4} alkynylene-, —C_{2˜4} alkynylene-O— or —O—C_{2˜4} alkynylene-; preferably, W is selected from —C_{1˜4} alkylene-, —C_{1˜4} alkylene-O—, —O—C_{1˜4} alkylene-, —C_{2˜4} alkenylene-, —C_{2˜4} alkenylene-O— or —O—C_{2˜4} alkenylene-; more preferably, W is selected from —C_{1˜3} alkylene-, —C_{1˜3} alkylene-O—, —O—C_{1˜3} alkylene- or —C_{2˜3} alkenylene-; further preferably, W is selected from ethylene,

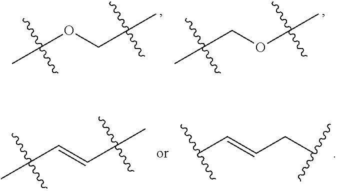

9. The compound according to claim 2, wherein Ring C is selected from benzene ring or 6-membered nitrogen-containing aromatic heterocycle; wherein, the benzene ring and the aromatic heterocycle can be further substituted by one, two or three R^{C1}, each R^{C1} is independently selected from hydrogen, halogen, cyano or —C_{1˜6} alkyl; preferably, Ring C is selected from benzene ring or pyridine ring.

10. The compound according to claim 2, wherein each R² is independently selected from halogen, cyano, ═O, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C₂₋₆ alkynyl, —C_{0˜4} alkylene-OR²¹ or —C_{0˜4} alkylene-NR²¹R²²; preferably, each R² is independently selected from halogen, cyano, —C_{1˜6} alkyl, halogen-substituted —C_{1˜6} alkyl, halogen-substituted —C_{2˜6} alkenyl, —C_{0˜4} alkylene-OR²¹ or —C_{0˜4} alkylene-NR²¹R²²; further preferably, each R² is independently selected from —C_{0˜4} alkylene-NR²¹R²²;
preferably, m1 is selected from 0 or 1 or 2; further preferably, m1 is selected from 0;
preferably, R²¹ and R²² are each independently selected from hydrogen, —C_{1˜6} alkyl, or halogen-substituted —C_{1˜6} alkyl; further preferably, R²¹ and R²² are each independently selected from hydrogen.

11. The compound according to claim 2, wherein
Ring A is selected from naphthalene ring or 8- to 10-membered aromatic heterocycle; wherein, the naphthalene ring or aromatic heterocycle can be further substituted by one, two or three R^{A1};
X¹ and X² are each independently selected from N or CH;
R¹ is selected from hydrogen or —NR¹¹R¹²;
m is selected from 0 or 1;
Ring B is selected from 6-membered nitrogen-containing heterocycloalkane, benzene ring, 6-membered nitrogen-containing aromatic heterocycle, 5-membered bridged cycloalkane, or 5-membered cycloalkane; wherein, the benzene ring, aromatic heterocycle, cycloalkane, and bridged cycloalkane can be further substituted by one, two or three R^{B1};
W is selected from —C_{1˜4} alkylene-, —C_{1˜4} alkylene-O—, —O—C_{1˜4} alkylene-, —C_{2˜4} alkenylene-, —C_{2˜4} alkenylene-O—, —O—C_{2˜4} alkenylene-, —C_{2˜4} alkynylene-, —C_{2˜4} alkynylene-O— or —O—C_{2˜4} alkynylene-;
Ring C is selected from benzene ring or 6-membered nitrogen-containing aromatic heterocycle; wherein, the benzene ring and aromatic heterocycle can be further substituted by one, two or three R^{C1}.

12. The compound according to claim 2, wherein
Ring A is selected from 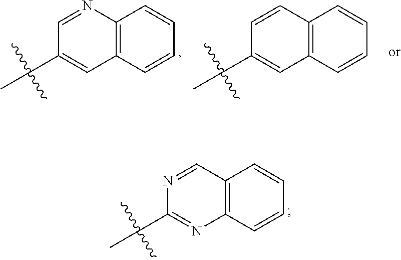 [SMILES: *c1cnc2ccccc2c1 ; *c1ccc2ccccc2c1 ; *c1ncc2ccccc2n1]
X¹ and X² are selected from N or CH;
R¹ is selected from hydrogen, 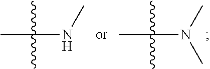
m1 is selected from 0;
m is selected from 1;
Ring B is selected from, 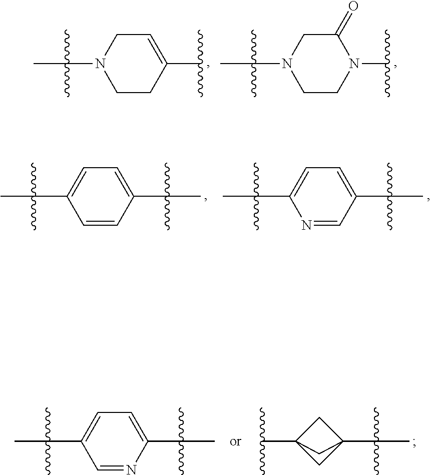 [SMILES: *C1=CCN(*)CC1 ; *N1CCN(*)C(=O)C1 ; *c1ccc(*)cc1 ; *c1ccc(*)nc1 ; *c1ccc(*)nc1 ; *C12CC(*)(C1)C2]
preferably, Ring B is selected from 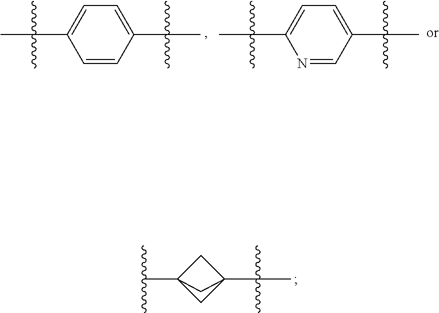 [SMILES: *c1ccc(*)cc1 ; *c1ccc(*)nc1 ; *C12CC(*)(C1)C2]
W is selected from ethylene, 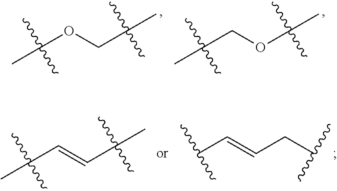
preferably,
W is selected from 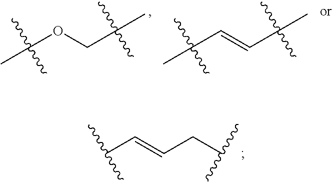
Ring C is selected from

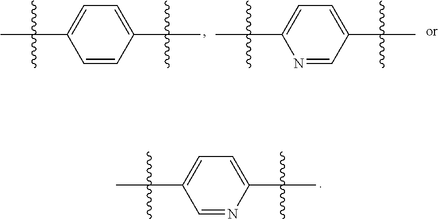 [SMILES: *c1ccc(*)cc1 ; *c1ccc(*)nc1 ; *c1ccc(*)nc1]

13. The compound according to claim 2, wherein X is selected from

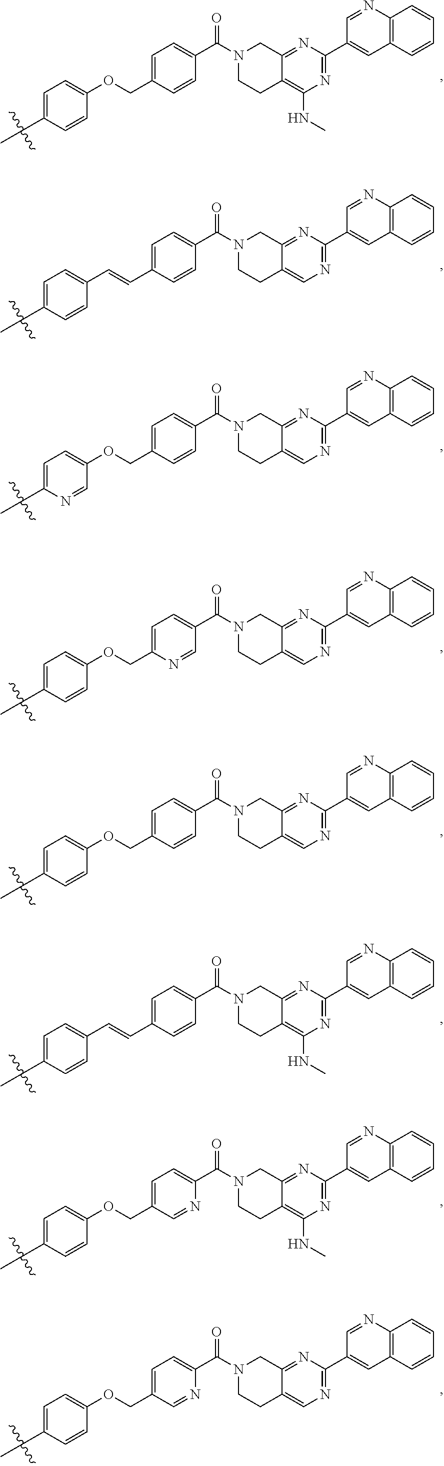

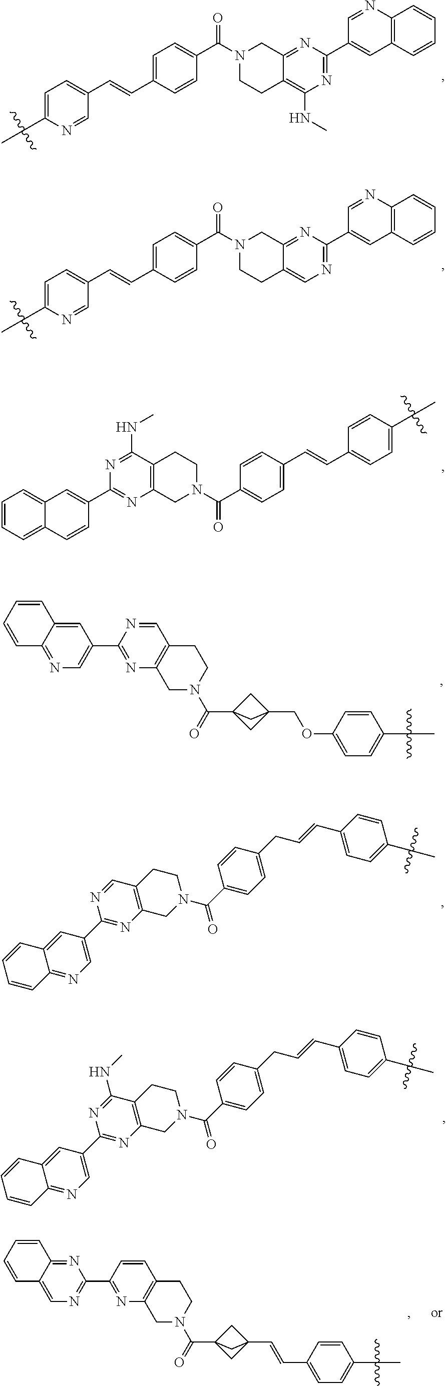

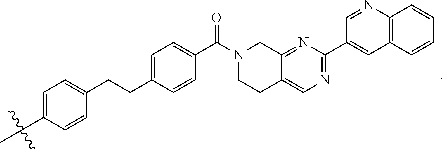 [SMILES: *c1ccc(CCc2ccc(C(=O)N3CCc4cnc(-c5cnc6ccccc6c5)nc4C3)cc2)cc1]

14. The compound according to claim 1, wherein X is optionally and independently substituted by one or more (e.g., 2, 3, 4, 5, 6, 7, 8, 9 or 10) R^{A1′},
preferably, each R^{A1′} is independently selected from halogen, cyano, —C_{1˜6} alkyl, —C_{2˜6} alkenyl, —C_{2˜6} alkynyl, halogen-substituted —C_{1˜6} alkyl, halogen-substituted —C_{2˜6} alkenyl, halogen-substituted —C_{2˜6} alkynyl, —C_{0˜4} alkylene-OR^{A2′} or —C_{0˜4} alkylene-NR^{A2′}R^{A3′};
R^{A2′} and R^{A3′} are each independently selected from hydrogen, —C_{1˜3} alkyl, —C_{2˜6} alkenyl, —C_{2˜6} alkynyl, halogen-substituted —C_{1˜3} alkyl, halogen-substituted —C_{2˜6} alkenyl, or halogen-substituted —C_{2˜6} alkynyl;
preferably, each R^{A1′} is independently selected from halogen, cyano, —C_{1˜3} alkyl, —C_{2˜4} alkenyl, halogen-substituted —C_{1˜3} alkyl, halogen-substituted —C_{2˜4} alkenyl, —C_{0˜4} alkylene-OR^{A2′} or —C_{0˜4}alkylene-NR^{A2′}R^{A3′};
R^{A2′} and R^{A3′} are each independently selected from hydrogen, —C_{1˜3} alkyl, —C_{2˜4} alkenyl, halogen-substituted —C_{1˜3} alkyl or halogen-substituted —C_{2˜4} alkenyl.

15. The compound according to claim 1, wherein
the Y is selected from -(L^{Y})_{q}-;
q is an integer selected from 1 to 30;
each L^{Y} is independently selected from structural fragments consisting of any one or more members selected from the group consisting of C(R)₂, C(O), O, S, S(O), S(O)₂, NR, —CR═CR—, —C≡C—, C_{3˜10} cycloalkane, 3- to 10-membered heterocycloalkane, C_{6˜10} aromatic ring, 5- to 10-membered aromatic heterocycle, 5- to 12-membered spiro ring, 5- to 12-membered spiro heterocycle, 5- to 12-membered bridged ring, and 5- to 12-membered bridged heterocycle; wherein the cycloalkane, heterocycloalkane, aromatic ring, aromatic heterocycle, spiro ring, spiro heterocycle, bridged ring, and bridged heterocycle can be further substituted by one, two, or three R^{YL};
each R^{YL} is independently selected from the group consisting of hydrogen, halogen, cyano, nitro, C_{1˜6} alkyl, halogen-substituted C_{1˜6} alkyl, —OR, and —N(R)₂;
each R is independently selected from the group consisting of hydrogen, halogen, —C_{1˜6} alkyl, halogen-substituted —C_{1˜6} alkyl, —C_{0˜2} alkylene-(C_{3˜10} carbocyclic group), and —C_{0˜2} alkylene-(3- to 10-membered heterocycloalkyl).

16. The compound according to claim 1, wherein
Y is selected from 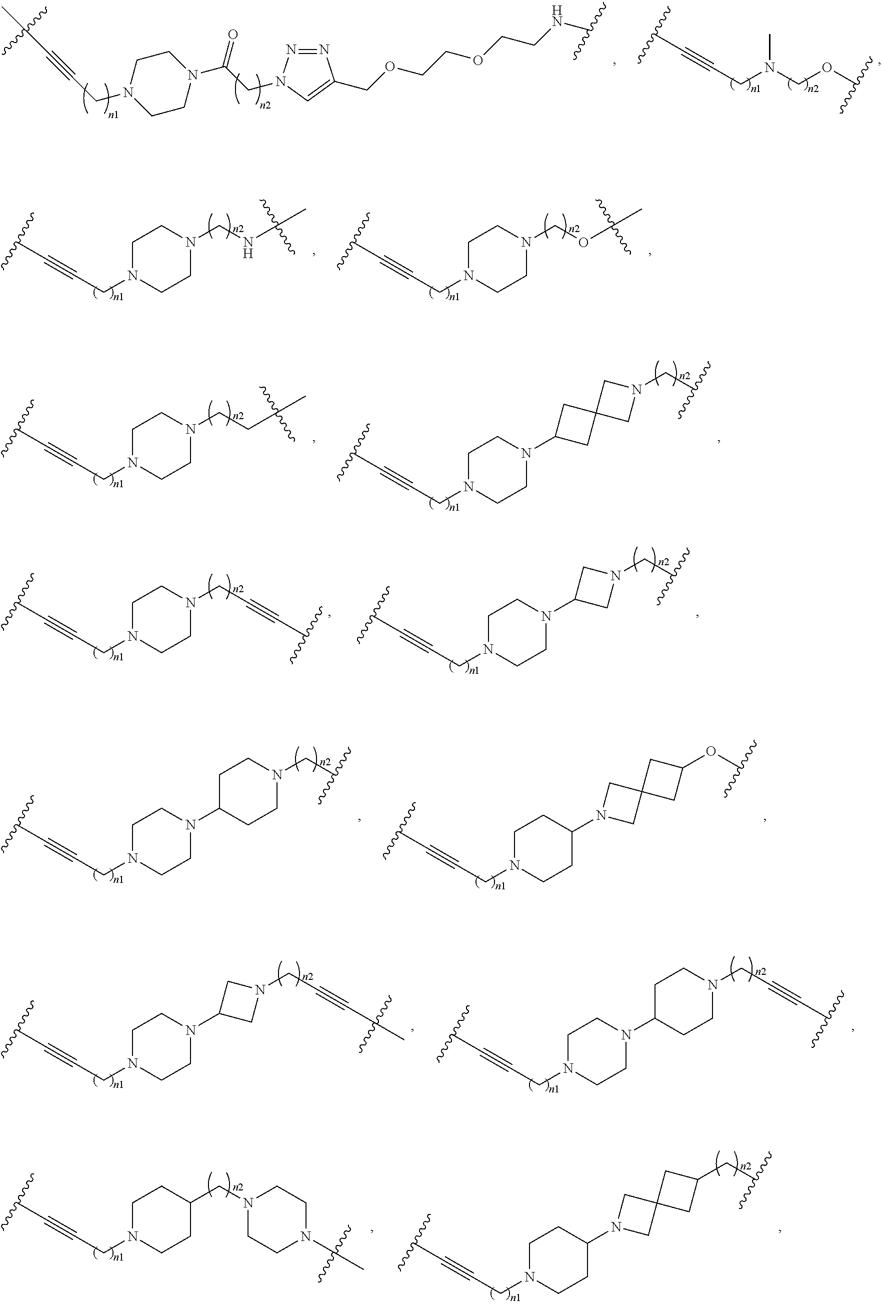 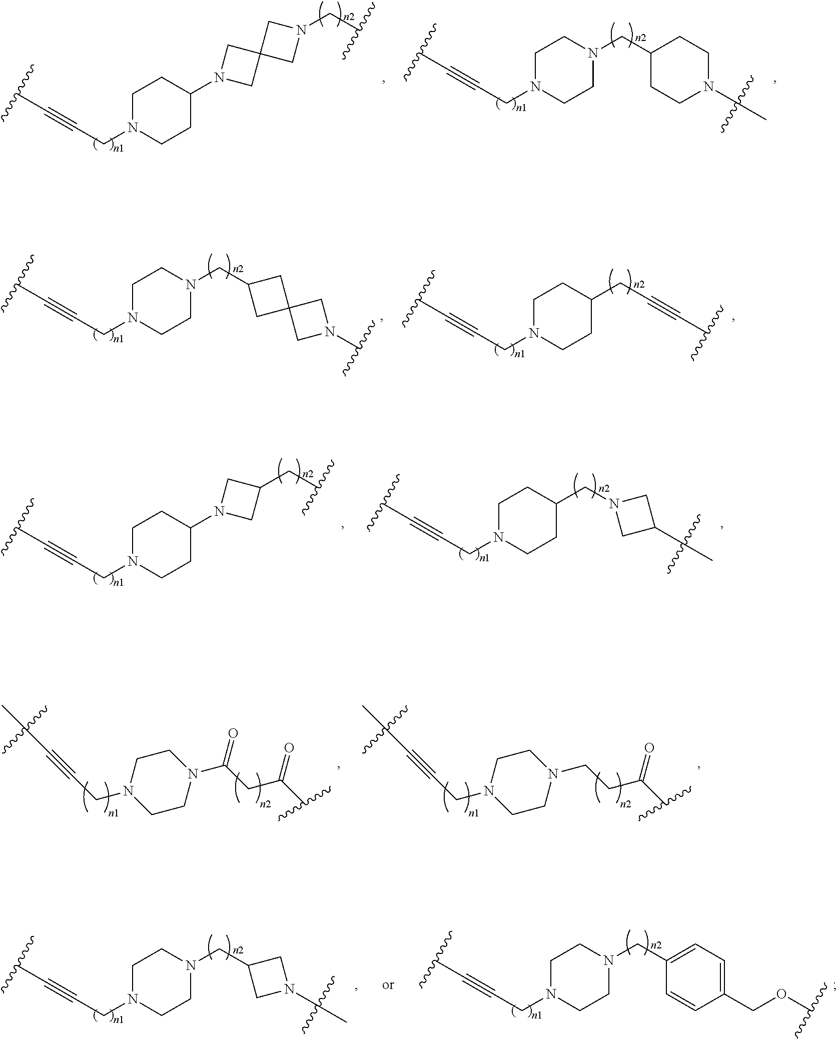
wherein, n1 in each structural fragment is independently an integer selected from 0 to 10, n2 in each structural fragment is independently an integer selected from 0 to 10; preferably, n1 in each structural fragment is independently selected from 0, 1, 2, 3, 4, 5, 6, 7, 8, and n2 in each structural fragment is independently selected from 0, 1, 2, 3, 4, 5, 6, 7, 8.

17. The compound according to claim 1, wherein:
Y is selected from

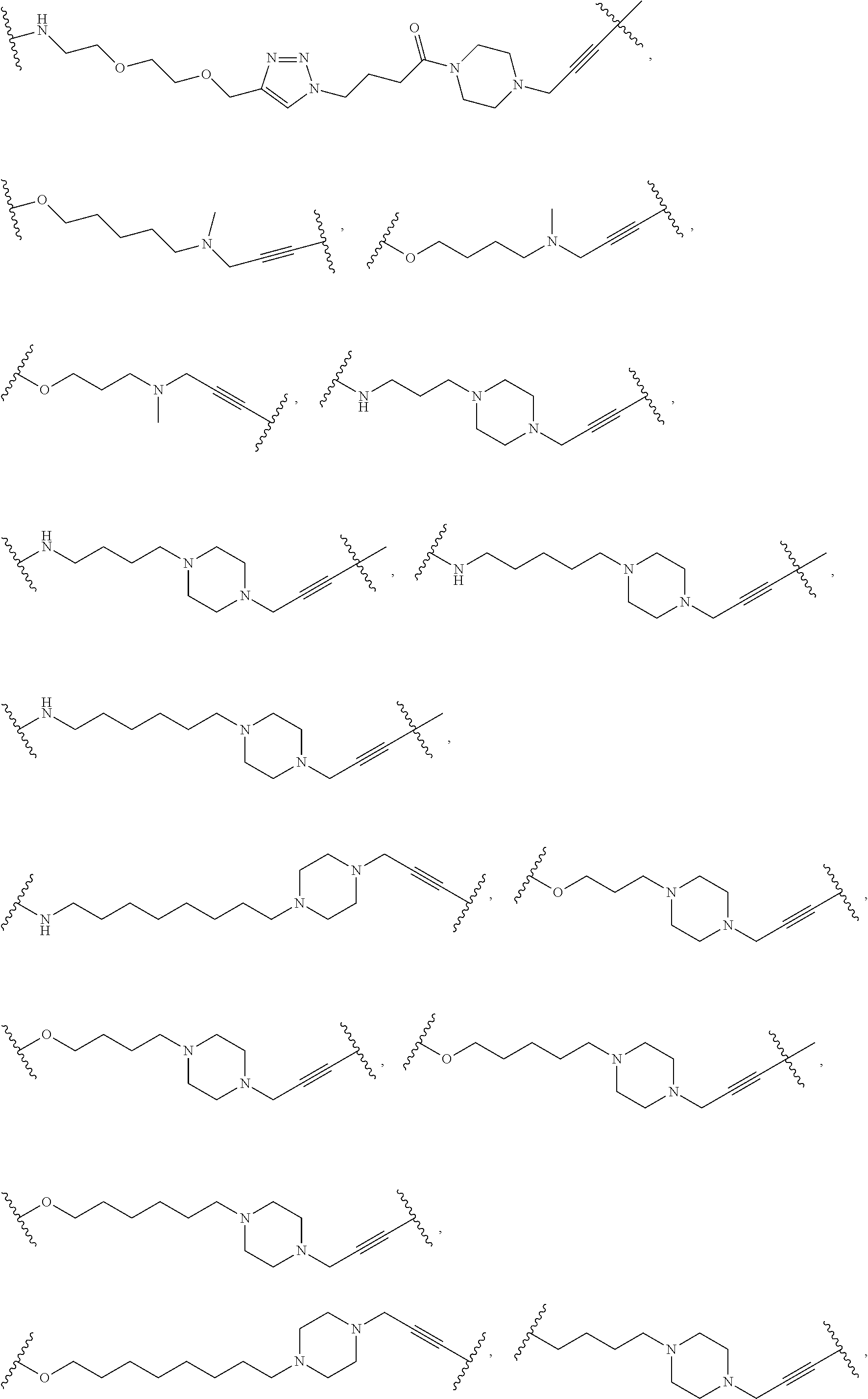

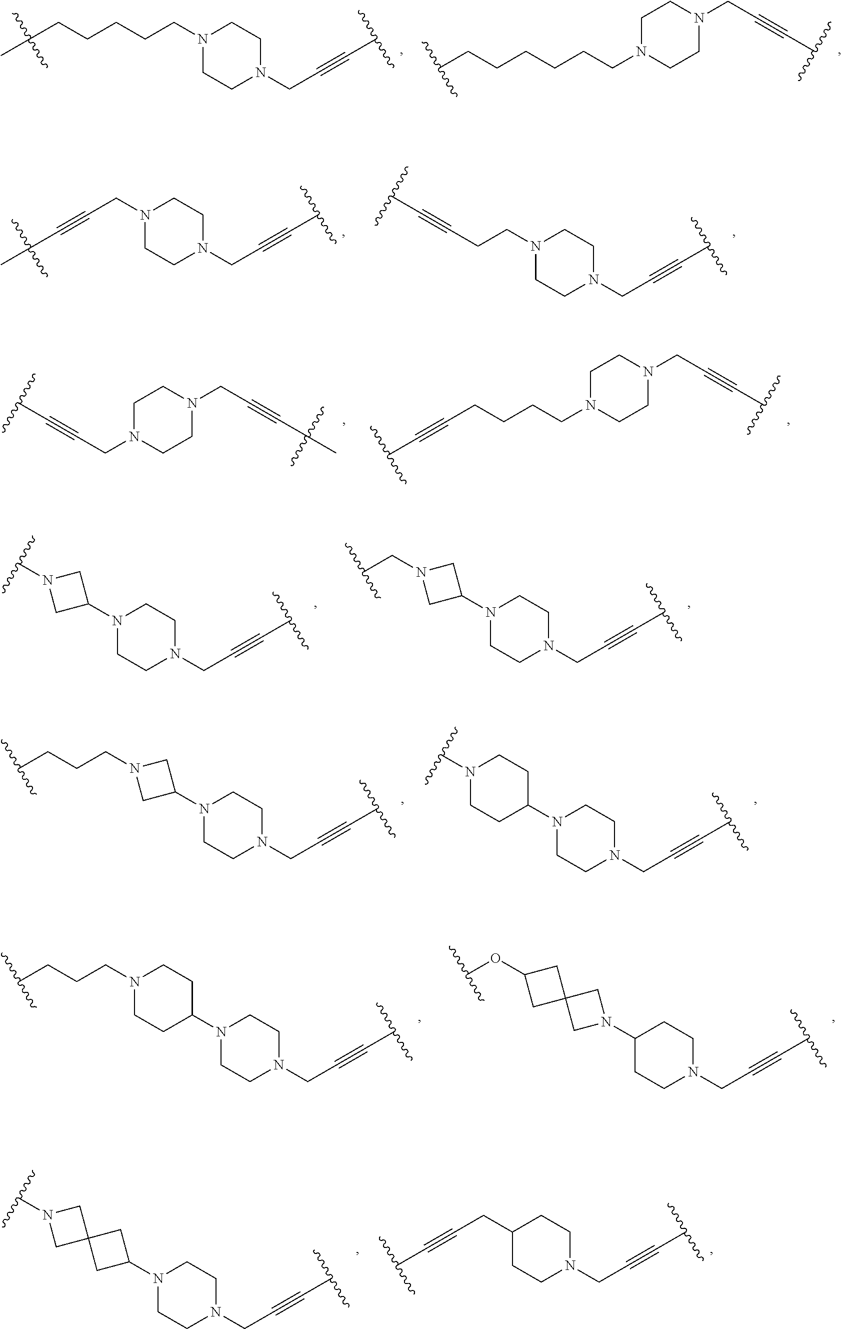

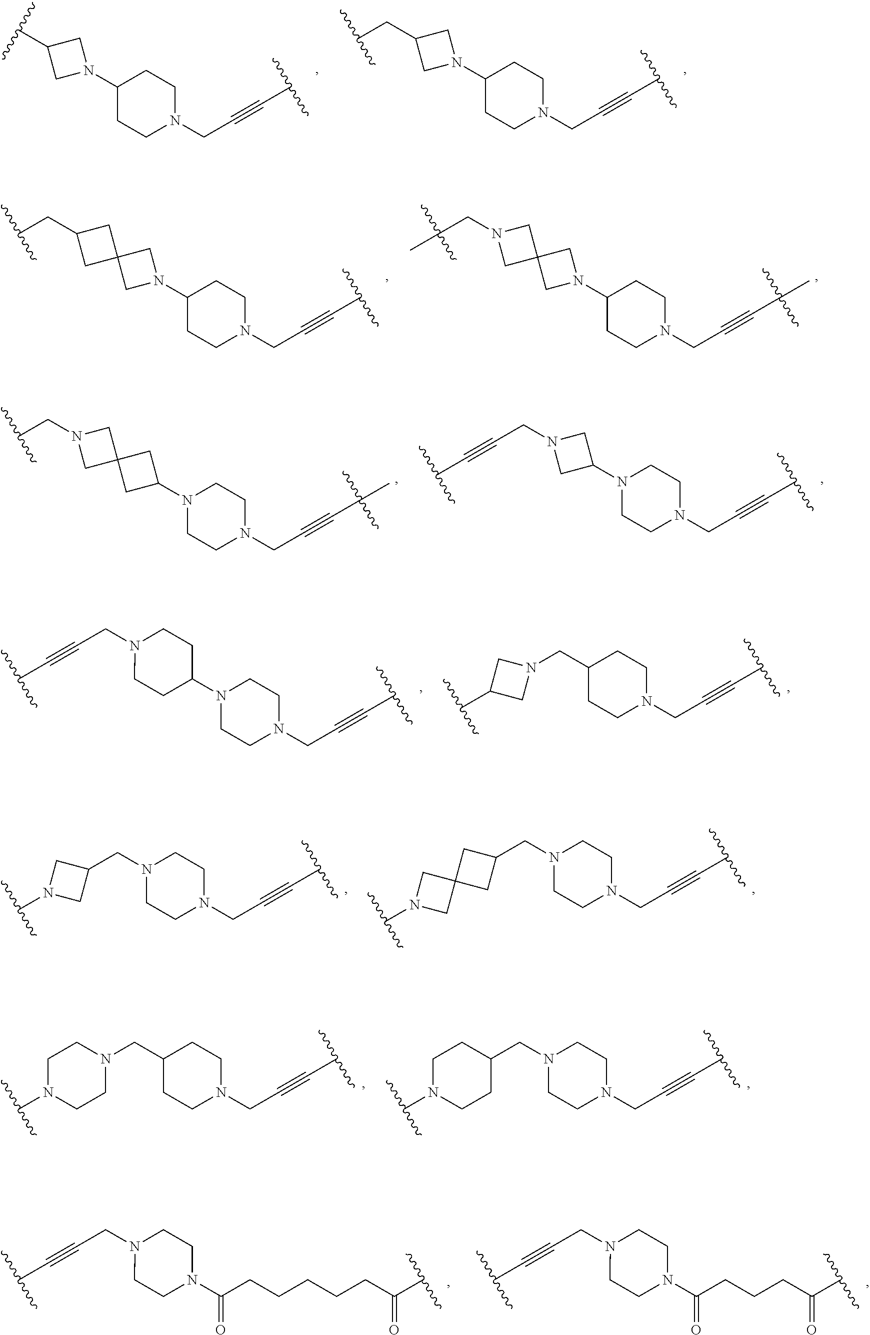

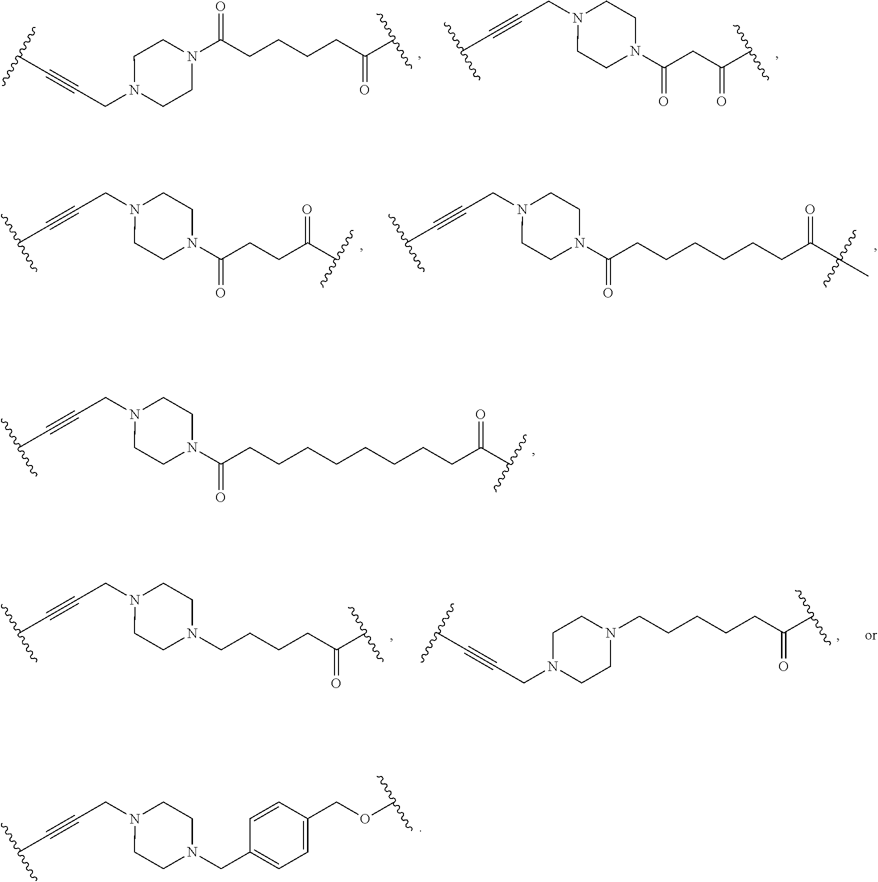

18. The compound according to claim 1, wherein the E3 ubiquitin-protein ligase is selected from the group consisting of CRBN, von Hippel-Lindau (VHL), XIAP, MDM2, and cIAP-1.

19. The compound according to claim 1, wherein
Z is selected from

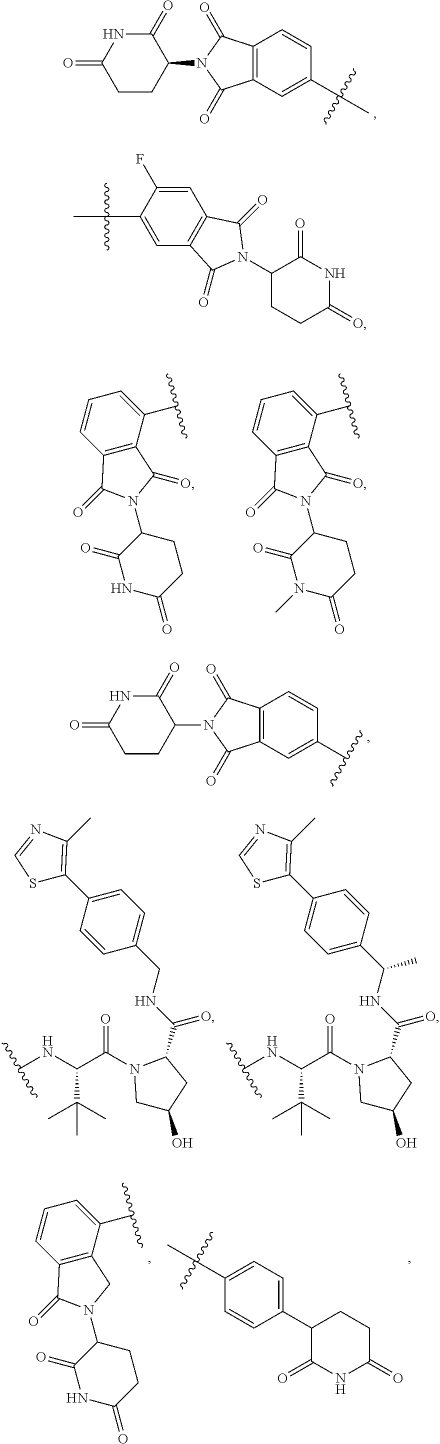

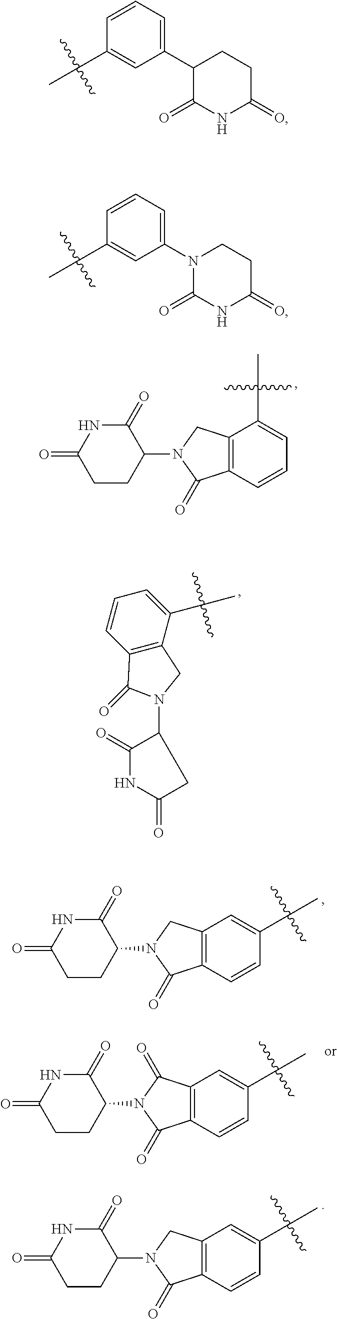

20. The compound according to claim 1, wherein the compound is specifically:

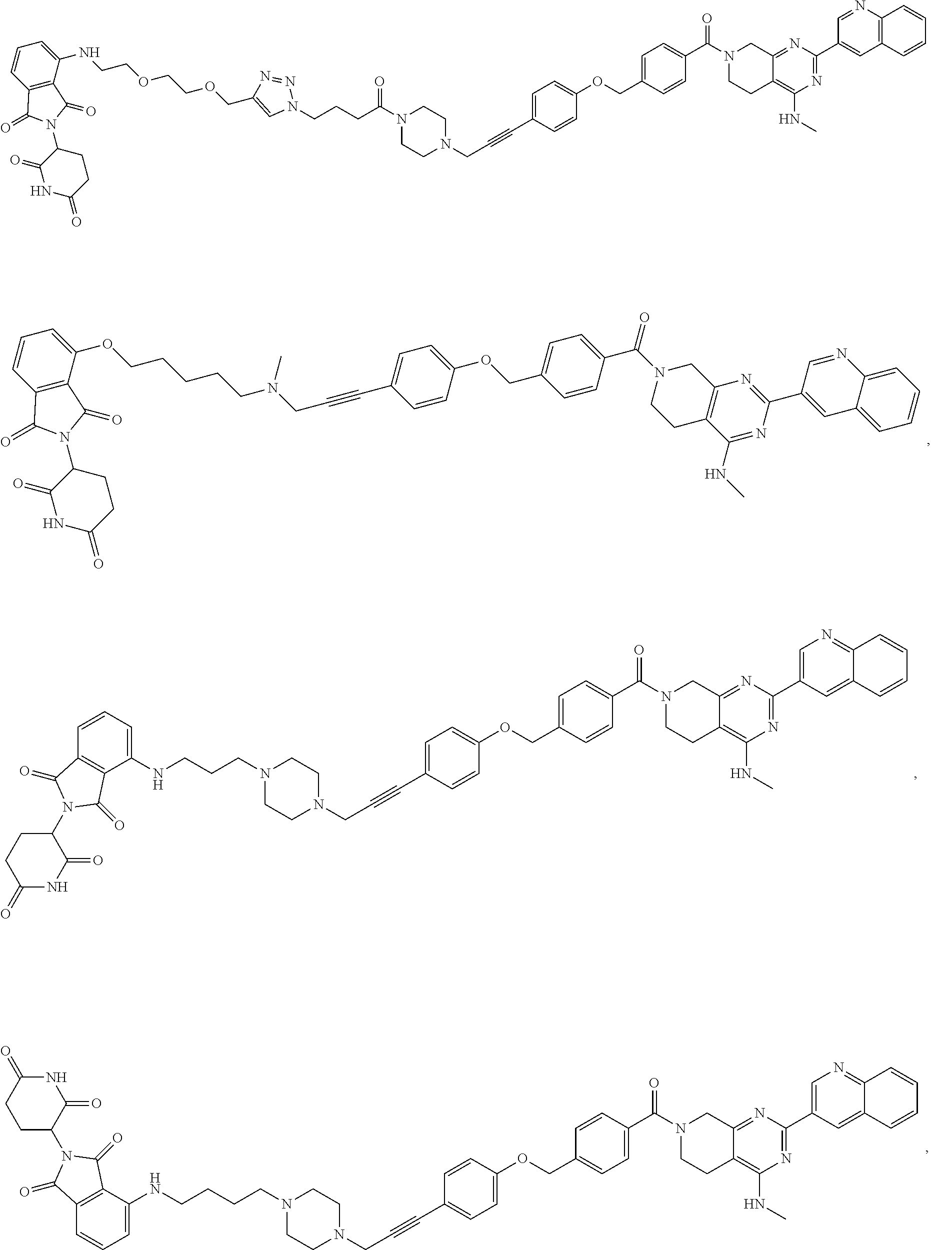 [SMILES: CNc1nc(-c2cnc3ccccc3c2)nc2c1CCN(C(=O)c1ccc(COc3ccc(C#CCN4CCN(C(=O)CCCn5cc(COCCOCCNc6cccc7c6C(=O)N(C6CCC(=O)NC6=O)C7=O)nn5)CC4)cc3)cc1)C2 ; CNc1nc(-c2cnc3ccccc3c2)nc2c1CCN(C(=O)c1ccc(COc3ccc(C#CCN(C)CCCCCOc4cccc5c4C(=O)N(C4CCC(=O)NC4=O)C5=O)cc3)cc1)C2 ; CNc1nc(-c2cnc3ccccc3c2)nc2c1CCN(C(=O)c1ccc(COc3ccc(C#CCN4CCN(CCCNc5cccc6c5C(=O)N(C5CCC(=O)NC5=O)C6=O)CC4)cc3)cc1)C2 ; CNc1nc(-c2cnc3ccccc3c2)nc2c1CCN(C(=O)c1ccc(COc3ccc(C#CCN4CCN(CCCCNc5cccc6c5C(=O)N(C5CCC(=O)NC5=O)C6=O)CC4)cc3)cc1)C2]

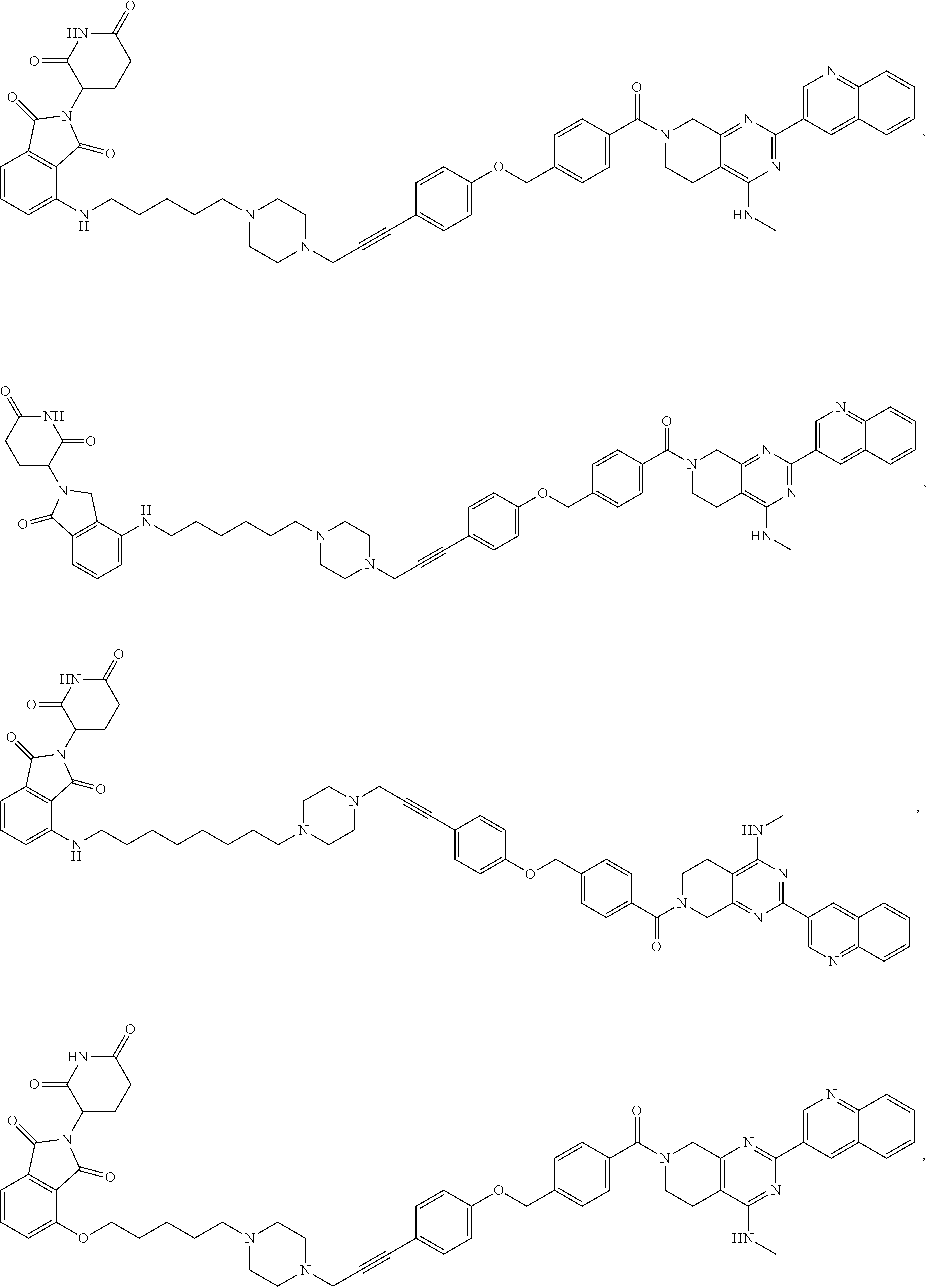 [SMILES: CNc1nc(-c2cnc3ccccc3c2)nc2c1CCN(C(=O)c1ccc(COc3ccc(C#CCN4CCN(CCCCCNc5cccc6c5C(=O)N(C5CCC(=O)NC5=O)C6=O)CC4)cc3)cc1)C2 ; CNc1nc(-c2cnc3ccccc3c2)nc2c1CCN(C(=O)c1ccc(COc3ccc(C#CCN4CCN(CCCCCCNc5cccc6c5CN(C5CCC(=O)NC5=O)C6=O)CC4)cc3)cc1)C2 ; CNc1nc(-c2cnc3ccccc3c2)nc2c1CCN(C(=O)c1ccc(COc3ccc(C#CCN4CCN(CCCCCCCCNc5cccc6c5C(=O)N(C5CCC(=O)NC5=O)C6=O)CC4)cc3)cc1)C2 ; CNc1nc(-c2cnc3ccccc3c2)nc2c1CCN(C(=O)c1ccc(COc3ccc(C#CCN4CCN(CCCCCOc5cccc6c5C(=O)N(C5CCC(=O)NC5=O)C6=O)CC4)cc3)cc1)C2]

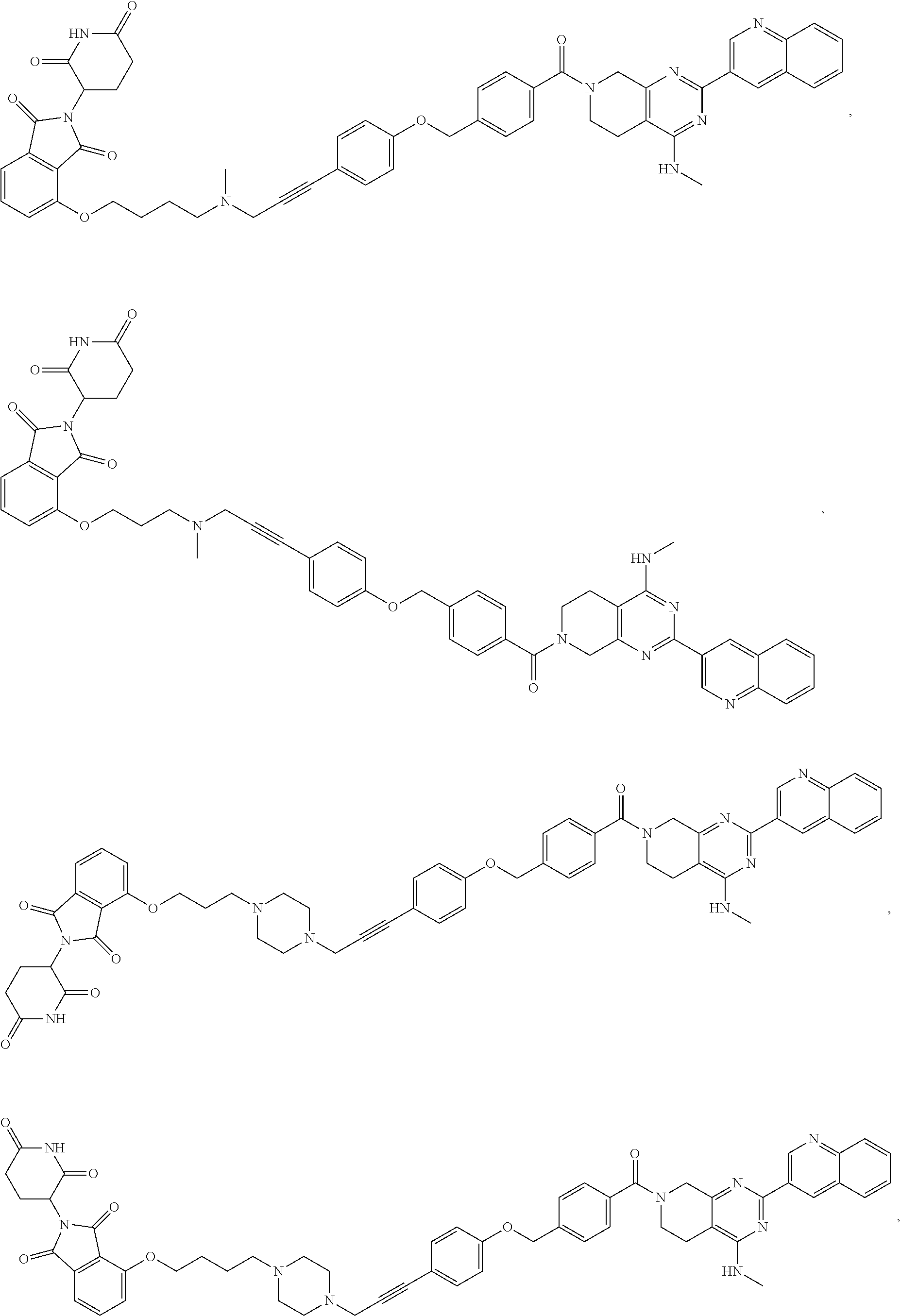 [SMILES: CNc1nc(-c2cnc3ccccc3c2)nc2c1CCN(C(=O)c1ccc(COc3ccc(C#CCN(C)CCCCOc4cccc5c4C(=O)N(C4CCC(=O)NC4=O)C5=O)cc3)cc1)C2 ; CNc1nc(-c2cnc3ccccc3c2)nc2c1CCN(C(=O)c1ccc(COc3ccc(C#CCN(C)CCCOc4cccc5c4C(=O)N(C4CCC(=O)NC4=O)C5=O)cc3)cc1)C2 ; CNc1nc(-c2cnc3ccccc3c2)nc2c1CCN(C(=O)c1ccc(COc3ccc(C#CCN4CCN(CCCOc5cccc6c5C(=O)N(C5CCC(=O)NC5=O)C6=O)CC4)cc3)cc1)C2 ; CNc1nc(-c2cnc3ccccc3c2)nc2c1CCN(C(=O)c1ccc(COc3ccc(C#CCN4CCN(CCCCOc5cccc6c5C(=O)N(C5CCC(=O)NC5=O)C6=O)CC4)cc3)cc1)C2]

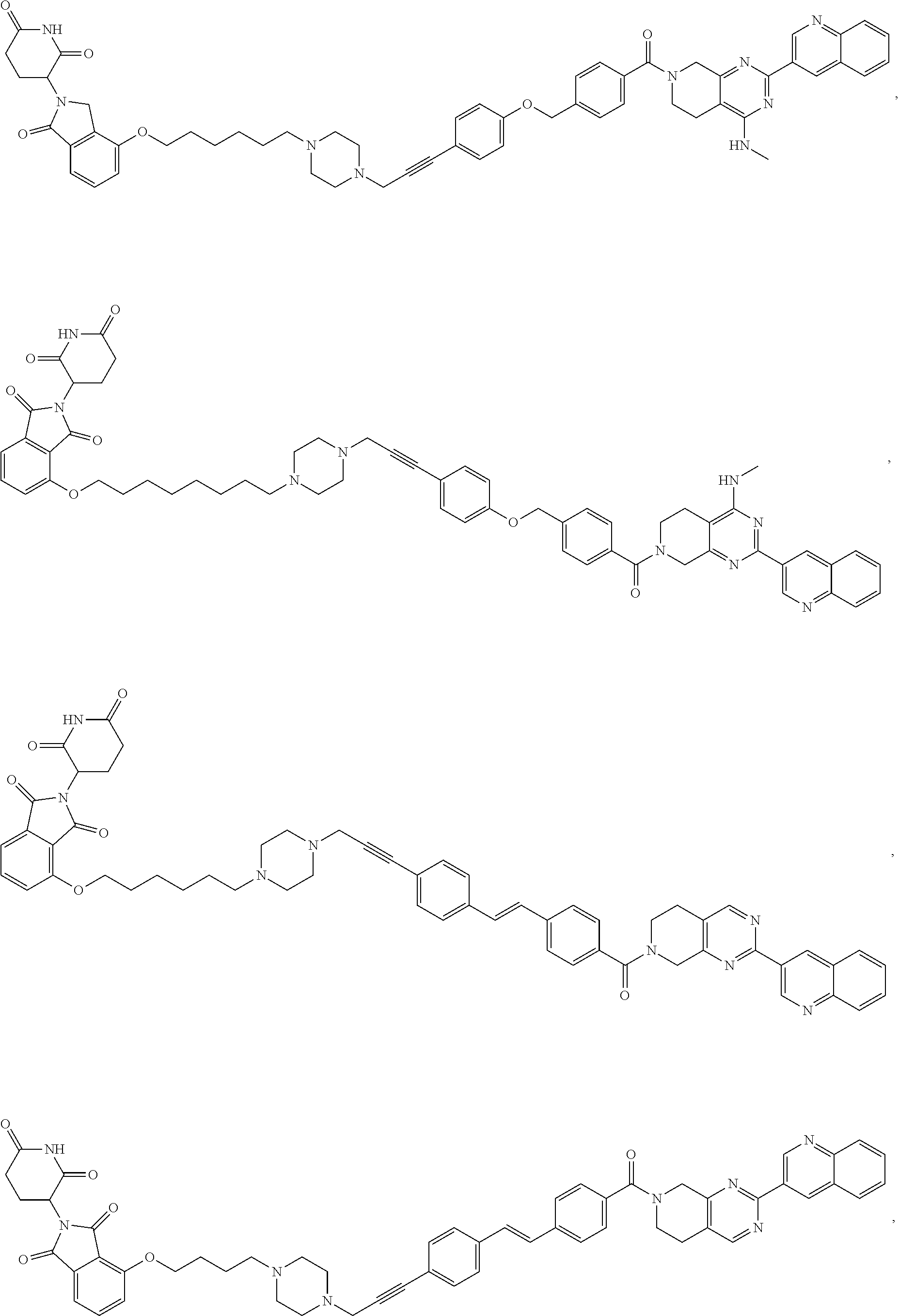 [SMILES: CNc1nc(-c2cnc3ccccc3c2)nc2c1CCN(C(=O)c1ccc(COc3ccc(C#CCN4CCN(CCCCCCOc5cccc6c5CN(C5CCC(=O)NC5=O)C6=O)CC4)cc3)cc1)C2 ; CNc1nc(-c2cnc3ccccc3c2)nc2c1CCN(C(=O)c1ccc(COc3ccc(C#CCN4CCN(CCCCCCCCOc5cccc6c5C(=O)N(C5CCC(=O)NC5=O)C6=O)CC4)cc3)cc1)C2 ; O=C1CCC(N2C(=O)c3cccc(OCCCCCCN4CCN(CC#Cc5ccc(/C=C/c6ccc(C(=O)N7CCc8cnc(-c9cnc%10ccccc%10c9)nc8C7)cc6)cc5)CC4)c3C2=O)C(=O)N1 ; O=C1CCC(N2C(=O)c3cccc(OCCCCN4CCN(CC#Cc5ccc(/C=C/c6ccc(C(=O)N7CCc8cnc(-c9cnc%10ccccc%10c9)nc8C7)cc6)cc5)CC4)c3C2=O)C(=O)N1]

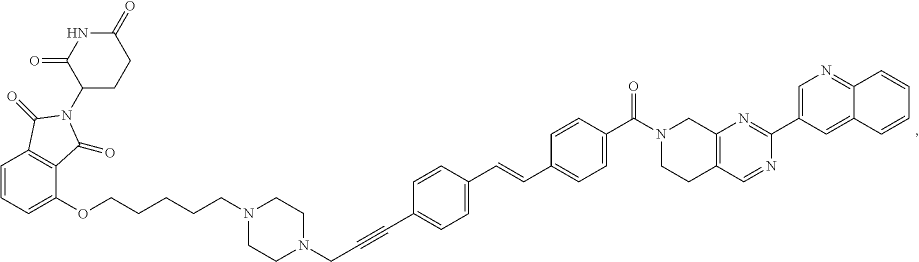 [SMILES: O=C1CCC(N2C(=O)c3cccc(OCCCCCN4CCN(CC#Cc5ccc(/C=C/c6ccc(C(=O)N7CCc8cnc(-c9cnc%10ccccc%10c9)nc8C7)cc6)cc5)CC4)c3C2=O)C(=O)N1]

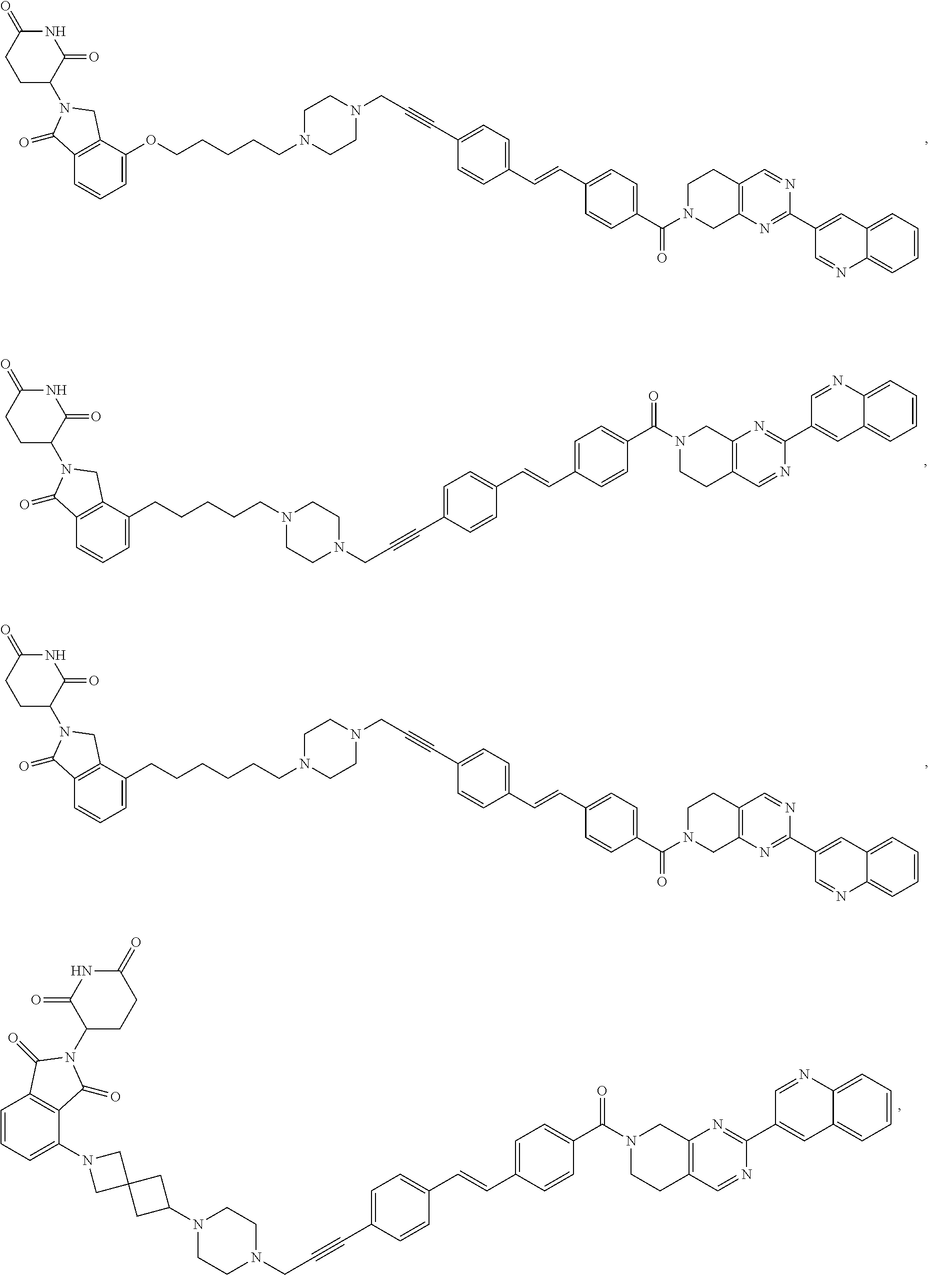

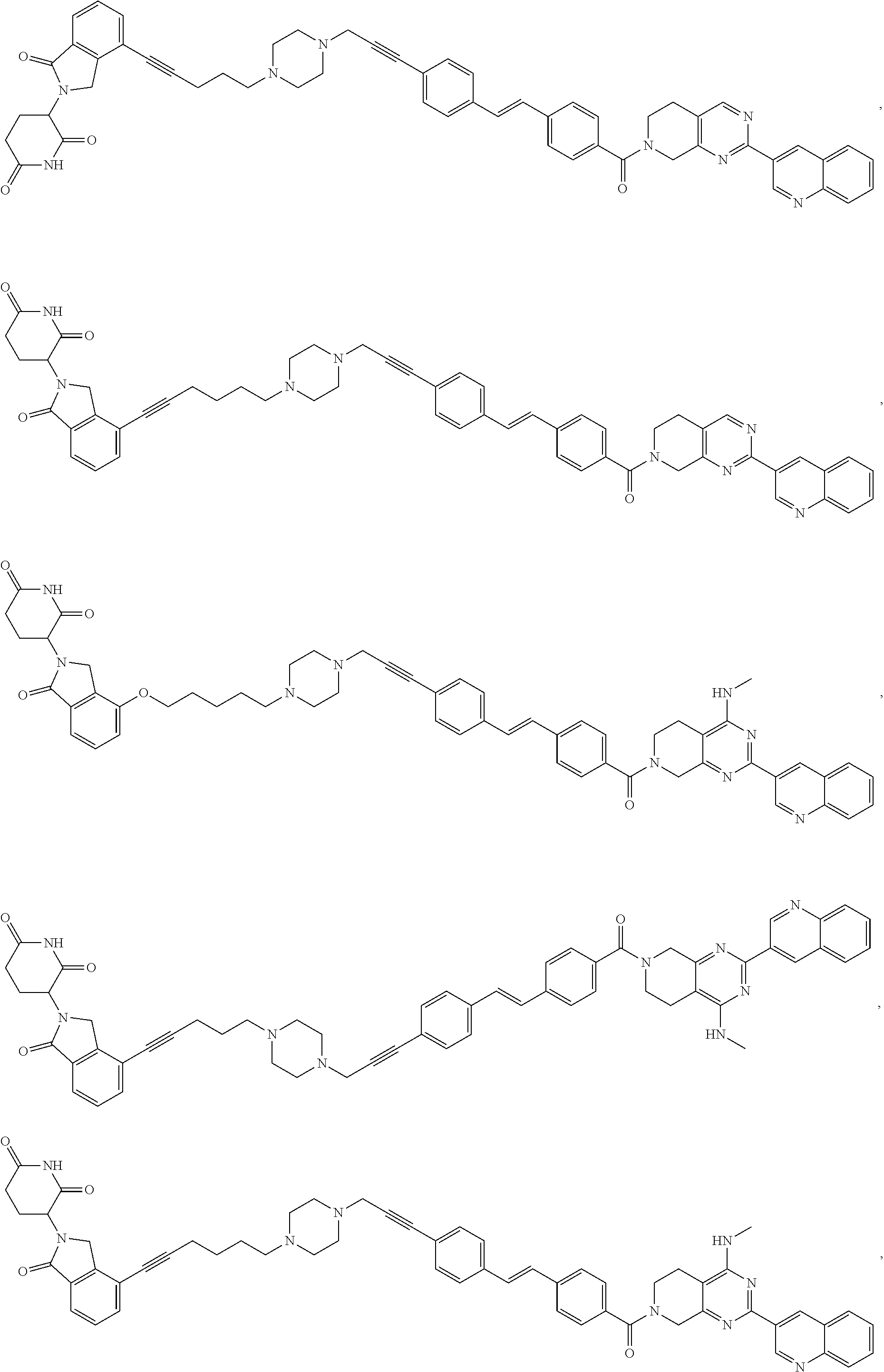

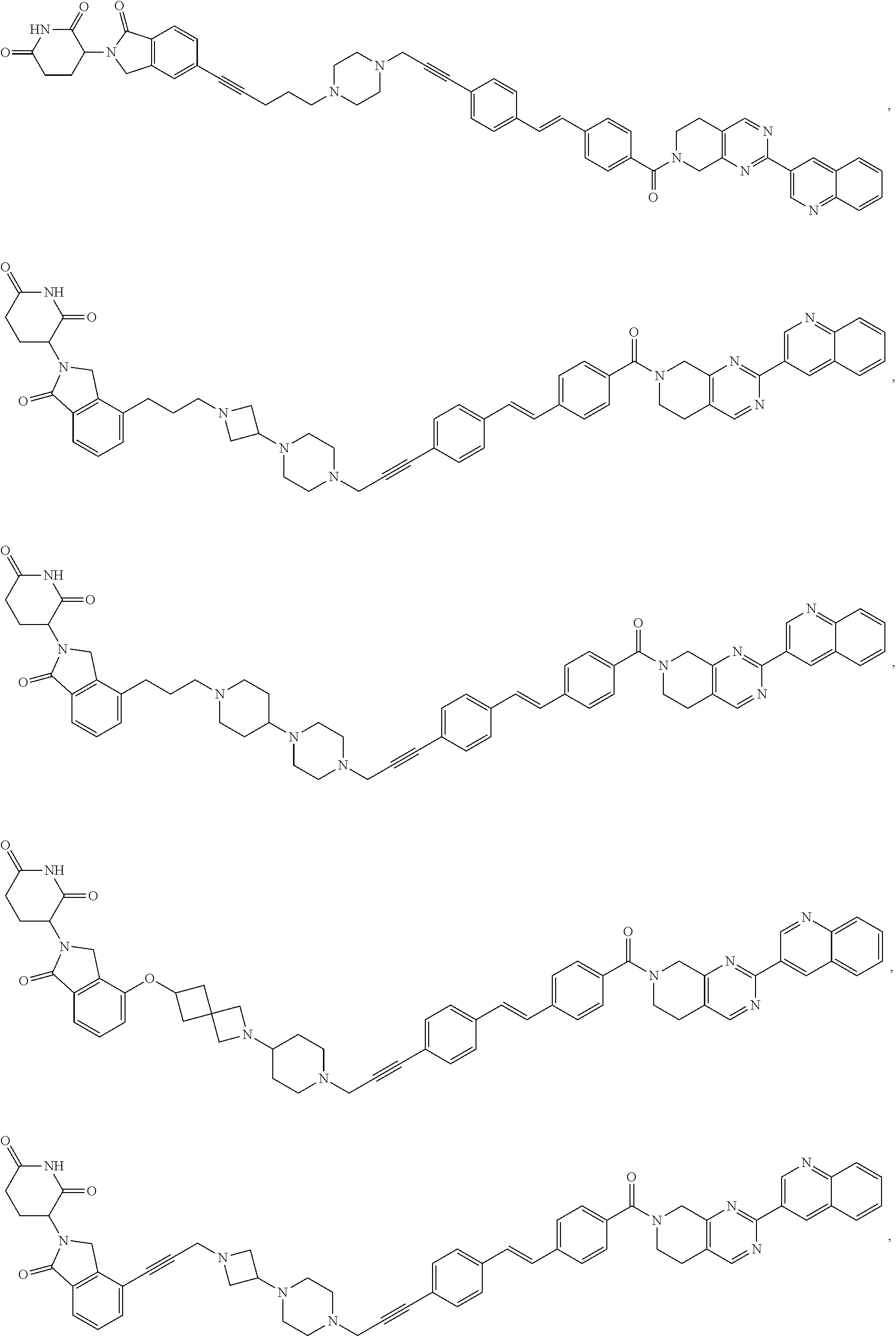

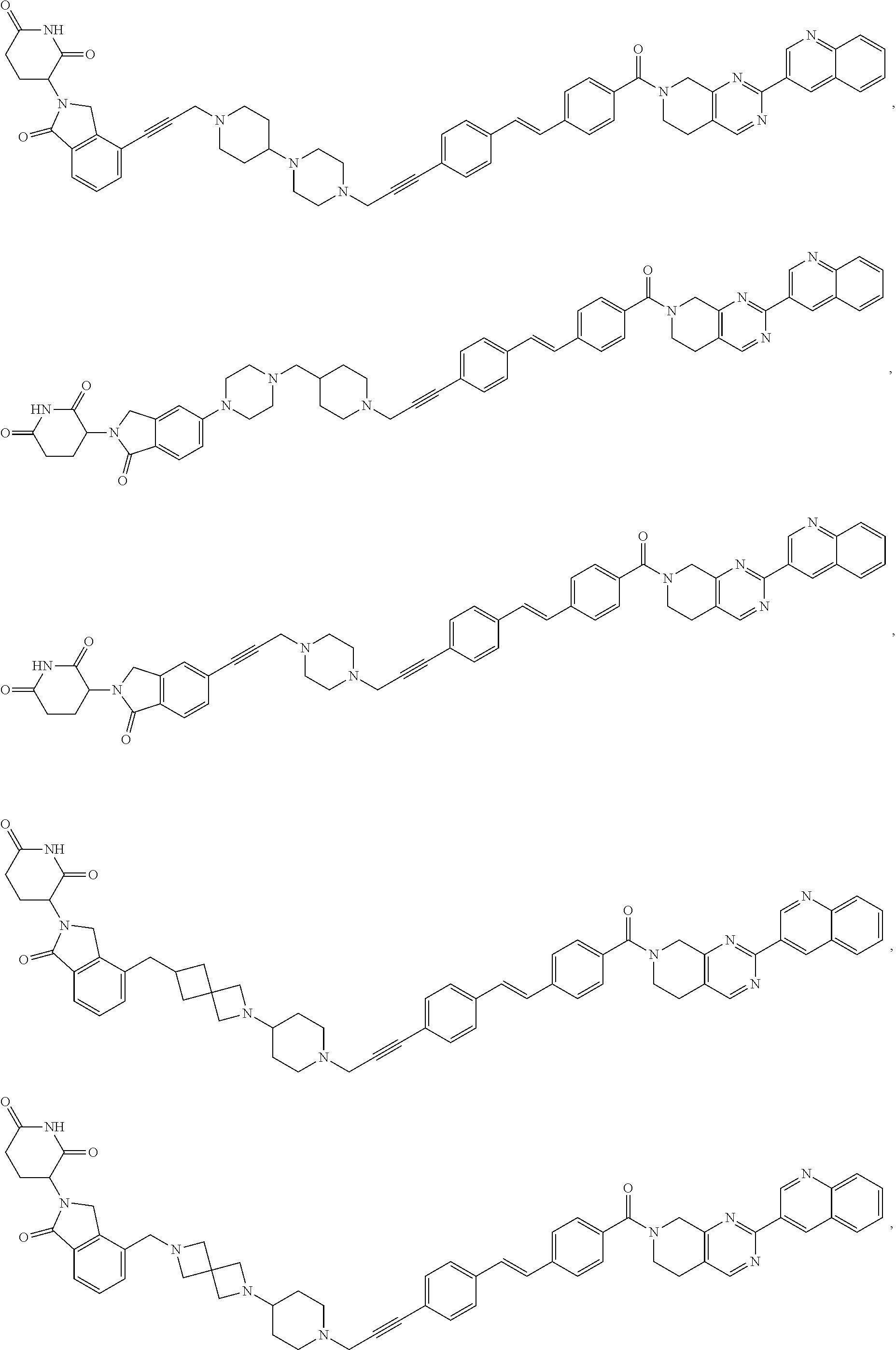

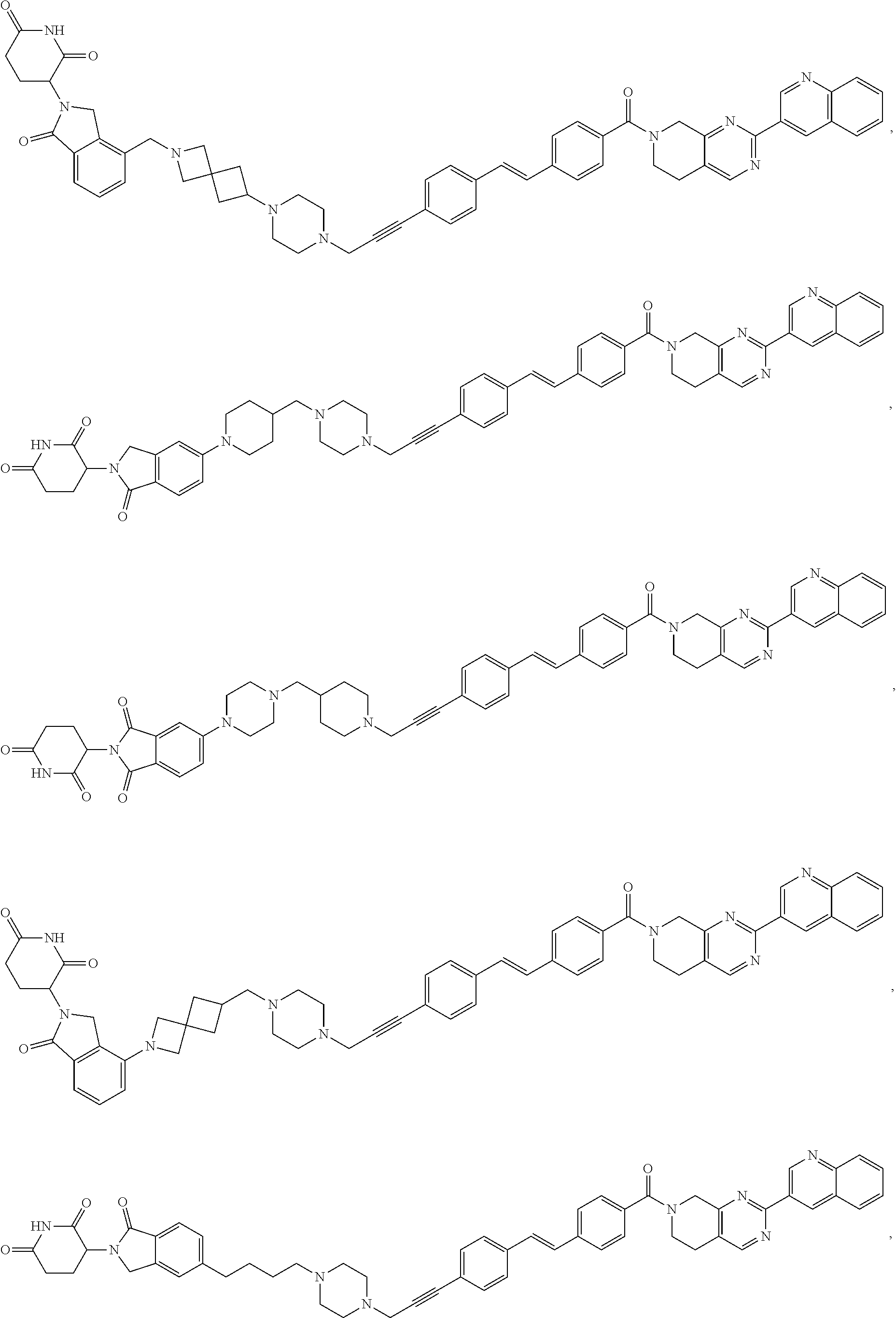

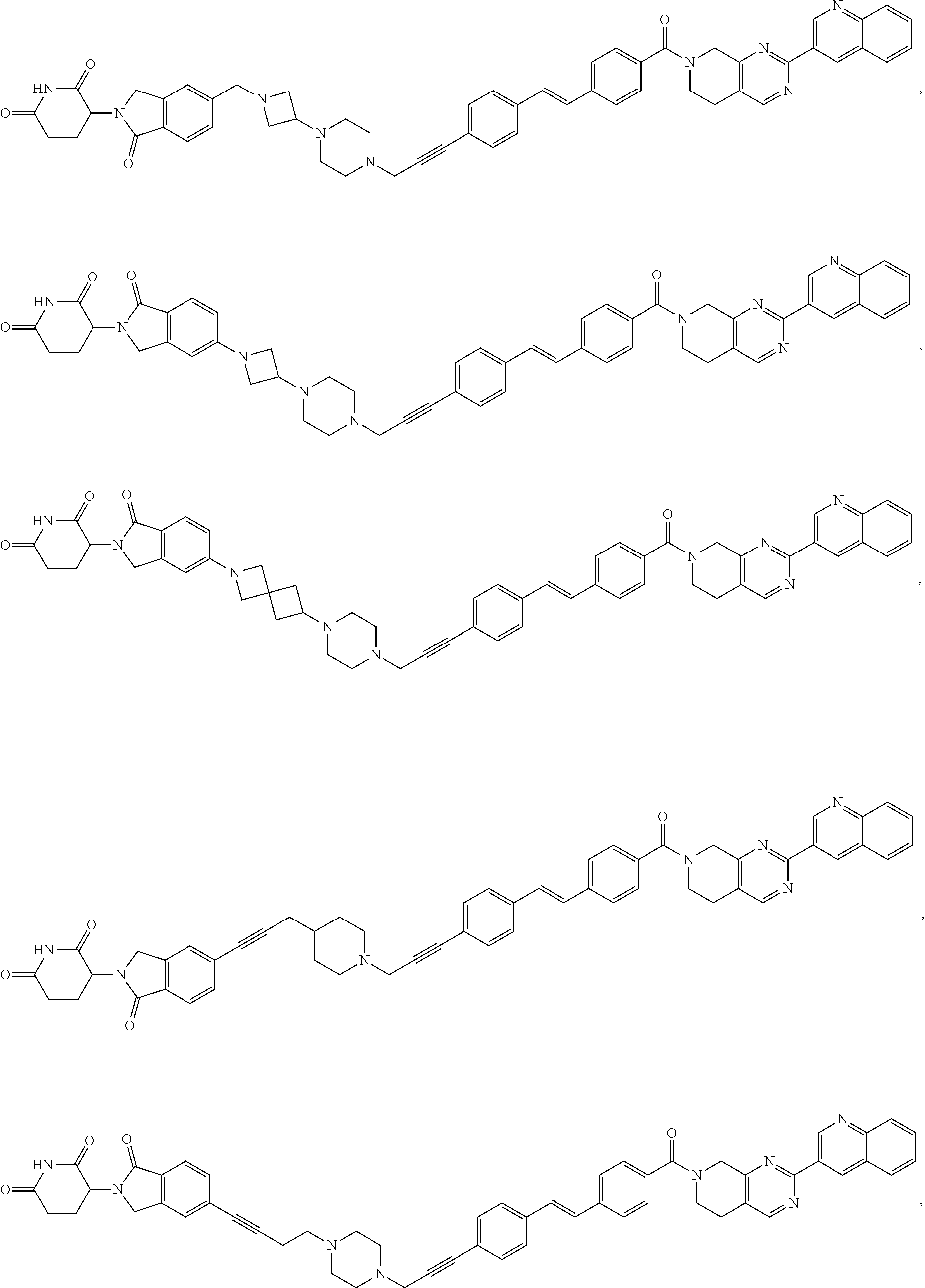

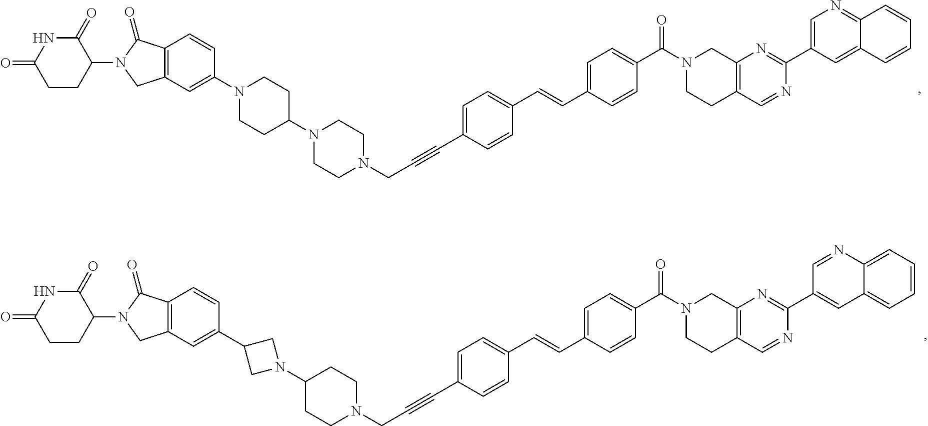 [SMILES: O=C1CCC(N2Cc3cc(N4CCC(N5CCN(CC#Cc6ccc(/C=C/c7ccc(C(=O)N8CCc9cnc(-c%10cnc%11ccccc%11c%10)nc9C8)cc7)cc6)CC5)CC4)ccc3C2=O)C(=O)N1 ; O=C1CCC(N2Cc3cc(C4CN(C5CCN(CC#Cc6ccc(/C=C/c7ccc(C(=O)N8CCc9cnc(-c%10cnc%11ccccc%11c%10)nc9C8)cc7)cc6)CC5)C4)ccc3C2=O)C(=O)N1]

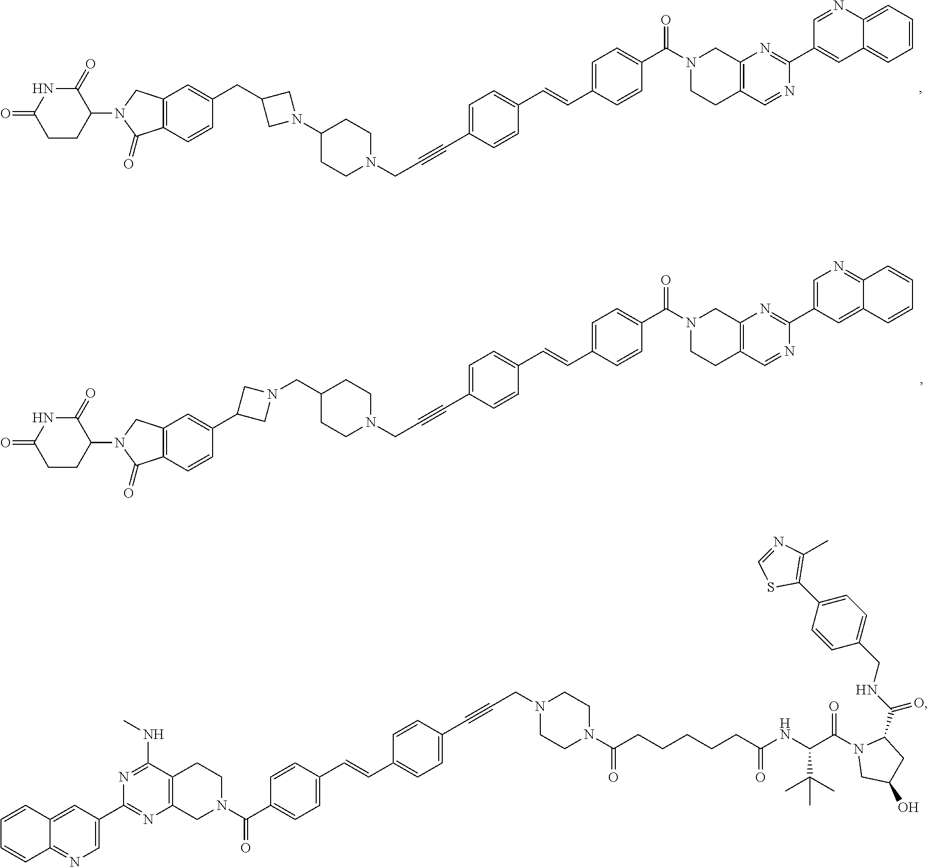 [SMILES: O=C1CCC(N2Cc3cc(CC4CN(C5CCN(CC#Cc6ccc(/C=C/c7ccc(C(=O)N8CCc9cnc(-c%10cnc%11ccccc%11c%10)nc9C8)cc7)cc6)CC5)C4)ccc3C2=O)C(=O)N1 ; O=C1CCC(N2Cc3cc(C4CN(CC5CCN(CC#Cc6ccc(/C=C/c7ccc(C(=O)N8CCc9cnc(-c%10cnc%11ccccc%11c%10)nc9C8)cc7)cc6)CC5)C4)ccc3C2=O)C(=O)N1 ; CNc1nc(-c2cnc3ccccc3c2)nc2c1CCN(C(=O)c1ccc(/C=C/c3ccc(C#CCN4CCN(C(=O)CCCCCC(=O)N[C@H](C(=O)N5C[C@H](O)C[C@H]5C(=O)NCc5ccc(-c6scnc6C)cc5)C(C)(C)C)CC4)cc3)cc1)C2]

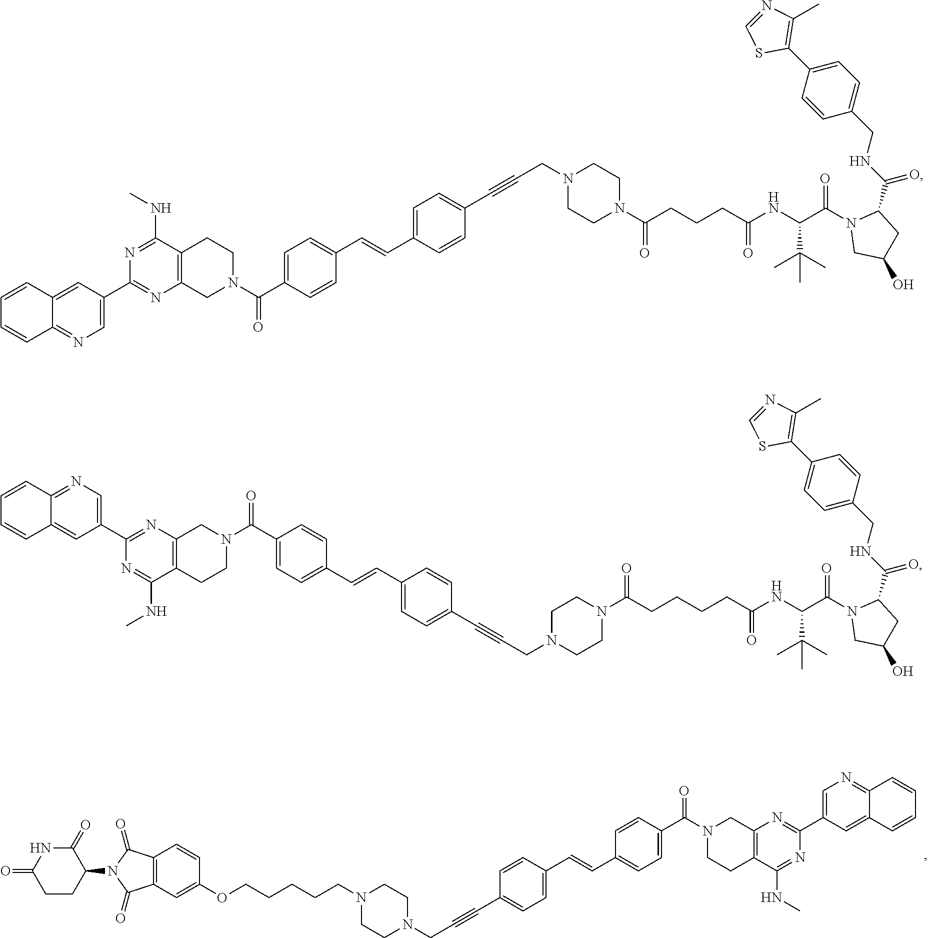 [SMILES: CNc1nc(-c2cnc3ccccc3c2)nc2c1CCN(C(=O)c1ccc(/C=C/c3ccc(C#CCN4CCN(C(=O)CCCC(=O)N[C@H](C(=O)N5C[C@H](O)C[C@H]5C(=O)NCc5ccc(-c6scnc6C)cc5)C(C)(C)C)CC4)cc3)cc1)C2 ; CNc1nc(-c2cnc3ccccc3c2)nc2c1CCN(C(=O)c1ccc(/C=C/c3ccc(C#CCN4CCN(C(=O)CCCCC(=O)N[C@H](C(=O)N5C[C@H](O)C[C@H]5C(=O)NCc5ccc(-c6scnc6C)cc5)C(C)(C)C)CC4)cc3)cc1)C2 ; CNc1nc(-c2cnc3ccccc3c2)nc2c1CCN(C(=O)c1ccc(/C=C/c3ccc(C#CCN4CCN(CCCCCOc5ccc6c(c5)C(=O)N([C@H]5CCC(=O)NC5=O)C6=O)CC4)cc3)cc1)C2]

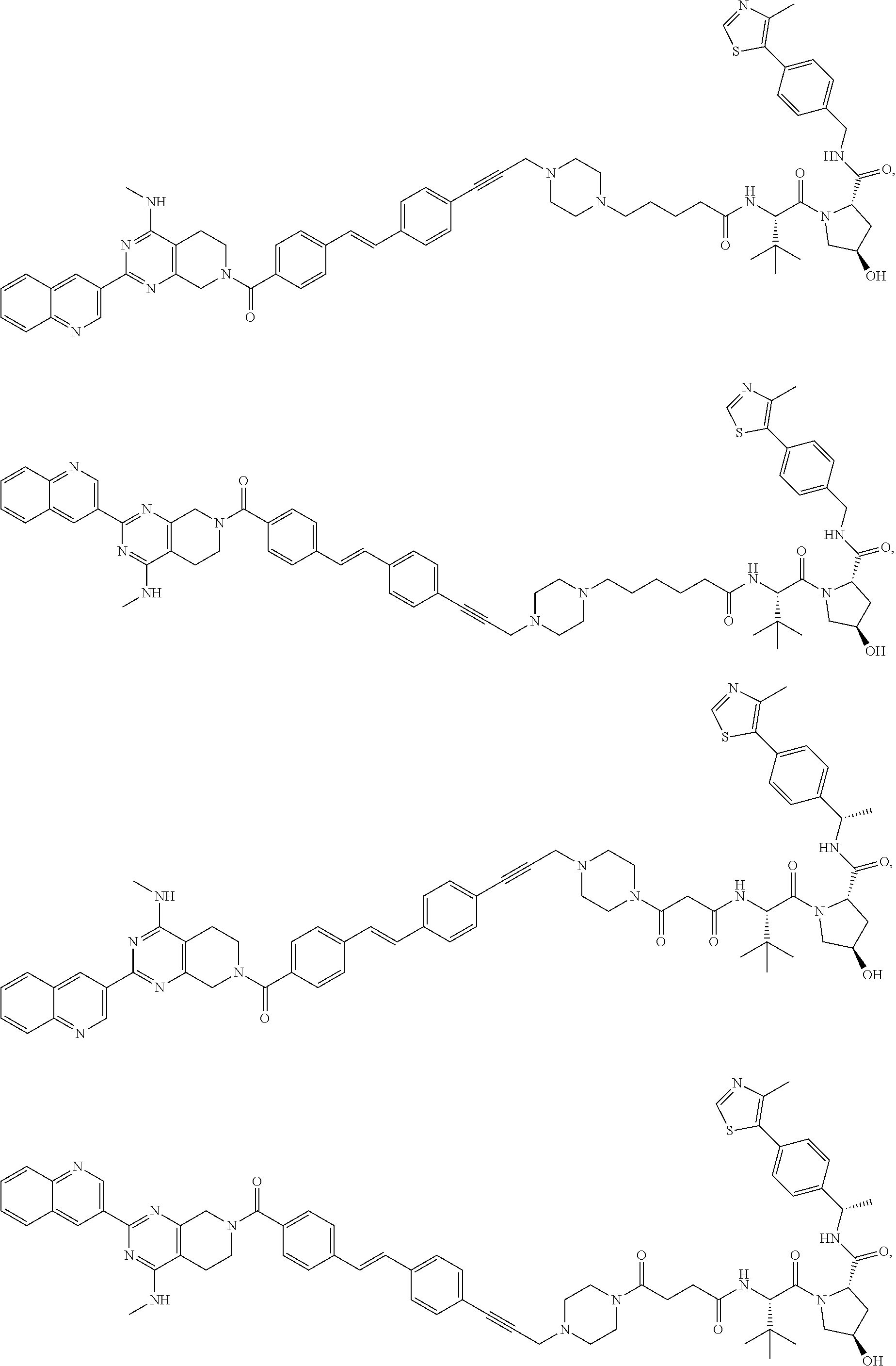

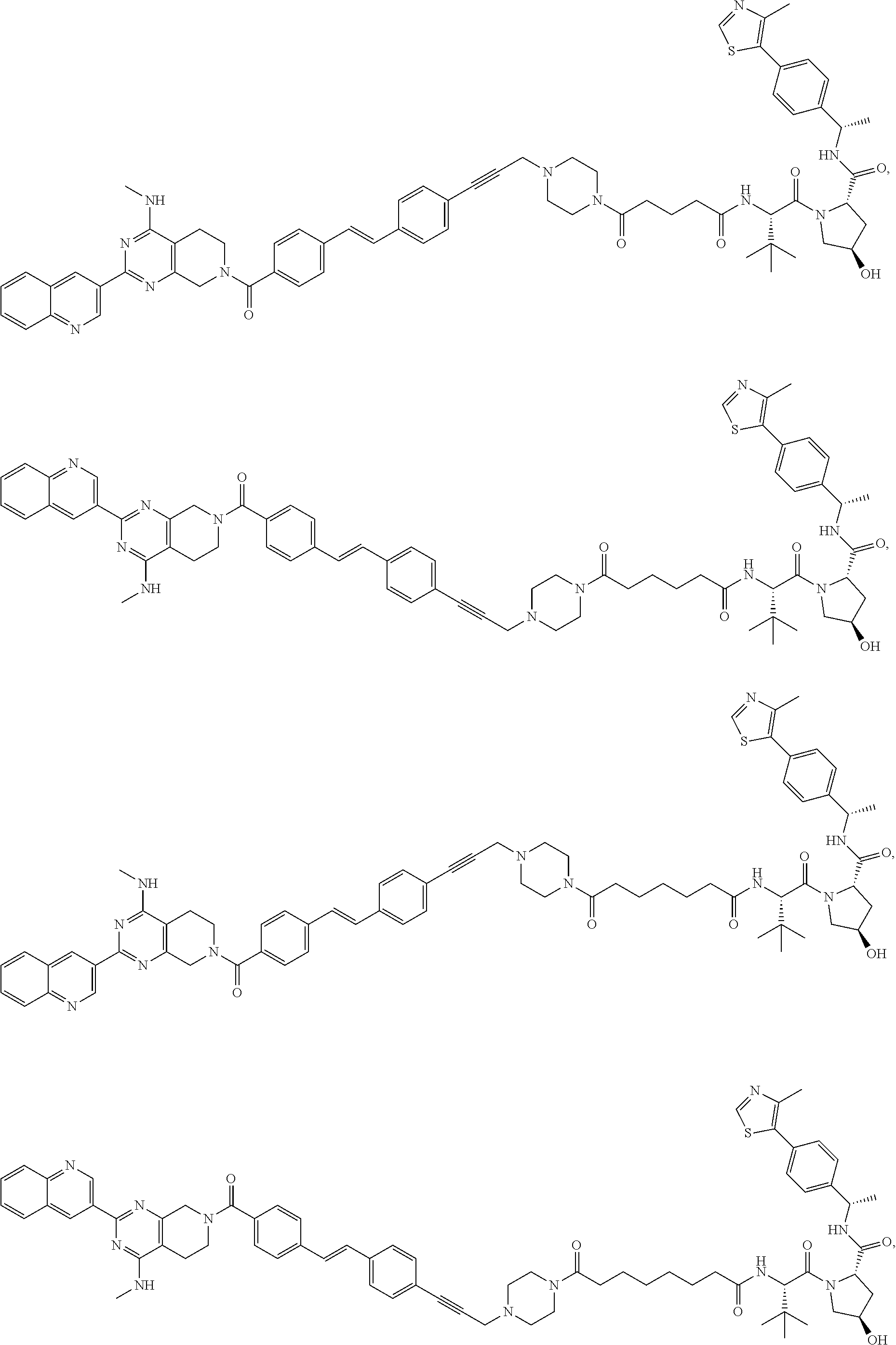

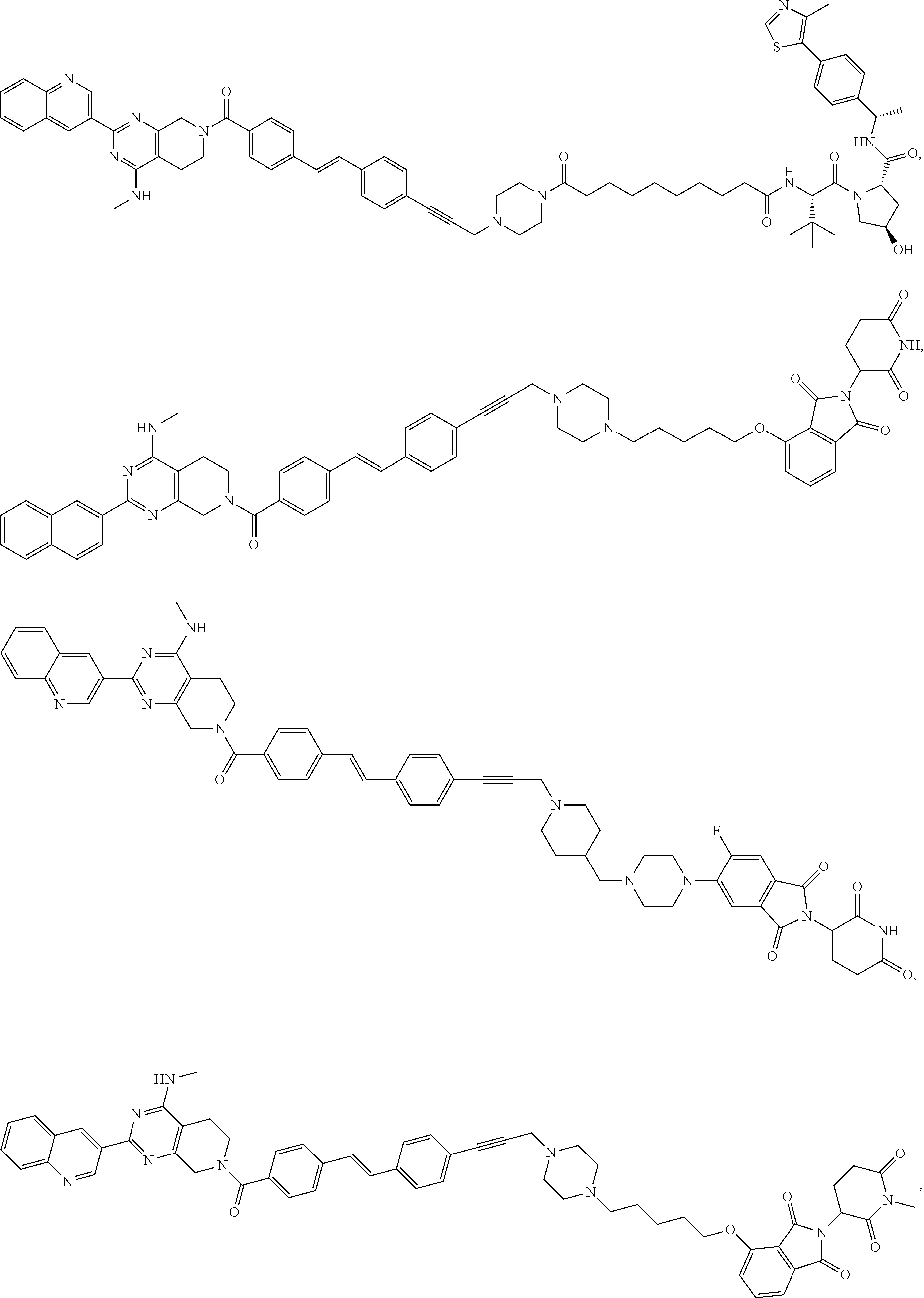

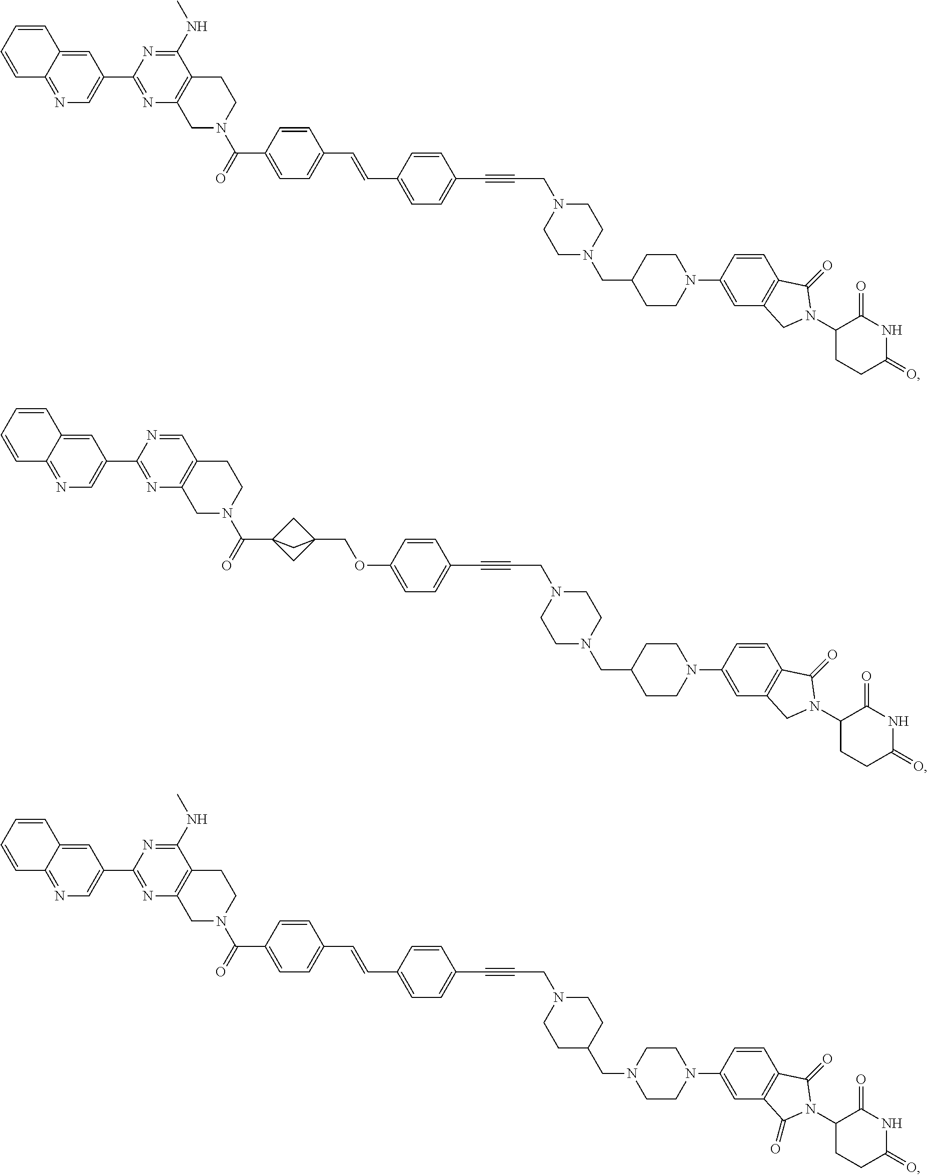 [SMILES: CNc1nc(-c2cnc3ccccc3c2)nc2c1CCN(C(=O)c1ccc(/C=C/c3ccc(C#CCN4CCN(CC5CCN(c6ccc7c(c6)CN(C6CCC(=O)NC6=O)C7=O)CC5)CC4)cc3)cc1)C2 ; O=C1CCC(N2Cc3cc(N4CCC(CN5CCN(CC#Cc6ccc(OCC78CC(C(=O)N9CCc%10cnc(-c%11cnc%12ccccc%12c%11)nc%10C9)(C7)C8)cc6)CC5)CC4)ccc3C2=O)C(=O)N1 ; CNc1nc(-c2cnc3ccccc3c2)nc2c1CCN(C(=O)c1ccc(/C=C/c3ccc(C#CCN4CCC(CN5CCN(c6ccc7c(c6)C(=O)N(C6CCC(=O)NC6=O)C7=O)CC5)CC4)cc3)cc1)C2]

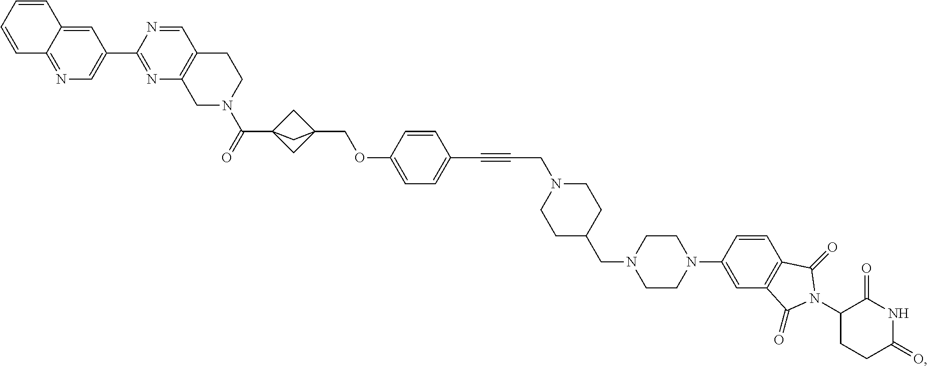 [SMILES: O=C1CCC(N2C(=O)c3ccc(N4CCN(CC5CCN(CC#Cc6ccc(OCC78CC(C(=O)N9CCc%10cnc(-c%11cnc%12ccccc%12c%11)nc%10C9)(C7)C8)cc6)CC5)CC4)cc3C2=O)C(=O)N1]

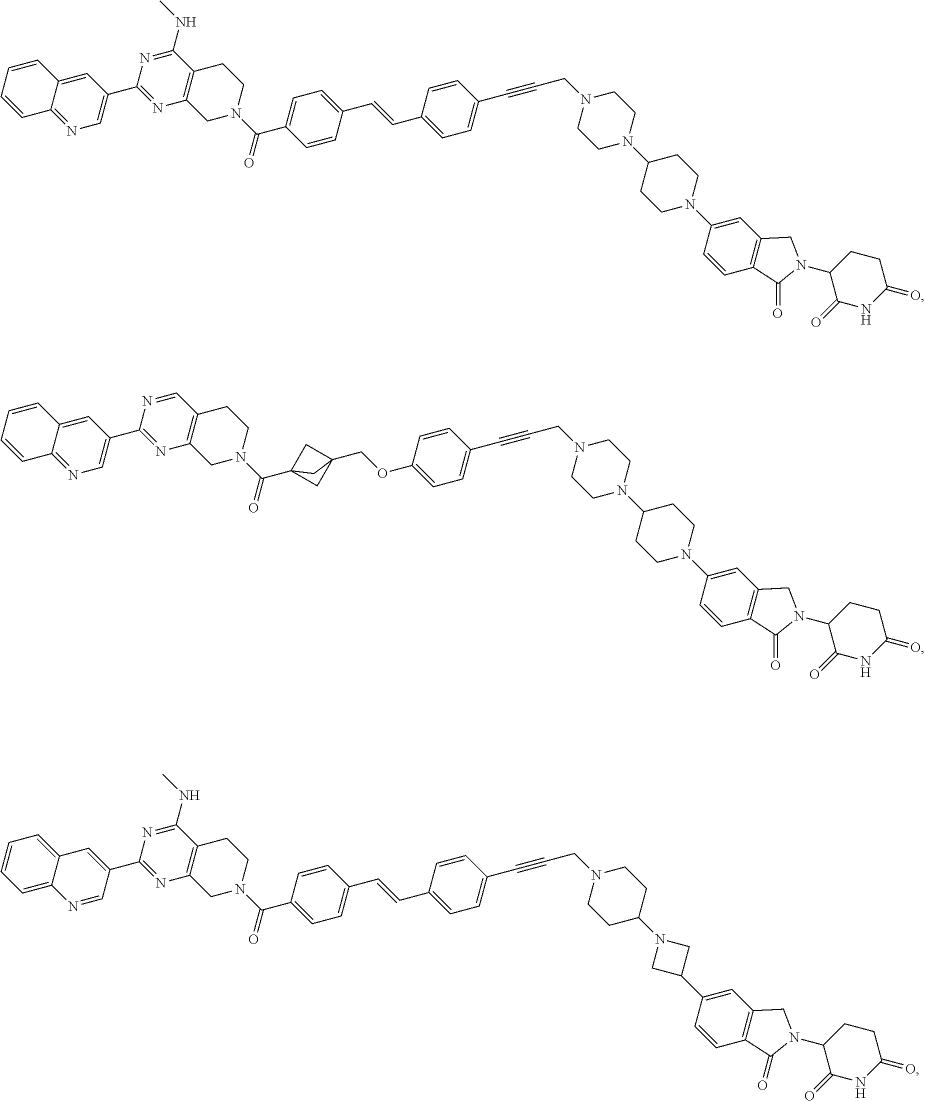 [SMILES: CNc1nc(-c2cnc3ccccc3c2)nc2c1CCN(C(=O)c1ccc(/C=C/c3ccc(C#CCN4CCN(C5CCN(c6ccc7c(c6)CN(C6CCC(=O)NC6=O)C7=O)CC5)CC4)cc3)cc1)C2 ; O=C1CCC(N2Cc3cc(N4CCC(N5CCN(CC#Cc6ccc(OCC78CC(C(=O)N9CCc%10cnc(-c%11cnc%12ccccc%12c%11)nc%10C9)(C7)C8)cc6)CC5)CC4)ccc3C2=O)C(=O)N1 ; CNc1nc(-c2cnc3ccccc3c2)nc2c1CCN(C(=O)c1ccc(/C=C/c3ccc(C#CCN4CCC(N5CC(c6ccc7c(c6)CN(C6CCC(=O)NC6=O)C7=O)C5)CC4)cc3)cc1)C2]

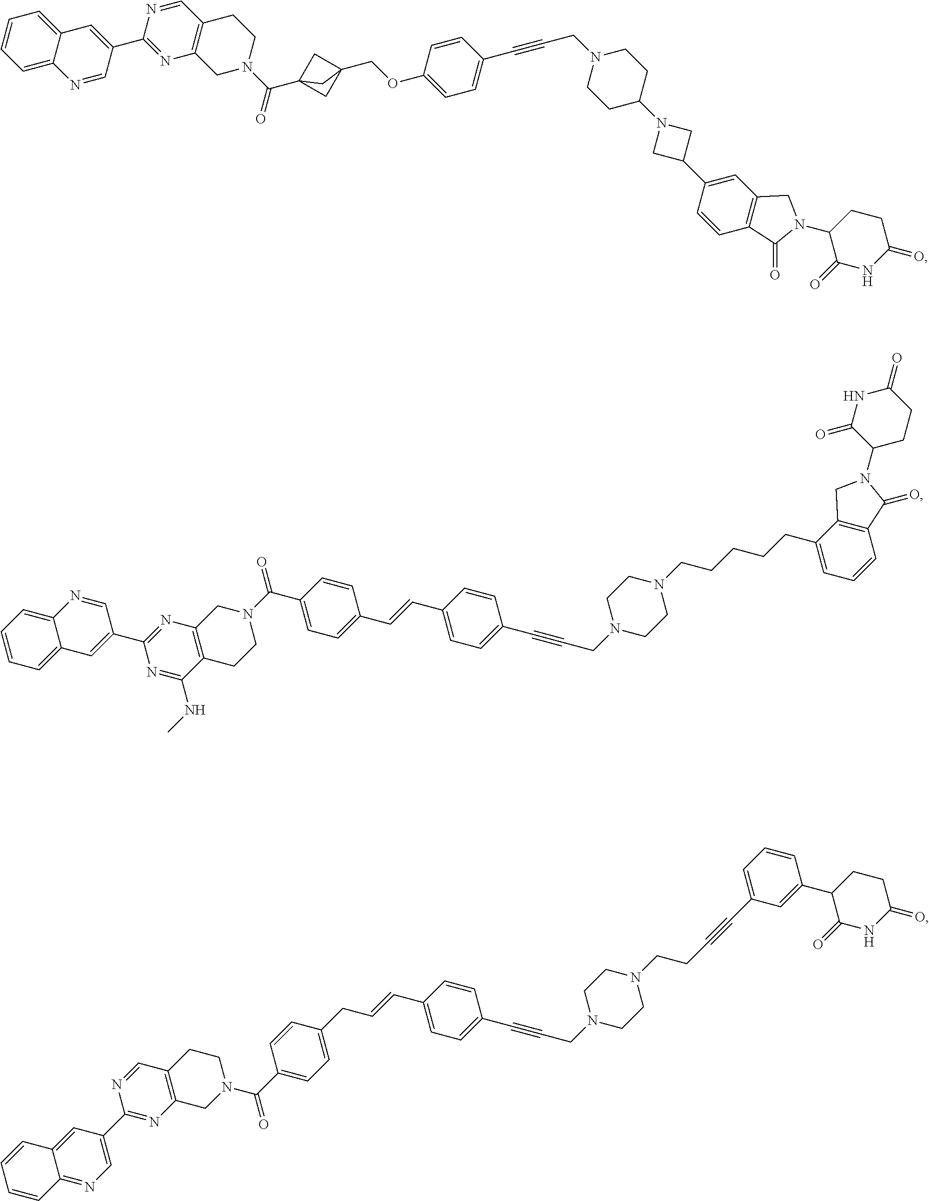 [SMILES: O=C1CCC(N2Cc3cc(C4CN(C5CCN(CC#Cc6ccc(OCC78CC(C(=O)N9CCc%10cnc(-c%11cnc%12ccccc%12c%11)nc%10C9)(C7)C8)cc6)CC5)C4)ccc3C2=O)C(=O)N1 ; CNc1nc(-c2cnc3ccccc3c2)nc2c1CCN(C(=O)c1ccc(/C=C/c3ccc(C#CCN4CCN(CCCCCc5cccc6c5CN(C5CCC(=O)NC5=O)C6=O)CC4)cc3)cc1)C2 ; O=C1CCC(c2cccc(C#CCCN3CCN(CC#Cc4ccc(/C=C/Cc5ccc(C(=O)N6CCc7cnc(-c8cnc9ccccc9c8)nc7C6)cc5)cc4)CC3)c2)C(=O)N1]

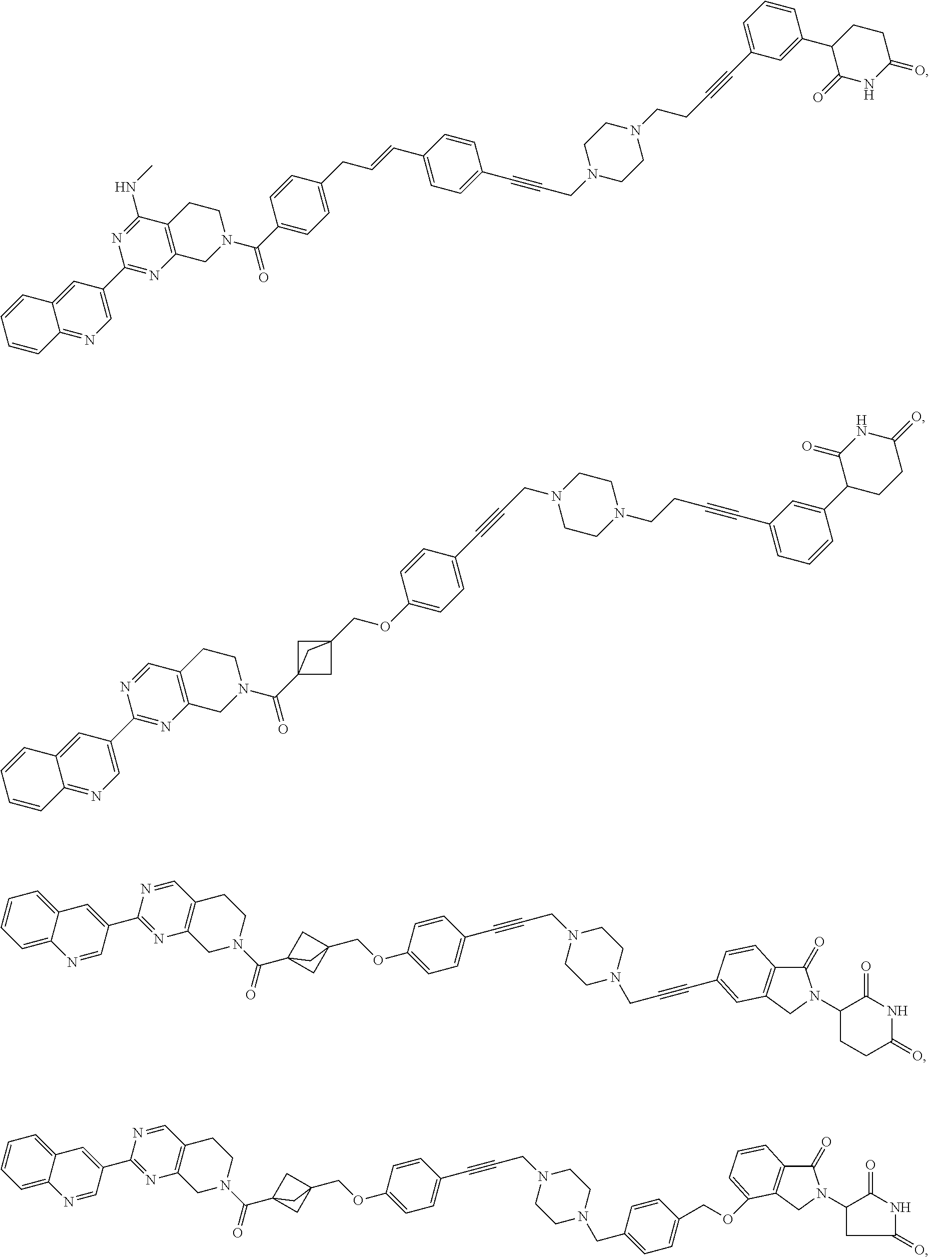 [SMILES: CNc1nc(-c2cnc3ccccc3c2)nc2c1CCN(C(=O)c1ccc(C/C=C/c3ccc(C#CCN4CCN(CCC#Cc5cccc(C6CCC(=O)NC6=O)c5)CC4)cc3)cc1)C2 ; O=C1CCC(c2cccc(C#CCCN3CCN(CC#Cc4ccc(OCC56CC(C(=O)N7CCc8cnc(-c9cnc%10ccccc%10c9)nc8C7)(C5)C6)cc4)CC3)c2)C(=O)N1 ; O=C1CCC(N2Cc3cc(C#CCN4CCN(CC#Cc5ccc(OCC67CC(C(=O)N8CCc9cnc(-c%10cnc%11ccccc%11c%10)nc9C8)(C6)C7)cc5)CC4)ccc3C2=O)C(=O)N1 ; O=C1CC(N2Cc3c(OCc4ccc(CN5CCN(CC#Cc6ccc(OCC78CC(C(=O)N9CCc%10cnc(-c%11cnc%12ccccc%12c%11)nc%10C9)(C7)C8)cc6)CC5)cc4)cccc3C2=O)C(=O)N1]

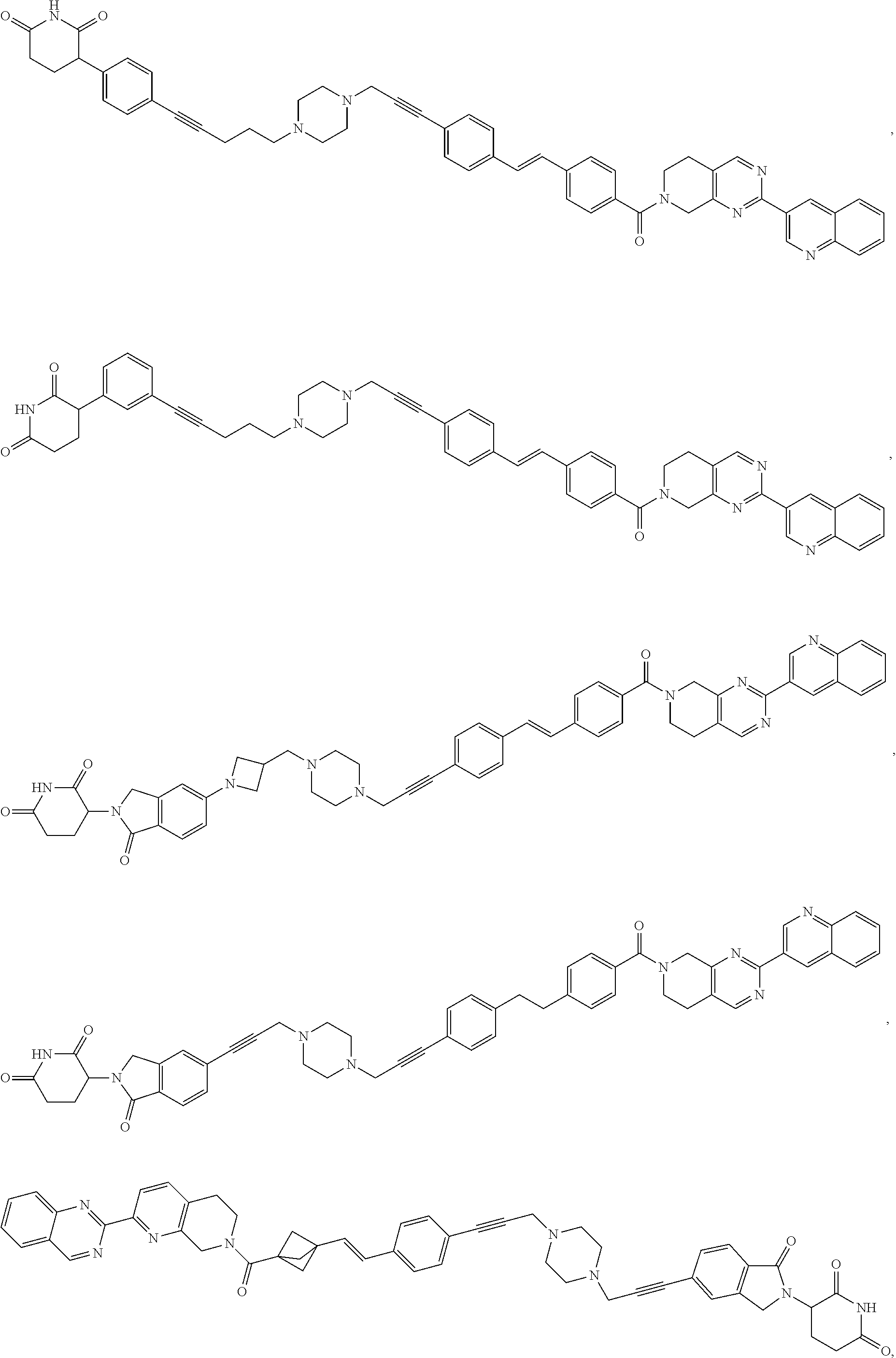

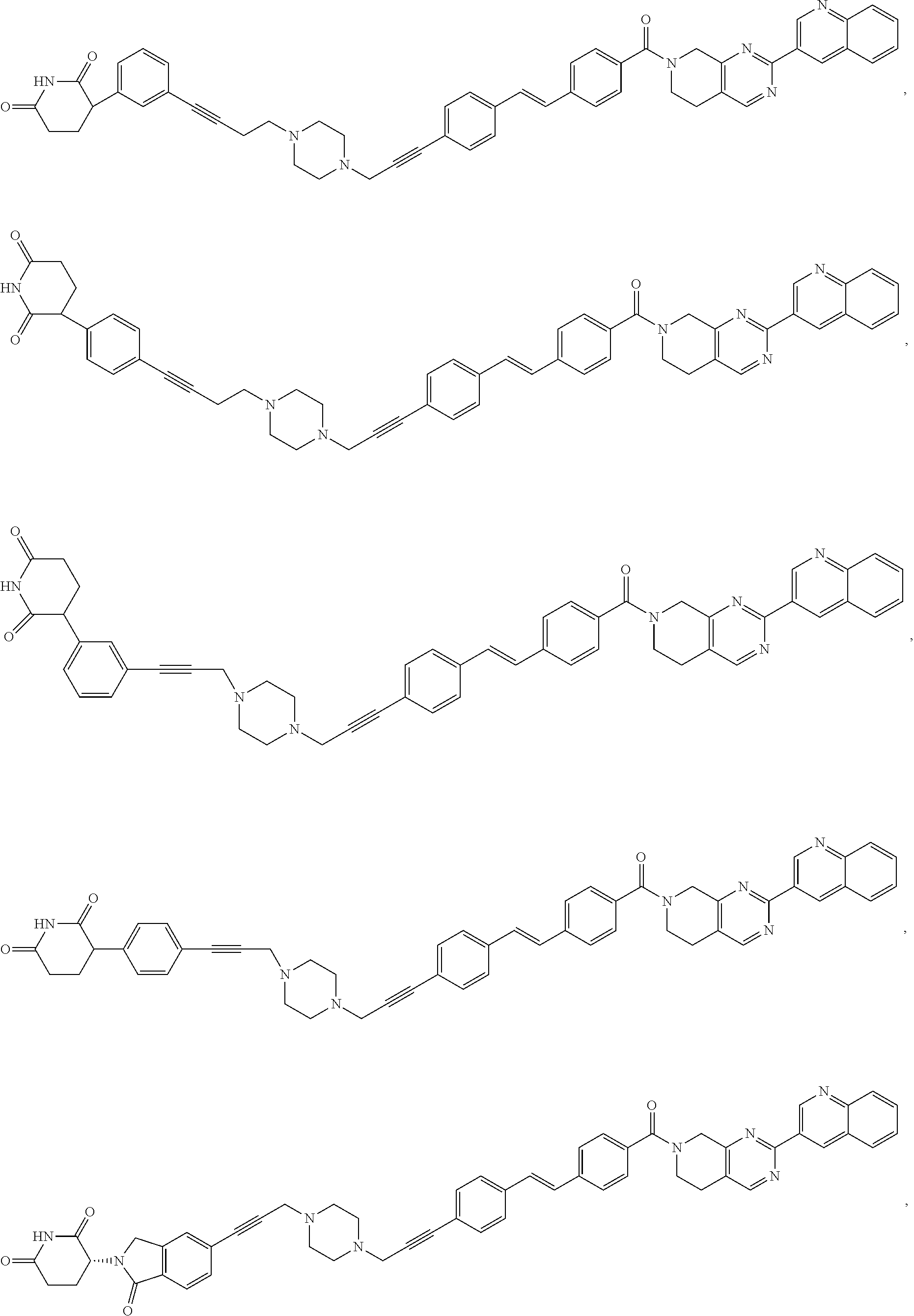

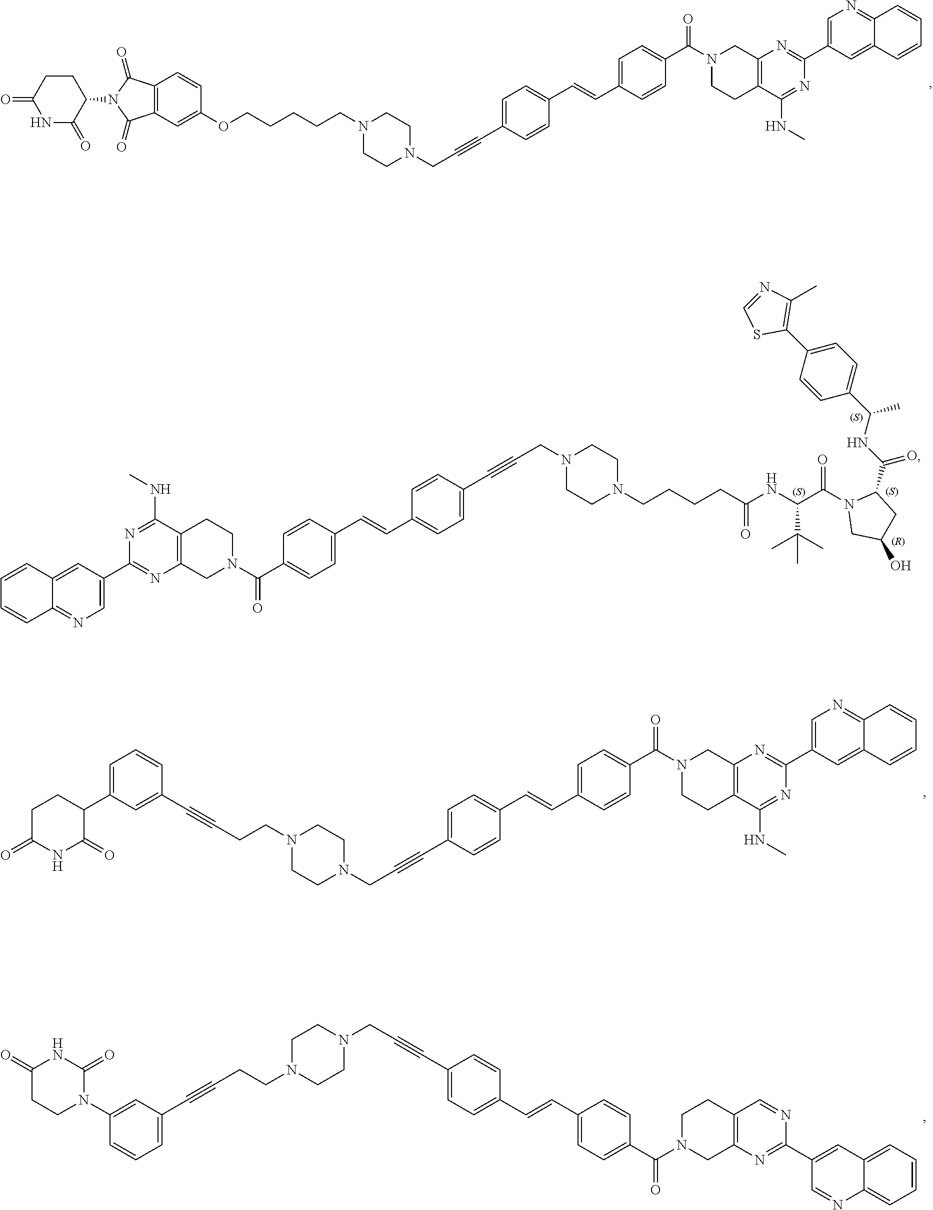

21. The compound according to claim 1, wherein the compound is optionally and independently substituted by one or more (for example, 2, 3, 4, 5, 6, 7, 8, 9 or 10) R^{YL′};
preferably, R^{YL′} is independently selected from the group consisting of halogen, cyano, nitro, C_{1˜6} alkyl, halogen-substituted C_{1˜6} alkyl, —OR′, and —N(R′)₂; each R′ is independently selected from the group consisting of hydrogen, halogen, —C_{1˜6} alkyl, halogen-substituted —C_{1˜6} alkyl, —C_{0˜2} alkylene-(C_{3˜10} carbocyclic group), and —C_{0˜2} alkylene-(3- to 10-membered heterocycloalkyl);
preferably, R^{YL′} is independently selected from the group consisting of halogen, cyano, nitro, C_{1˜6} alkyl, halogen-substituted C_{1˜6} alkyl, —OR′, and —N(R′)₂; each R′ is independently selected from the group consisting of hydrogen, halogen, —C_{1˜6} alkyl, halogen-substituted —C_{1˜6} alkyl, —C_{0˜2} alkylene-(C_{3˜8} carbocyclic group), and —C_{0˜2} alkylene-(3- to 8-membered heterocycloalkyl);
preferably, R^{YL′} is independently selected from the group consisting of halogen, cyano, nitro, C_{1˜3} alkyl, halogen-substituted C_{1˜3} alkyl, —OR′ and —N(R′)₂; each R′ is independently selected from the group consisting of hydrogen, halogen, —C_{1˜3} alkyl, halogen-substituted —C_{1˜3} alkyl, —C_{0˜2} alkylene-(C_{3˜6} carbocyclic group) and —C_{0˜2} alkylene-(3- to 6-membered heterocycloalkyl).

22. A pharmaceutical composition, comprising a preparation made from the compound, or deuterated compound thereof, or stereoisomer thereof, or tautomer thereof, or polymorph thereof, or solvate thereof, or N-oxide thereof, or isotopically labeled compound thereof, or metabolite thereof, or prodrug thereof, or pharmaceutically acceptable salt thereof according to claim 1.

23. The pharmaceutical composition according to claim 22, further comprising one or more of a pharmaceutically acceptable carrier, excipient, and vehicle.

24. A method for preventing and/or treating a disease associated with BCL-XL activity comprising administering an effective amount of the compound, or deuterated compound thereof, or stereoisomer thereof, or tautomer thereof, or polymorph thereof, or solvate thereof, or N-oxide thereof, or isotopically labeled compound thereof, or metabolite thereof, or prodrug thereof, or pharmaceutically acceptable salt thereof according to claim 1 to a subject in need thereof.

25. The method according to claim 24, wherein the disease associated with BCL-XL activity is selected from the group consisting of autoimmune disease, bladder cancer, brain cancer, breast cancer, bone marrow cancer, cervical cancer, colorectal cancer, esophageal cancer, hepatocellular carcinoma, follicular lymphoma, lymphoid malignancy of T-cell or B-cell origin, melanoma, myeloma, oral cancer, ovarian cancer, non-small cell lung cancer, prostate cancer, leukemia, small cell lung cancer, or spleen cancer; the leukemia is preferably chronic lymphocytic leukemia, lymphoblastic leukemia or granulocytic leukemia.

26. A method for preventing and/or treating a cancer, comprising administering an effective amount of the compound, or deuterated compound thereof, or stereoisomer thereof, or tautomer thereof, or polymorph thereof, or solvate thereof, or N-oxide thereof, or isotopically labeled compound thereof, or metabolite thereof, or prodrug thereof, or pharmaceutically acceptable salt thereof according to claim 1 to a subject in need thereof.

27. (canceled)

28. (canceled)

29. A method for preventing and/or treating a disease associated with BCL-XL activity comprising administering an effective amount of the pharmaceutical composition according to claim 22 to a subject in need thereof.
